(19) United States
(12) Patent Application Publication
(10) Pub. No.: US 2024/0229063 A1
(43) Pub. Date: Jul. 11, 2024

(54) TRANSGENIC CORN EVENT ZM_CSM63715 AND METHODS FOR DETECTION AND USES THEREOF

(71) Applicant: Monsanto Technology LLC, St. Louis, MO (US)

(72) Inventors: Matthew Bauer, Manchester, MO (US); Sarah Brown, Weldon Spring, MO (US); Robert Thomas Gaeta, Eureka, MO (US); Michael Edward Goley, St. Charles, MO (US); Shirley Guo, Chesterfield, MO (US); Erin Lyn Hall, Collinsville, IL (US); Andrei Kouranov, Chesterfield, MO (US); Clayton Tyler Larue, Chesterfield, MO (US); Mingsheng Peng, Wildwood, MO (US); Linda Rymarquis, High Ridge, MO (US); Oscar Cornelius Sparks, JR., Florissant, MO (US); Xudong Ye, Chesterfield, MO (US)

(21) Appl. No.: 18/541,484

(22) Filed: Dec. 15, 2023

Related U.S. Application Data
(60) Provisional application No. 63/476,272, filed on Dec. 20, 2022.

(51) Int. Cl. C12N 15/82; C12N 9/22; C12N 15/11; C12Q 1/6827; C12Q 1/686; C12Q 1/6895; G01N 33/569; G01N 33/573

(52) U.S. Cl. CPC C12N 15/8274; C12N 9/22; C12N 15/111; C12N 15/8213; C12Q 1/6827; C12Q 1/686; C12Q 1/6895; G01N 33/56961; G01N 33/573; C12N 2310/20; C12Q 2600/13; G01N 2333/90206

(57) ABSTRACT

A transgenic corn event, Zm_CSM63715, is provided. Transgenic plant cells, plant parts, plants, seeds, progeny plants, and agricultural and commodity products containing event Zm_CSM63715 are also provided. Recombinant DNA molecules unique to the event Zm_CSM63715, and methods of using and detecting Zm_CSM63715 are also provided. Corn plants containing the event Zm_CSM63715 exhibit tolerance to PPO inhibitors.

### CROSS-REFERENCE TO RELATED APPLICATIONS

This application claims the priority of U.S. Provisional Appl. Ser. No. 63/476,272, filed Dec. 20, 2022, the entire disclosure of which is incorporated herein by reference.

### INCORPORATION OF SEQUENCE LISTING

The sequence listing contained in the file named “MONS555US_ST26.xml”, which is 269 kilobytes (measured in MS-Windows) and was created on Oct. 19, 2023, is filed herewith by electronic submission, and is incorporated herein by reference in its entirety.

### FIELD OF THE INVENTION

The present disclosure relates generally to the fields of agriculture, plant biotechnology and molecular biology. More specifically, the disclosure relates to compositions and methods for providing herbicide tolerance in transgenic corn plants. More specifically, recombinant DNA molecules of corn event Zm_CSM63715 are provided. Also provided are transgenic corn plants, plant parts, seeds, cells, and agricultural products comprising the corn event Zm_CSM63715, as well as methods of producing and using transgenic corn plants, plant parts, seeds, cells, and agricultural products comprising corn event Zm_CSM63715, methods of detecting corn event Zm_CSM63715, and methods of controlling weeds. Transgenic corn plants, plant parts, seeds and cells comprising corn event Zm_CSM63715 exhibit tolerance to a variety of PPO herbicides.

### BACKGROUND OF THE INVENTION

Increasing sustainable crop production is crucial to meet the need for food for the growing global population, feed for increased demand on animal-based diets in developing nations, and expanded use of crop products to produce biofuel, fiber, and other agricultural product-based commodities, while using limited natural resources. In agricultural systems, the effective management of weedy species in agricultural fields is essential for maintaining favorable crop growing conditions and yield. Weeds compete with crops for space, nutrients, water, and light and can contaminate harvests, and present one of the major challenges to sustainable crop production. Averaged across seven years from 2007 to 2013, weed interference in corn in the United States and Canada caused an average of 50% yield loss, which equates to a loss of 148 tons of corn valued at over $26.7 billion annually (Soltani et al., 2016). Moreover, yield losses due to weeds are influenced by weather. Under drought conditions, numerous weed species exhibit increased competitiveness with corn (Patterson, 1995; Steckel & Sprague, 2004). Selective herbicides had significantly contributed to weed management before the deployment of herbicide tolerant crops.

Application of herbicides provides an important tool to reduce weed pressure, improve productivity and increase security for global crop production.

Corn (Zea mays) is an important crop in many areas of the world. The introduction of genetically modified crops containing herbicide tolerance traits has successfully provided additional tools available to farmers to better control weeds. Transgenic herbicide tolerance enables the use of an herbicide in a crop growing environment without crop injury or with minimal crop injury (e.g., less than about 10% injury). Transgenic corn traits have been used to impart tolerance to glyphosate, glufosinate, and 2,4-D and are used broadly in commercial corn production for weed management. However, weeds have evolved resistance to herbicides and weed resistance continues to present a challenge in corn production today. Therefore, there is a need for additional herbicide tolerance trait options to manage weeds effectively and to sustain crop productivity. One of the solutions is to employ herbicide(s) with new or different mode(s) of action, and/or employ multiple herbicides with different modes of action.

Protoporphyrinogen IX oxidase (PPO) catalyzes the oxidation of protoporphyrinogen IX to produce protoporphyrin IX. This enzymatic step is conserved across prokaryotes and eukaryotes for the synthesis of tetrapyrroles such as heme. In plants, the production of chlorophyll is dependent on this PPO catalyzed reaction because protoporphyrin IX is an important precursor for chlorophyll synthesis. Because of the important role of PPO in plant chlorophyll synthesis, diverse protoporphyrinogen oxidase (PPO)-inhibiting herbicides have been developed and used for weed control in agriculture since the 1960s. Application of the PPO-inhibiting herbicides to sensitive plants results in the blockage of heme and chlorophyll biosynthetic pathways in the plastids, leading to the accumulation of pathway intermediates which leak from the plastids and undergo non-specific oxidation to protoporphyrin IX in the cytosol. In the presence of oxygen and light, protoporphyrin IX rapidly generates singlet oxygen, resulting in uncontrolled membrane lipid peroxidation and plant death. Development of a PPO-inhibiting herbicide tolerance trait through biotechnology will provide additional tools and additional herbicide mode of action to the farmers for diversifying weed control system to control weed and to reduce/prevent development of herbicide resistance. Such a PPO herbicide tolerance trait could either be deployed alone, or could be stacked with other herbicide tolerance traits.

Combinations of herbicide tolerance traits are desirable to provide weed control options that increase grower flexibility and enable the use of multiple herbicide modes of action for controlling challenging weeds. Combining multiple desired traits in the genome can be achieved by several approaches: 1) by making crosses between two parents each having a desired trait at a randomly inserted site, and identifying progeny plants that have combination of the desired traits; 2) by retransforming a transgenic plant comprising one or more desired trait(s) with one or more genes for additional desired traits, either through random integration or through targeted integration of the one or more genes for additional desired traits; 3) by inserting multiple genes as a single DNA molecule into one location, or locus, in the genome, which provides a useful tool in weed control that is much simpler and less expensive to maintain during subsequent breeding into a diverse pool of elite germplasms; and 4) by targeting one or more desired traits to a specific genomic location (site directed integration) carrying one or more desired traits, in a new transformation event, followed by crosses between the new event and another event carrying the one or more desired event at the specific genomic location, resulting in progeny plants that have combination of the desired traits at one location and segregate together.

The expression of transgenes in a transgenic plant, plant part, seed, cell or progeny, and thus their effectiveness, may be influenced by many factors, such as the regulatory elements used in the transgenes' expression cassettes, the combination and/or interaction of these regulatory elements, the chromosomal location of the transgene insertion site, the chromatin structure of the genome at or near the transgene insertion site, and the presence or proximity of any endogenous cis and/or trans regulatory elements or genes close to the transgene insertion site. In addition, the performance of the traits in the transgenic plant is further complicated when the transgenic insert comprises multiple expression cassettes, each having a different transgene conferring a distinct trait. These differences or factors may result in variation in the level of transgene expression or in the spatial or temporal pattern of transgene expression among different transgenic insertion events of the same expression cassettes. Furthermore, different transgenic events can also vary in terms of the molecular quality of the events. For example, a transgenic event may contain two or more copies of the transgene insertion at one or more chromosomal locations, or a transgenic insertion may be truncated relative to the intended insertion or contain vector backbone sequences, or a transgene may be inserted into an endogenous gene or in a repeated region. In the case of site directed integration of desired traits, the machinery for site directed integration, such as gRNA or nuclease, which has to be excised from the commercial events, may not be completely removed. Such characteristics may result in undesirable outcomes, such as gene silencing, altered pattern and/or expression of the transgene, and/or altered pattern and/or expression of endogenous genes. There may also be undesirable phenotypic or agronomic differences among different events.

Even in the case of targeted sequence insertion, variability in the level of transgene expression between independent but genetically identical targeted sequence insertion (TSI) events was observed in a subset of transgenic events (Verkest et al., 2019). This expression variability and silencing occurred independently of the transgene sequence and could be attributed to DNA methylation that was further linked to different DNA methylation mechanisms. Transgene integration into targeted loci through Cre-lox mediated recombination has also been reported to produce a large percentage of targeted integration events that showed a partial spatial pattern of transgene expression due to differential silencing (Day et al., 2000). The fact that a considerable variation in transgene expression was observed shows that even when integration events are targeted, selection remains necessary similarly to the practice for random integration events in order to identify targeted insertion events with stable and desirable gene of interest expression over generations.

A commercially useful transgenic event requires that the transgene(s) in the transgenic insert express in the manner necessary for that trait to be successful, and involves rigorous testing, evaluation, and selection. Such tests include in vitro and/or in planta testing different regulatory elements (e.g., promoters, introns, leaders, and 3′ UTRs) and combinations of different regulatory elements for desirable spatial and temporal expression of the transgene(s), as well as examining whether to target the product of the transgene(s) (protein(s)) to subcellular compartments such as chloroplasts to select for the best expression cassette(s). For site directed integration of a transgene, once a targeted insertion strategy/method is chosen, the target sites are identified, screened and selected. The selected combinations of expression cassette(s), targeting sites and gRNA are then used for transformation to produce transgenic plants.

For these reasons, the performance of different transformation events from the same transformation construct can vary widely, and the identification of transformation events conferring the most beneficial traits or characteristics without other potential off-types or concerns is needed to select a superior event for commercial use. Therefore, a large number of individual transgenic events must be produced and analyzed to select an event having superior commercial properties, which can be a significant undertaking that involves analysis and selection among many different transformation events.

To establish a transgenic event for commercial use requires rigorous molecular characterization, greenhouse testing, and field trials over multiple years, in multiple locations and under a variety of conditions, allowing extensive agronomic, phenotypic, and molecular data to be obtained. The resulting data are then analyzed to select an event that is suitable for commercial purposes. The commercial event, once identified as having the desired transgene expression, molecular characteristics, efficacy and field performance, can then be introgressed into other corn genetic backgrounds using plant breeding methods. The resulting corn varieties contain the new traits combined with other desirable qualities such as native traits, disease tolerance traits, insect control traits, high-yielding germplasm or traits, and/or one or more other transgenic herbicide tolerance traits.

### SUMMARY OF THE INVENTION

Recombinant DNA molecules are provided herein. Examples of such recombinant DNA molecules include recombinant DNA molecules comprising a nucleotide sequence selected from the group consisting of SEQ ID NO:10; SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; a polynucleotide having a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO: 9; and a complete complement of any of the foregoing. In some embodiments, the recombinant DNA molecule is derived from a corn plant, seed, plant part, plant cell, progeny plant, or commodity product comprising corn event Zm_CSM63715, a representative sample of seed comprising the event having been deposited as ATCC Accession No. PTA-127361. In some embodiments, the recombinant DNA molecule is comprised in a corn plant, seed, plant part, plant cell, or progeny plant comprising corn event Zm_CSM63715, or a commodity product produced therefrom, a representative sample of seed comprising the event having been deposited as ATCC Accession No. PTA-127361. The recombinant DNA molecule can be formed by the insertion of a heterologous nucleic acid molecule into the genomic DNA of a corn plant or corn cell. The recombinant DNA molecule can comprise an amplicon diagnostic for the presence of corn event Zm_CSM63715.

DNA molecules that function as DNA probes are provided. An example of such a DNA molecule is a DNA molecule comprising a polynucleotide segment of sufficient length to function as a DNA probe that hybridizes specifically under stringent hybridization conditions with corn event Zm_CSM63715 DNA in a sample. Detecting hybridization of the DNA molecule under the stringent hybridization conditions is diagnostic for the presence of corn event Zm_CSM63715 in the sample.

Also provided is a DNA molecule comprising a polynucleotide segment of sufficient length to function as a DNA probe specific for detecting in a sample at least one of: a 5′ junction sequence between flanking corn genomic DNA and the transgenic insert of corn event Zm_CSM63715; a 3′ junction sequence between the transgenic insert of corn event Zm_CSM63715 and flanking corn genomic DNA; SEQ ID NO:9; and a fragment of SEQ ID NO:9 comprising a sufficient length of contiguous nucleotides of SEQ ID NO:9 to identify the sequence as a fragment of the transgenic insert of Zm_CSM63715.

The DNA probe can comprise SEQ ID NO:16. Alternatively, the DNA probe can comprise a nucleotide sequence selected from the group consisting of SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; and a complement of any of the foregoing. The sample can be derived from a corn plant, seed, plant part, plant cell, progeny plant, or commodity product.

A pair of DNA molecules is provided. The pair of DNA molecules comprises a first DNA molecule and a second DNA molecule. The first and the second DNA molecules comprise a fragment of SEQ ID NO:10 or a complement thereof and function as DNA primers when used together in an amplification reaction with DNA comprising corn event Zm_CSM63715 to produce an amplicon diagnostic for corn event Zm_CSM63715 in a sample. For example, the first and the second DNA molecules can comprise SEQ ID NO:14 and SEQ ID NO:15. The amplicon can comprise a nucleotide sequence selected from the group consisting of: SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; and a fragment of any of SEQ ID NO:1, SEQ ID NO:2, SEQ ID NO:3, SEQ ID NO:4, SEQ ID NO:5, SEQ ID NO:6, SEQ ID NO:7, SEQ ID NO:8, wherein the fragment is at least 10 nucleotides in length and comprises nucleotides 1,000-1,001 or 4,552-4,553 of SEQ ID NO:10.

Methods of detecting the presence of corn event Zm_CSM63715 in a sample derived from a corn seed, plant, plant part, plant cell, progeny plant, or commodity product, are provided. In a first example of such a method, the method comprises: a) contacting the sample with any of the DNA molecules that functions as DNA probes specific for corn event Zm_CSM63715 described herein; b) subjecting the sample and the DNA molecule that functions as a probe to stringent hybridization conditions; and c) detecting the hybridization of the DNA molecule that functions as a probe to a DNA molecule in the sample. The hybridization of the DNA molecule that functions as a probe to the DNA molecule in the sample is diagnostic for the presence of corn event Zm_CSM63715 in the sample.

Another method of detecting the presence of corn event Zm_CSM63715 in a sample derived from a corn seed, plant, plant part or plant cell, progeny plant or commodity product is provided. The method comprises: a) contacting the sample with any of the pairs of DNA molecules that can be used as primers to produce an amplicon diagnostic for corn event Zm_CSM63715 described herein; b) performing an amplification reaction sufficient to produce a DNA amplicon; and c) detecting the presence of the DNA amplicon. The DNA amplicon comprises at least one of: a 5′ junction sequence between flanking corn genomic DNA and the transgenic insert of corn event Zm_CSM63715, a 3′ junction sequence between flanking corn genomic DNA and the transgenic insert of corn event Zm_CSM63715, SEQ ID NO: 9, and a fragment of SEQ ID NO: 9 comprising a sufficient length of contiguous nucleotides of SEQ ID NO: 9 to identify the sequence as a fragment of the transgenic insert of Zm_CSM63715. The presence of the DNA amplicon indicates the presence of corn event Zm_CSM63715 in the sample. The DNA amplicon can be at least 10 nucleotides in length, at least 11 nucleotides in length, at least 12 nucleotides in length, at least 13 nucleotides in length, at least 14 nucleotides in length, at least 15 nucleotides in length, at least 16 nucleotides in length, at least 17 nucleotides in length, at least 18 nucleotides in length, at least 19 nucleotides in length, at least 20 nucleotides in length, at least 25 nucleotides in length, at least 30 nucleotides in length, at least 35 nucleotides in length, at least 40 nucleotides in length, at least 45 nucleotides in length, at least 50 nucleotides in length, at least 60 nucleotides in length, at least 70 nucleotides in length, at least 80 nucleotides in length, at least 90 nucleotides in length, or at least 100 nucleotides in length. The DNA amplicon can comprise a nucleotide sequence selected from the group consisting of SEQ ID NO:10; SEQ ID NO:9; SEQ ID NO:8; SEQ ID NO:7; SEQ ID NO:6; SEQ ID NO:5; SEQ ID NO:4; SEQ ID NO:3; SEQ ID NO:2; SEQ ID NO:1; and a fragment of any of SEQ ID NO:10, SEQ ID NO:8, SEQ ID NO:7, SEQ ID NO:6, SEQ ID NO:5, SEQ ID NO:4, SEQ ID NO:3, SEQ ID NO:2, and SEQ ID NO:1 that is at least 10 nucleotides in length and comprises nucleotides 1,000-1,001 or 4,552-4,553 of SEQ ID NO:10.

A further method of detecting the presence of corn event Zm_CSM63715 in a sample of DNA derived from a corn seed, plant, plant part, plant cell, progeny plant or commodity product is provided. The method comprises: a) contacting the sample with any of the DNA molecules that function as probes specific for corn event Zm_CSM63715 described herein; and performing a sequencing reaction to produce a target sequence. The target sequence comprises a nucleotide sequence selected from the group consisting of SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; a complete complement of any thereof; and a fragment of any of SEQ ID NO:1, SEQ ID NO:2, SEQ ID NO:3, SEQ ID NO:4, SEQ ID NO:5, SEQ ID NO:6, SEQ ID NO:7, SEQ ID NO:8, and SEQ ID NO:10 that is at least 10 nucleotides long and comprises nucleotides 1,000-1,001 or 4,552-4,553 of SEQ ID NO:10.

Another method of detecting the presence of corn event Zm_CSM63715 in a sample derived from a corn seed, plant, plant part, cell, progeny plant or commodity product is provided. The method comprises: a) contacting the sample with an antibody specific for the PPO (protoporphyrinogen oxidase) protein encoded by corn event Zm_CSM63715; and b) detecting binding of the antibody to the protein in the sample. The binding of the antibody indicates the presence of corn event Zm_CSM63715 in the sample.

DNA detection kits for detecting the presence of corn event Zm_CSM63715 in a sample are provided. One example of such a DNA detection kit is a kit comprising any of the pairs of DNA primers that can be used as primers to produce an amplicon diagnostic for corn event Zm_CSM63715 described herein. Another example of a DNA detection kit is a kit comprising any of the DNA molecules that function as probes specific for corn event Zm_CSM63715 described herein.

Also provided are protein detection kits for detecting the presence of corn event Zm_CSM63715 in a sample. One example of such a kit is a kit comprising an antibody specific for the PPO protein encoded by corn event Zm_CSM63715. Detecting binding of the antibody to the protein encoded by corn event Zm_CSM63715 in a sample is diagnostic for the presence of corn event Zm_CSM63715 in the sample.

Methods of determining the zygosity of a corn plant, plant part, plant seed, or plant cell comprising corn event Zm_CSM63715 are provided. One example of such a method comprises: a) contacting a sample comprising DNA derived from the corn plant, plant part, plant seed, or plant cell with a first primer set capable of producing a first amplicon diagnostic for the presence of corn event Zm_CSM63715, and a second primer set capable of producing a second amplicon diagnostic for the wildtype corn genomic DNA not comprising corn event Zm_CSM63715; b) performing a nucleic acid amplification reaction; and c) detecting the first amplicon and the second amplicon. The presence of both amplicons indicates that the plant, plant part, seed or cell is heterozygous for corn event Zm_CSM63715. The presence of only the first amplicon indicates that the plant, plant part, seed, or cell is homozygous for corn event Zm_CSM63715. For example, the first primer set can comprise SEQ ID NO:14 and SEQ ID NO:15, and the second primer set can comprise SEQ ID NO:20 and SEQ ID NO:21 or SEQ ID NO: 15 and SEQ ID NO: 21.

Another method of determining the zygosity of a corn plant, plant part, plant seed, or plant cell comprising corn event Zm_CSM63715 is provided. The method comprises: a) contacting a sample comprising DNA derived from the corn plant, plant part, plant seed, or plant cell with a probe set comprising at least a first probe that specifically hybridizes to corn event Zm_CSM63715, and at least a second probe that specifically hybridizes to corn genomic DNA that was disrupted by insertion of the heterologous DNA of corn event Zm_CSM63715 but does not hybridize to corn event Zm_CSM63715.; and b) hybridizing the probe set with the sample under stringent hybridization conditions. Detecting hybridization of only the first probe under the hybridization conditions is diagnostic for a corn plant, plant part, seed or plant cell homozygous for corn event Zm_CSM63715. Detecting hybridization of both the first probe and the second probe under the hybridization conditions is diagnostic for a corn plant, plant part, seed, or plant cell heterozygous for corn event Zm_CSM63715. For example, the probe set can comprise SEQ ID NO:16 and SEQ ID NO:22.

DNA constructs are provided. One example of a DNA construct provided herein is a DNA construct comprising an expression cassette, wherein the expression cassette comprises in operable linkage: i) a ubiquitin (UBQ) promoter, a leader sequence, and an intron sequence from Andropogon gerardii, ii) a chloroplast transit peptide coding sequence of APG6 (Albino and Pale Green 6) from Arabidopsis thaliana, iii) a codon-optimized protoporphyrinogen oxidase coding sequence from Enterobacter cloacae, and iv) a 3′ UTR sequence of an alpha tubulin gene from Arundo donax. For example, the DNA construct can comprise SEQ ID NO:9. The DNA construct can further comprise at the 5′ or 3′ end of said construct: a) at least 50 contiguous nucleotides of SEQ ID NO: 11 or SEQ ID NO:164; or b) at least 50 contiguous nucleotides of SEQ ID NO: 12 or SEQ ID NO:165.

Another DNA construct is provided. The DNA construct comprises a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO: 9. The DNA construct comprises at the 5′ or 3′ end of said construct (i) at least 50 contiguous nucleotides of SEQ ID NO: 11 or SEQ ID NO:164; or (ii) at least 50 contiguous nucleotides of SEQ ID NO: 12 or SEQ ID NO:165.

Any of the DNA constructs can comprise at the 5′ end of said construct one or more nucleotide sequences selected from SEQ ID NOs:44-103. Any of the DNA constructs can comprise at the 3′ end of said construct one or more nucleotide sequences selected from SEQ ID NOs:104-163.

Methods for controlling or preventing weed growth in an area or provided. One example of such a method comprises planting corn comprising event Zm_CSM63715 in the area and applying an effective amount of a PPO herbicide to control weeds in the area without injury to the corn or with less than about 10% injury to the corn. The effective amount of the PPO herbicide can be about 0.0009 lb/acre to about 1.5 lb/acre over a growing season.

Methods for controlling volunteer corn comprising corn event Zm_CSM63715 in an area are provided. One example of such a method comprises applying an herbicidally effective amount of at least one herbicide other than a PPO herbicide, wherein the herbicide application prevents growth of corn comprising corn event Zm_CSM63715. The herbicide other than a PPO herbicide can be selected from the group consisting of pyrithiobac, trifluralin, fluometuron, trifloxysulfuron, FOP herbicides such as quizalofop or fluazifop, DIM herbicides such as clethodim or sethoxydim, fenoxaprop, glyphosate, giufosinate, and combinations of any thereof.

Methods of obtaining a seed of a corn plant or a corn plant that is tolerant to PPO herbicides are provided. One example of such a method comprises: a) obtaining a population of progeny seed or plants grown therefrom, at least one of which comprises corn event Zm_CSM63715; and b) identifying at least a first progeny seed or plant grown therefrom that comprises corn event Zm_CSM63715. Identifying the progeny seed or plant grown therefrom that comprises corn event Zm_CSM63715 can comprise: a) growing the progeny seed or plant to produce progeny plants; b) treating the progeny plants with an effective amount of a PPO herbicide; and c) selecting a progeny plant that is tolerant to the PPO herbicide. The effective amount of the PPO herbicide can be about 0.0009 lb/acre to about 1.5 lb/acre over a growing season. Alternatively, or in addition, identifying the progeny seed or plant grown therefrom that comprises corn event Zm_CSM63715 can comprise detecting the presence of corn event Zm_CSM63715 in a sample derived from the progeny seed or plant grown therefrom. Alternatively, or in addition, identifying the progeny seed or plant grown therefrom that comprises corn event Zm_CSM63715 comprises detecting the presence of the PPO protein encoded by corn event Zm_CSM63715 in a sample derived from the progeny seed or plant grown therefrom.

Methods for improving tolerance to PPO herbicides in a corn plant are provided. One example of such a method comprises: a) inserting any of the DNA constructs described herein into the genome of a corn cell; b) generating a corn plant from the corn cell; and c) selecting a corn plant comprising the DNA construct. The selecting can comprise treating the corn cell or plant with an effective amount of a PPO herbicide. The effective amount of the PPO herbicide can be about 0.0009 lb/acre to about 1.5 lb/acre over a growing season.

Corn plants, plant seeds, plant parts, and plant cells comprising a recombinant DNA molecule are provided. The recombinant DNA molecule comprises a sequence selected from the group consisting of SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO: 9; and a complete complement of any of the foregoing. The corn plant, plant seed, plant part, or plant cell expresses a PPO herbicide tolerance gene. The corn plant, plant seed, plant part, or plant cell is tolerant to one or more PPO herbicides. The corn plant, plant seed, plant part, or plant cell can further comprise at least one additional transgene for tolerance to at least one additional herbicide. The corn plant, plant seed, plant part, or plant cell can comprise corn event Zm_CSM63715, a representative sample of seed comprising the event having been deposited under ATCC Accession No. PTA-127361. The corn plant, plant seed, plant part, or plant cell of any one of claims can be further defined as a progeny plant of any generation of a corn plant comprising corn event Zm_CSM63715, or a corn plant part, plant seed, or plant cell derived therefrom.

A further corn plant, plant part, plant seed, or plant cell is provided. The corn plant, plant part, plant seed, or plant cell comprises corn event Zm_CSM63715, a representative sample of seed comprising corn event Zm_CSM63715 having been deposited under ATCC Accession No. PTA-127361.

The corn plant part can comprise a microspore, pollen, an anther, silk, spike, an ovule, an ovary, a pod, a flower, a cob, an embryo, a stem, a leaf, a root, or a callus.

A further corn plant, plant part, plant seed, or plant cell is provided. The corn plant, plant seed, plant part, or plant cell is tolerant to one or more PPO herbicides and comprises any of the DNA constructs described herein.

Any of the corn plants, plant seeds, plant parts, or plant cells can be obtained by any of the methods of obtaining seed of a corn plant or a corn plant that is tolerant to PPO herbicides, or by any of the methods of improving tolerance to a PPO herbicide in a plant described herein.

A further corn plant, plant cell, plant part, or plant seed is provided. The corn plant, plant cell, plant part, or plant seed comprises a recombinant DNA construct integrated in chromosome 8. The recombinant DNA construct confers tolerance to at least one PPO herbicide. The recombinant DNA construct is integrated in a position of said chromosome flanked by at least 50 contiguous nucleotides of SEQ ID NO:11 or SEQ ID NO:164 and at least 50 contiguous nucleotides of SEQ ID NO:12 or SEQ ID NO:165. The at least 50 contiguous nucleotides of SEQ ID NO:11 or SEQ ID NO:164 can comprise one or more nucleotide sequences selected from SEQ ID NOs:44-103. The at least 50 contiguous nucleotides of SEQ ID NO:12 or SEQ ID NO:165 comprise one or more nucleotide sequences selected from SEQ ID NOs:104-163.

With respect to any of the corn plants, plant parts, or plant seeds tolerant to one or more PPO herbicides described herein, any of the methods for controlling or preventing weed growth in an area comprising applying an effective amount of a PPO herbicide described herein, any of the methods of obtaining a seed of a corn plant or a corn plant that is tolerant to PPO herbicides described herein, and any of the methods of improving tolerance to PPO herbicides in a corn plant described herein, the PPO herbicide can be selected from the group consisting of diphenylethers, N-phenylphthalimides, oxadiazoles, oxazolidinediones, phenylpyrazoles, pyrimidinediones, thiadiazoles, triazolinones, benzoxazinone derivatives, other PPO herbicides, and combinations of any thereof. The diphenylether can be selected from the group consisting of acifluorfen, bifenox, ethoxyfen, fluorodifen, fluoronitrofen, furyloxyfen, halosafen, chlomethoxyfen, chlornitrofen, ethoxyfen-ethyl, fluoroglycofen, lactofen, nitrofen, oxyfluorfen, fomesafen, a salt of any thereof, and an ester of any thereof. The N-phenylphthalimide can be selected from the group consisting of cinidon-ethyl, flumiclorac, flumiclorac-pentyl, and flumioxazin. The oxadiazole can be selected from the group consisting of oxadiargyl and oxadiazon. The oxazolidinedione can be pentoxazone. The phenylpyrazole can be selected from the group consisting of fluazolate, pyraflufen, and pyraflufen-ethyl. The pyrimidinedione can be selected from the group consisting of benzfendizone, butafenacil, epyrifencacil (S-3100), flupropacil, flufenoximacil, saflufenacil, and tiafenacil. The thiadiazole can be selected from the group consisting of fluthiacet-methyl and thidiazimin. The triazolinone can be selected from the group consisting of azafenidin, bencarbazone, carfentrazone, its salts and esters, and sulfentrazone. The benzoxazinone derivative can be 1,5-dimethyl-6-thioxo-3-(2,2,7-trifluoro-3,4-dihydro-3-oxo-4-prop-2-ynyl-2H-1,4-benzoxazin-6-yl)-1,3,5-triazinane-2,4-dione (trifludimoxazin). The other PPO herbicide can be selected from the group consisting of chlorphthalim, flufenpyr, flufenpyr-ethyl, flumipropyn, pyraclonil, profluazol, pyridin-2-ylmethyl[(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl[(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl[(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate, cyanomethyl[(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate; methyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, methyl (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate (flufenoximacil), methyl (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, methyl 2-{[(Z)-{2-chloro-4-fluoro-5-[3-methyl-2,6-dioxo-4-(trifluoromethyl)-3,6-dihydropyrimidin-1(2H)-yl]benzylidene}amino]oxy}propanoate, 2-{1[(Z)-{2-chloro-4-fluoro-5-[3-methyl-2,6-dioxo-4-(trifluoromethyl)-3,6-dihydropyrimidin-1(2H)-yl]benzylidene}amino]oxy}propanoic acid, ethyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, ethyl (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, ethyl (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoic acid, (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoic acid, (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoic acid, methyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}-2-methylpropanoate, ethyl 2-{[(E)-{2-chloro-4-fluoro-5-1[3-methyl-2,6-dioxo-4-(trifluoromethyl)-3,6-dihydropyrimidin-1(2H)-yl]benzylidene}amino]oxy}-2-methylpropanoate, methyl 2-{1[(E)-{2-chloro-4-fluoro-5-[3-methyl-2,6-dioxo-4-(trifluoromethyl)-3,6-dihydropyrimidin-1(2H)-yl]benzylidene}amino]oxy}butanoate, methyl (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoate, methyl (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoate, 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoic acid, (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoic acid, (2S)-2-{1[(E)-{2-chloro-4-fluoro-5-[3-methyl-2,6-dioxo-4-(trifluoromethyl)-3,6-dihydropyrimidin-1(2H)-yl]benzylidene}amino]oxy}butanoic acid, ethyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoate, methyl 2-({(E)-[2-chloro-5-(3,5-dimethyl-2,6-dioxo-4-sulfanylidene-1,3,5-triazinan-1-yl)-4-fluorobenzylidene]amino}oxy)propanoate methyl (2R)-2-({(E)-[2-chloro-5-(3,5-dimethyl-2,6-dioxo-4-sulfanylidene-1,3,5-triazinan-1-yl)-4-fluorobenzylidene]amino}oxy)propanoate, methyl (2S)-2-({(E)-[2-chloro-5-(3,5-dimethyl-2,6-dioxo-4-sulfanylidene-1,3,5-triazinan-1-yl)-4-fluorobenzylidene]amino}oxy)propanoate, 2-({(E)-[2-chloro-5-(3,5-dimethyl-2,6-dioxo-4-sulfanylidene-1,3,5-triazinan-1-yl)-4-fluorobenzylidene]amino}oxy)propanoic acid, (2R)-2-({(E)-[2-chloro-5-(3,5-dimethyl-2,6-dioxo-4-sulfanylidene-1,3,5-triazinan-1-yl)-4-fluorobenzylidene]amino}oxy)propanoic acid, (2S)-2-({(E)-[2-chloro-5-(3,5-dimethyl-2,6-dioxo-4-sulfanylidene-1,3,5-triazinan-1-yl)-4-fluorobenzylidene]amino}oxy)propanoic acid, ethyl 2-({(E)-[2-chloro-5-(3,5-dimethyl-2,6-dioxo-4-sulfanylidene-1,3,5-triazinan-1-yl)-4-fluorobenzylidene]amino}oxy)propanoate, ethyl (2R)-2-({(E)-[2-chloro-5-(3,5-dimethyl-2,6-dioxo-4-sulfanylidene-1,3,5-triazinan-1-yl)-4-fluorobenzylidene]amino}oxy)propanoate, ethyl (2S)-2-({(E)-[2-chloro-5-(3,5-dimethyl-2,6-dioxo-4-sulfanylidene-1,3,5-triazinan-1-yl)-4-fluorobenzylidene]amino}oxy)propanoate, methyl 2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoate, methyl (2R)-2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoate, methyl (2S)-2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoate, 2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoic acid, (2R)-2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoic acid, (2S)-2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoic acid, ethyl 3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylate, methyl 3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylate, 3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylic acid, (5R)-3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylic acid, (5S)-3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylic acid, ethyl (5S)-3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylate, ethyl (5R)-3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylate, ethyl 3-{2-chloro-4-fluoro-5-[3-methyl-2,6-dioxo-4-(trifluoromethyl)-3,6-dihydropyrimidin-1(2H)-yl]phenyl}-5-propyl-4,5-dihydro-1,2-oxazole-5-carboxylate, ethyl 3-{2-chloro-4-fluoro-5-[3-methyl-2,6-dioxo-4-(trifluoromethyl)-3,6-dihydropyrimidin-1(2H)-yl]phenyl}-5-ethyl-4,5-dihydro-1,2-oxazole-5-carboxylate, 3-[4-chloro-2-fluoro-5-(5-{[(isopropylideneamino)oxy]carbonyl}-5-methyl-4,5-dihydro-1,2-oxazol-3-yl)phenyl]-1-methyl-6-(trifluoromethyl)pyrimidine-2,4(1H,3H)-dione, ethyl 3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylate, methyl 3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylate, 3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylic acid, (5R)-3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylic acid, (5S)-3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylic acid, 3-[4-chloro-2-fluoro-5-(5-{[(isopropylideneamino)oxy]carbonyl}-5-methyl-4,5-dihydro-1,2-oxazol-3-yl)phenyl]-1,5-dimethyl-6-sulfanylidene-1,3,5-triazinane-2,4-dione, ethyl 3-{5-[3-amino-2,6-dioxo-4-(trifluoromethyl)-3,6-dihydropyrimidin-1(2H)-yl]-2-chloro-4-fluorophenyl}-5-methyl-4,5-dihydro-1,2-oxazole-5-carboxylate, 3-{5-[3-amino-2,6-dioxo-4-(trifluoromethyl)-3,6-dihydropyrimidin-1(2H)-yl]-2-chloro-4-fluorophenyl}-5-methyl-4,5-dihydro-1,2-oxazole-5-carboxylic acid, methyl 3-{2-chloro-4-fluoro-5-[3-methyl-2,6-dioxo-4-(trifluoromethyl)-3,6-dihydropyrimidin-1(2H)-yl]phenyl}-3a,4,5,6-tetrahydro-6aH-cyclopenta[d][1,2]oxazole-6a-carboxylate, ethyl 3-{2-chloro-4-fluoro-5-[3-methyl-2,6-dioxo-4-(trifluoromethyl)-3,6-dihydropyrimidin-1(2H)-yl]phenyl}-3a,4,5,6-tetrahydro-6aH-cyclopenta[d][1,2]oxazole-6a-carboxylate, methyl 3-{2-bromo-4-fluoro-5-[3-methyl-2,6-dioxo-4-(trifluoromethyl)-3,6-dihydropyrimidin-1(2H)-yl]phenyl}-3a,4,5,6-tetrahydro-6aH-cyclopenta[d][1,2]oxazole-6a-carboxylate, 2-ethoxy-2-oxoethyl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, {[(1-{2-chloro-4-fluoro-5-[3-methyl-2,6-dioxo-4-(trifluoromethyl)-3,6-dihydropyrimidin-1(2H)-yl]phenoxy}cyclopropyl)carbonyl]oxy}acetic acid, 2-methoxy-2-oxoethyl 1-{2-chloro-4-fluoro-5-1[3-methyl-2,6-dioxo-4-(trifluoromethyl)-3,6-dihydropyrimidin-1(2H)-yl]phenoxy}cyclopropanecarboxylate, and cyclopropylmethyl (2-{2-chloro-4-fluoro-5-1[3-methyl-2,6-dioxo-4-(trifluoromethyl)-3,6-dihydropyrimidin-1(2H)-yl]phenoxy}phenoxy)acetate.

A method of producing a progeny corn plant comprising corn event Zm_CSM63715 is provided. The method comprises: a) sexually crossing a first corn plant that comprises corn event Zm_CSM63715 with itself or a second corn plant; b) collecting one or more seeds produced from the cross; c) growing one or more seeds to produce one or more progeny plants; and d) selecting at least a first progeny plant or seed comprising corn event Zm_CSM63715. Inbred or hybrid corn plants and seeds comprising corn event Zm_CSM63715 that are produced by the method are also provided herein.

A nonliving or nonregenerable corn plant material is provided. The nonliving or nonregenerable corn plant material comprises any of the recombinant DNA molecules provided herein or any of the DNA constructs provided herein.

Another nonliving or nonregenerable corn plant material is provided. The nonliving or nonregenerable corn plant material comprises corn event Zm_CSM63715, a representative sample of seed comprising the corn event corn event Zm_CSM63715 having been deposited under ATCC Accession No. PTA-127361.

A commodity product is provided. The commodity product comprises any of the recombinant DNA molecules provided herein or any of the DNA constructs provided herein. The commodity product can be produced from a transgenic corn plant, plant part, plant seed, or plant cell comprising the corn event Zm_CSM63715. The commodity product can comprise whole or processed seeds; viable or nonviable seeds; viable plant parts (such as roots and leaves); viable plant cells; processed plant parts; processed plant tissues; dehydrated plant tissues; dehydrated plant parts; frozen plant tissues; frozen plant parts; food for human consumption such as corn oil, corn meal, corn flour, corn grits, corn flakes, corn bran, corn starch, sweetener such as high fructose corn syrup (HFCS), glucose and dextrose, beverage alcohol, brewer grits for beer production, fiber; animal feed such as corn, corn biomass; industrial alcohol; fuel ethanol; corn pollen; corn plastic; dried distillers grains (DDGs); or bio-degradable packing material.

A method of producing a commodity product is provided. The method comprises: a) obtaining a transgenic corn plant, plant part, or plant seed comprising corn event Zm_CSM63715; and b) producing a commodity product from the transgenic corn plant, plant part, or plant seed.

A method of controlling, preventing, or reducing the development of herbicide-tolerant weeds is provided. The method comprises cultivating in a crop growing environment a corn plant comprising transgenes that provide tolerance to (i) a PPO herbicide and (ii) herbicides with at least three additional herbicide modes of action, the three additional herbicide modes of action each being different from one another.

A further method for controlling, preventing, or reducing the development of herbicide-tolerant weeds is provided. The method comprises: a) cultivating in a crop growing environment a corn plant comprising any of the DNA constructs provided herein, and at least three additional transgenes for providing tolerance to herbicides with at least three additional herbicide modes of action, the three additional herbicide modes of action each being different from one another; and b) applying to the crop growing environment at least one herbicide selected from the group consisting of dicamba, glufosinate, 2,4-D, PPO inhibitor, glyphosate, and any combination thereof, wherein the corn plant is tolerant to the at least one herbicide.

In any of the methods for controlling, preventing, or reducing the development of herbicide-tolerant weeds the transgenes that provide tolerance to the herbicides with the at least three additional herbicide modes of action can be present at a single genomic location in the corn plant.

Also provided are methods of reducing loci for corn breeding by site-directed insertion of a transgene that provides tolerance to a PPO herbicide at a genomic location in a corn plant that is within about 3-8 cM of a locus in the genome of the corn plant that comprises transgenes for tolerance to at least three additional herbicide modes of action, the three additional herbicide modes of action each being different from one another.

In any of the corn plants, plant seeds, plant parts, or plant cells described herein that comprise at least one additional transgene for tolerance to at least one additional herbicide, and in any of the methods for controlling, preventing, or reducing the development of herbicide-tolerant weeds or methods for reducing loci for corn breeding, the additional transgenes can be selected from the group consisting of FT_T, dicamba monooxygenase (DMO), phosphinothricin N-acetyltransferase (PAT), 5-enolpyruvylshikimate-3-phosphate synthase (EPSPS), and combinations of any thereof. For example, the FT_T transgene can comprise a polynucleotide sequence encoding a protein having the amino acid sequence of SEQ ID NO:171. The DMO transgene can comprise a polynucleotide sequence encoding a protein having the amino acid sequence of SEQ ID NO:169. The PAT transgene can comprise a polynucleotide sequence encoding a protein having the amino acid sequence of SEQ ID NO:167. The EPSPS transgene can comprise a polynucleotide sequence encoding a protein having the amino acid sequence of SEQ ID NO:173. The additional transgenes can provide tolerance to herbicides having modes of action selected from the group consisting of inhibitors of glutamine synthetase, inhibitors of acetyl CoA carboxylase (ACCase) in the aryloxyphenoxy propionate (FOP) group, inhibitors of EPSPS, synthetic auxins, and combinations of any thereof. The inhibitor of acetyl CoA carboxylase (ACCase) in the aryloxyphenoxy propionate (FOP) group can be selected from the group consisting of chlorazifop, clodinafop, clodinafop-ethyl, clodinafop-propargyl, clofop, cyhalofop, cyhalofop-butyl, diclofop, diclofop-methyl, diclofop-P, diclofop-P-methyl, fenoxaprop, fenoxaprop-P, fenoxaprop-P-ethyl, fenthiaprop, fluazifop, fluazifop-butyl, fluazifop-P, fluazifop-P-butyl, haloxyfop, haloxyfop-etotyl, haloxyfop-methyl, haloxyfop-P, haloxyfop-P-methyl, isoxapyrifop, metamifop, propaquizafop, quizalofop, quizalafop-ethyl, quizalofop-P, quizalafop-P-ethyl, quizalafop-P-tefuryl, trifop, and combinations of any thereof. The synthetic auxin can be selected from the group consisting of dicamba, 2,4-D, dichlorprop, mecoprop, 2,4,5-T (2,4,5-trichlorophenoxyacetic acid), and combinations of any thereof. The inhibitor of glutamine synthetase can comprise glufosinate. The inhibitor of 5-enolpyruvylshikimate-3-phosphate synthase (EPSPS) can comprise glyphosate.

In any of the corn plants, plant seeds, plant parts, or plant cells described herein that comprise at least one additional transgene for tolerance to at least one additional herbicide, and in any of the methods for controlling, preventing, or reducing the development of herbicide-tolerant weeds or methods for reducing loci for corn breeding, the corn plant, plant seed, plant part, or plant cell can further comprise corn event MON87429.

In any of the corn plants, plant seeds, plant parts, or plant cells described herein that comprise at least one additional transgene for tolerance to at least one additional herbicide, and in any of the methods for controlling, preventing, or reducing the development of herbicide-tolerant weeds or methods for reducing loci for corn breeding, the corn plant, plant seed, plant part, or plant cell can further comprise a recombinant DNA molecule comprising a sequence selected from the group consisting of SEQ ID NO:212; SEQ ID NO:213; SEQ ID NO:214; SEQ ID NO:215; SEQ ID NO:216; SEQ ID NO:217; SEQ ID NO:218; SEQ ID NO:219; SEQ ID NO:220; SEQ ID NO:221; a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:212 or the full length of SEQ ID NO: 213; and a complete complement of any of the foregoing.

A corn plant, plant seed, plant part, plant cell or progeny plant is provided. The corn plant, plant seed, plant part, plant cell or progeny plant comprises a recombinant nucleic acid molecule. The recombinant nucleic acid molecule comprises a target corn genomic nucleic acid sequence having at least 85% sequence identity, at least 90% sequence identity, or at least 95% sequence identity, to a nucleic acid molecule selected from the group consisting of SEQ ID NOs:174-190. The recombinant nucleic acid molecule further comprises a DNA sequence of interest, wherein the DNA sequence of interest is inserted into said target corn genomic nucleic acid sequence. In some embodiments, the corn plant, seed, plant part, plant cell, or progeny plant comprises a recombinant nucleic acid molecule, said recombinant nucleic acid molecule comprising a target corn genomic nucleic acid sequence having a sequence selected from the group consisting of SEQ ID NOs:174-190. The DNA sequence of interest can comprise a gene of agronomic interest. For example, the gene of agronomic interest can confer herbicide tolerance in plants. In some embodiments, the target corn genomic nucleic acid sequence is at least 1 kb from the MON87429 insertion site. In some embodiments, the target corn genomic nucleic acid sequence maps to within 5 cM of the MON87429 insertion site. In some embodiments, the target corn genomic nucleic acid sequence is more than 1 kb from a gene, is more than 1 kb from a repressive chromatin mark, is more than 200 nucleotides from a small RNA hotspot, is more than 1 kb from a long repeat region, has DNA methylation less than or equal to 10% of genome-wide population average, and/or has a redundancy score less than or equal to 30%.

A method of generating a recombinant corn plant cell is provided. The method comprises: a) obtaining a corn plant, seed, or cell, wherein said plant, seed, or cell comprises a target corn genomic nucleic acid molecule having at least 85% sequence identity, at least 90% sequence identity, or at least 95% sequence identity to a nucleic acid molecule selected from the group consisting of SEQ ID NOs:174-190, or a complement thereof; b) introducing into the corn plant, seed, or cell a site-specific nuclease that can specifically bind to and cleave the target corn genomic nucleic acid molecule; c) introducing a DNA sequence of interest into the corn plant, seed, or cell; and d) selecting recombinant corn plants, seeds or cells comprising the DNA sequence of interest inserted in the target corn genomic nucleic acid molecule. The site-specific nuclease can be selected from the group consisting of an RNA-guided nuclease, a zinc finger nuclease, and a TALEN. In some embodiments, the RNA-guided nuclease is Cas12a. The method can further comprise introducing into the corn plant, seed, or cell a guide polynucleotide comprising a nucleic acid sequence that is substantially complementary to the target corn genomic nucleic acid, wherein the guide polynucleotide and the RNA-guided nuclease form a complex that can bind to and cleave the corn genomic nucleic acid molecule. For example, the guide polynucleotide comprises a nucleotide sequence having at least 85% sequence identity, at least 90% sequence identity, or at least 95% sequence identity to a nucleic acid molecule selected from the group consisting of SEQ ID NOs:195-211. The guide polynucleotide can further comprise SEQ ID NO: 23. In some embodiments, the target corn genomic nucleic acid sequence is at least 1 kb from the MON87429 insertion site. In some embodiments, the target corn genomic nucleic acid sequence maps to within 5 cM of the MON87429 insertion site. In some embodiments, the target corn genomic nucleic acid sequence is more than 1 kb from a gene, is more than 1 kb from a repressive chromatin mark, is more than 200 nucleotides from a small RNA hotspot, is more than 1 kb from a long repeat region, has DNA methylation less than or equal to 10% of genome-wide population average, and/or has a redundancy score less than or equal to 30%.

A recombinant DNA molecule is provided. The recombinant DNA molecule comprises a DNA sequence having at least 85% sequence identity, at least 90% sequence identity, or at least 95% sequence identity to a nucleic acid molecule selected from the group consisting of SEQ ID NOs:195-211. In some embodiments, the recombinant DNA molecule comprises a nucleic acid molecule selected from the group consisting of SEQ ID NOs:195-211. The DNA sequence can be operably linked to a heterologous promoter sequence. The recombinant DNA molecule can further comprise SEQ ID NO: 23.

A recombinant RNA molecule is provided. The recombinant RNA molecule comprises an RNA sequence that is at least 85% complementary, at least 90% complementary, or at least 95% complementary, to a nucleic acid molecule selected from the group consisting of SEQ ID NOs:195-211. In some embodiments, the RNA sequence is 100% complementary to a nucleic acid molecule selected from the group consisting of SEQ ID NOs:195-211.

In certain embodiments, the present disclosure provides a method for controlling or preventing weed growth in an area, the method comprising planting corn comprising event Zm_CSM63715 and event MON87429 in the area and applying an effective amount of at least one herbicide selected from the group consisting of a PPO herbicide, dicamba, glufosinate, 2,4-D, glyphosate, a FOP herbicide, and combinations of any thereof to control weeds in the area without injury to the corn or with less than about 10% injury to the corn.

### BRIEF DESCRIPTION OF THE DRAWINGS

FIG. 1 illustrates the sequence of the corn event Zm_CSM63715. Horizontal lines correspond to the positions of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:11, and SEQ ID NO:12 relative to SEQ ID NO:10. The horizontal arrows (SEQ ID NO:14, SEQ ID NO:15, SEQ ID NO:20, and SEQ ID NO:21) represent the approximate positions of illustrative primer pairs that can be used to detect corn event Zm_CSM63715. The horizontal lines labeled SEQ ID NO:16 and SEQ ID NO:22 represent the approximate positions of illustrative DNA probes that can be used to detect corn event Zm_CSM63715 or wildtype corn sequence. “RB” refers to the Agrobacterium T-DNA right border; “LB” refers to the Agrobacterium T-DNA left border. “Promoter” represents a promoter element; “Leader” represents a leader (5′ UTR) element; “Intron” represents an intron element; “CTP” represents a chloroplast transit peptide element; “3′ UTR” represents a 3′ UTR; “PPO” represents a protoporphyrinogen oxidase coding element; and “Lox” represents a lox recombination site. The horizontal line labeled SEQ ID NO:13 represents the relative location or position of the wildtype corn genome where the transgene (SEQ ID NO:9) was inserted. The dashed line represents a 19-nucleotide deletion in the corn genome at the site of transgene (SEQ ID NO:9) insertion.

FIG. 2 is a diagrammatic representation of the T-DNA cassettes in the Agrobacterium Ti plasmid used to transform and generate corn event Zm_CSM63715 before and after T-DNA integration, and after Cre-mediated excision of the marker cassettes. “RB” refers to the Agrobacterium T-DNA right border; “LB” refers to the Agrobacterium T-DNA left border. “CP4”, “Cpf1”, “PPO”, and “gRNA” represent the CP4 selectable marker cassette, the Cpf1 nuclease cassette, the protoporphyrinogen oxidase cassette, and the gRNA cassette, respectively. “Lox” represents a lox recombination site. “5′ Flank” and “3′ Flank” represent the 5′ and 3′ flanking corn genomic sequences at the site of T-DNA integration, respectively. A: “T-DNA Before Integration” represents T-DNA comprising the CP4, Cpf1, gRNA and PPO cassettes before transformation; B: “Inserted T-DNA After Integration” represents T-DNA comprising the CP4, Cpf1, gRNA and PPO cassettes integrated into the corn genome after transformation; C: “Inserted T-DNA After Cre-Excision” represents the integrated T-DNA cassette after the CP4, Cpf1, and gRNA cassettes were excised, leaving behind one of the two Lox sites, the PPO cassette and the left and right borders.

FIG. 3 illustrates the approximate timelines for the research, testing, and development, leading to selection of the commercial event corn event Zm_CSM63715. “POC” stands for Proof of Concept; “TFN” strands for Transformation; “GH” stands for Greenhouse; “SA” stands for South America; “NA” stands for North America.

FIG. 4 is a diagrammatic representation of the breeding process to produce marker-free corn events from Type 1 constructs. “R₀ Transformants” are the initial transgenic events generated by transformation with the binary transformation vector, which was hemizygous for the T-DNA allele comprising the CP4, Cpf1, gRNA and PPO cassettes. The R0 transformants were cross-pollinated with a transgenic corn line comprising a transgene cassette for the expression of Cre-recombinase, resulting in an F1 generation, wherein many of the progeny lost the CP4, Cpf1 and gRNA cassettes flanked by the two Lox sites due to Cre-recombinase excision. Hemizygous T-DNA positive, CP4, Cpf1 and gRNA negative (also known as marker-free) and Cre-free plants were selected and self-pollinated, resulting in an F₂ generation. F₂ plants homozygous for the inserted T-DNA allele without the CP4, Cpf1, gRNA cassettes and lacking the Cre-recombinase transgene cassette were selected and self-pollinated giving rise to an F₃ generation. The F₃ generation plants were self-pollinated to produce a pure line of F₄ Gold Standard Seed. F₂ marker-free plants homozygous for the inserted T-DNA allele were also cross-pollinated with elite lines to produce R₁ populations for hybrid efficacy and agronomic trials. Subsequent “R” generations (R₁, R₂, and R₃) represent successive generations produced through self-pollination of plants derived from the initial R₀ transformant that resulted in the corn event Zm_CSM63715. The R₃ generation plants which were marker-free and homozygous for the T-DNA insertion were used for inbred efficacy and agronomic trials.

FIG. 5 is a diagrammatic representation of the breeding process to produce marker-free events from Type 2 constructs. “R₀ Transformants” are the initial transgenic events generated by transformation with the binary transformation vector, which was hemizygous for the T-DNA allele comprising the CP4, Cre, Cpf1, gRNA and PPO cassettes. The R0 transformants were self-pollinated, resulting in an R₁ generation, wherein many of the progeny lost the CP4, Cre, Cpf1 and gRNA cassettes flanked by the two Lox sites due to auto excision. Hemizygous T-DNA positive, CP4, Cre, Cpf1 and gRNA negative (also known as marker-free) plants were selected and self-pollinated, resulting in an R₂ generation. R₂ plants homozygous for the inserted T-DNA allele without the CP4, Cre, Cpf1, gRNA cassettes were selected and self-pollinated giving rise to an R₃ generation. R₁ plants hemizygous for PPO and marker genes were also cross-pollinated with a Cre-line to produce F1 progeny as a backup in case auto excision failed. Progeny plants hemizygous for PPO, and negative for the CP4, Cpf1, gRNA cassettes were selected and selfed to produce F2 progeny.

### BRIEF DESCRIPTION OF THE SEQUENCES

SEQ ID NO:1 is a 30-nucleotide sequence representing the 5′ junction region of the corn genomic DNA and the integrated transgene insert. SEQ ID NO:1 corresponds to nucleotide positions 986-1,015 of SEQ ID NO:10.

SEQ ID NO:2 is a 30-nucleotide sequence representing the 3′ junction region of the integrated transgene insert and the corn genomic DNA. SEQ ID NO:2 corresponds to nucleotide positions 4,538-4,567 of SEQ ID NO:10.

SEQ ID NO:3 is a 60-nucleotide sequence representing the 5′ junction region of the corn genomic DNA and the integrated transgene insert. SEQ ID NO:3 corresponds to nucleotide positions 971-1,030 of SEQ ID NO:10.

SEQ ID NO:4 is a 60-nucleotide sequence representing the 3′ junction region of the integrated transgene insert and the corn genomic DNA. SEQ ID NO:4 corresponds to nucleotide positions 4,523-4,582 of SEQ ID NO:10.

SEQ ID NO:5 is a 100-nucleotide sequence representing the 5′ junction region of the corn genomic DNA and the integrated transgene insert. SEQ ID NO:5 corresponds to nucleotide positions 951-1,050 of SEQ ID NO:10.

SEQ ID NO:6 is a 100-nucleotide sequence representing the 3′ junction region of the integrated transgene insert and the corn genomic DNA. SEQ ID NO:6 corresponds to nucleotide positions 4,503-4,602 of SEQ ID NO:10.

SEQ ID NO:7 is a 1,050-nucleotide sequence representing the 5′ genomic flank region of the corn genomic DNA and 50 bp of the integrated transgene insert. SEQ ID NO:7 corresponds to nucleotide positions 1-1,050 of SEQ ID NO:10.

SEQ ID NO:8 is a 1,050-nucleotide sequence representing 50 bp of the 3′ junction region of the integrated transgene insert and the 3′ genomic flank region of the corn genomic DNA. SEQ ID NO:8 corresponds to nucleotide positions 4,503-5,552 of SEQ ID NO:10.

SEQ ID NO:9 is a 3,552-nucleotide sequence corresponding to the transgene insert of corn event Zm_CSM63715. SEQ ID NO:9 corresponds to nucleotide positions 1,001-4,552 of SEQ ID NO:10.

SEQ ID NO:10 is a 5,552-nucleotide sequence corresponding to the contig nucleotide sequence of the 5′ corn genomic DNA sequence (SEQ ID NO:11), the transgene insert in event Zm_CSM63715 (SEQ ID NO:9), and the 3′ corn genomic DNA sequence (SEQ ID NO:12).

SEQ ID NO:11 is a 1,000-nucleotide sequence representing the 5′ flanking corn genomic DNA up to the transgene insert (SEQ ID NO:9). SEQ ID NO:11 corresponds to nucleotide positions 1-1,000 of SEQ ID NO:10.

SEQ ID NO:12 is a 1,000-nucleotide sequence representing the 3′ flanking corn genomic DNA after the transgene insert (SEQ ID NO:9). SEQ ID NO:12 corresponds to nucleotide positions 4,553-5,552 of SEQ ID NO:10.

SEQ ID NO:13 is a 2,019-nucleotide sequence representing wildtype corn genomic DNA at the location where the transgenic sequence (SEQ ID NO:9) was inserted in event Zm_CSM63715. A 19-nucleotide fragment of SEQ ID NO:13 (nucleotides 1,001-1,019) was deleted in event Zm_CSM63715 due to insertion of the T-DNA.

SEQ ID NO:14 is a 30-nucleotide sequence corresponding to a thermal amplification primer referred to as SQ21524 used in event-specific assay and zygosity assay to detect corn event Zm_CSM63715 DNA in a sample, and is identical to the reverse complement of the nucleotide sequence corresponding to positions 1,053-1,082 of SEQ ID NO:10.

SEQ ID NO:15 is a 30-nucleotide sequence corresponding to a thermal amplification primer referred to as SQ51880 used in event-specific assay and zygosity assay to detect corn event Zm_CSM63715 DNA in a sample, and is identical to the nucleotide sequence corresponding to positions 956-985 of SEQ ID NO:10.

SEQ ID NO:16 is a 16-nucleotide sequence corresponding to a 6FAM-MGB probe referred to as PB10269 used in event-specific assay and zygosity assay to detect corn event Zm_CSM63715 DNA in a sample, and is identical to the nucleotide sequence corresponding to positions 11,108-11,123 of SEQ ID NO:10.

SEQ ID NO:17 is a 24-nucleotide sequence corresponding to a thermal amplification primer referred to as SQ20222 used as an internal control for the event assay for corn event Zm_CSM63715 and hybridizes to a region of the corn genome.

SEQ ID NO:18 is a 28-nucleotide sequence corresponding to a thermal amplification primer referred to as SQ20221 used as an internal control for the event assay for corn event Zm_CSM63715 and hybridizes to a region of the corn genome.

SEQ ID NO:19 is a 17-nucleotide sequence corresponding to a VIC-MGB probe referred to as PB50298 used as an internal control for the event assay for corn event Zm_CSM63715 and hybridizes to a region of the corn genome.

SEQ ID NO:20 is a 34-nucleotide sequence corresponding to a thermal amplification forward primer referred to as SQ52146 used in a zygosity assay for detection of the wildtype (WT) allele DNA in a sample and hybridizes to a region of the corn genome. It corresponds to positions 948-981 of SEQ ID NO:10.

SEQ ID NO:21 is a 32-nucleotide sequence corresponding to a thermal amplification reverse primer referred to as SQ52147 used in a zygosity assay for detection of the WT allele DNA in a sample and hybridizes to a region of the corn genome. It corresponds to positions 4606-4637 of SEQ ID NO:10.

SEQ ID NO:22 is a 24-nucleotide sequence corresponding to a probe referred to as PB50707 used in a zygosity assay for detection of the WT allele DNA in a sample, and hybridizes to a region of the corn genome. It corresponds to positions 4555-4578 of SEQ ID NO:10.

SEQ ID NO:23 is a 21-nucleotide downstream mature crRNA scaffold sequence.

SEQ ID NOs:24-32 are the nucleotide sequences for the genetic elements in the transgenic insert of corn event Zm_CSM63715 and are further described in Table 1A hereinbelow.

SEQ ID NOs:33 and 34 are the nucleotide and amino acid sequences of LbCpf1 (also known as LbCas12a) of Lachnospiraceae bacterium ND2006, respectively.

SEQ ID NO:35 is the amino acid sequence for LbCas12a_V1 (G532R/K595R).

SEQ ID NO:36 is the amino acid sequence for LbCas12a_V2 (G532R/K538V/Y542R).

SEQ ID NO:37 is the amino acid sequence for Cas12a of Francisella_novicida (FnCas12a).

SEQ ID NO:38 is the nucleotide sequence for the gRNA repeat for LbCas12a.

SEQ ID NO:39 is the nucleotide sequence for the gRNA repeat for FnCas12a.

SEQ ID NO:40 is the nucleotide sequence for the gRNA gRNA_5F-63.

SEQ ID NO:41 is the nucleotide sequence for the gRNA gRNA_3F-4.

SEQ ID NOs:42 and 43 are the codon-optimized coding sequence and amino acid sequence of the protoporphyrinogen oxidase (PPO) from Enterobacter cloacae, respectively.

SEQ ID NOs:44-103 are 50-nucleotide sequences in the 5′ flank genomic sequence of event Zm_CSM63715. SEQ ID NOs:44-63 are based on the genomic sequence of the transformation germplasm; SEQ ID NOs:64-103 are based on the genomic sequence of corn B73 germplasm.

SEQ ID NOs:104-163 are 50-nucleotide sequences in the 3′ flank genomic sequence of event Zm_CSM63715. SEQ ID NOs:104-123 are based on the genomic sequence of the transformation germplasm; SEQ ID NOs:124-163 are based on the genomic sequence of corn B73 germplasm.

SEQ ID NO:164 is a 5,000-nucleotide sequence representing corn genomic DNA that flanks the transgenic insert at the 5′ end of the insert. Nucleotides 4,001-5,000 of SEQ ID NO:164 are identical to nucleotides SEQ ID NO: 11. The remaining nucleotides of SEQ ID NO:164 (nucleotides 1-4,000) are based on the genomic sequence of corn B73 germplasm.

SEQ ID NO:165 is a 5,000-nucleotide sequence representing corn genomic DNA that flanks the transgenic insert at the 3′ end of the insert. Nucleotides 1-1,000 of SEQ ID NO:165 are identical to nucleotides SEQ NO: 12. The remaining nucleotides of SEQ ID NO:165 (nucleotides 1,001-5,000) are based on the genomic sequence of corn B73 germplasm.

SEQ ID NOs:166 and 167 are the nucleotide and amino acid sequences of the phosphinothricin N-acetyltransferase gene (PAT).

SEQ ID NOs:168-169 are the nucleotide and amino acid sequences of the dicamba monooxygenase (DMO) gene.

SEQ ID NOs:170-171 are the nucleotide and amino acid sequences of the FT_T gene.

SEQ ID NOs:172-173 are the nucleotide and amino acid sequences of the 5-enolpyruvylshikimate-3-phosphate synthase (EPSPS) gene.

SEQ ID NOs:174-190 are the nucleotide sequences of gRNA target sites.

SEQ ID NO:191 is the nucleotide sequence of a nuclear localization signal (NLS) from tomato heat stress transcription factor HSFA1.

SEQ ID NO:192 is the nucleotide sequence of a polyubiquitin promoter from Zea mays Cv. Mexicana.

SEQ ID NO:193 is the 3′ UTR sequence of a lipid transfer protein from Oryza sativa.

SEQ ID NO:194 is a 36-nucleotide upstream pre-crRNA scaffold (also called direct repeat) sequence.

SEQ ID NOs:195-211 are 21-nucleotide gRNA spacer sequences corresponding to SEQ ID NOs:174-190.

SEQ ID NO:212 is the nucleotide sequence of corn event MON87429 corresponding to the contig nucleotide sequence of 5′ flanking corn genomic sequence+transgenic insert+3′ flanking corn genomic sequence.

SEQ ID NO:213 is the nucleotide sequence of the transgenic insert in corn event MON87429.

SEQ ID NOs:214-217 are the 5′ junction sequences in corn event MON87429.

SEQ ID NOs:218-221 are the 3′ junction sequences in corn event MON87429.

### DETAILED DESCRIPTION OF THE INVENTION

The following definitions, descriptions, and methods are provided to better define the invention and to guide those of ordinary skill in the art in the practice of the invention. Unless otherwise noted, terms are to be understood according to conventional usage by those of ordinary skill in the relevant art.

Herbicide tolerance is an important agronomic trait for effective weed control to maintain favorable crop growing conditions and crop yields, and is achieved by engineering of herbicide tolerance transgenes in crop plants using modern plant biotechnology techniques. Corn event Zm_CSM63715 confers tolerance to PPO herbicides and provides another mode of action for weed control and herbicide-resistant weed management.

Corn event Zm_CSM63715 is provided. The event Zm_CSM63715 was produced by Agrobacterium-mediated transformation of corn seed-derived embryo explants with a DNA construct comprising four transgene cassettes. The first cassette encoded a protoporphyrinogen oxidase (PPO) from Enterobacter cloacae for conferring tolerance to PPO herbicides. The second cassette encoded a 5-enolpyruvylshikimate-3-phosphate synthase (EPSPS, also known as CP4) from Agrobacterium tumefaciens CP4 strain as a selectable marker for selection of transgenic events. The third cassette encoded a Cpf1 nuclease from Lachnospiraceae bacterium ND2006 (LbCpf1 or LBCas12a) for site directed integration of the transgenes. The fourth cassette encoded a gRNA for directing the Cpf1 nuclease to its corn genomic target region. The CP4, Cpf1 and gRNA cassettes were flanked by two lox sites, and were removed when crossed to a transgenic corn line producing the Cre enzyme.

Plant transformation techniques, such as Agrobacterium-mediated or biolistic transformation, can be used to insert foreign DNA (also known as transgenic DNA) randomly into a chromosome in a plant cell to produce a genetically engineered plant cell, also referred to as a “transgenic” or “recombinant” cell. Using these transformation techniques, many individual cells can be transformed, each resulting in a unique “transgenic event” or “event” due to the random insertion of the foreign DNA into the genome. A transgenic plant can then be regenerated from each individual transgenic cell. This results in every cell of the transgenic plant containing the uniquely inserted transgenic event as a stable part of its genome. The transgenic plant can then be used to produce progeny plants, each containing the unique transgenic event. The term “transgenic” refers to a plant, plant part, plant cell, seed, progeny plant, or DNA molecule, construct, or sequence comprising a transgene-e.g., a “transgenic cell” refers to a cell comprising a transgene.

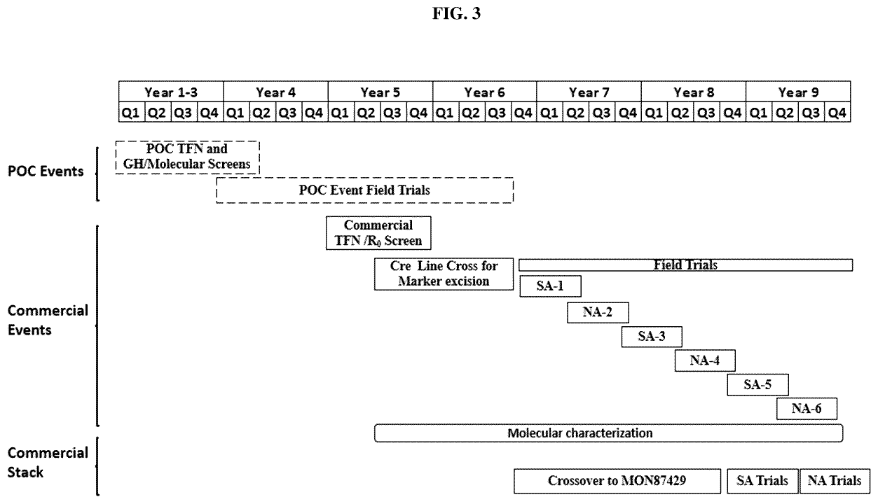

Corn event Zm_CSM63715 was produced and identified by a complex research and development process. This process included: (i) design and selection of DNA constructs comprising the four transgene cassettes based on design and testing of individual transgene cassettes with combinations of different expression elements; (ii) identification and screening of different transgene target sites, followed by design and testing of different guide RNAs for efficient cutting at the target sites; (iii) transformation of thousands of corn cells with the DNA constructs; (iv) regeneration of a large population of transgenic events; and (v) rigorous multi-year construct and event selection involving molecular characterization of the large number of transgenic events, greenhouse and field trials for herbicide tolerance efficacy and agronomic performance at different locations and in different geographies. Corn event Zm_CSM63715 was thus produced and selected as a uniquely superior event useful for broad-scale agronomic commercial purposes. FIG. 3 illustrates the approximate timelines for the research, testing and development, leading to selection of the commercial event corn event Zm_CSM63715.

Detailed molecular characterization was conducted on the transgenic events. Event Zm_CSM63715 was selected based on stringent molecular criteria, as well as other selection criteria such as herbicide tolerance efficacy and agronomic performance. The results from such molecular analyses confirmed that: (1) event Zm_CSM63715 contains a single T-DNA inserted at the targeted location, with one copy of the transgenic insert comprising only the PPO cassette; (2) no additional elements from the transformation construct were present other than the PPO expression cassette and one lox site between the left and right borders of the T-DNA, such as the transformation construct backbone sequence or the CP4, Cpf1 and gRNA cassettes; (3) the transgenic DNA was inserted in an intergenic region, far away from any endogenous genes or repeat regions; (4) the transgenic event produced the correct sized transcript and protein for the PPO transgene by northern hybridization and western hybridization analyses, respectively; (5) the event did not contain the Cre cassette. Furthermore, DNA sequence analyses were performed to: (1) determine the 5′ and 3′ transgenic insert-to-plant genome junctions; (2) confirm the organization of the elements within the insert; (3) verify the complete nucleotide sequence of the inserted transgenic DNA (SEQ ID NO:9); and (4) determine the PPO protein levels in different tissues such as leaf, root, silk and seed, and PPO protein levels in leaves over multiple generations.

In addition, primers and probes were designed, and thermal amplification assays were developed for producing specific amplicons diagnostic for the presence of event Zm_CSM63715 in a sample. As used herein, the 5′ and 3′ designations in reference to the junction, direction and side of the transgenic event insertion is relative to the 5′ to 3′ direction of the transgene, with the 5′ junction and genomic sequence being upstream of the transgene, and the 3′ junction and genomic sequence being downstream of the transgene.

As used herein, an “expression cassette” or “cassette” or “transgene cassette” is a recombinant DNA molecule or sequence comprising a combination of distinct elements for expressing an RNA and/or protein encoded by the coding sequence of a transgene in a transformed plant cell or transformed plant comprising the transgene. As provided herein, an “expression cassette” or “cassette” or “transgene cassette” includes one or more regulatory element(s) operably linked to a coding or transcribable DNA sequence. The regulatory elements can include a promoter, a leader, 5′ untranslated region (5′ UTR), intron and/or a 3′ untranslated region (3′ UTR) region. The “expression cassette” or “cassette” or “transgene cassette” is recombinant and heterologous with respect to the transformed plant cell genome. For purposes of the present disclosure, such an “expression cassette” or “cassette” or “transgene cassette” is a recombinant DNA molecule or sequence that encodes a protein for conferring tolerance to at least one class of herbicides as described herein. Table 1A provides a list of the genetic elements contained in the transgene cassette in the transgenic insert (SEQ ID NO:9) of corn event Zm_CSM63715.

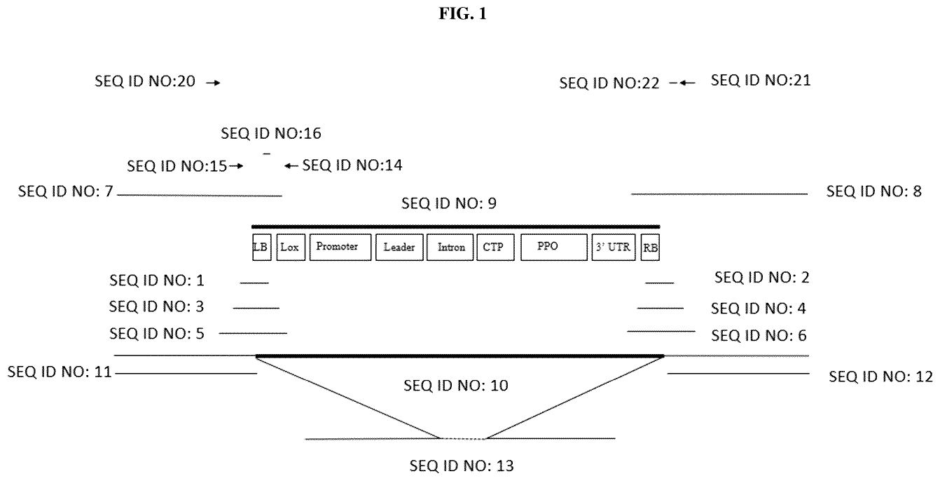

Insertion of the transgenic DNA into the genome of the corn plant is accomplished by plant transformation methods known in the art and creates a new transgenic genomic DNA sequence, known as a “transgenic event” or an “event.” The DNA sequence of the event consists of the inserted foreign DNA (referred to as “transgenic insert”) and the genomic DNA adjacent to, or “flanking,” the transgenic insert on either side of the insertion location. As used herein, the term “flanking” in reference to a transgenic event refers to the plant genomic sequence(s) adjacent to the transgenic DNA insertion in the genome of a transformed plant, plant part, plant tissue, or plant cell comprising the transgenic event on the 5′ and/or 3′ end(s) of the transgenic event insertion. Likewise, “flanking DNA” refers to a length of genomic DNA sequence adjacent to the transgenic DNA insertion in the genome of the transformed event on the 5′ and/or 3′ end(s) of the insertion. A “5′ flank”, therefore, means the corn genomic DNA sequence adjacent to and upstream (or on the 5′ end) of the transgenic DNA insertion. For example, a “5′ flank” can include the corn genomic DNA sequence immediately adjacent to and upstream (on the 5′ end) of the transgenic insertion, or any corn genomic DNA sequence upstream (on the 5′ end) of the transgenic insertion that is not immediately adjacent to the transgenic insertion but is within about 5000 nucleotides, within about 3000 nucleotides, or within about 1000 nucleotides upstream of the transgenic insertion. Likewise, a “3′ flank” means the corn genomic DNA sequence adjacent to and downstream (or on the 3′ end) of the transgenic insert. For example, a “3′ flank” can include the corn genomic DNA sequence immediately adjacent to and downstream (on the 3′ end) of the transgenic insertion, or any corn genomic DNA sequence downstream (on the 3′ end) of the transgenic insertion that is not immediately adjacent to the transgenic insertion but is within about 5000 nucleotides, within about 3000 nucleotides, or within about 1000 nucleotides downstream of the transgenic insertion. The DNA sequence of an event is unique to and specific for the event and can be readily identified when compared to other DNA sequences, such as that of other events or untransformed corn genomic DNA. Corn event Zm_CSM63715 has the new and unique DNA sequence provided as SEQ ID NO:10, which comprises a contiguous sequence comprising the 5′ corn genomic flanking sequence provided as SEQ ID NO:11, the transgenic insert sequence provided as SEQ ID NO:9, and the 3′ corn genomic flanking sequence provided as SEQ ID NO:12 (FIG. 1). Corn event Zm_CSM63715 is thus a DNA molecule that is an integral part of the chromosome of transgenic corn cells and plants comprising the event and as such is static and may be passed on to progeny cells and plants. As is described further in the Examples hereinbelow, various gene editing tools exist that would permit modification of the transgenic insert and/or the flanking genomic DNA of corn event Zm_CSM63715, such as by deletion, insertion, transposition, or substitution of nucleic acid sequence(s); the event is still uniquely characterized by the presence of heterologous DNA at the particular position in the genome occupied by corn event Zm_CSM63715 relative to flanking portions of the native corn genome.

TABLE 1A
Elements and Description of Corn Event Zm_CSM63715.

| | | Position in | |
|---|---|---|---|
| | SEQ | SEQ ID | |
| Element | ID NO | NO: 10 | Description |
| 5′ Flank sequence | 11 | 1-1000 | Corn genomic DNA sequence flanking the 5′ end |
| | | | of the transgenic insert. |
| Left border region | 24 | 1004-1192 | Left border sequence from Agrobacterium |
| | | | tumefaciens. |
| | | | SEQ ID NO: 24 is the 442-nucleotide, full-length |
| | | | T-DNA left border sequence. A portion of this |
| | | | sequence was truncated during T-DNA insertion |
| | | | into the corn genomic DNA. Only nucleotides |
| | | | 254-442 of SEQ ID NO: 24 are present in SEQ ID |
| | | | NO: 10. |
| Intervening sequence 1 | — | 1193-1198 | Sequence used in DNA cloning. |
| RS-P1.lox | 25 | 1199-1232 | Lox recombination site. |
| Intervening sequence 2 | — | 1233-1238 | Sequence used in DNA cloning. |
| P-ANDge.Ubq | 26 | 1239-2089 | Promoter sequence from a ubiquitin gene from |
| | | | Andropogon gerardi. |
| L-ANDge.Ubq | 27 | 2090-2243 | 5′ UTR leader sequence of a ubiquitin gene from |
| | | | Andropogon gerardi. |
| I-ANDge.Ubq | 28 | 2244-3244 | Intron sequence of a ubiquitin gene from |
| | | | Andropogon gerardi. |
| Intervening sequence 3 | — | 3245-3250 | Sequence used in DNA cloning. |
| TS-At.APG6 | 29 | 3251-3454 | APG6 (Albino and Pale Green 6) chloroplast |
| | | | transit peptide sequence from Arabidopsis |
| | | | thaliana. |
| CR- | 30 | 3455-3991 | Codon-optimized coding sequence of a |
| ENTcl.PPO_H_N90 | | | protoporphyrinogen oxidase (PPO) from |
| | | | Enterobacter cloacae. |
| Intervening sequence 4 | — | 3992-4005 | Sequence used in DNA cloning. |
| T-ARUdo.TubA | 31 | 4006-4503 | 3′ UTR sequence of an alpha tubulin gene from |
| | | | Giant Cane (Arundo donax). |
| Intervening sequence 5 | — | 4504-4509 | Sequence used in DNA cloning. |
| Right border region | 32 | 4510-4552 | Left border sequence from Agrobacterium |
| | | | tumefaciens. |
| | | | SEQ ID NO: 43 is the full-length T-DNA left |
| | | | border sequence and is 442 nucleotides long. |
| | | | However, since a portion of this sequence was |
| | | | truncated at the time of the insertion into the corn |
| | | | genomic DNA, only nucleotides 1-232 of SEQ ID |
| | | | NO: 43 are present in SEQ ID NO: 10. |
| 3′ Flank sequence | 12 | 4553-5552 | Corn genomic DNA sequence flanking the 3′ end |
| | | | of the transgenic insert. |

Progeny of the original transformed cell and plant that comprise corn event Zm_CSM63715 are provided. Such progeny may be produced by selfing of a corn plant comprising the corn event Zm_CSM63715, or by sexual cross or outcrossing between a corn plant comprising corn event Zm_CSM63715 and another plant that does or does not contain the event, or by any other method known in the art including any plant cell or tissue culture method, wherein the progeny includes the corn event Zm_CSM63715. The other plant may be a transgenic plant comprising the same and/or different event(s) or may be a non-transgenic plant, and each parental plant in a cross or outcross may be the same or different germplasm or breeding line. Corn event Zm_CSM63715 is passed from the original parent through each generation to the progeny. A “transgenic plant” or “plant”, therefore, can be the original transformant plant regenerated from the transformed plant cell and comprising the transgenic DNA and event, or a progeny plant of the original transformant plant, which may be separated from the transformant by one or more generations, that retains the transgenic DNA and event at the same specific location and sequence context in the plant's genome. The transformant or progeny plant may be homozygous or heterozygous for event Zm_CSM63715. In addition, a “transgenic plant” may comprise a plant having the transgenes stably inserted into the genome of at least one cell of the plant (i.e., corn event Zm_CSM63715 in at least one cell of the plant), and the plant may be chimeric or non-chimeric with respect to the transgenes and/or event. A transgenic plant is chimeric with respect to a transgene if not all cells of the plant comprise the transgenes.

The present disclosure describes introduction of event Zm_CSM63715 into corn, and thus the term “corn event Zm_CSM63715” is used to refer to the event herein. However, those of skill in the art will understand that event Zm_CSM63715 could be introduced into other varieties or related corn species by crosses, such as Zea diploperennis, Zea perennis, Zea luxurians, and Zea nicaraguensis.

Corn event Zm_CSM63715 provides to corn cells, plants, plant parts, seeds and progeny that comprise the event tolerance to PPO herbicides. The terms “PPO herbicide”, “PPO inhibitor”, and “PPO-inhibiting herbicide” are used interchangeably herein and refer to chemical agents that target and inhibit the enzymatic activity of a protoporphyrinogen oxidase (PPO). Corn event Zm_CSM63715 provides tolerance to various PPO herbicides, including, but not limited to flumioxazin, epyrifenacil (also referred to as S-3100 or rapidicil; IUPAC name: ethyl[(3-{2-chloro-5-[3,6-dihydro-3-methyl-2,6-dioxo-4-(trifluoromethyl)pyrimidin-1(2H)-yl]-4-fluorophenoxy}-2-pyridyl)oxy]acetate), lactofen, acifluorfen, pyraflufen, pyraflufen-ethyl, oxadiazon, butafenacil, pyridin-2-ylmethyl[(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl[(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl[(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate, cyanomethyl[(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate, methyl (2R)-2-{[(E)-({2-chloro-4-fluoro-5-[3-methyl-2,6-dioxo-4-(trifluoromethyl)-3,6-dihydropyrimidin-1(2H)-yl]phenyl}methylidene)amino]oxy}propanoate (flufenoximacil), cyclopropylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate, fomesafen, saflufenacil, sulfentrazone, tiafenacil, and trifludimoxazin.

Corn event Zm_CSM63715 is characterized as a single copy insertion into a site-directed locus in the corn genome, resulting in two new loci or junction sequences (e.g., the sequences set forth in SEQ ID NO:1, SEQ ID NO:2, SEQ ID NO:3, SEQ ID NO:4, SEQ ID NO:5, SEQ ID NO:6, SEQ ID NO:7 and SEQ ID NO:8) spanning portions of the inserted DNA and the corn genomic DNA that are not known to appear or exist naturally in the corn genome or other transgenic corn events, i.e., they are unique to event Zm_CSM63715. SEQ ID NO:1, SEQ ID NO:3, SEQ ID NO:5 and SEQ ID NO:7 span the 5′ junction of the corn genomic sequence and the transgenic DNA insert, and SEQ ID NO:2, SEQ ID NO:4, SEQ ID NO:6 and SEQ ID NO:8 span the 3′ junction. These junction sequences are useful in detecting the presence of the event Zm_CSM63715 in corn cells, seed, plants, plant parts, progeny, and plant products, such as corn commodity products. Polynucleotide or DNA molecular probes and/or primer pairs are described herein for use in identifying the presence of these various junction sequences in biological samples containing or derived from, or suspected of containing or being derived from, corn cells, seeds, plants, plant parts, progeny, or commodity products that contain the event Zm_CSM63715.

As used herein, the term “derived” or “derived from” in reference to a particular DNA molecule, amplicon or sequence in relation to a corn plant, plant part, seed, progeny, cell and/or corn plant product, such as a commodity product, means that the DNA molecule, amplicon or sequence is taken, purified, isolated, or made, directly or indirectly, from such corn plant, plant part, seed, progeny, cell and/or corn plant product, such as a commodity product. Alternatively, the term “derived” or “derived from” in reference to a corn plant product, such as a commodity product, in relation to corn plant, plant part, seed, progeny, or cell, means that the corn plant product is taken, purified, isolated, or made, directly or indirectly, from such corn plant, plant part, seed, progeny, or cell.

“Capable of being detected” refers to the ability of a particular DNA molecule, segment or sequence to be detected in a sample, such as by amplification and determining its presence, size or sequence such as by DNA sequence analysis, and/or binding of a probe to the target DNA molecule, segment or sequence.

A “sample” is intended to refer to any composition comprising or derived from, either directly or indirectly, a biological sample, source, or material. The sample may generally comprise corn DNA and/or substantially or completely pure, purified, or isolated corn DNA. A “biological sample” contains biological materials, including but not limited to DNA obtained or derived from, either directly or indirectly, the genome of a corn cell(s), tissue(s), seed(s), plant(s), plant part(s) and/or corn plant product(s), such as a commodity product(s). Such corn cell(s), tissue(s), seed(s), plant(s), plant part(s) and/or corn plant product(s), such as a commodity product(s), may comprise corn event Zm_CSM63715, or DNA molecule(s) and/or DNA segment(s) comprising corn event Zm_CSM63715. In some embodiments, a sample or biological sample may comprise corn cell(s), corn tissue(s), corn seed(s), corn plant(s), corn plant part(s) and/or corn plant product(s), whose cells or cellular membranes have been fractured (e.g., disrupted or opened) to release the contents of the corn cell(s) including genomic DNA or proteins and/or make the contents of the corn cell(s) including genomic DNA or proteins accessible or usable for assays or testing. “Directly” refers to directly obtaining DNA by a skilled artisan from the corn genome by fracturing corn cells (or by obtaining samples of corn that contain fractured corn cells) and exposing or using the genomic DNA or protein from corn cells for the purposes of detection. “Indirectly” refers to obtaining by a skilled artisan a target or specific reference DNA (e.g., a novel and unique junction segment(s) described herein as being diagnostic for the presence of the event Zm_CSM63715) in a particular sample, by means other than by obtaining directly via fracturing of corn cells or obtaining a sample of corn that contains fractured corn cells. Such indirect means include, but are not limited to, amplification of a DNA segment that contains a DNA sequence targeted by a particular probe(s) and/or primer set(s) designed to bind with specificity to or near the target sequence, or amplification of a DNA segment comprising all or part of a target sequence that can be measured and characterized (e.g., measured by migration or separation from other segments of DNA and/or identification in an effective matrix, such as an agarose or acrylamide gel or the like, or characterized by direct sequence analysis of the amplicon(s), or cloning of the amplicon(s) into a vector(s) and direct sequencing of the inserted amplicon(s) present within such vector(s)).

As used herein, the term “recombinant” refers to a non-naturally occurring DNA, protein, combination, or organism that would not normally be found or exist in nature, and is created by human intervention. As used herein, a “recombinant DNA molecule” is a DNA molecule comprising a combination of DNA molecules that would not naturally occur together and is the result of human intervention. Two or more elements of such combination of DNA sequences may be operably linked to one another. For example, a recombinant DNA molecule may comprise a combination of at least two DNA molecules heterologous with respect to each other, such as a DNA molecule that comprises a coding sequence operably linked to a heterologous promoter and/or other regulatory expression element(s), and/or a transgene and a heterologous plant genomic DNA adjacent to the transgene, and/or a DNA molecule that is artificially synthesized and comprises a polynucleotide sequence that deviates from any polynucleotide sequence that would normally exist in nature. A recombinant DNA molecule may comprise all or part of a junction sequence of the genome of the event and all or part of the transgenic insert of the genome of the event, and/or may comprise a recombinant or heterologous DNA fragment of corn event Zm_CSM63715. Examples of recombinant DNA molecules include a DNA molecule comprising at least one polynucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9 and SEQ ID NO:10; a polynucleotide having a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO: 9; and a complete complement of any of the foregoing. Such recombinant DNA molecules can be derived from a corn plant, seed, plant part, plant cell, progeny plant, or commodity product comprising corn event Zm_CSM63715.

Alternatively, such recombinant DNA molecules can be comprised in a corn plant, seed, plant part, plant cell, or progeny plant comprising corn event Zm_CSM63715, or a commodity product produced therefrom. A representative sample of seed comprising corn event Zm_CSM63715 has been deposited as ATCC Accession No. PTA-127361. Such recombinant DNA molecules can be formed by the insertion of a heterologous nucleic acid molecule into the genomic DNA of a corn plant or corn cell. Such recombinant DNA molecules can be an amplicon diagnostic for the presence of corn event Zm_CSM63715.

As used herein, a “recombinant” in reference to a plant, plant part, seed, plant cell, or progeny is a plant, plant part, seed, plant cell or progeny that would not normally exist in nature, is the result of human intervention, and contains a transgenic DNA molecule stably integrated into the genome of the plant, plant part, seed, plant cell, or progeny. As a result of such genomic insertion, the recombinant or transgenic plant, plant part, seed, plant cell, or progeny is something new and distinctly different from any related wildtype or naturally occurring plant, plant part, seed, plant cell or progeny. An example of a recombinant plant is a corn plant containing the corn event Zm_CSM63715.

As used herein, the term “transgene” refers to a DNA molecule artificially incorporated into an organism's genome as a result of human intervention, such as by plant transformation methods. A transgene may be heterologous to the organism. The term “transgenic insert” as used herein refers to the foreign or heterologous DNA inserted by plant transformation techniques into the corn genome to produce corn event Zm_CSM63715. The sequence for the transgenic insert of corn event Zm_CSM63715 is provided as SEQ ID NO:9.

As used herein, the term “heterologous” in reference to a combination of two or more DNA sequences or elements means that the two or more DNA sequences or elements do not normally exist together as such combination in nature without human intervention. For example, a DNA molecule may be from a first species or a recombinant DNA molecule, and inserted into the genome of a second species. The DNA molecule would thus be heterologous to the genome and the organism. As used herein, the term “heterologous” in reference to a DNA molecule, construct, sequence or protein in relation to a plant, microorganism, plant cell or plant genome means that the DNA molecule, construct, sequence or protein does not exist in nature as part of such a plant, microorganism, plant cell or plant genome, and/or does not exist in the same physical or genomic location, context or orientation as part of such a plant, microorganism, plant cell or plant genome in nature, without human intervention.

As used herein, the term “chimeric” refers to a single DNA molecule produced by fusing a first DNA molecule to a second DNA molecule, where neither first nor second DNA molecule would normally be found in that configuration fused to the other. The chimeric DNA molecule is thus a new DNA molecule not normally found in nature. An example of a chimeric DNA molecule is a DNA molecule comprising at least one sequence selected from SEQ ID NO:1-10.

As used herein, the term “isolated” in reference to a molecule means that the molecule is at least partially separated from other molecules that are normally associated with it in its native or natural state. In some embodiments, the term “isolated” refers to a DNA molecule that is at least partially separated from the nucleic acids or polynucleotide or DNA sequence(s) that normally flank and are covalently linked to the sequence of the DNA molecule in its native or natural state. An “isolated” DNA molecule may have a DNA sequence corresponding to a portion of the genome of a plant cell without other genomic DNA sequence(s) that normally flank and are covalently linked to the DNA sequence in nature. Such an “isolated” DNA molecule may comprise all or part of a transgene and/or transgenic event, which may comprise all or part of corn event Zm_CSM63715 or the transgenes or expression cassettes described herein. Nucleic acid sequences or elements, such as a coding sequence, intron sequence, 5′ UTR, promoter sequence, 3′ UTR, and the like, that are naturally found within the DNA of the genome of an organism are not considered to be “isolated” so long as the element is within the genome of the organism and at the location within the genome in which it is naturally found. However, each of these elements, and subparts of these elements, would be “isolated” within the scope of this disclosure so long as the element or subpart is not within the genome of the organism, and at the location within the genome of the organism, in which it is naturally found. An “isolated” DNA molecule may be any recombinant DNA molecule or amplification product or amplicon, and/or may comprise any DNA sequence removed from its natural or biological state and covalently fused to another DNA molecule or sequence with which it is not associated in nature. Such an isolated DNA molecule could be created by the use of biotechnology techniques, such as by making a recombinant DNA or integrating a foreign or heterologous DNA molecule into the chromosome of a cell, plant, or seed. Thus, any DNA molecule comprising a transgenic, recombinant, chimeric or artificial nucleotide sequence, transgene or expression cassette would be considered to be an “isolated” DNA molecule since these sequences are not naturally occurring, regardless of whether the sequence, transgene or expression cassette is present within a plasmid, vector or construct used to transform plant cells, within the genome of a plant, plant part, plant tissue, plant cell or progeny, or is present in detectable amounts in tissues, progeny, biological samples or commodity products derived from a plant, plant part, plant tissue, progeny or plant cell. A recombinant DNA molecule or sequence, or any fragment derived therefrom, comprising all or part of a transgene or junction sequence of the corn event Zm_CSM63715 would therefore also be considered to be “isolated.” An “isolated” DNA molecule may be extracted or purified from a transgenic plant(s), plant part(s), plant cell(s) and/or tissue(s), or may be present in a homogenate, extract or lysate from any such transgenic plant(s), plant part(s), plant cell(s) and/or tissue(s), or may be produced as an amplicon or amplification product from plant genomic DNA and/or extracted or purified DNA from transgenic plant(s), plant part(s), plant cell(s) and/or tissue(s), or a homogenate, extract or lysate from plant(s), plant part(s), plant cell(s) and/or tissue(s). For the purposes of this disclosure, any transgenic polynucleotide or DNA sequence, i.e., the nucleotide sequence of the DNA inserted into the genome of a plant or bacterium, or present in an extrachromosomal vector, would be considered to be an “isolated” nucleotide or DNA sequence whether it is present within the plasmid or similar structure used to transform the cells, within the genome of the plant or bacterium, or present in detectable amounts in tissues, progeny, biological samples or commodity products derived from the plant or bacterium. An “isolated” DNA molecule is a chemical or biochemical molecule, regardless of whether the molecule is referred to as a nucleic acid, a nucleic acid sequence, a polynucleotide sequence, a DNA sequence, a nucleic acid molecule, a polynucleotide molecule, a DNA molecule, or the like. An “isolated” molecule can provide industrial applicability when present in a plant cell or in a plant genome or when present outside of a plant cell, and therefore, provides and exhibits (and is intended to provide and exhibit) utility regardless of where the molecule is located.

As used herein, the term “correspond” or “corresponding”, or the like, when used in the context of a nucleotide position, mutation, insertion and/or substitution in any given polynucleotide (e.g., SEQ ID NO:9) with respect to a reference polynucleotide sequence (e.g., SEQ ID NO:10) refers to the position(s) of the polynucleotide residue(s) in the given sequence that has identity to the residue(s) in the reference nucleotide sequence when the given polynucleotide is aligned to the reference polynucleotide sequence using a global or local sequence alignment algorithm.

DNA molecules, fragments, and their corresponding DNA sequences, as well as methods of detection are provided. As used herein, the terms “DNA”, “DNA molecule” and “nucleic acid molecule” refer to a deoxyribonucleic acid (DNA) molecule. A DNA molecule may be of genomic or synthetic origin and/or comprise a recombinant or heterologous DNA molecule or sequence. A DNA molecule may be described by convention from the 5′ (upstream) end to the 3′ (downstream) end. As used herein, the term “DNA sequence” refers to the polynucleotide sequence of a DNA molecule, i.e., the sequence of consecutive nucleotides in the DNA molecule. As used herein in reference to nucleotides of a polynucleotide or DNA sequence or molecule, the terms “consecutive” and “contiguous” are interchangeable and synonymous and refer to the 5′ to 3′ order of nucleotides in a polynucleotide or DNA sequence, strand or molecule without any gap or interruption between them. The nomenclature used is that required by Title 37 of the United States Code of Federal Regulations § 1.822 and set forth in the tables in WIPO Standard ST.25 (1998), Appendix 2, Tables 1 and 3. By convention, DNA sequences and fragments thereof are disclosed with reference to the 5′ to 3′ direction of only one strand of the two complementary DNA sequence strands of a DNA molecule. By implication and intent, the complementary sequences of the sequences provided here (the sequences of the complementary strand), also referred to in the art as the reverse complementary or reverse complement sequences, are within the scope of the present disclosure and are expressly intended to be within the scope of the subject matter claimed. As used herein references to SEQ ID NOs:1-10 and fragments thereof include and refer to the sequence of the complementary strand and fragments thereof.

Also provided is a nucleic acid molecule comprising a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to the full length of any one of SEQ ID NOs:1-10.

For example, a nucleic acid molecule is provided comprising a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to the full length of SEQ ID NO:10 or to the full length of SEQ ID NO: 9.

A DNA molecule, or a fragment derived therefrom, can also be extracted from plant(s), plant part(s), seed(s), progeny or plant cell(s), or a homogenate, extract or lysate from plant(s), plant part(s), plant cell(s) or seed(s) or progeny, or can be produced as an amplicon from extracted, purified or isolated DNA from plant part(s), plant cell(s) and/or tissue(s), progeny, or a homogenate, extract or lysate from plant(s), plant part(s), plant cell(s), progeny and/or seeds, which may further comprise corn event Zm_CSM63715.

As used herein, the term “percent sequence identity” or “% sequence identity” refers to the percentage of identical nucleotides or amino acids in a linear polynucleotide or polypeptide sequence of a reference (“query”) sequence (or its complementary strand) as compared to a test (“subject”) sequence (or its complementary strand) when the two sequences are optimally aligned (with appropriate nucleotide or amino acid insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and by computerized implementations of these algorithms such as GAP, BESTFIT, FASTA, and TFASTA available as part of the Sequence Analysis software package of the GCG® Wisconsin Package@ (Accelrys Inc., San Diego, Calif.), MEGAlign (DNAStar Inc., 1228 S. Park St., Madison, Wis. 53715), and MUSCLE (version 3.6) (Edgar, “MUSCLE: multiple sequence alignment with high accuracy and high throughput” Nucleic Acids Research 32(5):1792-7 (2004)) for instance with default parameters. An “identity fraction” for aligned segments of a test sequence and a reference sequence is the number of identical components that are shared by the two aligned sequences divided by the total number of components in the portion of the reference sequence segment being aligned, that is, the entire reference sequence or a smaller defined part of the reference sequence. Percent sequence identity is represented as the identity fraction multiplied by 100. The comparison of one or more sequences may be to a full-length sequence or a portion thereof, or to a longer sequence. Corn plants, progeny, seeds, cells, plant parts and commodity products comprising a detectable amount of a polynucleotide having a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO:9 are within the scope of the present disclosure.

As used herein, the term “fragment” refers to a smaller piece or sequence of a larger or whole DNA molecule or sequence. For example, a fragment of any one of SEQ ID NOs:1-12 and SEQ ID NOs:164-165 may include a sequence that is at least about 10 consecutive nucleotides, at least about 11 consecutive nucleotides, at least about 12 consecutive nucleotides, at least about 13 consecutive nucleotides, at least about 14 consecutive nucleotides, at least about 15 consecutive nucleotides, at least about 16 consecutive nucleotides, at least about 17 consecutive nucleotides, at least about 18 consecutive nucleotides, at least about 19 consecutive nucleotides, at least about 20 consecutive nucleotides, at least about 21 consecutive nucleotides, at least about 22 consecutive nucleotides, at least about 23 consecutive nucleotides, at least about 24 consecutive nucleotides, at least about 25 consecutive nucleotides, at least about 30 consecutive nucleotides, at least about 35 consecutive nucleotides, at least about 40 consecutive nucleotides, at least about 45 consecutive nucleotides, at least about 50 consecutive nucleotides, at least about 60 consecutive nucleotides, at least about 70 consecutive nucleotides, at least about 80 consecutive nucleotides, at least about 90 consecutive nucleotides, at least about 100 consecutive nucleotides, at least about 150 consecutive nucleotides, at least about 200 consecutive nucleotides, at least about 250 consecutive nucleotides, at least about 300 consecutive nucleotides, at least about 400 consecutive nucleotides, or at least about 500 consecutive nucleotides of the larger, whole or complete DNA molecule or sequence.

For example, a “fragment” of the transgenic insert sequence (SEQ ID NO: 9) of corn event Zm_CSM63715 can comprise at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 60, at least about 70, at least about 80, at least about 90, at least about 100, at least about 150, at least about 200, at least about 250, at least about 300, at least about 400, or at least about 500 consecutive nucleotides of SEQ ID NO: 9. In addition, the present disclosure encompasses nucleotide sequences that are at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to SEQ ID NO: 9 or any fragment thereof.

Similarly, a fragment of the 5′ flank (SEQ ID NO:11 or SEQ ID NO:164) or 3′ flank (SEQ ID NO:12 or SEQ ID NO:165) of corn event Zm_CSM63715 can comprise at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 60, at least about 70, at least about 80, at least about 90, at least about 100, at least about 150, at least about 200, at least about 250, at least about 300, at least about 400, or at least about 500 consecutive nucleotides of SEQ ID NO:11 or SEQ ID NO:164; or SEQ ID NO:12 or SEQ ID NO:165. In addition, the present disclosure encompasses nucleotide sequences that are at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to SEQ ID NO:11 or 12, or SEQ ID NO:164 or 165, or any fragment of either thereof.

As used herein, the term “about” indicates a value or a range of values which would be understood as an equivalent of a stated value and can be greater or lesser than the value or range of values stated. Each value or range of values preceded by the term “about” is also intended to encompass the embodiment of the stated absolute value or range of values.

The term “or” is used herein to mean “and/or” unless explicitly indicated to refer to alternatives only or the alternatives are mutually exclusive. Thus, the term “and/or” as used herein in a phrase such as “X and/or Y” is intended to include “X and Y”, “X or Y”, “X” (alone), and “Y” (alone). Likewise, the term “and/or” as used in a phrase such as “X, Y, and/or Z” is intended to encompass each of the following embodiments: X (alone); Y (alone); Z (alone); X and Y; X and Z; Y and Z; X, Y, and Z; X, Y, or Z; X or Z; Y or Z; Y or Z.

When used in conjunction with the word “comprising” or other open language, the words “a” and “an” denote “one or more,” unless specifically noted otherwise. The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and also covers other unlisted steps.

Corn event Zm_CSM63715 is characterized as a transgenic insertion into a locus in the corn genome, resulting in two new junctions (or joining or connection points). The DNA sequence of the region spanning the connection by phosphodiester bond linkage of one end of the transgenic insert to the flanking corn genomic DNA is referred to herein as a “junction.” In other words, a junction is the connection point or covalent linkage of one end of the transgenic insert and the flanking genomic DNA as one contiguous molecule, and is formed by the insertion of a heterologous nucleic acid molecule into the corn genomic DNA. One junction is found at the 5′ end of the transgenic insert and the other is found at the 3′ end of the transgenic insert, referred to herein as the 5′ and 3′ junctions, respectively. A “junction sequence” refers to a DNA sequence of any length of consecutive nucleotides that spans the 5′ or 3′ junction of a transgenic event in the plant genome. For a “junction sequence” to be specific to a junction between a transgenic event and a flanking genomic sequence, the junction sequence will generally comprise a sufficient number of consecutive nucleotides at one end of the insertion and a sufficient number of consecutive nucleotides of the flanking genomic sequence. According to some embodiments, a “junction sequence” may comprise (i) at least five (5) consecutive nucleotides, at least ten (10) consecutive nucleotides, at least fifteen (15) consecutive nucleotides, at least twenty (20) consecutive nucleotides, at least twenty five (25) consecutive nucleotides, at least thirty (30) consecutive nucleotides, at least thirty five (35) consecutive nucleotides, at least forty (40) consecutive nucleotides, at least forth five (45) consecutive nucleotides, or at least fifty (50) consecutive nucleotides at one end of the insertion and (ii) at least five (5) consecutive nucleotides, at least ten (10) consecutive nucleotides, at least fifteen (15) consecutive nucleotides, at least twenty (20) consecutive nucleotides, at least twenty five (25) consecutive nucleotides, at least thirty (30) consecutive nucleotides, at least thirty five (35) consecutive nucleotides, at least forty (40) consecutive nucleotides, at least forth five (45) consecutive nucleotides, or at least fifty (50) consecutive nucleotides of the flanking genomic DNA sequence, although it is understood that any length of consecutive nucleotides spanning a junction of a transgenic event in a plant genome may be a junction sequence. Junction sequences of corn event Zm_CSM63715 are apparent to, and a variety of junction sequences of corn event Zm_CSM63715 can be determined by, one of skill in the art using SEQ ID NO:10. In SEQ ID NO:10, the 5′ junction is at nucleotides 1,000-1,001, and the 3′ junction is at nucleotides 4,552-4,553. Illustrative junction sequences of corn event Zm_CSM63715 are provided as SEQ ID NOs:1-8. FIG. 1 illustrates the physical arrangement and locations of the illustrative junction sequences, arranged from 5′ to 3′ (left to right), relative to SEQ ID NO:10. The DNA sequence for the transgenic insert of corn event Zm_CSM63715 is provided as SEQ ID NO:9. The DNA sequence of the transgenic insert and the corn genomic DNA flanking each side of the transgenic insert is provided as SEQ ID NO:10. The 5′ junction sequences are provided as SEQ ID NO:1, SEQ ID NO:3, SEQ ID NO:5, and SEQ ID NO:7. The 3′ junction sequences are provided as SEQ ID NO:2, SEQ ID NO:4, SEQ ID NO:6, and SEQ ID NO:8. The junction sequences of corn event Zm_CSM63715 may be present as part of the genome of a plant, seed, plant part, progeny or plant cell containing corn event Zm_CSM63715, or a DNA molecule containing all or part of event Zm_CSM63715. The identification of any one or more of the junction sequences in a DNA molecule or a sample from a plant, plant part, seed, progeny, cell or commodity product indicates that the DNA molecule or plant, plant part, seed, progeny, cell or commodity product contains or comprises event Zm_CSM63715, or was obtained from a corn plant, plant part, seed, progeny, cell or commodity product containing or comprising event Zm_CSM63715, and is diagnostic for the presence of corn event Zm_CSM63715.

The junction sequences described herein are diagnostic for the presence of all or part of corn event Zm_CSM63715. Thus, the identification or detection, directly or indirectly, of one or more of SEQ ID NO:1, SEQ ID NO:2, SEQ ID NO:3, SEQ ID NO:4, SEQ ID NO:5, SEQ ID NO:6, SEQ ID NO:7, SEQ ID NO:8, and SEQ ID NO:10 in a sample or DNA molecule derived from a corn plant, plant part, seed, progeny, cell, or a commodity product is diagnostic that the corn plant, plant part, seed, progeny, cell, or a commodity product has or comprises all or part of corn event Zm_CSM63715. The identification or detection, directly or indirectly, of a 5′ junction sequence and/or a 3′ junction sequence (each as provided or described herein) in a sample or DNA molecule derived from a corn plant, plant part, seed, progeny, cell, or a commodity product is diagnostic that the corn plant, plant part, seed, progeny, cell, or a commodity product has or comprises corn event Zm_CSM63715. The present disclosure thus provides a DNA molecule that comprises at least one of the nucleotide sequences provided as SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10. Any segment of DNA derived from transgenic corn event Zm_CSM63715 that is sufficient to include at least one of the sequences provided as SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10 is within the scope of the present disclosure. In addition, any DNA or polynucleotide molecule or sequence comprising a sequence complementary to any of the sequences described herein is also within the scope of the present disclosure.

Polynucleotide molecules are provided, which may be single or double stranded, that can be used either as primers or probes for detecting the presence of DNA comprising all or part of event Zm_CSM63715 in a sample derived from a corn plant, plant part, seed, progeny, cell, or a commodity product. Such primers or probes are specific for a target polynucleotide sequence and, as such, are useful for the identification of corn event Zm_CSM63715 polynucleotide by the methods described herein. A primer or probe can hybridize to a target polynucleotide sequence to allow for specific detection or amplification of a polynucleotide molecule that comprises, or is covalently linked and associated with, the target polynucleotide sequence. The primers and/or probes may be chosen to identify and distinguish detection of a particular transgenic event and not only the presence of a transgene in a plant genome. The target polynucleotide sequence may comprise all or part of corn event Zm_CSM63715, a junction sequence and/or flanking genomic DNA. Probes and primers according to the present disclosure may have (i) complete or 100% sequence complementarity (i.e., 100% complementary) to a target polynucleotide sequence or (ii) incomplete sequence complementarity to a target polynucleotide, such as at least 60% complementary, at least 65% complementary, at least 70% complementary, at least 75% complementary, at least 80% complementary, at least 85% complementary, at least 90% complementary, at least 95% complementary, or at least 99% complementary to the target polynucleotide sequence as long as the probe or primer has sufficient complementarity to the target polynucleotide sequence to hybridize to the target polynucleotide sequence under stringent hybridization conditions that are suitable and necessary for use of the probe or primer in the relevant amplification or detection assay, reaction or method. As understood in the art, the percentage complementarity of a primer or probe may be lower if the length of the primer or probe is longer, and depends on the stringency and use. Provided are illustrative polynucleotide molecules that can be used either as primers or probes for detecting the presence of corn event Zm_CSM63715 in a sample. Detection of the presence of corn event Zm_CSM63715 may be done by using methods known in the art, such as thermal or isothermal amplification of nucleic acids or nucleic acid hybridization techniques (such as Northern analysis and Southern analysis).

A “probe” is a nucleic acid molecule that is complementary to a strand of a target nucleic acid and is useful in hybridization detection methods. Probes include not only deoxyribonucleic or ribonucleic acids but also polyamides and other probe materials that bind specifically to a target DNA sequence and the detection of such binding can be useful in detecting the presence or absence of the target DNA sequence. A probe may be attached to a conventional detectable label or reporter molecule, such as a radioactive isotope, ligand, chemiluminescent agent, or enzyme. Such a probe is complementary to a strand of a target nucleic acid and, in the case of the present disclosure, to a strand of DNA from event Zm_CSM63715 whether from an event Zm_CSM63715-containing plant or from a sample that includes event Zm_CSM63715 DNA.

Provided herein is a DNA molecule comprising a polynucleotide segment of sufficient length to function as a DNA probe that hybridizes specifically under stringent hybridization conditions with corn event Zm_CSM63715 DNA in a sample, wherein detecting hybridization of the DNA molecule under the stringent hybridization conditions is diagnostic for the presence of corn event Zm_CSM63715 in the sample. Also provided is a DNA molecule comprising a polynucleotide segment of sufficient length to function as a DNA probe specific for detecting in a sample at least one of: (i) a 5′ junction sequence between flanking corn genomic DNA and the transgenic insert of corn event Zm_CSM63715; (ii) a 3′ junction sequence between the transgenic insert of corn event Zm_CSM63715 and flanking corn genomic DNA; (iii) SEQ ID NO:9; and (iv) a fragment of SEQ ID NO:9 comprising a sufficient length of contiguous nucleotides of SEQ ID NO:9 to identify the sequence as a fragment of the transgenic insert of Zm_CSM63715. An illustrative DNA sequence useful as a probe for detecting corn event Zm_CSM63715 is provided as SEQ ID NO:16. Other DNA sequences useful as probes for detecting corn event Zm_CSM63715 include SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; and a complement of any of the foregoing.

A “primer” is a DNA molecule or oligonucleotide that is designed for use in specific annealing or hybridization methods that involve an in vitro amplification reaction. A pair of primers may be used with template DNA (such as a sample of corn event Zm_CSM63715 genomic DNA) in a thermal amplification reaction (such as polymerase chain reaction (PCR)) or any other suitable amplification method known in the art to produce an amplification product or amplicon, where the amplicon produced from such reaction would have a DNA sequence corresponding to sequence of the template DNA located between the two sites where the primers hybridized to the template DNA.

DNA amplification reactions, methods and techniques are known to those skilled in art. DNA amplification can be accomplished by any of the various nucleic acid amplification methods known in the art, including thermal and isothermal amplification methods such as polymerase chain reaction (PCR) and loop-mediated isothermal amplification (LAMP). Amplification methods are known in the art and are described, inter alia, in U.S. Pat. Nos. 4,683,195 and 4,683,202 and in PCR Protocols: A Guide to Methods and Applications, ed. Innis et al., Academic Press, San Diego, 1990. PCR amplification methods have been developed to amplify up to 22 kb (kilobase) of genomic DNA and up to 42 kb of bacteriophage DNA (Cheng et al., 1994). These methods as well as other methods known in the art of DNA amplification may be used in the practice of the present disclosure. Examples of DNA amplification methods include PCR, Recombinase Polymerase Amplification (RPA) (see for example U.S. Pat. No. 7,485,428), Strand Displacement Amplification (SDA) (see for example, U.S. Pat. Nos. 5,455,166 and 5,470,723), Transcription-Mediated Amplification (TMA) (see for example, Guatelli et al., 1990), Rolling Circle Amplification (RCA) (see for example, Fire and Xu, 1995; Liu, et al., 1996; Lizardi, et al., 1998; U.S. Pat. Nos. 5,714,320 and 6,235,502), Helicase Dependent Amplification (HDA) (see for example Vincent et al., 2004; U.S. Pat. No. 7,282,328), Multiple Displacement Amplification (MDA) (see for example Dean et al., 2002) and Loop-Mediated Isothermal Amplification (LAMP) (see for example Notomi et al., 2000). A sequence of the heterologous DNA insert and/or flanking genomic DNA sequence from corn event Zm_CSM63715 can be verified or tested by amplifying such DNA molecules from corn seed containing event Zm_CSM63715 DNA or corn plants grown from the corn seed containing event Zm_CSM63715 DNA, using primers derived from the sequences provided herein, followed by standard DNA sequencing of the PCR amplicon or a cloned DNA fragment thereof.

As used herein, an “amplification product” or “amplified DNA” or “amplicon” refers to the nucleic acid or DNA molecule or segment produced by a nucleic acid amplification reaction or method as further described herein, which is directed to a target nucleic acid or DNA molecule that is part of a template nucleic acid molecule. Amplification or amplifying refers to making multiple copies of a target DNA molecule or segment from a template DNA. For example, to determine whether a corn plant, plant part, seed, progeny or plant cell, resulting from selfing or outcross of a parent comprising corn event Zm_CSM63715 contains corn event Zm_CSM63715, DNA may be extracted from the corn plant tissue sample and subjected to an amplification reaction or method using a pair of primers that are specific for a target sequence that is uniquely associated or part of corn event Zm_CSM63715, such as, for example, a first primer derived from a genomic DNA sequence in the region flanking the heterologous inserted DNA of corn event Zm_CSM63715 that is elongated by polymerase 5′ to 3′ in the direction of the inserted DNA, and a second primer derived from the heterologous inserted DNA molecule that is elongated by the polymerase 5′ to 3′ in the direction of the flanking genomic DNA from which the first primer is derived. The amplicon may range in length depending on the length of the intervening polynucleotide or DNA sequence between the two primer target sequences in the template DNA molecule. Alternatively, a primer pair can be derived from the genomic sequence on both sides of the inserted heterologous DNA so as to produce an amplicon that includes the entire insert polynucleotide sequence (e.g., a forward primer targeted to the genomic portion on the 5′ end of SEQ ID NO:10 (i.e. upstream of SEQ ID NO:9) and a reverse primer targeted to the genomic portion on the 3′ end of SEQ ID NO:10 (i.e. downstream of SEQ ID NO:9) that amplifies a DNA molecule comprising the inserted DNA sequence (SEQ ID NO:9) identified herein in the corn event Zm_CSM63715 genome. The use of the term “amplicon” specifically excludes primer dimers that may be formed in a DNA amplification reaction.

Provided herein is a pair of DNA molecules comprising a first DNA molecule and a second DNA molecule, wherein the first and the second DNA molecules comprise a fragment of SEQ ID NO:10 or a complement thereof and function as DNA primers when used together in an amplification reaction with DNA comprising corn event Zm_CSM63715 to produce an amplicon diagnostic for corn event Zm_CSM63715 in a sample. For example, the first and second DNA molecules can comprise SEQ ID NO:14 and SEQ ID NO:15. The amplicon described herein may comprise a DNA sequence comprising one or more of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or a fragment of any of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10 wherein the fragment is at least 10 nucleotides in length and comprises nucleotides 1,000-1,001 or 4,552-4,553 of SEQ ID NO:10. According to present embodiments, the sequence of an amplicon comprises at least one junction sequence or two junction sequences, such as a 5′ junction sequence and/or a 3′ junction sequences for corn event Zm_CSM63715. Amplification and detection of such an amplicon is indictive or diagnostic for corn event Zm_CSM63715.

For practical purposes, one should design primers which produce amplicons of a limited size range, for example, between 100 to 1000 bases. Smaller (shorter polynucleotide length) sized amplicons in general are more reliably produced in thermal amplification reactions, allow for shorter cycle times, and can be easily separated and visualized on agarose gels or adapted for use in endpoint TaqMan®-like assays. Smaller amplicons can be produced and detected by methods known in the art of DNA amplicon detection. In addition, amplicons produced using the primer pairs can be cloned into vectors, propagated, isolated, and sequenced or can be sequenced directly with methods well established in the art. Any primer pair of forward and reverse primers, which may be identical or complementary to part of SEQ ID NO:10, such as SEQ ID NOs:14 and 15, that is useful in a DNA amplification method to produce an amplicon diagnostic for corn event Zm_CSM63715 or progeny thereof is an aspect of the disclosure. Any single isolated DNA polynucleotide primer molecule comprising at least 15 contiguous nucleotides of SEQ ID NO:10, or its complement that is useful in a DNA amplification method to produce an amplicon diagnostic for corn event Zm_CSM63715 or progeny thereof is an aspect of the disclosure. Any single isolated DNA polynucleotide primer molecule comprising at least 15 contiguous nucleotides of SEQ ID NO:11 or SEQ ID NO:12, or its complement that is useful in a DNA amplification method to produce an amplicon diagnostic for plants comprising corn event Zm_CSM63715 or progeny thereof is an aspect of the disclosure. Any single isolated DNA polynucleotide primer molecule comprising at least 15 contiguous nucleotides of SEQ ID NO:9, or its complement that is useful in a DNA amplification method to produce an amplicon diagnostic for corn event Zm_CSM63715 or progeny thereof is an aspect of the disclosure.

A primer is typically designed to hybridize specifically to a complementary target DNA strand to form a hybrid between the primer and the target DNA strand. Hybridization or binding of a primer to the complementary target DNA strand is a point of recognition by a polymerase to begin extension of the primer (i.e., polymerization of additional nucleotides into a lengthening nucleotide molecule) using the target DNA strand as a template. Primer pairs refer to use of two primers binding opposite strands of a double stranded nucleotide segment for the purpose of amplifying the polynucleotide segment between the positions targeted for binding by the individual members of the primer pair, typically in a thermal amplification reaction or other conventional nucleic-acid amplification methods. Primer pairs are typically designed to hybridize to different nearby target positions of a template DNA molecule on opposing strands of the template DNA molecule such that the intervening region or sequence between the two primers can be specifically amplified for use or detection through multiple rounds of amplification.

To detect the presence or absence of corn event Zm_CSM63715, the target positions and/or the intervening region or sequence of a template DNA molecule may comprise at least one junction sequence and/or at least a portion of the insert of corn event Zm_CSM63715. To detect the absence of corn event Zm_CSM63715, the target positions and/or the intervening region or sequence of a template DNA molecule may comprise corn genomic DNA that does not include a junction sequence or any portion of the insert of corn event Zm_CSM63715. Thus, the presence or absence of an amplicon with a primer pair may be diagnostic of the presence or absence, respectively, of corn event Zm_CSM63715 in a DNA molecule or sample, or vice versa. This may also be possible with more than one primer pair. For example, a first primer pair may produce a first amplicon if corn event Zm_CSM63715 is present, and a second primer pair may produce a second amplicon if corn event Zm_CSM63715 is absent or not present. Alternatively, the size of an amplicon produced in an amplification reaction may also be diagnostic of the presence or absence of corn event Zm_CSM63715 in a DNA molecule or sample-e.g., a primer pair may produce a first amplicon of a first size if corn event Zm_CSM63715 is present or a second amplicon of a second size if corn event Zm_CSM63715 is absent and not present; or a first primer pair may produce a first amplicon of a first size if corn event Zm_CSM63715 is present, and a second primer pair may produce a second amplicon of a second size if corn event Zm_CSM63715 is absent or not present.

According to some of these embodiments, at least two primer pairs may be used wherein at least one of the primer pairs is used as an internal control and is not associated with corn event Zm_CSM63715.

According to present embodiments, a primer pair to detect the presence or absence of all or part of corn event Zm_CSM63715 in a DNA molecule or sample comprises a first primer and a second primer, wherein the first primer is complementary to a 5′ flanking genomic DNA sequence and the second primer is complementary to a sequence within the transgenic insert; or wherein the first primer is complementary to a 5′ flanking genomic DNA sequence and the second primer is complementary to a 3′ flanking genomic DNA sequence; or wherein the first primer is complementary to a sequence within the transgenic insert and the second primer is complementary to a 3′ flanking genomic DNA sequence. Each reference in this paragraph to a primer complementary to a 5′ flanking genomic DNA sequence, a 3′ flanking genomic DNA sequence, or a sequence within the transgenic insert of corn event Zm_CSM63715 is also intended to potentially include a primer complementary to the reverse complement or opposing strand of the respective 5′ flanking genomic DNA sequence, 3′ flanking genomic DNA sequence, or sequence within the transgenic insert of corn event Zm_CSM63715.

Illustrative DNA molecules useful as primers are provided as SEQ ID NO:14 and SEQ ID NO:15. The primer pair SEQ ID NO:14 and SEQ ID NO:15 can be useful as a first primer (corresponding to a sequence within the transgenic insert) and a second primer (complementary to a 3′ flanking genomic DNA sequence), wherein each primer has sufficient length of consecutive nucleotides of SEQ ID NO:10 or a sequence complementary to SEQ ID NO:10 to function as DNA primers that, when used together in an amplification reaction with template DNA derived from corn event Zm_CSM63715, hybridize to opposite strands of the template DNA and produce an amplicon diagnostic for corn event Zm_CSM63715 DNA in a sample. The primer pair SEQ ID NO:20 (corresponding to a 5′ flanking genomic DNA sequence) and SEQ ID NO:21 (complementary to a 3′ flanking genomic DNA sequence) are useful as a first primer and a second primer, wherein each primer has sufficient length of consecutive nucleotides of a locus within the corn genome to function as DNA primers that, when used together in a thermal amplification reaction with template DNA, to produce an amplicon indictive or diagnostic for the wildtype DNA for the zygosity of Zm_CSM63715 event DNA in a sample. An amplicon diagnostic for event Zm_CSM63715 comprises a sequence not naturally found in the corn genome.

A primer may further comprise an oligo tail sequence such as those used in the Kompetitive Allele-Specific PCR (KASP™) method. The allele-specific primers each harbor a unique tail sequence that corresponds with a universal FRET (fluorescence resonant energy transfer) cassette; one labelled with FAM™ dye and the other with HEX™ dye. During thermal cycling, the relevant allele-specific primer binds to the template and elongates, thus attaching the tail sequence to the newly synthesized strand. The complement of the allele-specific tail sequence is then generated during subsequent rounds of PCR, enabling the FRET cassette to bind to the DNA. The FRET cassette is no longer quenched and emits fluorescence.

Methods for designing and using primers and probes are well known in the art. DNA molecules comprising fragments of SEQ ID NOs:1-10 are useful as primers and probes for detecting corn event Zm_CSM63715 and can readily be designed by one of skill in the art using the sequences provided herein. Such probes and primers are selected to be of sufficient length and sequence complementarity to a target sequence to hybridize specifically to a target sequence under stringency hybridization conditions. Probes and primers may have a complete sequence complementarity or identity with the target sequence, although probes and primers differing from the target sequence in terms of identity or complementarity but retaining the ability to form a stable double-stranded structure under particular hybridization conditions or reaction conditions and to hybridize to the target sequence may be designed by conventional methods.

Any conventional nucleic acid hybridization or amplification method can be used to identify or detect the presence of a target DNA from a transgenic plant, such as corn event Zm_CSM63715, in a sample. Polynucleotide molecules or DNA molecules, also referred to as “polynucleotide segment or fragment of sufficient length” or “sufficient length of contiguous or consecutive nucleotides” therefore are capable of specifically hybridizing to a target DNA sequence under certain hybridization conditions or reaction conditions. As used herein, the term “of sufficient length” refers to any length that is sufficient to be useful in a detection method of choice. Probes and primers are generally at least about 8 nucleotides, at least about 10 nucleotides, at least about 12 nucleotides, at least about 14 nucleotides, at least about 16 nucleotides, at least about 18 nucleotides, at least about 20 nucleotides, at least about 22 nucleotides, at least about 24 nucleotides, at least about 26 nucleotides, at least about 28 nucleotides, or at least about 30 nucleotides or more in length. Such probes and primers hybridize specifically to a target DNA sequence under stringent hybridization conditions.

As used herein, two nucleic acid molecules are capable of specifically hybridizing to one another if the two molecules are capable of forming an anti-parallel, double-stranded nucleic acid structure. A nucleic acid molecule is the “complement” of another nucleic acid molecule if they exhibit complete complementarity. As used herein, two nucleic acid molecules exhibit “complete complementarity” and are “completely complementary” if every nucleotide of the first nucleic acid molecule is complementary to every nucleotide of the second nucleic acid molecule when they are aligned. Two molecules are “minimally complementary” if they can hybridize to one another with sufficient stability to permit them to remain annealed to one another under at least conventional “low-stringency” conditions. Similarly, the molecules are “complementary” if they can hybridize to one another with sufficient stability to permit them to remain annealed to one another under conventional “high-stringency” conditions. Conventional stringency conditions are described by Haymes et al., In: Nucleic Acid Hybridization, A Practical Approach, IRL Press, Washington, DC (1985), and by M R Green and J Sambrook, Molecular cloning: a laboratory manual, 4^{th} Edition, Cold Spring Harbor Laboratory Press, Cold Spring Harbor, N.Y. (2012). Departures from complete complementarity are therefore permissible, as long as such departures do not completely preclude the capacity of the molecules to form a double-stranded structure. In order for a nucleic acid molecule to serve as a primer or probe, it need only be sufficiently complementary in sequence to be able to form a stable double-stranded structure under the particular solvent and salt concentrations and other conditions employed.

As used herein, a substantially homologous or complementary sequence in relation to a reference nucleic acid sequence is a nucleic acid sequence that will specifically hybridize to the reference nucleic acid sequence or its complement to which it is being compared under high stringency conditions. As used herein, “stringent hybridization conditions” refers to conditions under which a polynucleotide will hybridize to its target sequence, typically in a complex mixture of nucleic acids, but to essentially no other sequences, “Stringent conditions” or “stringent hybridization conditions” when referring to a polynucleotide probe, refer to conditions under which a probe will hybridize to its target sequence to a detectably greater degree than to other sequences (e.g., at least 2-fold over background). Stringent conditions are sequence-dependent and will be different in different circumstances. Longer sequences hybridize specifically at higher wenperatures, Generally, stringent conditions are selected to be about 5-10° C. lower than the thermal melting point (Tₘ) for the specific sequence at a defined ionic strength and pH. The Tₘ.is the temperature (under defined ionic strength, pH. and nucleic acid concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to L) M sodium ion concentration or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. By controlling the stringency of the hybridization and/or washing conditions, target sequences that are 100% complementary to the probe can be identified (homologous probing). Alternatively, stringency conditions can be adjusted to allow some mismatching in sequences so that lower degrees of identity are detected (heterologous probing).

Appropriate stringency conditions which promote DNA hybridization, for example, 6x sodium chloride/sodium citrate (SSC) at about 45° C., followed by a wash of 2×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed. Regarding the amplification of a target polynucleotide (e.g., by PCR) using a particular amplification primer pair, “stringent conditions” or “stringent hybridization conditions” are conditions that permit the primer pair to hybridize to the target polynucleotide to which a primer having the corresponding wildtype sequence (or its complement) would bind and to produce an identifiable amplification product (the amplicon) having a corn Zm_CSM63715 event specific region in a DNA thermal amplification reaction. The term “specific for” a target sequence indicates that a probe or primer hybridizes under stringent hybridization conditions only to the target sequence in a sample comprising the target sequence.

A polynucleotide molecule or DNA molecule of the present disclosure, such as a primer or a probe, will specifically hybridize to at least one of the nucleic acid molecule sequences selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, a polynucleotide having a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to SEQ ID NO:10, or a complete complement of or fragment of any of the foregoing under stringent hybridization conditions, or under moderately stringent hybridization conditions if the sequence of the polynucleotide molecule is not identical to the at least one of the nucleic acid molecules. The hybridization of a nucleic acid molecule, such as a primer or probe, to the target DNA molecule can be detected by any number of methods known to those skilled in the art, which can include, but are not limited to, fluorescent tags, radioactive tags, antibody-based tags, and chemiluminescent tags.

An illustrative DNA molecule or polynucleotide useful as a probe for detecting corn event Zm_CSM63715 is provided as SEQ ID NO:16. In some embodiments, a DNA molecule that functions as a probe comprises a nucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, a complement of any of the foregoing or a fragment of any of the foregoing. In other embodiments, a DNA molecule comprises a polynucleotide segment of sufficient length to function as a DNA probe specific for at least one of: a) a 5′ junction sequence between flanking corn genomic DNA and the transgenic insert of corn event Zm_CSM63715; b) a 3′ junction sequence between the transgenic insert of corn event Zm_CSM63715 and flanking corn genomic DNA; c) SEQ ID NO:9; or d) a fragment of SEQ ID NO:9 comprising a sufficient length of contiguous nucleotides of SEQ ID NO:9 to identify the sequence as a fragment of the transgenic insert of Zm_CSM63715 in a sample of DNA.

A diagnostic amplicon produced by the methods described herein may be detected by a plurality of techniques known in the art, such as sequencing, restriction mapping, Southern analysis, or any other suitable polynucleotide or DNA hybridization, blotting, polymerization and/or amplification-based approach or technique. One method is Genetic Bit Analysis (Nikiforov et al., 1994) where a DNA oligonucleotide is designed that overlaps both the adjacent flanking genomic DNA sequence and the inserted DNA sequence-i.e., a junction sequence. The oligonucleotide is immobilized in wells of a microtiter plate. Following PCR of the region of interest (using, for example, one primer in the inserted sequence and one in the adjacent flanking genomic sequence), a single-stranded PCR product can be hybridized to the immobilized oligonucleotide and serve as a template for a single base extension reaction using a DNA polymerase and labeled dideoxynucleotide triphosphates (ddNTPs) specific for the expected next base. Readout may be fluorescent or ELISA-based. A signal indicates presence of the transgene/genomic junction sequence due to successful amplification, hybridization, and single base extension.

Another method is the pyrosequencing technique as described by Winge (2000). In this method, an oligonucleotide is designed that overlaps the adjacent genomic DNA and insert DNA junction. The oligonucleotide is hybridized to single-stranded PCR product from the region of interest (one primer in the inserted sequence and one in the flanking genomic sequence) and incubated in the presence of a DNA polymerase, ATP, sulfurylase, luciferase, apyrase, adenosine 5′ phosphosulfate and luciferin. DNTPs are added individually and the incorporation results in a light signal that is measured. A light signal indicates the presence of the transgene/genomic sequence due to successful amplification, hybridization, and single or multi-base extension.

Fluorescence Polarization as described by Chen et al. (1999) is a method that can be used to detect the amplicon of the present invention. Using this method an oligonucleotide is designed that overlaps the genomic flanking and inserted DNA junction. The oligonucleotide is hybridized to single-stranded PCR product from the region of interest (one primer in the inserted DNA and one in the flanking genomic DNA sequence) and incubated in the presence of a DNA polymerase and a fluorescent-labeled ddNTP. Single base extension results in incorporation of the ddNTP. Incorporation can be measured as a change in polarization using a fluorometer. A change in polarization indicates the presence of the transgene/genomic sequence due to successful amplification, hybridization, and single base extension.

Real-time polymerase chain reaction (PCR) has the ability to monitor the progress of the PCR as it occurs (i.e., in real time). Data are collected throughout the PCR process, rather than at the end of the PCR. In real-time PCR, reactions are characterized by the point in time during cycling when amplification of a target is first detected rather than the amount of target accumulated after a fixed number of cycles. In a real-time PCR assay, a positive reaction is detected by accumulation of a fluorescent signal. The higher the starting copy number of the nucleic acid target, the sooner a significant increase in fluorescence is observed. The cycle threshold (Ct value) is defined as the number of cycles required for the fluorescent signal to cross the threshold (i.e., exceeds background level). Ct levels are inversely proportional to the amount of target nucleic acid in the sample (i.e., the lower the Ct value, the greater the amount of target nucleic acid in the sample).

Taqman® (PE Applied Biosystems, Foster City, CA) is a method of detecting and quantifying the presence of a DNA sequence using real-time PCR and is fully described in the instructions provided by the manufacturer. Briefly, a FRET oligonucleotide probe is designed that overlaps the genomic flanking and insert DNA junction. The FRET probe and PCR primers (one primer in the insert DNA sequence and one in the flanking genomic sequence) are cycled in the presence of a thermal stable polymerase and dNTPs. Hybridization of the FRET probe results in cleavage and release of the fluorescent moiety away from the quenching moiety on the FRET probe. A fluorescent signal indicates the presence of the transgene/genomic sequence due to successful amplification and hybridization.

Molecular beacons have been described for use in sequence detection as described in Tyangi et al. (1996). Briefly, a FRET oligonucleotide probe is designed that overlaps the flanking genomic and insert DNA junction. The unique structure of the FRET probe results in it containing secondary structure that keeps the fluorescent and quenching moieties in close proximity. The FRET probe and PCR primers (one primer in the insert DNA sequence and one in the flanking genomic sequence) are cycled in the presence of a thermostable polymerase and dNTPs. Following successful PCR amplification, hybridization of the FRET probe to the target sequence results in the removal of the probe secondary structure and spatial separation of the fluorescent and quenching moieties. A fluorescent signal results and indicates the presence of the flanking/transgene insert sequence due to successful amplification and hybridization.

Other detection methods known in the art may be used. For example, microfluidics (see, e.g., U.S. Patent Publication No. 2006/068398; U.S. Pat. No. 6,544,734) provide methods and devices that can be used to separate and amplify DNA samples or molecules. Optical dyes can be used to detect and measure specific DNA molecules (see, e.g., WO/05017181). Nanotube devices (see, e.g., WO/06024023) that comprise an electronic sensor for the detection of DNA molecules or nanobeads that bind specific DNA molecules can then be detected. Nanopore sequencing technology, such as that described in Wang et al. (2021), Tayler et al. (2018), or Pearson et al. (2019), can also be used for event detection.

The DNA molecules and corresponding nucleotide sequences provided herein are therefore useful for, among other things, identifying corn event Zm_CSM63715, detecting the presence of DNA derived from the transgenic corn event Zm_CSM63715 in a sample, and monitoring samples for the presence and/or absence of corn event Zm_CSM63715 or plant parts derived from corn plants comprising event Zm_CSM63715.

Provided are proteins that can be used to produce antibodies for detecting the presence of corn event Zm_CSM63715 in a sample. Such antibodies are specific for the PPO protein that is encoded by corn event Zm_CSM63715. Methods for preparing a polyclonal antibody or a monoclonal antibody are well-known to those skilled in the art, and can be used to make antibodies specific for the PPO protein encoded by corn event Zm_CSM63715. For example, Lermontova et al (1997) described antibodies to a PPO protein. The DNA sequence encoding the PPO protein is provided in SEQ ID NO:10 and the start positions and stop positions of the coding sequences are indicated in Table 1A. The DNA sequence encoding the protein and the protein encoded by the sequence are useful to produce antibodies for detecting the presence of corn event Zm_CSM63715 by the methods described herein. Detection for the presence of corn event Zm_CSM63715 may be done by using any protein detection techniques known in the art, such as western blot analysis, immuno-precipitation, enzyme-linked immunosorbent assay (ELISA), antibody attachment to a detectable label or reporter molecule (such as a radioactive isotope, ligand, chemiluminescent agent, or enzyme), or enzymatic action on a reporter molecule. One method provides for contacting a sample with an antibody that binds to the PPO protein encoded by corn event Zm_CSM63715 and then detecting the presence or absence of antibody binding. The binding of such antibody is diagnostic for the presence of one or more proteins encoded by corn event Zm_CSM63715.

Nucleic acid or protein detection kits for detecting the presence of corn event Zm_CSM63715 are provided. Variations on such kits can also be developed using the compositions and methods disclosed herein and the methods well known in the art for protein and nucleic acid detection for identification of corn event Zm_CSM63715. Protein and nucleic acid detection kits can be applied to methods for breeding with plants comprising corn event Zm_CSM63715. Such kits contain primers and/or probes or antibodies which are specific to corn event Zm_CSM63715. Such DNA primers and/or probes may comprise fragments of one or more of SEQ ID NOs:1-10, or antibodies specific for a protein encoded by the corn event Zm_CSM63715. The kits can also contain instructions for using the primers, probes, or antibodies for detecting the presence of corn event Zm_CSM63715. Kits may optionally also comprise reagents for performing the detection or diagnostic reactions described herein.

One example of a detection kit comprises at least one DNA molecule of sufficient length of contiguous nucleotides of SEQ ID NO:10 to function as a DNA probe useful for detecting the presence or absence of corn event Zm_CSM63715 in a sample. The DNA derived from transgenic corn plants comprising event Zm_CSM63715 would comprise a DNA molecule having at least one sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10, a complement of any of the foregoing, or a fragment of any of the foregoing. An illustrative DNA molecule sufficient for use as a probe is one comprising the sequence provided as SEQ ID NO:16. Other probes may be readily designed by one of skill in the art. The probe can include a junction sequence that spans the 5′ or 3′ junction between the corn genomic DNA and the transgenic insert of corn event Zm_CSM63715.

Another example of a detection kit comprises at least one primer pair that specifically hybridize to a target DNA and amplify a diagnostic amplicon under the appropriate reaction conditions useful for detecting the presence or absence of corn event Zm_CSM63715 in a sample. A kit that contains DNA primers that are homologous or complementary to any portion of the corn genomic region as set forth in SEQ ID NO:11 or 12 and to any portion of the inserted transgenic DNA as set forth in SEQ ID NO:9 is an object of the present disclosure. The kit may provide an agarose gel-based detection method or any number of methods of detecting the amplicon that are known in the art. Such a method may also include sequencing the amplicon or a fragment thereof. Illustrative DNA molecules sufficient for use as a primer pair are ones comprising the sequences provided as SEQ ID NO:14 and SEQ ID NO:15, and SEQ ID NO:20 and SEQ ID NO:21 respectively, wherein the primer pair SEQ ID NO:14 and SEQ ID NO:15 will produce an amplicon diagnostic for the presence of event Zm_CSM63715 in a sample; and the primer pair SEQ ID NO:20 and SEQ ID NO:21 will produce an amplicon indictive of wildtype DNA, therefore, diagnostic for the absence of event Zm_CSM63715 in a sample. Other primer pairs may be readily designed by one of skill in the art.

Another example of a detection kit comprises an antibody specific for the PPO protein encoded by corn event Zm_CSM63715. For example, such a kit may utilize a lateral flow strip comprising reagents activated when the tip of the strip is contacted with an aqueous solution. Illustrative protein sufficient for use in antibody production is the PPO protein encoded by the sequence provided as SEQ ID NO:10, or any fragment thereof. Detection of binding of the antibody to the PPO protein encoded by corn event Zm_CSM63715 in a sample is diagnostic for the presence of corn event Zm_CSM63715 in the sample.

The detection kits provided herein are useful for, among other things, identifying corn event Zm_CSM63715, selecting plant varieties or hybrids comprising corn event Zm_CSM63715, detecting the presence of DNA derived from the transgenic corn plant comprising event Zm_CSM63715 in a sample, and monitoring samples for the presence and/or absence of corn plants comprising event Zm_CSM63715, or plant parts derived from corn plants comprising event Zm_CSM63715.

Corn plants, progeny, seeds, cells, and plant parts comprising corn event Zm_CSM63715 are provided, as well as commodity products produced using these. As used herein, the term “corn” or “maize” means plant species within Zea mays and all plant varieties belonging to the genus Zea that can be bred with Zea mays plants, including wild corn species such as Zea dipioperennis. The term “corn” is intended to include corn plants, plant parts, plant cells, plant tissue, seeds, progeny plants, and/or corn commodity products. These corn plants, plant parts, plant cells, plant tissues, seeds, progeny plants and commodity products contain or comprise corn event Zm_CSM63715 or are derived from a transgenic corn plant, plant part, plant cell, plant tissue, seed, progeny plant or commodity product containing or comprising event Zm_CSM63715. These corn plants, plant parts, plant cells, plant tissues, seeds, progeny plants and commodity products contain a detectable amount of a polynucleotide or DNA molecule comprising at least one junction sequence and/or heterologous transgenic insert sequence of corn event Zm_CSM63715, such as a polynucleotide or nucleic acid or DNA molecule having or comprising at least one of the sequences provided as SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, a polynucleotide comprising at least 16 consecutive nucleotides of SEQ ID NO:1, at least 16 consecutive nucleotides of SEQ ID NO:2, at least 31 consecutive nucleotides of SEQ ID NO:3, at least 35 consecutive nucleotides of SEQ ID NO:4, at least 51 consecutive nucleotides of SEQ ID NO:5, or at least 51 consecutive nucleotides of SEQ ID NO:6, a polynucleotide comprising a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO: 9, and a complete complement of any of the foregoing. In some embodiments, the corn plant, plant part, plant cell, plant tissue, or seed is further defined as a progeny plant of any generation of a corn plant comprising corn event Zm_CSM63715, or a corn plant part, plant seed, or plant cell derived therefrom.

The corn plants, plant parts, plant cells, plant tissues, seeds, progeny plants and commodity products express or comprise a PPO herbicide tolerance gene, and are tolerant to one or more PPO herbicides including, for example, but not limited to flumioxazin, epyrifenacil, lactofen, acifluorfen, pyraflufen, pyraflufen-ethyl, oxadiazon, butafenacil, pyridin-2-ylmethyl[(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl[(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl[(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate, cyanomethyl[(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate, methyl (2R)-2-{[(E)-({2-chloro-4-fluoro-5-[3-methyl-2,6-dioxo-4-(trifluoromethyl)-3,6-dihydropyrimidin-1(2H)-yl]phenyl}methylidene)amino]oxy}propanoate (flufenoximacil), cyclopropylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate, fomesafen, saflufenacil, sulfentrazone, tiafenacil, trifludimoxazin, and combinations of any thereof.

The present disclosure provides corn plants, progeny, seeds, plant cells, and plant parts such as microspores, pollen, anthers, silk, spike, ovules, ovaries, flowers, pods, cobs, embryos, stems, leaves, roots, and calli derived from a transgenic corn plant comprising corn event Zm_CSM63715. A representative sample of seed comprising corn event Zm_CSM63715 has been deposited according to the Budapest Treaty for the purpose of enabling the present disclosure. The ATCC repository has assigned the Accession No. PTA-127361 to the seed comprising corn event Zm_CSM63715.

Any of the corn plants, plant seeds, plant parts, or plant cells can further comprise at least one additional transgene for tolerance to at least one additional herbicide. For example, the additional transgenes can be selected from the group consisting of FT_T, dicamba monooxygenase (DMO), phosphinothricin N-acetyltransferase (PAT), 5-enolpyruvylshikimate-3-phosphate synthase (EPSPS), and combinations of any thereof. An illustrative PAT coding sequence and its corresponding amino acid sequence from Streptomyces viridochromogenes are provided as SEQ ID NO:166 and SEQ ID NO:167, respectively. An illustrative DMO coding sequence and its corresponding amino acid sequence from Pseudomonas maltophilia are provided as SEQ ID NO:168 and SEQ ID NO:169, respectively. An illustrative FT_T coding sequence and its corresponding amino acid sequence from Sphingobium herbicidovorans are provided as SEQ ID NO:170 and SEQ ID NO:171, respectively. An illustrative EPSPS coding sequence and its corresponding amino acid sequence from Agrobacterium CP4 strain are provided as SEQ ID NO:172 and SEQ ID NO:173, respectively. For example, the corn plant, plant seed, plant part, or plant cell can further comprise corn event MON87429.

The additional transgenes can provide tolerance to herbicides having modes of action selected from the group consisting of inhibitors of glutamine synthetase (e.g., glufosinate), inhibitors of acetyl CoA carboxylase (ACCase) in the aryloxyphenoxy propionate (FOP) group (e.g., chlorazifop, clodinafop, clodinafop-ethyl, clodinafop-propargyl, clofop, cyhalofop, cyhalofop-butyl, diclofop, diclofop-methyl, diclofop-P, diclofop-P-methyl, fenoxaprop, fenoxaprop-P, fenoxaprop-P-ethyl, fenthiaprop, fluazifop, fluazifop-butyl, fluazifop-P, fluazifop-P-butyl, haloxyfop, haloxyfop-etotyl, haloxyfop-methyl, haloxyfop-P, haloxyfop-P-methyl, isoxapyrifop, metamifop, propaquizafop, quizalofop, quizalafop-ethyl, quizalofop-P, quizalafop-P-ethyl, quizalafop-P-tefuryl, trifop, and combinations of any thereof), inhibitors of EPSPS (e.g., glyphosate), synthetic auxins (e.g., dicamba, 2,4-D, dichlorprop, mecoprop, 2,4,5-T (2,4,5-trichlorophenoxyacetic acid), and combinations of any thereof), and combinations of any thereof.

A microorganism is provided. The microorganism comprises a polynucleotide molecule having the nucleotide sequence of SEQ ID NO:9, or a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:9. An example of such a microorganism is an Agrobacterium cell. Another example of such a microorganism is an E. coli cell.

A plant cell is provided comprising a polynucleotide molecule as described herein. For example, a plant cell is provided having a nucleotide sequence present in its genome, wherein the nucleotide sequence is selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and a nucleic acid molecule comprising a polynucleotide having a nucleotide sequence that is at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, or at least 99.9% identical to the full length of SEQ ID NO:10 or the full length of SEQ ID NO: 9.

Plant cells and microorganisms of the present disclosure are useful in many industrial applications, including but not limited to: (i) use as research tools for scientific inquiry or industrial research; (ii) use in culture for producing endogenous or recombinant carbohydrate, lipid, nucleic acid, enzymes or protein products or small molecules that may be used for subsequent scientific research or as industrial products; and (iii) for the plant cells of the present disclosure, use with modem plant tissue culture techniques to produce transgenic plants or plant tissue cultures that may then be used for agricultural research or production. The production and use of such transgenic plant cells utilize modern microbiological techniques and human intervention to produce a man-made, unique plant cell. In this process, a recombinant DNA is inserted into a plant cell's genome to create a transgenic plant cell that is separate and unique from naturally occurring plant cells.

This transgenic plant cell can then be cultured much like bacteria and yeast cells using modern microbiology techniques and may exist in an undifferentiated, unicellular state. The new plant cell's genetic composition and phenotype is a technical effect created by the integration of a heterologous DNA into the genome of the cell.

Provided are methods of using a plant cell, such as transgenic plant cells. These include (i) methods of producing transgenic cells by integrating a recombinant DNA into the genome of the cell and then using this cell to derive additional cells possessing the same heterologous DNA; (ii) methods of culturing cells that contain recombinant DNA using modem microbiology techniques; (iii) methods of producing and purifying endogenous or recombinant carbohydrate, lipid, nucleic acid, enzymes or protein products from cultured cells; and (iv) methods of using modern plant tissue culture techniques with transgenic plant cells to produce transgenic plants or transgenic plant tissue cultures.

Plants, progeny, seeds, cells, and plant parts may contain one or more additional desirable trait(s). Such desirable traits may be transgenic traits, native traits, or traits produced by other methods such as genome editing, base editing, prime editing or other conventional mutagenesis methods. Desirable traits may be combined with corn event Zm_CSM63715 by, for example, crossing a corn plant comprising corn event Zm_CSM63715 with another corn plant containing the additional trait(s), or transgenic events. Such traits or transgenic events include, but are not limited to, increased insect resistance, increased water use efficiency, increased yield performance, increased drought resistance, increased disease resistance, increased seed quality, improved nutritional quality, hybrid seed production, and/or increase herbicide tolerance, in which the trait is measured with respect to a corn plant lacking such transgenic trait. For example, the Zm_CSM63715 event could be stacked by breeding or by site directed introgression with other events or combinations of events known in the art including, but not limited to:
MON00603 (also known as NK603 or MON603; Roundup Ready™ 2 Maize for herbicide tolerance; deposited as ATCC PTA-2478 and described in US Patent Application Publication No. 2007/292854 and U.S. Pat. No. 6,825,400, the entire contents and disclosure of each of which are incorporated herein by reference in their entirety),
MON89034 (YieldGard™ VT Pro™ for insect resistance; deposited as ATCC PTA-7455 and described in PCT Publication No. WO2007/140256 and US Patent Application Publication No. US2008/260932, the entire contents and disclosure of each of which are incorporated herein by reference in their entirety),
MON88017 (YieldGard™ VT™ Rootworm™ RR2 for herbicide tolerance and insect resistance; deposited as PTA-5582 and described in US Patent Application Publication No. 2008/028482 and PCT Publication No. WO2005/059103, the entire contents and disclosure of each of which are incorporated herein by reference in their entirety),
MON87427 (Roundup Ready™ Maize for herbicide tolerance, deposited as ATCC PTA-7899, described in U.S. Pat. No. 8,618,358 and PCT Publication No. WO2011/062904, the entire contents and disclosure of each of which are incorporated herein by reference in their entirety),
MON87411 (for insect resistance; deposited as ATCC No. PTA-12669 and described in U.S. Pat. No. 10,316,330 and PCT Publication No. WO2013/169923, the entire contents and disclosure of each of which are incorporated herein by reference in their entirety),
MON87429 (for herbicide tolerance; deposited as ATCC PTA-124635 and described in U.S. Pat. No. 10,920,239 and PCT Publication No. WO2019/152316, the entire contents and disclosure of each of which are incorporated herein by reference in their entirety),
MON87460 (Genuity® DroughtGard™ for abiotic stress tolerance; deposited as ATCC No. PTA-8910 and described in PCT Publication No. WO2009/111263 and US Patent Application Publication No. 2011/0138504, the entire contents and disclosure of each of which are incorporated herein by reference in their entirety),
MON87419 (for herbicide tolerance; deposited as ATCC PTA-120860 and described in U.S. Pat. No. 11,098,321 and PCT Publication WO2015/142571, the entire contents and disclosure of each of which are incorporated herein by reference in their entirety),
MON95275 (for insect resistance, deposited as ATCC PTA-126049 and described in US Patent Application Publication No. US2021/332380 and PCT Publication No.

WO2021/216571, the entire contents and disclosure of each of which are incorporated herein by reference in their entirety).
MON95379 (for insect resistance; deposited as ATCC PTA-125027 and described in PCT Publication WO2020/028172) and US Patent Application Publication No. US2020/032289, the entire contents and disclosure of each of which are incorporated herein by reference in their entirety.
MON00810 (for insect resistance; also known as MON810; described in US Patent Application Publication No. 2002/102582, the entire contents and disclosure of which are incorporated herein by reference in their entirety),
MON00021 (also known as GA21; Roundup Ready™ Maize, Agrisure™ GT for herbicide tolerance, deposited as ATCC 209033 and described in US Patent Application Publication No. 2005/086719 and PCT Publication No. WO1998/044140, the entire contents and disclosure of each of which are incorporated herein by reference in their entirety),
MON832 (Roundup Ready™ Maize for herbicide tolerance),
MON863 (YieldGard™ Rootworm R W, MaxGard™ for insect resistance; deposited as ATCC PTA-2605 and described in PCT Publication No. WO2004/011601 and US Patent Application Publication No. 2006/095986, the entire contents and disclosure of each of which are incorporated herein by reference in their entirety),
AGV-PY203-4 (GraINzyme Phytase for modified product quality),
ACS-ZM004-3 (Starlink™ Maize for herbicide tolerance and insect resistance),
ACS-ZM001-9 (InVigor™ Maize for pollination control system),
ACS-ZM005-4 (InVigor™ Maize for pollination control system),
ACS-ZM002-1 (Liberty Link™ Maize for herbicide tolerance),
ACS-ZM003-2 (Liberty Link™ Maize for herbicide tolerance),
DAS-40278-9 (Enlist™ Maize for herbicide tolerance, deposited as ATCC No. PTA-10244 and described in U.S. Pat. No. 11,098,322 and PCT Publication No.

WO2011/022469),
DAS-01507-1 (also known as TC1507; Herculex™ I, Herculex™ CB for herbicide tolerance and insect resistance; described in US Patent Application Publication No. 2005039226 and PCT Publication No WO2004/099447),
DAS-59122-7 (Herculex™ RW for herbicide tolerance and insect resistance; described in US Patent Application Publication No. 2006/070139),
DKB-89614-9 (Bt Xtra™ Maize for herbicide tolerance and insect resistance),
DP-32138-1 (32138 SPT maintainer for pollination control system; deposited as ATCC No. PTA-9158 and described in US Patent Application Publication No. 2009/0210970 and PCT Publication No. WO2009/103049),
DP-098140-6 (Optimum™ GAT™ for herbicide tolerance; deposited as ATCC No. PTA-8296 and described in US Patent Application Publication No. 2009/137395 and PCT Publication No. WO2008/112019),
MIR162 (Agrisure™ Viptera for insect resistance; deposited as ATCC No. PTA-6188 and described in US Patent Application Publication No. 2009/300784 and PCT Publication No.

WO2007/142840),
MIR604 (Agrisure™ RW for insect resistance, described in US Patent Application Publication No. 2008/167456 and PCT Publication No. WO2005/103301),
REN-00038-3 (also known as LY038, Mavera™ Maize for modified product quality; deposited as ATCC No. PTA-5623 and described in PCT Publication No. WO2005/061720 and U.S. Pat. No. 7,615,621, the entire contents and disclosure of each of which are incorporated herein by reference in their entirety),
SYN-E3272-5 (Enogen™ for modified product quality, described in U.S. Pat. No. 7,635,799 and PCT Application Publication No. WO2006/098952),
SYN-05307-1 (Agrisure® Duracade™ for insect resistance; deposited as ATCC No. PTA-9561 and described in PCT Publication No. WO2010/077816 and US Patent No. U.S. Pat. No. 10,100,371),
Bt10 (for herbicide tolerance and insect resistance),
SYN-BTO11-1 (Agrisure™ CB/LL for herbicide tolerance and insect resistance),
SYN-EV176-9 (NaturGard KnockOut™, Maximizer™ for herbicide tolerance and insect resistance),
MON89034 x DAS-01507-1 x MON603 x MIR162 x DAS-40278-9 (Power Core™ X MIR162 x Enlist™ for herbicide tolerance and insect resistance),
DAS-01507-1 x DAS-59122-7 (Herculex XTRA™ for herbicide tolerance and insect resistance),
DAS-01507-1 x DAS-59122-7 x MON603 (Herculex XTRA™ RR for herbicide tolerance and insect resistance),
DAS-01507-1 x MON603 (Herculex™ I RR for herbicide tolerance and insect resistance),
DAS-59122-7 x MON603 (Herculex™ RW Roundup Ready™ 2 for herbicide tolerance and insect resistance),
DAS-01507-1 x DAS-59122-7 x MON00810 x MIR604 x MON603 (Optimum™ Intrasect Xtreme for herbicide tolerance and insect resistance),
DAS-01507-1 x DAS-59122-7 x MON810 x MON603 (Optimum™ Intrasect XTRA for herbicide tolerance and insect resistance),
DAS-01507-1 x MIR604 x MON603 (Optimum™ TRIsect for herbicide tolerance and insect resistance),
DAS-01507-1 x MON810 x MON603 (Optimum™ Intrasect for herbicide tolerance and insect resistance),
MON00021 x MON810 (Roundup Ready™ YieldGard™ Maize for herbicide tolerance and insect resistance),
MON810 x MON88017 (YieldGard™ VT Triple for herbicide tolerance and insect resistance),
MON863 x MON810 (YieldGard™ Plus for insect resistance),
MON603 x MON810 x MON863 (YieldGard™ Plus with RR for herbicide tolerance and insect resistance),
MON863 x MON603 (YieldGard™ RW+RR for herbicide tolerance and insect resistance),
MON87427 x MON89034 x DAS-01507-1 x MON87411 x DAS-59122-7 x DAS-40278-9 (SmartStax™ Pro x Enlist™ for herbicide tolerance and insect resistance),
MON89034 x MON88017 (Genuity® VT Triple Pro™ for herbicide tolerance and insect resistance),
MON89034 x MON603 (Genuity® VT Double Pro™ for herbicide tolerance and insect resistance),
MON89034 x DAS-01507-1 x MON88017 x DAS-59122-7 (Genuity® SmartStax™ for herbicide tolerance and insect resistance),
MON89034 x DAS-01507-1 x MON603 (Power Core™ for herbicide tolerance and insect resistance),
MON603 x MON810 (YieldGard™ CB+RR for herbicide tolerance and insect resistance),
MON603 x ACS-ZM003-2 (Roundup Ready™ Liberty Link™ Maize for herbicide tolerance),
ACS-ZM003-2 x MON810 (Liberty Link™ Yieldgard™ Maize for herbicide tolerance and insect resistance),
REN-00038-3 x MON810 (Mavera™ YieldGard™ Maize for insect resistance and modified product quality),
SYN-05307-1 x MIR604 x SYN-BTO11-1 x DAS-01507-1 x MON00021 (Agrisure® Duracade™ 5122 for herbicide tolerance and insect resistance),
SYN-05307-1 x MIR604 x SYN-BTO11-1 x DAS-01507-1 x MON00021 x MIR162 (Agrisure® Duracade™ 5222 for herbicide tolerance and insect resistance),
SYN-BTO11-1 x DAS-59122-7 x MIR604 x DAS-01507-1 x MON00021 (Agrisure® 3122 for herbicide tolerance and insect resistance),
SYN-BTO11-1 x MON00021 (Agrisure™ GT/CB/LL for herbicide tolerance and insect resistance),
SYN-BTO11-1 x MIR162 (Agrisure® Viptera™ 2100 for herbicide tolerance and insect resistance),
SYN-BT011-1 x MIR162 x MON00021 (Agrisure® Viptera™ 3110 for herbicide tolerance and insect resistance),
SYN-BT011-1 x MIR162 x MIR604 (Agrisure® Viptera™ 3100 for herbicide tolerance and insect resistance),
SYN-BTO11-1 x MIR162 x DAS-01507-1 x MON00021 (Agrisure® Viptera™ 3220 for herbicide tolerance and insect resistance),
SYN-BTO11-1 x MIR604 (Agrisure™ CB/LL/RW for herbicide tolerance and insect resistance),
SYN-BTO11-1 x MIR604 x MON00021 (Agrisure™ 3000GT for herbicide tolerance and insect resistance), and/or MIR604 x MON00021 (Agrisure™ GT/RW for herbicide tolerance and insect resistance).

“MON87429” refers to corn event MON87429. Corn seed comprising event MON87429 has been deposited under ATCC Accession No. PTA-124635 and is fully described and characterized in U.S. Pat. No. 10,920,239 and PCT Publication No. WO2019/152316, the entire contents and disclosure of each of which are incorporated herein by reference in their entirety. Transgenic corn plants comprising corn event MON87429 comprise SEQ ID NO:212 (5′ corn genomic flank sequence+transgenic insert+3′ corn genomic flank sequence), SEQ ID NO:213 (transgenic insert), SEQ ID NOs:214-217 (5′ junction sequences), and SEQ ID NOs:218-221 (3′ junction sequences). The transgenic insert in corn plants comprising event MON87429 comprises four expression cassettes as shown in Table 1B. The first expression cassette comprises in operable linkage (1) a ubiquitin promoter, leader, and intron from Erianthus ravennae, (II) a phosphinothricin N-acetyltransferase coding sequence, and (III) a fructose-bisphosphate aldolase 3′ UTR from Setaria italica; the second expression cassette comprises in operable linkage (I) a ubiquitin promoter, leader, and intron from Coix lacryma-jobi, (II) an albino and pale green 6 chloroplast transit peptide coding sequence from Arabidopsis thaliana. (III) a dicamba monooxygenase coding sequence, and (IV) a metallothionein-like protein 3′ UTR from Oryza sativa; the third expression cassette comprises in operable linkage (1) a ubiquitin promoter, leader, and intron from Arundo donax, (II) a malate dehydrogenase chloroplast transit peptide coding sequence from Arabidopsis thaliana, (III) a FT_T protein coding sequence, and (IV) a no apical meristem protein 3′ UTR from Orza sativa; and the fourth expression cassette comprises in operable linkage (1) a CaMV % 35S promoter and leader, MI) a chlorophyll a/b-binding protein leader from Triticum aestivum, (III) an actin 1 intron from Orza saliva, (IV) a ShkG chloroplast transit peptide coding sequence from Arabidopsis thaliana (V) a glyphosate tolerant 5-enolpyruvylshikimate-3-phosphate synthase coding sequence from Agrobacterium sp strain CP4 (VI) a male tissue specific siRNA target from Zea mays, and (VII) a glycine-rich RNA binding protein 3UTR from Orza sativa.

Corn plants comprising event M0N87429 exhibit tolerance to inhibitors of acetyl CoA carboxylase (ACCase) in the aryloxyphenoxy propionate (FOP) group such as quizalofop and haloxyfop; synthetic auxins such as dicamba and 2,4-D); inhibitors of glutamine synthetase such as glufosinate; and inhibitors of 5-enolpyruvylshikimate-3-phosphate synthase (EPSPS) such as glyphosate.

TABLE 1B
Transgenic cassettes and elements in corn event MON87429.

| Cassette | Element | Description |
|---|---|---|
| 1 | P-Ea.ubq | Promoter, 5′ UTR, and intron sequences of a ubiquitin gene from Erianthus ravennae |
| | CS-Sv.pat | Coding sequence for the phosphinothricin N-acetyltransferase (PAT) gene |
| | T-Si.fba | 3′ UTR sequence of the fructose-bisphosphate aldolase gene from Seteria italica |
| 2 | P-Clj.ubq | Promoter, 5′ UTR, and intron sequences of a ubiquitin gene from Coix alcryma-jobi |
| | TS-At.apg6 | Codon optimized targeting sequence of the Albino and pale green 6 gene from |
| | | Arabidopsis thaliana |
| | CS-Sm.dmo | Coding sequence for the dicamba monooxygenase (DMO) protein |
| | T-Os.mt | 3′ UTR sequence of the metallothionein-like protein from Oryza sativa |
| 3 | P-Ad.ubq | Promoter, 5′ UTR, and intron sequences of a ubiquitin gene from Arundo donax |
| | TS-At.mdh | Sequence of the transit peptide of the malate dehydrogenase gene from Arabidopsis |
| | | thaliana |
| | CS-Sh.ft_t | Coding sequence for the FT_T protein |
| | T-Os.nam | 3′ UTR sequence of the no apical meristem protein from Oryza sativa |
| 4 | P-CaMV.35S | Promoter and leader from the 35S RNA of cauliflower mosaic virus |
| | L-Ta.cab | 5′ UTR leader sequence from chlorophyll a/b-binding protein of Triticum aestivum |
| | I-Os.act1 | Intron and UTR sequence of the Actin 1 protein from Oryza sativa |
| | TS-At.CTP2 | Transit peptide sequence of the ShkG gene from Arabidopsis thaliana |
| | CS-cp4epsps | Coding sequence for the 5-enolpyruvylshikimate-3-phosphate synthase (CP4-EPSPS) |
| | | protein |
| | mts.siRNA | Sequence of the male tissue specific siRNA target |
| | T-Os.grp3 | 3′ UTR sequence of the glycine-rich RNA-binding protein (Grp3) gene from Oryza sativa |

Any of the corn plants, plant parts, seeds, cells, progeny or commodity products described herein that comprise corn event Zm_CSM63715 can further comprise corn event MON87429.

Any of the corn plants, plant parts, seeds, cells, progeny or commodity products described herein can further comprise a recombinant DNA molecule comprising a sequence selected from the group consisting of SEQ ID NO:212; SEQ ID NO:213; SEQ ID NO:214; SEQ ID NO:215; SEQ ID NO:216; SEQ ID NO:217; SEQ ID NO:218; SEQ ID NO:219; SEQ ID NO:220; SEQ ID NO:221; a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:212 or the full length of SEQ ID NO: 213; and a complete complement of any of the foregoing.

Plants comprising both corn event Zm_CSM63715 and corn event MON87429 can be made by any method known in the art. For example, such plants can be made by crossing a corn plant comprising corn event Zm_CSM63715 with a corn plant comprising corn event MON87429 and selecting for progeny plants containing both events. Alternatively, one or both events can be inserted into the genome of a corn plant by site-directed insertion using a site-directed nuclease.

The term “site-specific nuclease” refers to any enzyme that can cleave a nucleotide sequence in a site-specific manner. Site-specific nucleases allow for the precise and/or targeted editing of a specific location in a genome of a plant. Site-specific nucleases include, for example, RNA guided nucleases, zinc-finger nucleases (ZFNs), and transcription activator-like effector nucleases (TALENs).

Some site-specific nucleases, such as zinc finger nucleases (ZFNs) and TALENs, are not RNA-guided and instead rely on their protein structure to determine their target site for causing a DSB (double stranded break) or nick, or they are fused, tethered or attached to a DNA-binding protein domain or motif. The protein structure of the site-specific nuclease (or the fused/attached/tethered DNA binding domain) targets the site-specific nuclease to the target site. ZFNs, and TALENs, may be designed, engineered and constructed according to known methods to target and bind to a target site. RNA-Guided nucleases are nucleases that form a complex (e.g., a ribonucleoprotein) with a guide RNA, which then guides the complex to a target site within a target sequence. One non-limiting example of guided nucleases are CRISPR nucleases. CRISPR (Clustered Regularly Interspaced Short Palindromic Repeats) nucleases are proteins found in bacteria that are guided by guide RNAs (“gRNAs”) to a target nucleic acid molecule, where the endonuclease can then cleave one or two strands the target nucleic acid molecule. Although the origins of CRISPR nucleases are bacterial, many CRISPR nucleases have been shown to function in eukaryotic cells. CRISPR editing systems comprising a CRISPR associated protein (nuclease) and cognate guide RNAs (that can be transcribed from guide DNA polynucleotides) may be used for targeted DNA cleavage or modification. The CRISPR-associated protein can be selected from a Type I CRISPR-associated protein, a Type II CRISPR-associated protein, a Type III CRISPR-associated protein, a Type IV CRISPR-associated protein, a Type V CRISPR-associated protein, or a Type VI CRISPR-associated protein, such as, but not limited to, Cas1, Cas1B, Cas2, Cas3, Cas4, Cas5, Cas6, Cas7, Cas8, Cas9 (also known as Csn1 and Csx12), Cas10, Cas12a (also known as Cpf1), Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, CasX, CasY, and Mad7.

As further described in Example 2 hereinbelow, corn event Zm_CSM63715 was integrated into the genome at a site close to the location of the MON87429. In order to accomplish this, bioinformatic analysis was first used to identify genomic target sites for site directed integration (SDI) of transgenes.

As used herein, the term “target site,” “genomic target site,” “target genomic nucleic acid,” or “target corn genomic nucleic acid,” refers to a polynucleotide sequence that is sufficiently unique in the corn genome to allow targeted genome modification by a site-specific nuclease. In one aspect, the sequence of the target site is changed from the wildtype sequence, namely the target site is edited. In another aspect, the target site is the site of insertion of a DNA sequence of interest.

The target site can comprise one or more of the criteria selected from the group consisting of: (i) the target site selected is more than 1 kb from a gene, (ii) the target site selected is more than 1 kb from a repressive chromatin mark (e.g., H3K27me3 peak), (iii) the target site selected is more than 200 nucleotides (nt) from a small RNA hotspot, (iv) the target site selected is more than 1 kb from a long repeat region, (v) the target site selected has low DNA methylation (less than or equal to 10% of genome-wide population average) (vi) the target site selected has low redundancy score (less than or equal to 30%). Target site selection criteria are further described in US Patent Application Publication No. 2020/0024610, the entire contents and disclosure of which are incorporated herein by reference in their entirety.

The target site comprises a sequence that is recognized by a site-specific nuclease. In some embodiments, the target site comprises a sequence that is recognized by a site-specific nuclease resulting in precise or targeted cleavage within the target site. For example, the site-specific nuclease can be selected from the group consisting of: an RNA-guided nuclease, a Zinc Finger nuclease, and a TALEN. In some embodiments, the target site comprises a PAM (Protospacer Adjacent Motif) sequence that is recognized by an RNA-guided nuclease (e.g., a CRISPR nuclease system). For example, the target site can comprise a PAM motif that is recognized by a Cas12a/Cpf1 CRISPR nuclease system. The target site can further comprise a sequence that is recognized by and hybridizes to a CRISPR guide RNA. In some embodiments, the target site comprises a sequence that is recognized by and hybridizes to a Cas12a/Cpf1 CRISPR guide RNA.

A DNA sequence of interest can be inserted at a target site using a site-specific nuclease. As used herein, the term “DNA sequence of interest” or “donor sequence” or “donor DNA” refers to a nucleic acid/DNA sequence that has been selected for targeted insertion into a corn genomic sequence. In one aspect, the corn genomic sequence is a genomic target site described above. A DNA sequence on interest can be of any length, for example between 2 and 50,000 nucleotides in length (or any integer value therebetween). In some embodiments, the DNA sequence is between about 1,000 and 5,000 nucleotides in length (or any integer value therebetween). In some embodiments, the DNA sequence is between about 5,000 and 10,000 nucleotides in length (or any integer value therebetween). In some embodiments, the DNA sequence is between about 10,000 and 15,000 nucleotides in length (or any integer value therebetween). In some embodiments, the DNA sequence is between about 15,000 and 20,000 nucleotides in length (or any integer value therebetween). In some embodiments, the DNA sequence is between about 20,000 and 25,000 nucleotides in length (or any integer value therebetween). In some embodiments, the DNA sequence is between about 25,000 and 30,000 nucleotides in length (or any integer value therebetween). In some embodiments, the DNA sequence is between about 30,000 and 35,000 nucleotides in length (or any integer value therebetween). In some embodiments, the DNA sequence is between about 35,000 and 40,000 nucleotides in length (or any integer value therebetween). In some embodiments, the DNA sequence is between about 40,000 and 45,000 nucleotides in length (or any integer value therebetween). In some embodiments, the DNA sequence is between about 45,000 and 50,000 nucleotides in length (or any integer value therebetween). A DNA sequence may comprise one or more gene expression cassettes that further comprise actively transcribed and/or translated gene sequences. For example, the DNA sequence of interest can comprise a gene expression cassette comprising a sequence selected from: an herbicide tolerance gene, an insecticidal resistance gene, a nitrogen use efficiency gene, a water use efficiency gene, a nutritional quality gene, a DNA binding gene, a selectable marker gene, a target site for a site-specific nuclease, and any combination thereof. Alternatively, the DNA sequence of interest may comprise a polynucleotide sequence which does not comprise a functional gene expression cassette or an entire gene (e.g., may comprise regulatory sequences such as a promoters, enhancers, etc.), or may not contain any identifiable gene expression elements or any actively transcribed gene sequence. In some embodiments, the DNA of interest will have at least one homology arm DNA sequence. The term “homology arm DNA sequence” refers to a polynucleotide sequence that has at least 80%, at least 85%, at least 90%, at least 91%, at least 92%, at least 93%, at least 94%, at least 95%, at least 96%, at least 97%, at least 98%, at least 99% or 100% sequence identity to a target sequence in a plant or plant cell. Further, the DNA sequence can be linear or circular, and can be single-stranded or double-stranded. It can be delivered to the cell as naked nucleic acid, as a complex with one or more delivery agents (e.g., liposomes, poloxamers, T-strand encapsulated with proteins, etc.) or contained in a bacterial or viral delivery vehicle, such as, for example, Agrobacterium tumefaciens or a Gemini Virus, or a nanovirus, respectively.

Once a specific target site is identified, a site-specific nuclease targeting the selected target site can be designed and introduced into the plant, seed, or plant cell. For example, a CRISPR-associated nuclease (e.g., Cas12a/Cpf1) and at least one RNA guide molecule that can hybridize to the target site can be designed and cloned into a plant expression vector and delivered to the plant, seed, or plant cell. If the genome modification is designed to induce a double-strand break (DSB) (i.e., induce a cleavage) with non-homologous end joining (NHEJ) repair for introduction of insertions and deletions (indels), then just the engineered CRISPR nuclease and at least one RNA guide molecule are delivered to the plant, seed or cell. If a DNA sequence of interest is to be incorporated at the target site, then the engineered CRISPR nuclease, at least one RNA guide molecule, and the DNA of interest are co-delivered to the plant, seed, or cell. The DNA of interest may integrate into the target site by NHEJ (Non-homologous End Joining) or by homology-dependent repair (HR). In the latter case, the DNA of interest will have at least one homology arm DNA sequence. An alternative to delivery of the engineered CRISPR nuclease as a DNA expression construct is the delivery of a ribonucleoprotein (RNP) complex of the CRISPR associated nuclease protein in complex with the guide RNA.

Following delivery of the site-specific nuclease to a plant cell, the cells or plants regenerated from the cells are sampled to confirm the presence of the intended site-specific genome modification including insertion of the DNA sequence of interest at or proximal to the target site. Methods of detecting the genome modification are known to one skilled in the art, and include PCR, TaqMan® PCR, droplet digital PCR (ddPCR™, Bio-Rad Laboratories, Hercules, Calif.), sequencing, Sanger sequencing, ABI 3730 DNA fragment analysis (Applied Biosystems, Grand Island, N.Y.), Southern analysis, Northern analysis, phenotypic analysis, or any other technique known to one in the art to detect genome modification.

As further described in Example 2 hereinbelow, seventeen target sites within 5 centimorgan upstream and downstream of event MON87429 were identified. The sequences of these target sites are provided herein as SEQ ID NOs. 174-190. Guide RNA spacer sequences corresponding to each of the target site sequences are provided as SEQ ID NOs. 195-211.

Corn plants, plant seeds, plant parts, plant cells, and progeny plants are provided. The plant, seed, plant part, plant cell, or progeny plant comprises a recombinant nucleic acid molecule. The recombinant nucleic acid molecule comprises a target corn genomic nucleic acid sequence having at least 85% sequence identity, at least 90% sequence identity, or at least 95% sequence identity, to a nucleic acid molecule selected from the group consisting of SEQ ID NOs. 174-190. The recombinant nucleic acid molecule further comprises a DNA sequence of interest. The DNA sequence of interest is inserted into said target corn genomic nucleic acid sequence. In some embodiments, the plant, seed, plant part, or progeny plant comprises a recombinant nucleic acid molecule comprising a target corn genomic nucleic acid sequence having a sequence selected from the group consisting of SEQ ID NOs:174-190.

The DNA sequence of interest can comprise a gene of agronomic interest. For example, the gene of agronomic interest can confer herbicide tolerance in plants.

In some embodiments, the target corn genomic nucleic acid sequence is at least 1 kb from the MON87429 insertion site. In some embodiments, the target corn genomic nucleic acid sequence maps to within 5 cM of the MON87429 insertion site. In some embodiments, the target corn genomic nucleic acid sequence is more than 1 kb from a gene, is more than 1 kb from a repressive chromatin mark, is more than 200 nucleotides from a small RNA hotspot, is more than 1 kb from a long repeat region, has DNA methylation less than or equal to 10% of genome-wide population average, and/or has a redundancy score less than or equal to 30%.

A method of generating a recombinant corn plant cell is provided. The method comprises: (a) obtaining a corn plant, seed, or cell, wherein said plant, seed, or cell comprises a target corn genomic nucleic acid molecule having at least 85% sequence identity, at least 90% sequence identity, or at least 95% sequence identity to a nucleic acid molecule selected from the group consisting of SEQ ID NOs: 174-190; (b) introducing into the corn plant, seed, or cell a site-specific nuclease that can specifically bind to and cleave the target corn genomic nucleic acid molecule; (c) introducing a DNA sequence of interest into the corn plant, seed, or cell; and (d) selecting recombinant corn plants, seeds or cells comprising the DNA sequence of interest inserted in the target corn genomic nucleic acid molecule. The site-specific nuclease can be selected from the group consisting of an RNA-guided nuclease, a zinc finger nuclease and a TALEN. For example, the RNA-guided nuclease can be Cas12a. The method can further comprise introducing into the corn plant, seed, or cell a guide polynucleotide comprising a nucleic acid sequence that is substantially complementary to the target corn genomic nucleic acid, wherein the guide polynucleotide and the RNA-guided nuclease form a complex that can bind to and cleave the corn genomic nucleic acid molecule. The guide polynucleotide can comprise a nucleotide sequence having at least 85% sequence identity, at least 90% sequence identity, or at least 95% sequence identity to a nucleic acid molecule selected from the group consisting of SEQ ID NOs:195-211. The guide polynucleotide can further comprise SEQ ID NO:23. In some embodiments, the target corn genomic nucleic acid sequence is at least 1 kb from the MON87429 insertion site. In some embodiments, the target corn genomic nucleic acid sequence maps to within 5 cM of the MON87429 insertion site. In some embodiments, the target corn genomic nucleic acid sequence is more than 1 kb from a gene, is more than 1 kb from a repressive chromatin mark, is more than 200 nucleotides from a small RNA hotspot, is more than 1 kb from a long repeat region, has DNA methylation less than or equal to 10% of genome-wide population average, and/or has a redundancy score less than or equal to 30%.

A recombinant DNA molecule is provided. The recombinant DNA molecule comprises a DNA sequence having at least 85% sequence identity, at least 90% sequence identity, or at least 95% sequence identity to a nucleic acid molecule selected from the group consisting of SEQ ID NOs:195-211. For example, the nucleic acid molecule can be selected from the group consisting of SEQ ID NOs:195-211. The DNA sequence can be operably linked to a heterologous promoter sequence. The recombinant DNA molecule can further comprise SEQ ID NO: 23.

A recombinant RNA molecule is provided. The recombinant RNA molecule comprises an RNA sequence that is at least 85% complementary, at least 90% complementary, or at least 95% complementary, to a nucleic acid molecule selected from the group consisting of SEQ ID NOs:195-211. In some embodiments, the RNA sequence is 100% complementary to a nucleic acid molecule selected from the group consisting of SEQ ID NOs:195-211.

The plants described herein can be used to produce progeny or offspring that comprise corn event Zm_CSM63715. Such progeny may include any plant, seed, and cell and/or regenerable plant part comprising corn event Zm_CSM63715 inherited or derived from an ancestor or parental corn plant(s), at least one of which comprises a DNA molecule having or comprising at least one polynucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, a polynucleotide comprising at least 16 consecutive nucleotides of SEQ ID NO:1, at least 16 consecutive nucleotides of SEQ ID NO:2, at least 31 consecutive nucleotides of SEQ ID NO:3, at least 35 consecutive nucleotides of SEQ ID NO:4, at least 51 consecutive nucleotides of SEQ ID NO:5, or at least 51 consecutive nucleotides of SEQ ID NO:6, or a polynucleotide having a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO: 9.

Corn plants, progeny, and seeds may be homozygous or heterozygous for the event Zm_CSM63715 and the transgenes of event Zm_CSM63715. Progeny may be grown from seeds produced by a corn plant comprising or containing event Zm_CSM63715 and/or from seeds produced by a plant fertilized with pollen from a corn plant comprising or containing event Zm_CSM63715 (i.e., fertilized with pollen comprising or containing event Zm_CSM63715).

Plants or progeny may also be obtained by tissue culture and regeneration methods from a protoplast, cell, embryo or reproductive or somatic tissue derived from a corn plant comprising or containing corn event Zm_CSM63715.

Progeny plants may be self-pollinated (also known as “selfing”) to generate a true breeding line of plants, i.e., plants homozygous for the corn event Zm_CSM63715 DNA. Alternatively, progeny plants may be outcrossed, i.e., bred with another plant, to produce a varietal or a hybrid seed or plant. The other plant may be transgenic or non-transgenic. A varietal or hybrid seed or plant of the present disclosure may thus be derived by crossing a first parent that lacks the specific and unique DNA of event Zm_CSM63715 with a second parent comprising event Zm_CSM63715, resulting in a hybrid comprising the specific and unique DNA of event Zm_CSM63715. Each parent can be a hybrid or an inbred/variety, so long as the cross or breeding results in a plant or seed of the present disclosure, i.e., a seed having at least one allele comprising the specific and unique DNA of event Zm_CSM63715 and/or at least 16 consecutive nucleotides of SEQ ID NO:1, at least 16 consecutive nucleotides of SEQ ID NO:2, at least 31 consecutive nucleotides of SEQ ID NO:3, at least 35 consecutive nucleotides of SEQ ID NO:4, at least 51 consecutive nucleotides of SEQ ID NO:5, or at least 51 consecutive nucleotides of SEQ ID NO:6, or a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO:9.

Sexually crossing one plant with another plant, i.e., cross-pollinating, may be accomplished or facilitated by human intervention, for example: by human hands collecting the pollen of one plant and contacting this pollen with the style or stigma of a second plant; by human hands and/or human actions removing, destroying, or covering the stamen or anthers of a plant (e.g., by manual intervention or by application of a chemical gametocide) so that natural self-pollination is prevented and cross-pollination would have to take place in order for fertilization to occur; by human placement of pollinating insects in a position for “directed pollination” (e.g., by placing beehives in orchards or fields or by caging plants with pollinating insects); by human opening or removing of parts of the flower to allow for placement or contact of foreign pollen on the style or stigma; by selective placement of plants (e.g., intentionally planting plants in pollinating proximity); and/or by application of chemicals to precipitate flowering or to foster receptivity (of the stigma for pollen).

Two different transgenic plants of the same or different genetic backgrounds may thus be crossed to produce inbred or hybrid offspring plants, plant parts and/or seeds that contain two independently segregating transgenes or events wherein at least one of those transgenes or events comprises or is contained within corn event Zm_CSM63715. For example, transgenic plants comprising corn event Zm_CSM63715 can be crossed with other transgenic corn plants to produce a plant having the characteristics of both transgenic parents.

Back-crossing to a parental plant and out-crossing with a non-transgenic plant are also contemplated, as is vegetative propagation. Descriptions of other breeding methods that are commonly used for different traits and crops are known in the art and can be found in one of several references, e.g., Fehr, in Breeding Methods for Cultivar Development, Wilcox J. ed., American Society of Agronomy, Madison WI (1987).

A plant part is provided. As used herein, a “plant part” refers to any part of a plant that is comprised of material directly from or derived from a plant comprising corn event Zm_CSM63715. Plant parts include but are not limited to microspores, pollen, anthers, silk, spikes, ovules, ovaries, flowers, cobs, pods, embryos, stems, leaves, roots, and calli, in whole or part. Plant parts may be viable or nonviable. Plant parts may be regenerable or non-regenerable.

Nonliving or nonregenerable corn plant materials are provided herein. The nonliving or nonregenerable corn plant material can comprise any of the recombinant DNA molecules characteristic of corn event Zm_CSM63715 described herein, or any of the DNA constructs described herein. The nonliving or nonregenerable corn plant material can comprise corn event Zm_CSM63715, a representative sample of seed comprising the corn event corn event Zm_CSM63715 having been deposited under ATCC Accession No. PTA-127361

Commodity products that comprise any of the DNA molecules characteristic of corn event Zm_CSM63715 or any of the DNA constructs described herein are provided. Such commodity products can be produced from plants comprising corn event Zm_CSM63715. The commodity products contain a detectable amount of DNA comprising a DNA sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO:9. As used herein, a “commodity product” refers to any composition or product which is comprised of material from plant, seed, cell, or plant part comprising corn event Zm_CSM63715. Commodity products may be viable or non-living plant material, that is, a material that is not living and derived from a plant, seed, cell, or plant part comprising corn event Zm_CSM63715. Nonviable commodity products include but are not limited to nonviable seeds, whole or processed seeds, processed plant tissues or plant parts, dehydrated plant tissues or parts, frozen plant tissues or parts, food for human consumption such as corn oil, corn meal, cereal, corn flour, corn grits, corn flakes, corn bran, corn starch, fiber, sweetener such as high fructose corn syrup (HFCS), glucose and dextrose, beverage alcohol, brewer grits for beer production; animal feed such as corn, corn biomass; industrial alcohol, fuel ethanol, corn pollen, corn plastic, dried distillers grains (DDGs), and bio-degradable packing materials. Viable commodity products include but are not limited to viable seeds, viable plant parts (such as root and leaf) and viable plant cells. A plant comprising event Zm_CSM63715 can thus be used to manufacture any commodity product typically acquired from a corn plant. Any such commodity product that is derived from the plants comprising event Zm_CSM63715 may contain at least a detectable amount of the specific and unique DNA corresponding to event Zm_CSM63715, and specifically may contain a detectable amount of a polynucleotide having a nucleotide sequence of at least 16 consecutive nucleotides of SEQ ID NO:1, at least 16 consecutive nucleotides of SEQ ID NO:2, at least 31 consecutive nucleotides of SEQ ID NO:3, at least 35 consecutive nucleotides of SEQ ID NO:4, at least 51 consecutive nucleotides of SEQ ID NO:5, or at least 51 consecutive nucleotides of SEQ ID NO:6, or a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO:9. Any standard method of detection for polynucleotide molecules may be used, including methods of detection disclosed herein.

Methods for producing such commodity products are also provided. Such methods comprise: (a) obtaining a transgenic corn plant, plant part, or plant seed comprising corn event Zm_CSM63715; and (b) producing a commodity product from the transgenic corn plant, plant part, or plant seed.

A plant tolerant to herbicides may be produced by sexually crossing a plant comprising event Zm_CSM63715 with another plant and thereby producing seed, which is then grown into progeny plants. For example, provided herein is a method of producing a progeny corn plant comprising event Zm_CSM63715, the method comprising: (a) sexually crossing a first corn plant that comprises corn event Zm_CSM63715 with itself or a second corn plant; (b) collecting one or more seeds produced from the cross; (c) growing one or more seeds to produce one or more progeny plants; and (d) selecting at least a first progeny plant or seed comprising corn event Zm_CSM63715. Inbred and hybrid corn plants comprising corn event Zm_CSM63715 produced by such methods are also provided.

The progeny plants may be analyzed using diagnostic methods to select for progeny plants that comprise event Zm_CSM63715 DNA or for progeny plants tolerant to the PPO herbicides such as flumioxazin, epyrifenacil, lactofen, acifluorfen, pyraflufen, pyraflufen-ethyl, oxadiazon, butafenacil, pyridin-2-ylmethyl[(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl[(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl[(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate, cyanomethyl[(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate, methyl (2R)-2-{[(E)-({2-chloro-4-fluoro-5-[3-methyl-2,6-dioxo-4-(trifluoromethyl)-3,6-dihydropyrimidin-1(2H)-yl]phenyl}methylidene)amino]oxy}propanoate (flufenoximacil), cyclopropylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate, fomesafen, saflufenacil, sulfentrazone, tiafenacil, and trifludimoxazin, and combinations of any thereof. The other plant used may or may not be transgenic. The progeny plant and/or seed produced may be varietal or hybrid seed.

A plant tolerant to PPO herbicides may be produced by selfing a plant comprising event Zm_CSM63715 comprising a polynucleotide having the nucleotide sequence of SEQ ID NOs:1-10, at least 16 consecutive nucleotides of SEQ ID NO:1, at least 16 consecutive nucleotides of SEQ ID NO:2, at least 31 consecutive nucleotides of SEQ ID NO:3, at least 35 consecutive nucleotides of SEQ ID NO:4, at least 51 consecutive nucleotides of SEQ ID NO:5, or at least 51 consecutive nucleotides of SEQ ID NO:6, and a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO:9, and thereby producing seed, which is then grown into progeny plants. These progeny plants may then be analyzed using diagnostic methods to select for progeny plants that comprise event Zm_CSM63715 DNA, or for progeny plants tolerant to the PPO herbicides such as flumioxazin, epyrifenacil, lactofen, acifluorfen, pyraflufen, pyraflufen-ethyl, oxadiazon, butafenacil, pyridin-2-ylmethyl[(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl[(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl[(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate, cyanomethyl[(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate, methyl (2R)-2-{[(E)-({2-chloro-4-fluoro-5-[3-methyl-2,6-dioxo-4-(trifluoromethyl)-3,6-dihydropyrimidin-1(2H)-yl]phenyl}methylidene)amino]oxy}propanoate (flufenoximacil), cyclopropylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate, fomesafen, saflufenacil, sulfentrazone, tiafenacil, and trifludimoxazin, and combinations of any thereof.

Corn event Zm_CSM63715 contains the PPO expression cassette that provide tolerance to PPO herbicides such as flumioxazin, epyrifenacil, lactofen, acifluorfen, pyraflufen, pyraflufen-ethyl, oxadiazon, butafenacil, pyridin-2-ylmethyl[(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl[(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl[(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate, cyanomethyl[(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate, methyl (2R)-2-{[(E)-({2-chloro-4-fluoro-5-[3-methyl-2,6-dioxo-4-(trifluoromethyl)-3,6-dihydropyrimidin-1(2H)-yl]phenyl}methylidene)amino]oxy}propanoate (flufenoximacil), cyclopropylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate, fomesafen, saflufenacil, sulfentrazone, tiafenacil, and trifludimoxazin, and combinations of any thereof. Corn plants, progeny, and seeds of event Zm_CSM63715 may contain one or more additional desirable trait(s) such as tolerance to inhibitors of acetyl CoA carboxylase (ACCase) in the aryloxyphenoxy propionate (FOP) group such as quizalofop and haloxyfop, synthetic auxins such as dicamba and 2,4-D; inhibitors of glutamine synthetase such as glufosinate; inhibitors of EPSPS such as glyphosate; or combinations of any thereof.

PPO herbicides include diphenylethers, N-phenylphthalimides, oxadiazoles, oxazolidinediones, phenylpyrazoles, pyrimidinediones, thiadiazoles, triazolinones, benzoxazinone derivatives, other PPO herbicides, and combinations of any thereof. Examples of diphenylethers include, but are not limited to, acifluorfen, bifenox, ethoxyfen, fluorodifen, fluoronitrofen, furyloxyfen, halosafen, chlomethoxyfen, chlornitrofen, ethoxyfen-ethyl, fluoroglycofen, lactofen, nitrofen, oxyfluorfen, fomesafen, a salt of any thereof, and an ester of any thereof. Examples of N-phenylphthalimides include, but are not limited to, cinidon-ethyl, flumiclorac, flumiclorac-pentyl, and flumioxazin. Examples of oxadiazoles include, but are not limited to, oxadiargyl and oxadiazon. Examples of oxazolidinediones include, but are not limited to, pentoxazone. Examples of phenylpyrazoles include, but are not limited to, fluazolate, pyraflufen, and pyraflufen-ethyl.

Examples of pyrimidinediones or phenyluracils include, but are not limited to, benzfendizone, butafenacil, epyrifencacil, flupropacil, flufenoximacil, saflufenacil, and tiafenacil. Examples of thiadiazoles include, but are not limited to, fluthiacet-methyl and thidiazimin. Examples of triazolinones include, but are not limited to, azafenidin, bencarbazone, carfentrazone, its salts and esters, and sulfentrazone. Examples of benzoxazinone derivatives include, but are not limited to, 1,5-dimethyl-6-thioxo-3-(2,2,7-trifluoro-3,4-dihydro-3-oxo-4-prop-2-ynyl-2H-1,4-benzoxazin-6-yl)-1,3,5-triazinane-2,4-dione (trifludimoxazin)). Examples of other PPO herbicides include, but are not limited to, chlorphthalim, flufenpyr, flufenpyr-ethyl, flumipropyn, pyraclonil, and profluazol. Further examples of other PPO herbicides include:

1) an herbicidally active compound of the general formula (I) or an agrochemically acceptable salt thereof

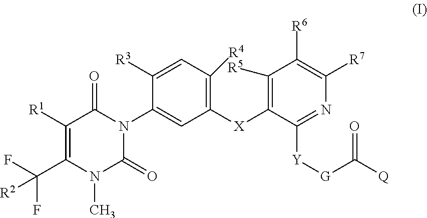

in which:
R¹ is hydrogen,
R² is hydrogen, fluorine, chlorine, bromine, trifluoromethyl, methoxy, ethoxy, prop-i-yloxy, or but-1-yloxy,
R³ is hydrogen, fluorine, chlorine, bromine, methoxy, ethoxy, prop-1-yloxy, prop-2-yloxy, but-1-yloxy, but-2-yloxy, 2-methylprop-1-yloxy, or 1,1-dimethyleth-1-yloxy,
R⁴ is fluorine, chlorine, bromine, cyano, NO₂, C(O)NH₂, C(S)NH₂, trifluoromethyl, difluoromethyl, pentafluoroethyl, ethynyl, propyn-1-yl, 1-butyn-1-yl, pentyn-1-yl, or hexyn-1-yl,
R⁵, R⁶ and R⁷ are independently hydrogen, fluorine, chlorine, bromine, iodine, cyano, methyl, ethyl, prop-1-yl, 1-methylethyl, but-1-yl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, trifluoromethyl, difluoromethyl, pentafluoroethyl, 2,2-difluoroethyl, 2,2,2-trifluoroethyl, methoxy, ethoxy, prop-1-yloxy, prop-2-yloxy, but-1-yloxy, but-2-yloxy, 2-methylprop-1-yloxy, 1,1-dimethyleth-1-yloxy, difluoromethoxy, trifluoromethoxy, pentafluoroethoxy, 2,2-difluoroethoxy, or 2,2,2-trifluoroethoxy,
G is methylene, (methyl)methylene, (ethyl)methylene, (prop-1-yl)methylene, (prop-2-yl)methylene, (but-1-yl)methylene, (but-2-yl)methylene, (pent-1-yl)methylene, (pent-2-yl)methylene, (pent-3-yl)methylene, (dimethyl)methylene, (diethyl)methylene, ethylene, n-propylene, (1-methyl)ethyl-1-ene, (2-methyl)ethyl-1-ene, n-butylene, 1-methylpropyl-1-ene, 2-methylpropyl-1-ene, 3-methylpropyl-1-ene, 1,1-dimethylethyl-1-ene, 2,2-dimethylethyl-1-ene, 1-ethylethyl-1-ene, 2-ethylethyl-1-ene, 1-(prop-1-yl)ethyl-1-ene, 2-(prop-1-yl)ethyl-1-ene, 1-(prop-2-yl)ethyl-1-ene, 2-(prop-2-yl)ethyl-1-ene, 1,1,2-trimethylethyl-1-ene, 1,2,2-trimethylethyl-1-ene, 1,1,2,2-tetramethylethyl-1-ene, n-pentylene, 1-methylbutyl-1-ene, 2-methylbutyl-1-ene, 3-methylbutyl-1-ene, 4-methylbutyl-1-ene, 1,1-dimethylpropyl-1-ene, 2,2-dimethylpropyl-1-ene, 3,3-dimethylpropyl-1-ene, 1,2-dimethylpropyl-1-ene, 1,3-dimethylpropyl-1-ene, 1-ethylpropyl-1-ene, n-hexylene, 1-methylpentyl-1-ene, 2-methylpentyl-1-ene, 3-methylpentyl-1-ene, 4-methylpentyl-1-ene, 1,1-dimethylbutyl-1-ene, 1,2-dimethylbutyl-1-ene, 1,3-di-methylbutyl-1-ene, 2,2-dimethylbutyl-1-ene, 2,3-dimethylbutyl-1-ene, 3,3-dimethylbutyl-1-ene, 1-ethylbutyl-1-ene, 2-ethylbutyl-1-ene, 1,1,2-trimethylpropyl-1-ene, 1,2,2-trimethylpropyl-1-ene, 1-ethyl-1-methylpropyl-1-ene, or 1-ethyl-2-methylpropyl-1-ene,
X and Y are independently O (oxygen) or S (sulfur)
and
Q is one of the following moieties Q-1 to Q-54, Q-56 to Q-57, Q-60 to Q-89, Q-91 to Q-129, Q-131 to Q-139, Q-141 to Q-144, Q-146 to Q-180, Q-182 to Q-185, Q-193 to Q-195, Q-200 to Q-208, Q-210 to Q-370, Q-395 to Q-440:

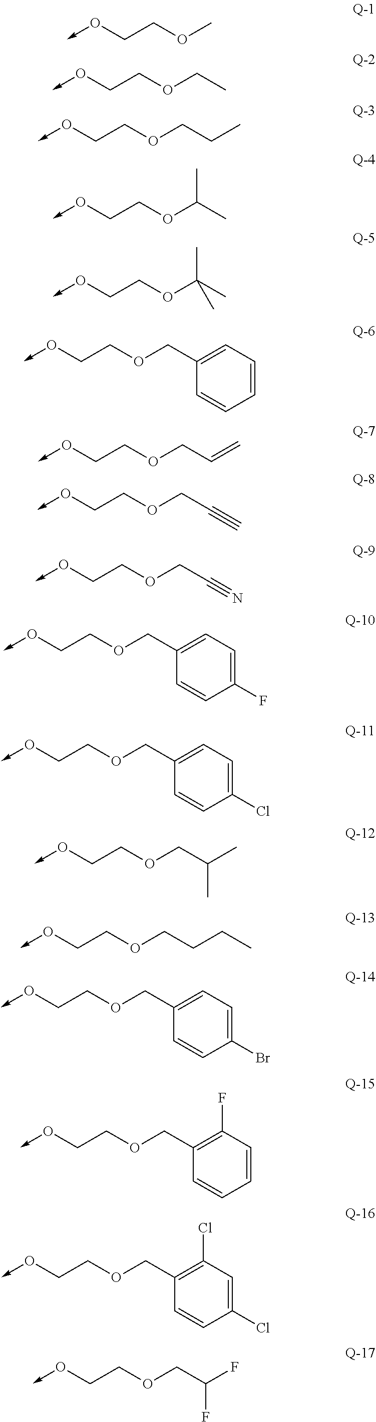

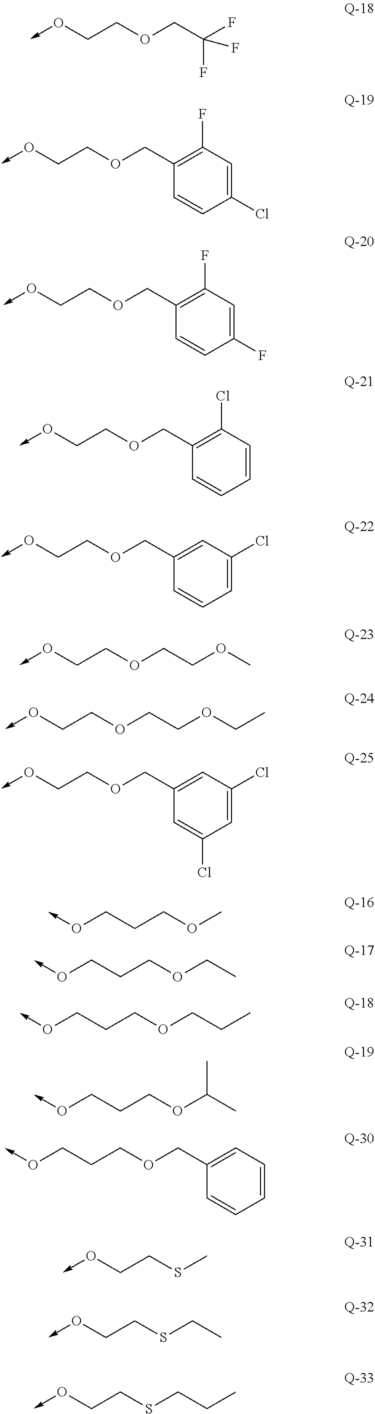

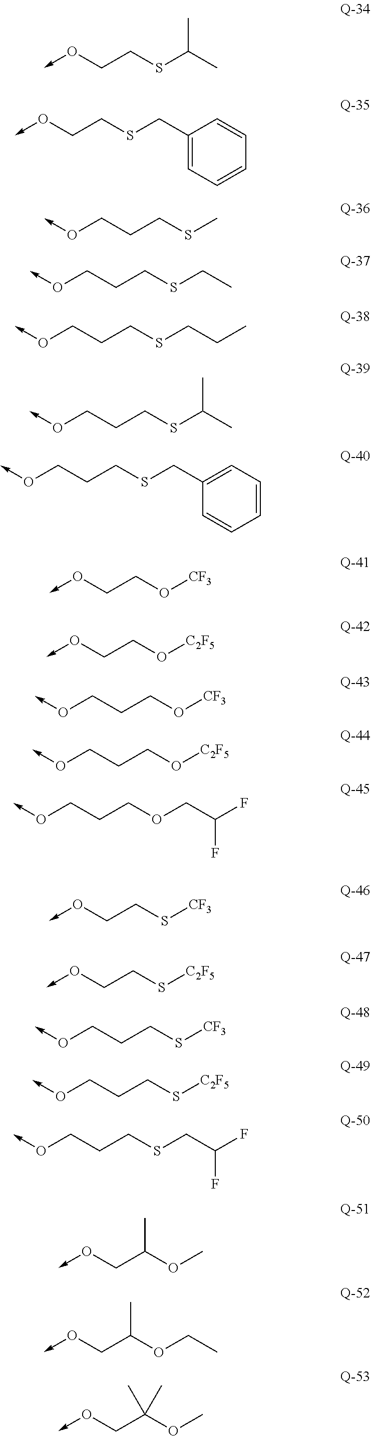

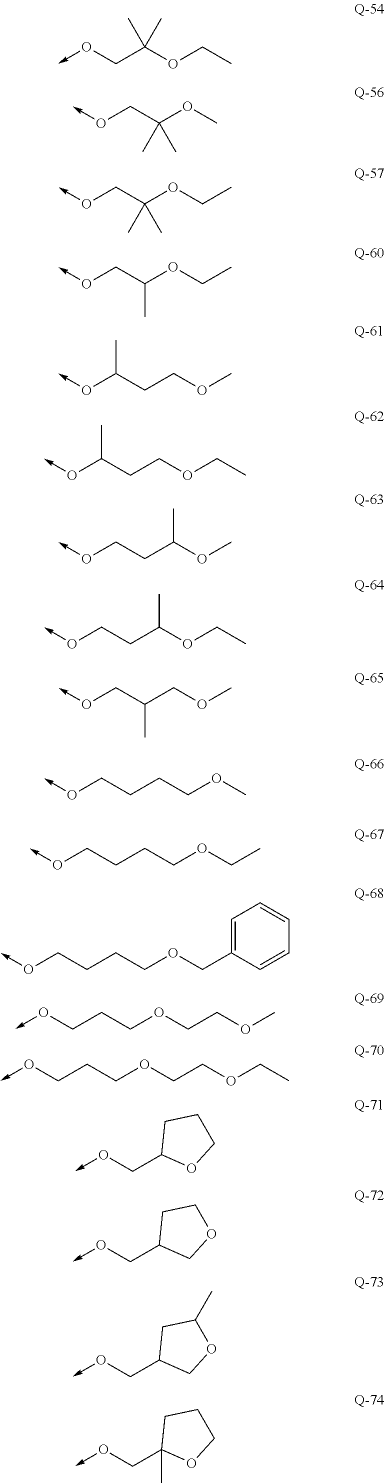

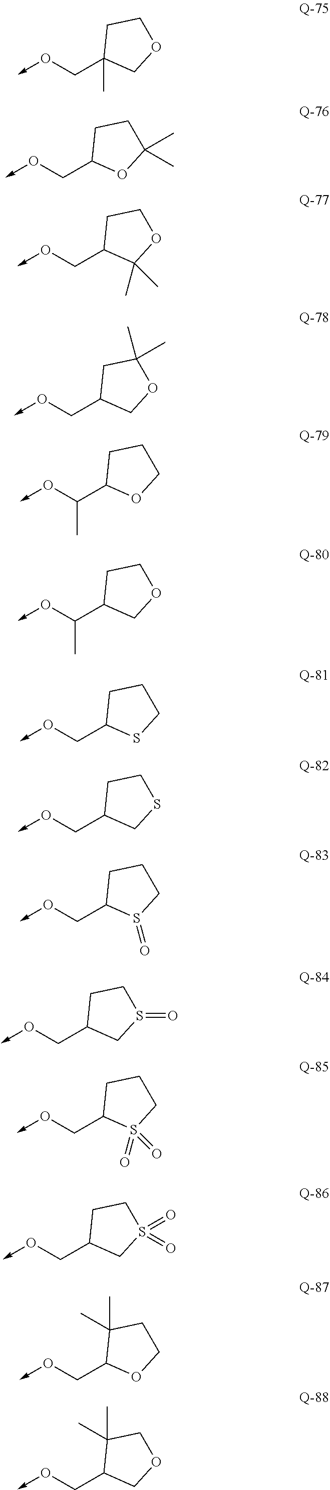

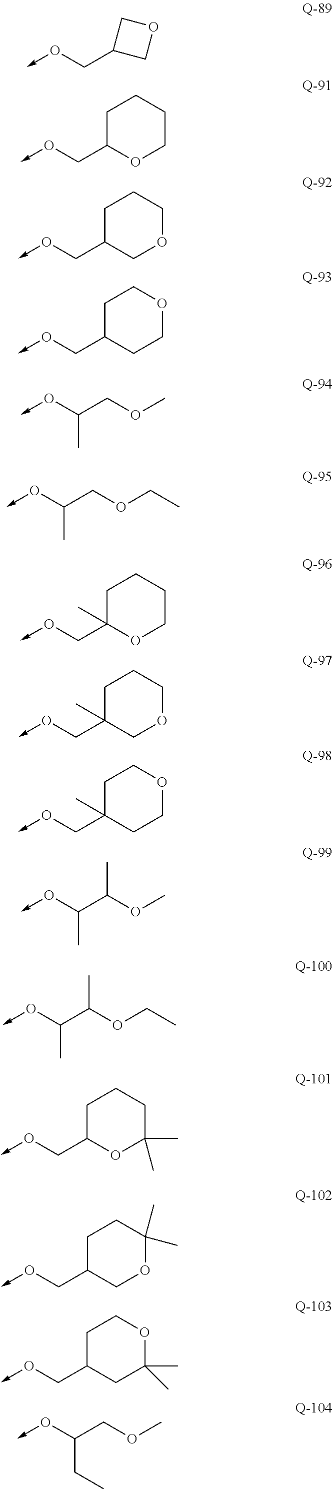

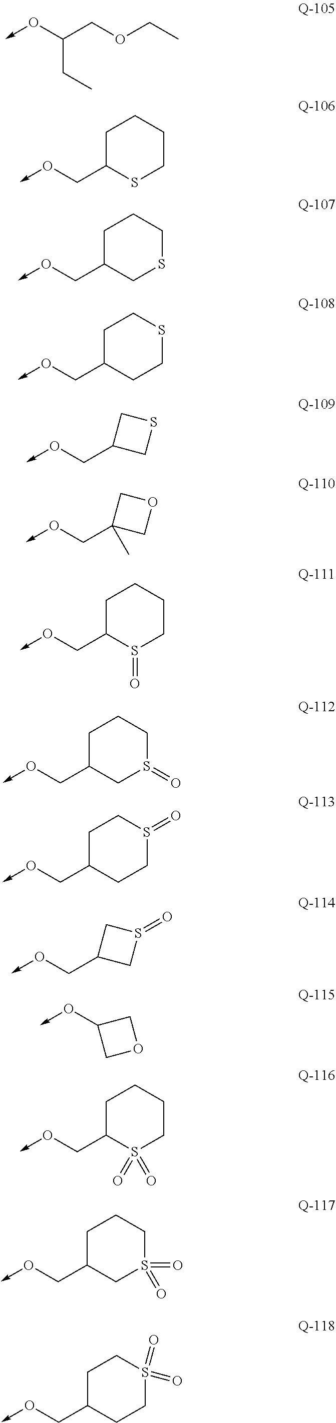

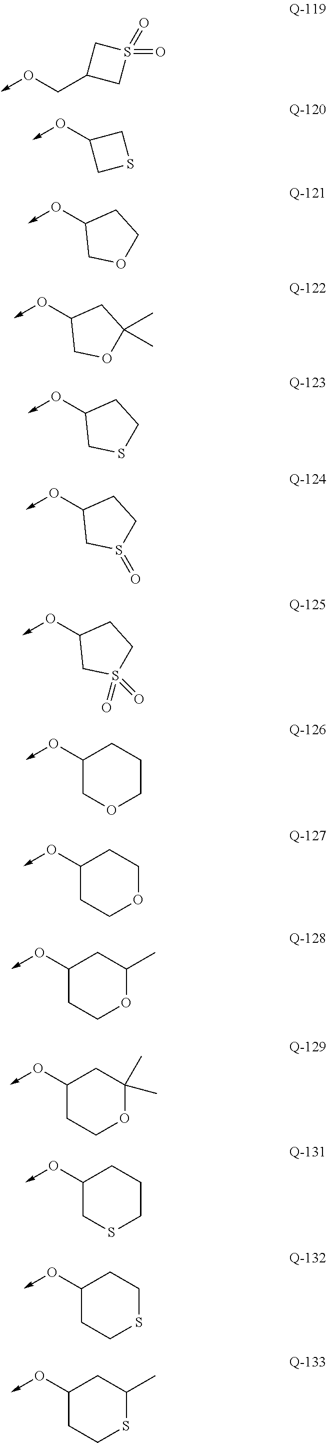

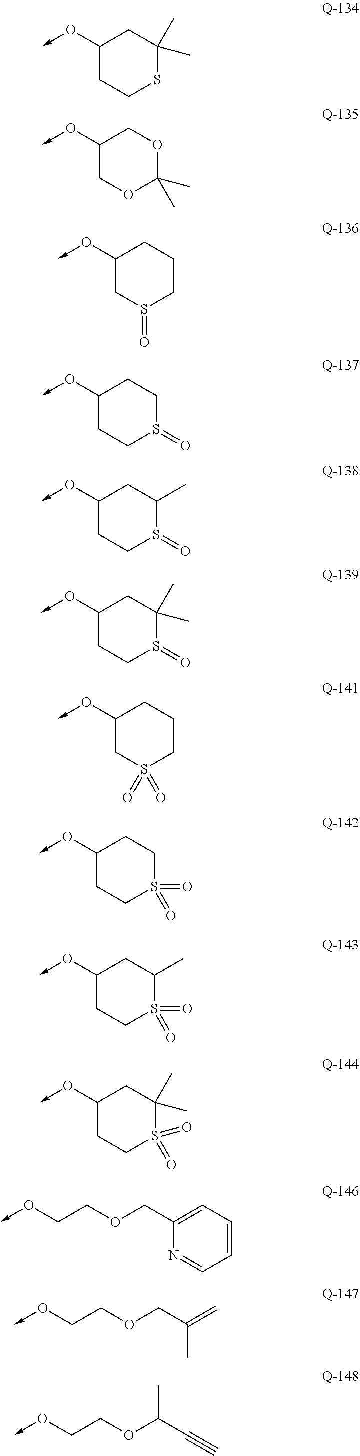

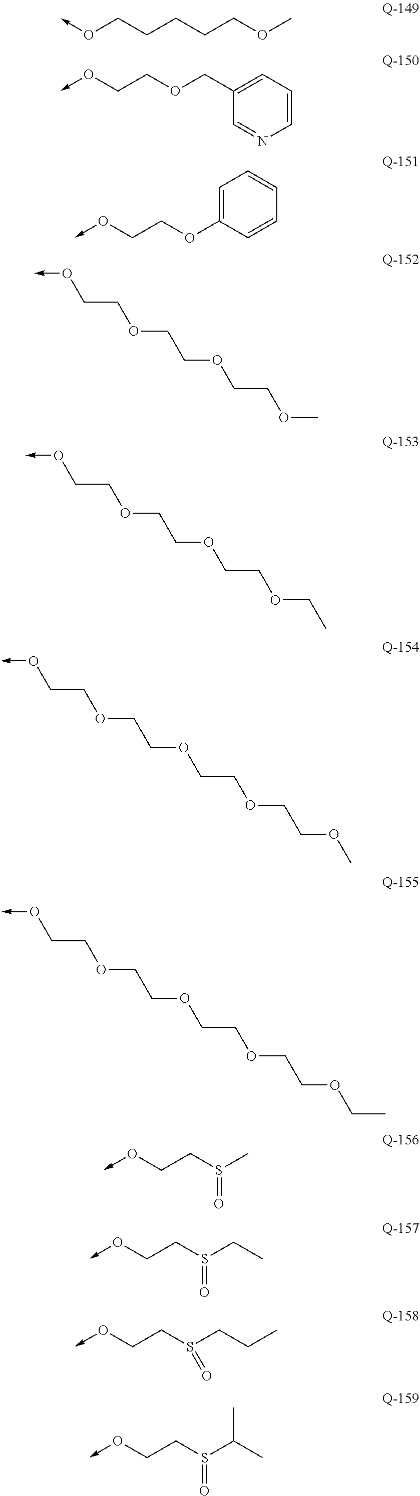

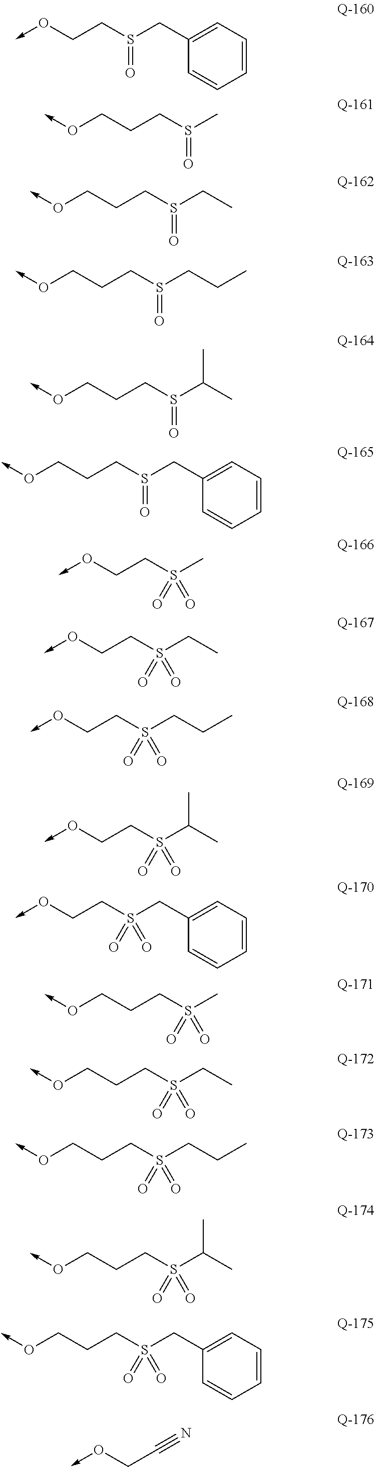

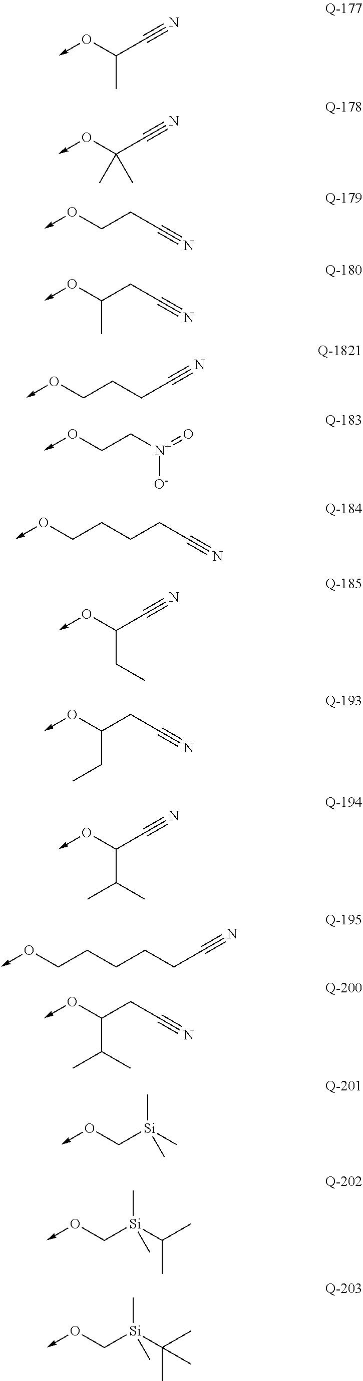

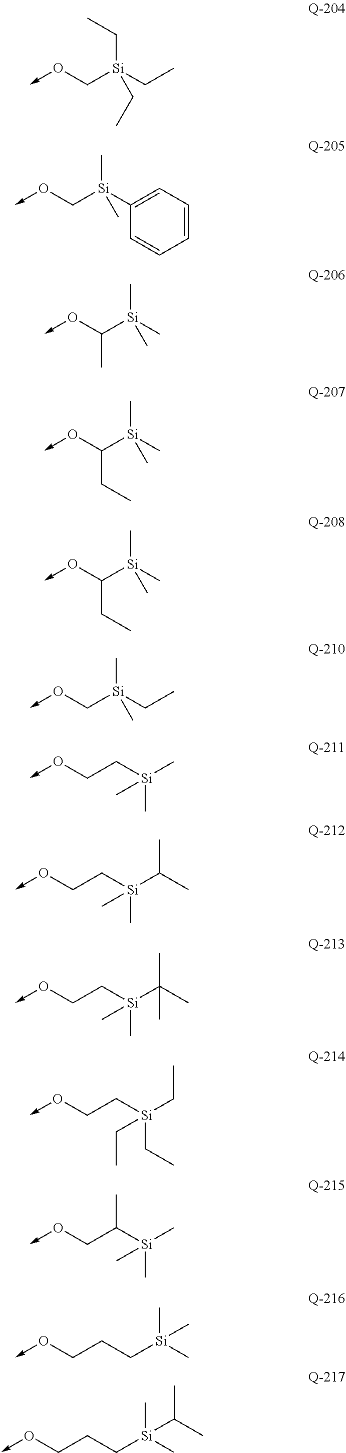

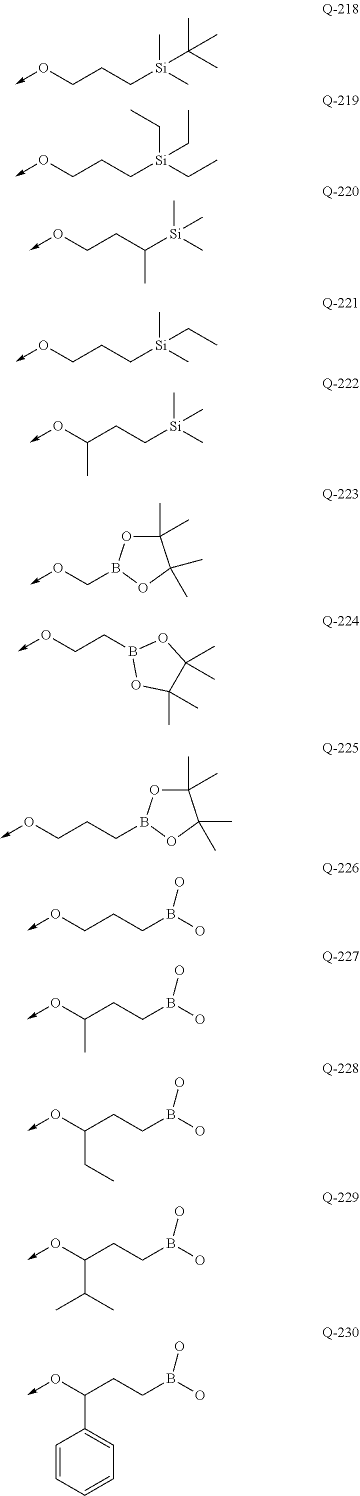

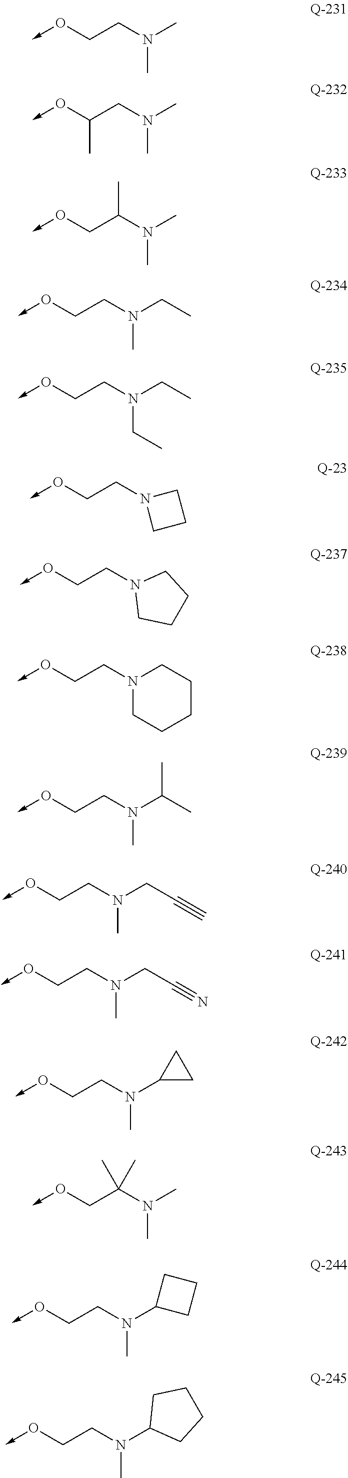

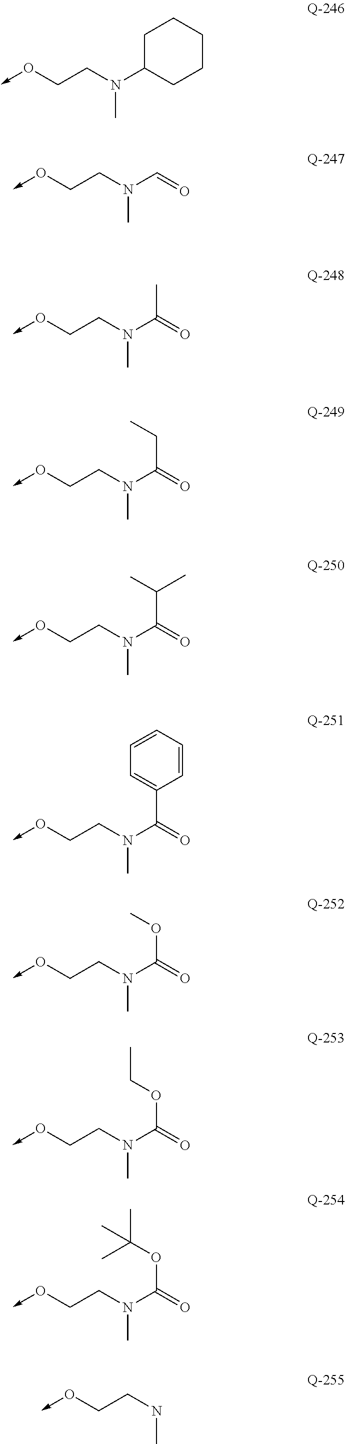

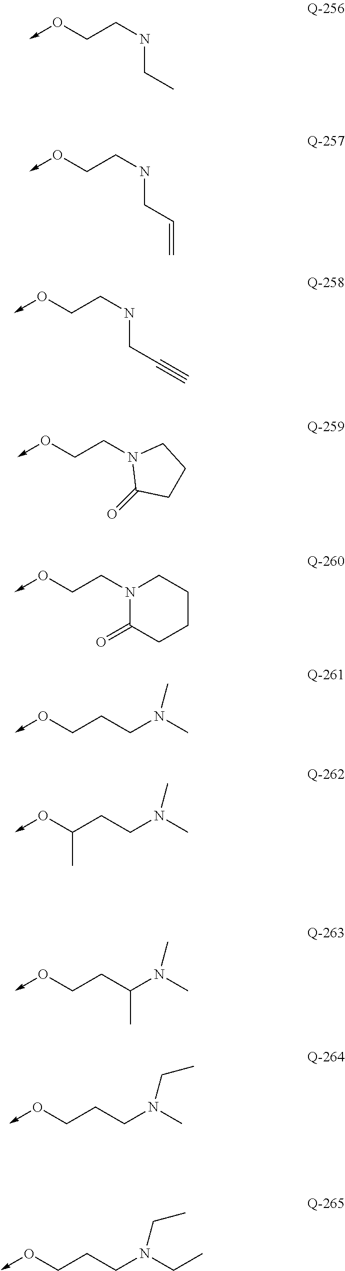

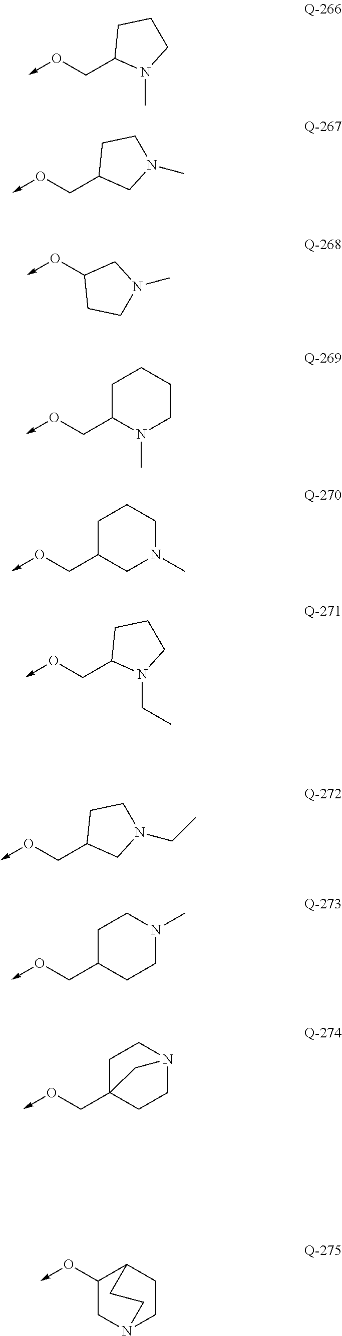

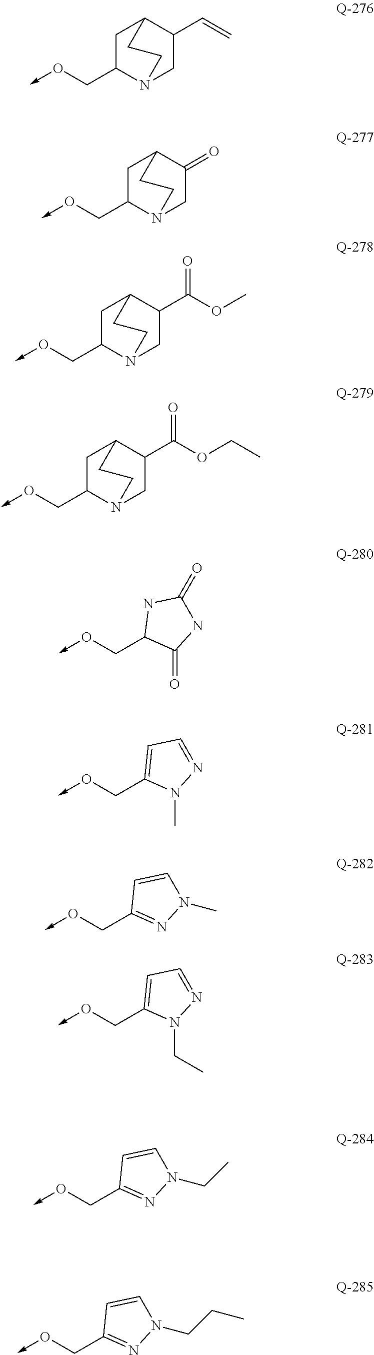

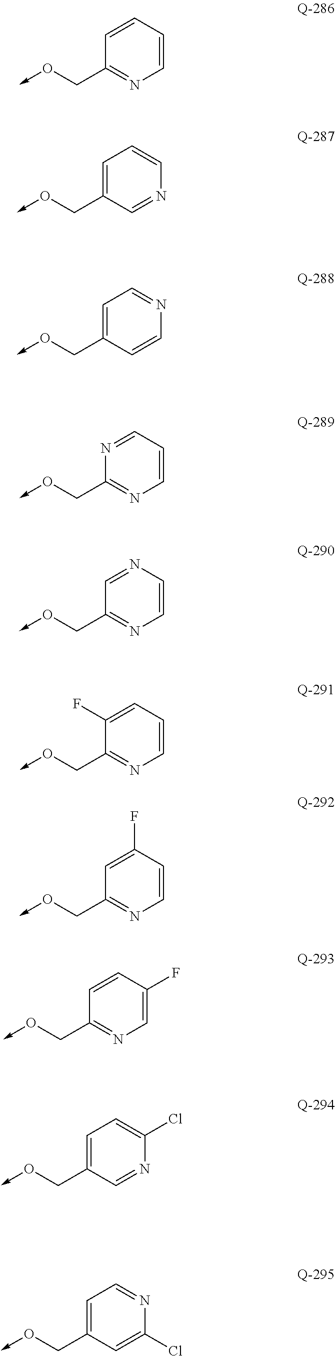

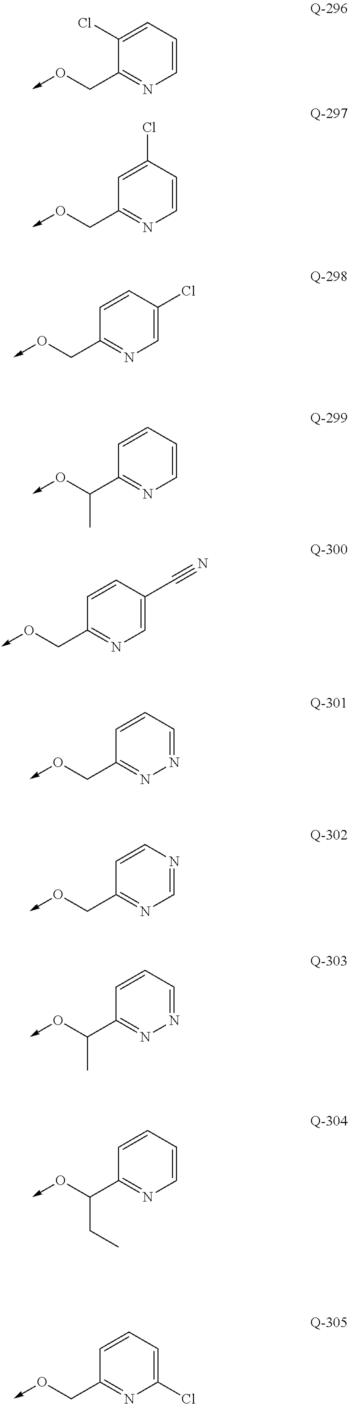

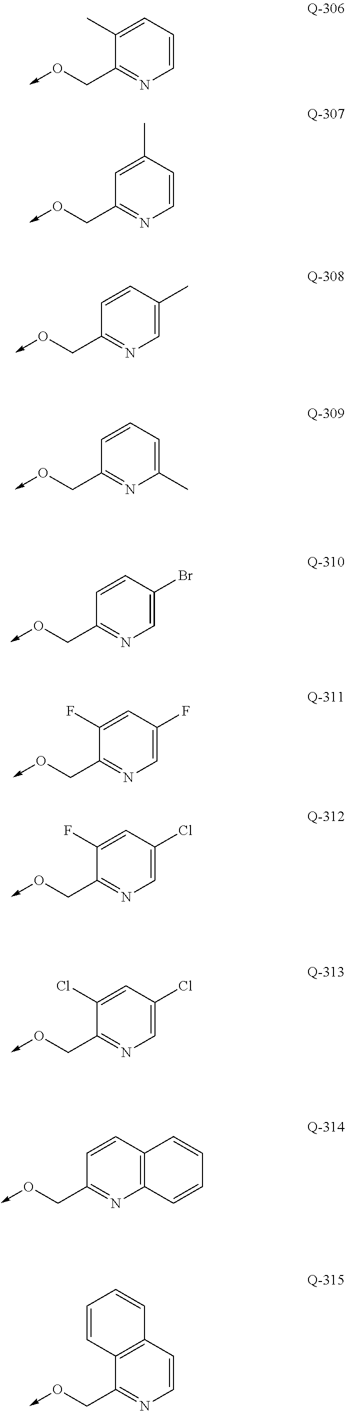

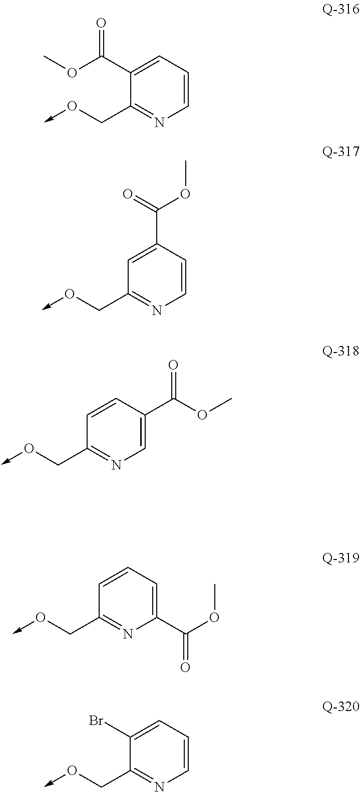

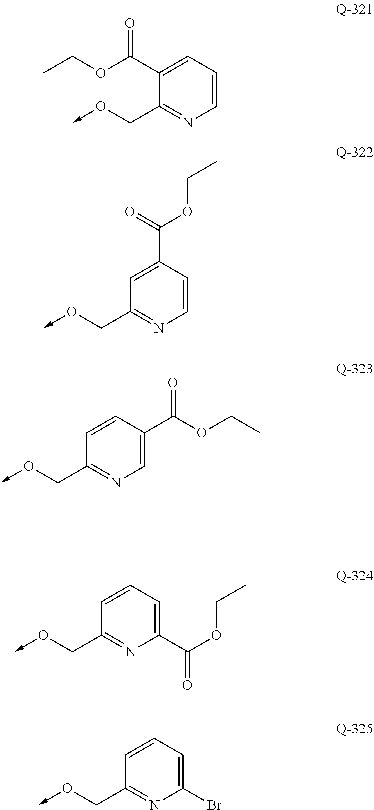

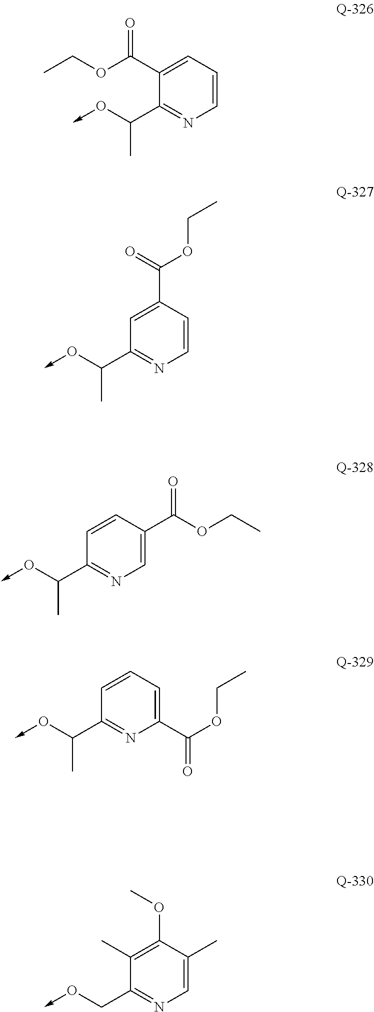

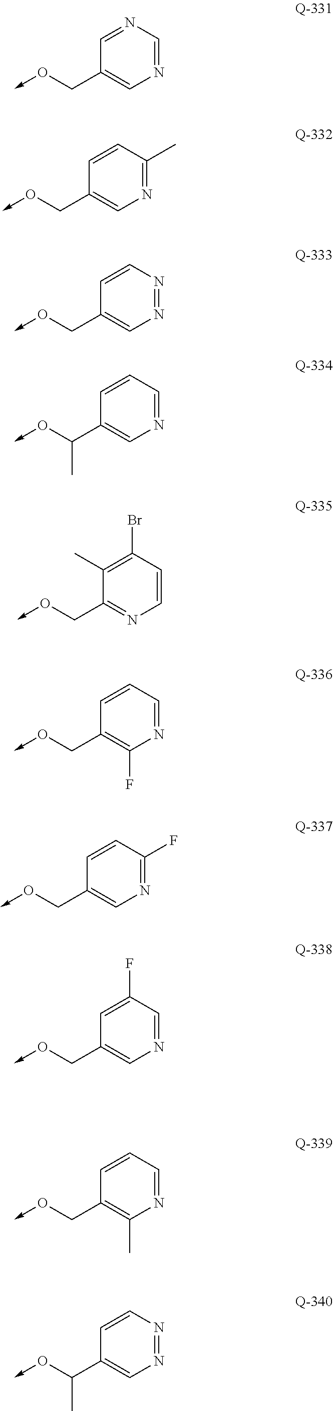

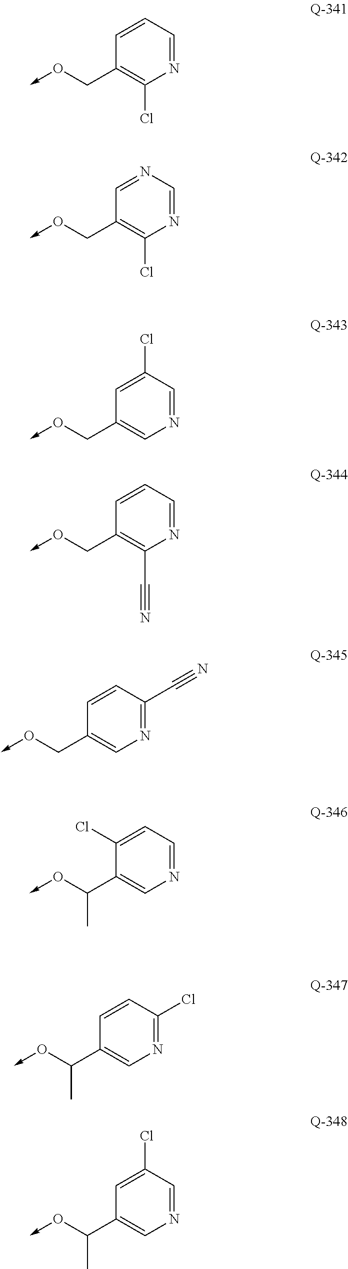

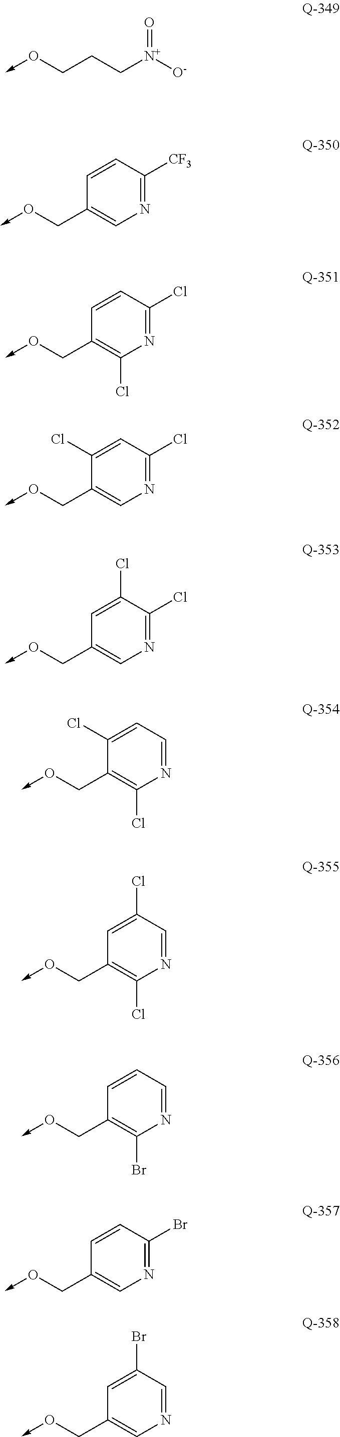

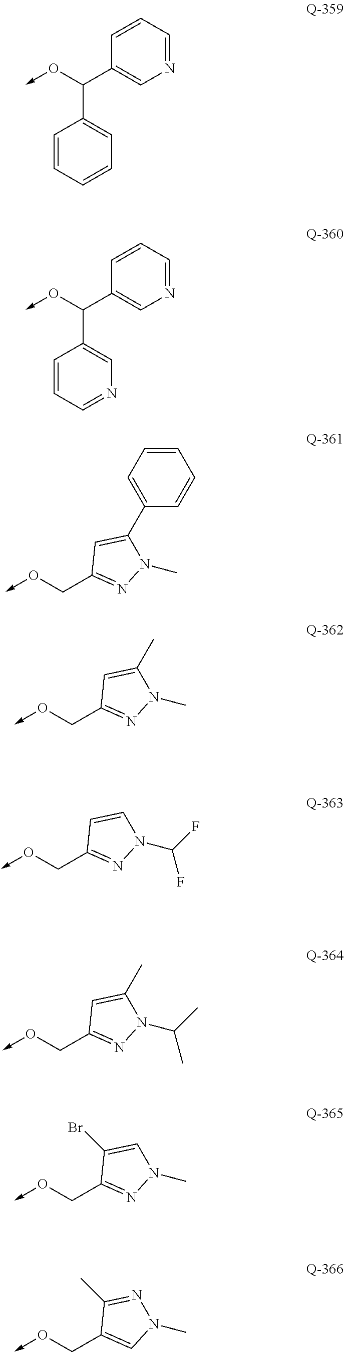

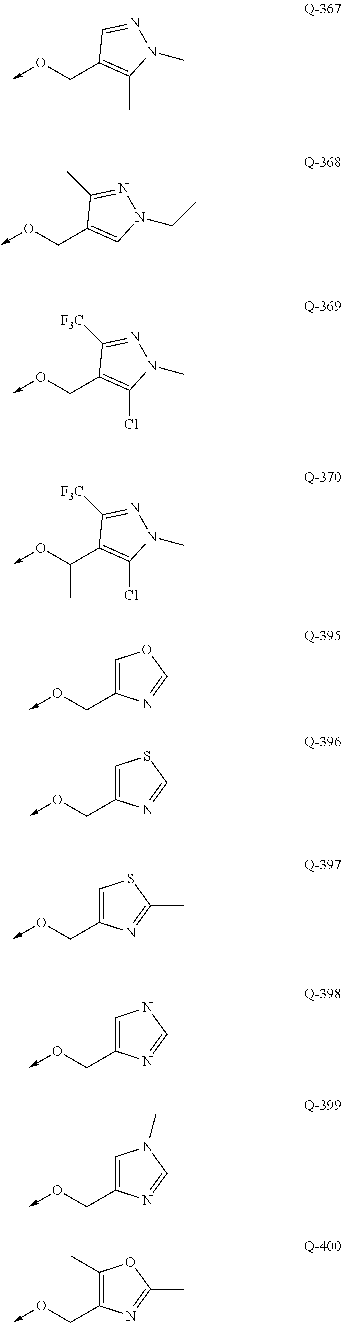

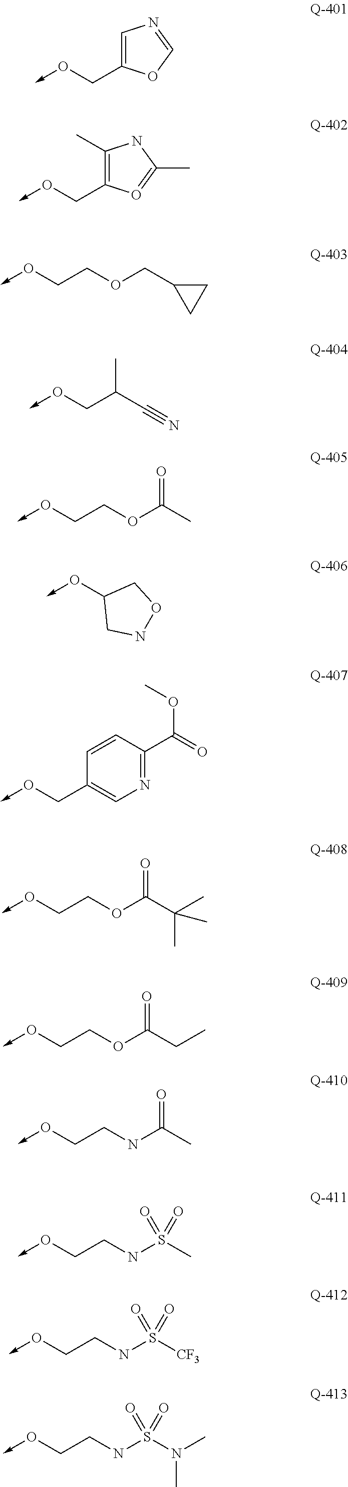

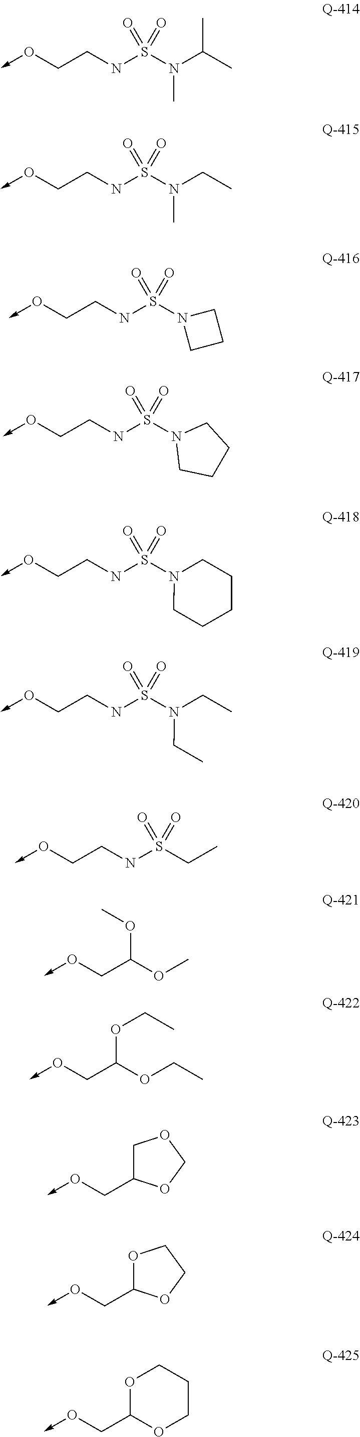

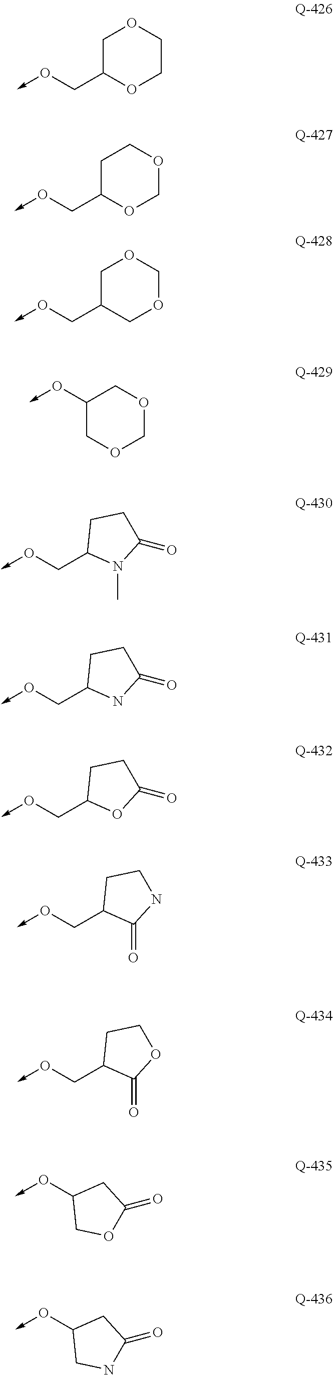

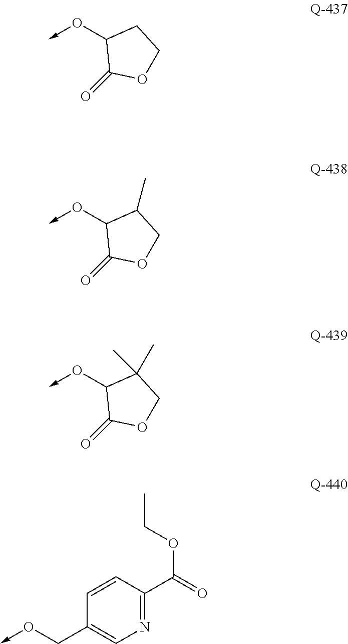

Examples of such herbicidally active compounds within the scope of formula (I) include:

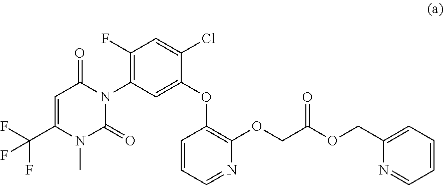 [SMILES: Cn1c(C(F)(F)F)cc(=O)n(-c2cc(Oc3cccnc3OCC(=O)OCc3ccccn3)c(Cl)cc2F)c1=O]

pyridin-2-ylmethyl[(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate;

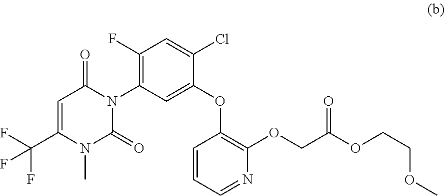 [SMILES: COCCOC(=O)COc1ncccc1Oc1cc(-n2c(=O)cc(C(F)(F)F)n(C)c2=O)c(F)cc1Cl]

2-methoxyethyl[(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate;

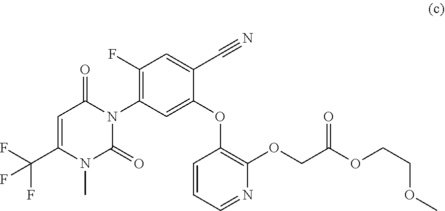 [SMILES: COCCOC(=O)COc1ncccc1Oc1cc(-n2c(=O)cc(C(F)(F)F)n(C)c2=O)c(F)cc1C#N]

2-methoxyethyl[(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate; and

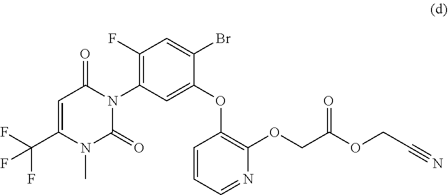 [SMILES: Cn1c(C(F)(F)F)cc(=O)n(-c2cc(Oc3cccnc3OCC(=O)OCC#N)c(Br)cc2F)c1=O]

cyanomethyl[(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate.
2) an herbicidally active compound of the general formula (II) or an agrochemically acceptable salt thereof

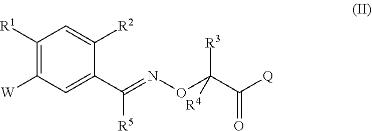

in which:
W represents a group W-1 to W-3

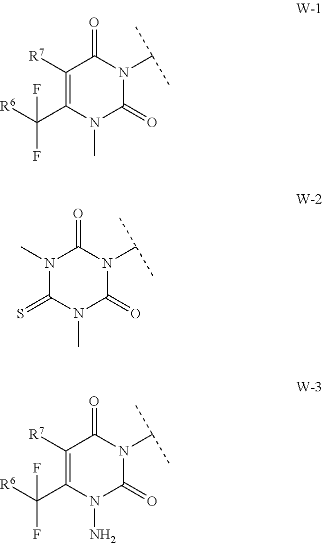

R¹ represents hydrogen, fluorine, chlorine, bromine, methoxy, ethoxy, prop-1-yloxy, prop-2-yloxy, but-1-yloxy, but-2-yloxy, 2-methylprop-1-yloxy, or 1,1-dimethyleth-1-yloxy,
R² represents fluorine, chlorine, bromine, cyano, nitro, C(O)NH₂, C(S)NH₂, trifluoromethyl, difluoromethyl, pentafluoroethyl, ethynyl, propyn-1-yl, 1-butyn-1-yl, pentyn-1-yl, or hexyn-1-yl,
R³ and R⁴ independently of each other represent hydrogen, (C₁-C₅)-alkyl, R¹³O-(C₁-C₅)-alkyl, (C₃-C₅)-cycloalkyl, (C₂-C₅)-alkenyl, aryl-(C₁-C₅)-alkyl, heteroaryl-(C₁-C₅)-alkyl, or heterocyclyl-(C₁-C₅)-alkyl, or
R³ and R⁴ together with the carbon atom to which they are bonded form a fully saturated or partly saturated 3—to 10-membered carbocyclic ring optionally having further substitution,
R⁵ represents hydrogen, (C₁-C₅)-alkyl, (C₁-C₅)-haloalkyl, R¹³O-(C₁-C₅)-alkyl, (C₂-C₈)-alkenyl, aryl-(C₁-C₅)-alkyl, heteroaryl-(C₁-C₅)-alkyl, heterocyclyl-(C₁-C₅)-alkyl, (C₃-C₈)-cycloalkyl, aryl, heteroaryl, or heterocyclyl,
R⁶ represents hydrogen, fluorine, chlorine, bromine, trifluoromethyl, difluoromethyl, methoxy, ethoxy, prop-1-yloxy, or but-1-yloxy,
R⁷ represents hydrogen or methyl, Q represents hydroxy or a group Q-1, Q-2

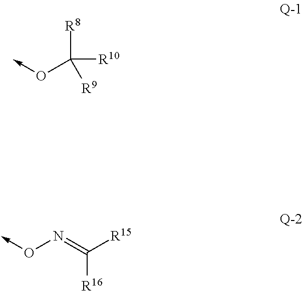

R⁸ represents hydrogen, (C₁-C₅)-alkyl, (C₁-C₅)-haloalkyl, aryl, aryl-(C₁-C₅)-alkyl, heteroaryl, (C₂-C₅)-alkynyl, (C₂-C₅)-alkenyl, C(O)R¹³, C(O)OR¹³, or (C₁-C₅)-alkoxy-(C₁-C₅)-alkyl,
R⁹ represents hydrogen or (C₁-C₅)-alkyl,
R¹⁰ represents hydrogen, halogen, cyano, nitro, (C₁-C₈)-alkyl, (C₁-C₈)-haloalkyl, (C₃-C₈)-cycloalkyl, (C₃-C₈)-cycloalkyl-(C₁-C₈)-alkyl, (C₃-C₈)-halocycloalkyl, (C₃-C₈)-halocycloalkyl-(C₁-C₈)-alkyl, (C₂-C₈)-alkenyl, (C₂-C₈)-alkynyl, aryl, aryl-(C₁-C₈)-alkyl, heteroaryl, heteroaryl-(C₁-C₈)-alkyl, heterocyclyl, heterocyclyl-(C₁-C₈)-alkyl, R¹¹R¹²N-(C₁-C₈)-alkyl, R¹³O-(C₁-C₈)-alkyl, cyano-(C₁-C₈)-alkyl, (C₁-C₈)-alkylcarbonyloxy-(C₁-C₈)-alkyl, (C₃-C₈)-cycloalkylcarbonyloxy-(C₁-C₈)-alkyl, arylcarbonyloxy-(C₁-C₈)-alkyl, heteroarylcarbonyloxy-(C₁-C₈)-alkyl, heterocyclylcarbonyloxy-(C₁-C₈)-alkyl, OR¹³, NR¹¹R¹², SR¹⁴, S(O)R¹⁴, SO₂R¹⁴, R¹⁴S-(C₁-C₈)-alkyl, R¹⁴(O)S-(C₁-C₈)-alkyl, R¹⁴(OS-(C₁-C₈)-alkyl, tris-[(C₁-C₈)-alkyl]silyl-(C₁-C₈)-alkyl, bis-[(C₁-C₈)-alkyl](aryl)silyl(C₁-C₈)-alkyl, [(C₁-C₈)-alkyl]-bis-(aryl)silyl-(C₁-C₈)-alkyl, tris-[(C₁-C₈)-alkyl]silyl, bis-hydroxyboryl-(C₁-C₈)-alkyl, bis-[(C₁-C₈)-alkoxy]boryl-(C₁-C₈)-alkyl, tetramethyl-1,3,2-Dioxaborolan-2-yl, tetramethyl-1,3,2-dioxaborolan-2-yl-(C₁-C₈)-alkyl, nitro-(C₁-C₈)-alkyl, C(O)OR¹³, C(O)R¹³, C(O)NR¹¹R¹², R¹³O(O)C-(C₁-C₈)-alkyl, R¹¹R¹²N(O)C-(C₁-C₈)-alkyl, or bis-(C₁-C₈)-alkoxy-(C₁-C₈)-alkyl, or R⁸ and R¹⁰ together with the carbon atom to which they are bonded form a fully saturated or partly saturated 3—to 10-membered monocyclic or bicyclic ring optionally interrupted by heteroatoms and optionally having further substitution, R¹¹ and R¹² independently of each other represent hydrogen, (C₁-C₅)-alkyl, (C₂-C₅)-alkenyl, (C₂-C₅)-alkynyl, (C₁-C₅)-cyanoalkyl, (C₁-C₁₀)-haloalkyl, (C₂-C₅)-haloalkenyl, (C₃-C₅)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₅)-alkoxy-(C₁-C₅)-alkyl, (C₁-C₅)-haloalkoxy-(C₁-C₅)-alkyl, (C₁-C₅)-alkylthio-(C₁-C₅)-alkyl, (C₁-C₅)-haloalkylthio-(C₁-C₅)-alkyl, (C₁-C₅)-alkoxy-(C₁-C₅)-haloalkyl, aryl, aryl-(C₁-C₅)-alkyl, heteroaryl, heteroaryl-(C₁-C₅)-alkyl, (C₃-C₅)-cycloalkyl-(C₁-C₅)-alkyl, (C₄-C₁₀)-cycloalkenyl-(C₁-C₅)-alkyl, C(O)R¹³, SO₂R¹⁴, heterocyclyl, (C₁-C₅)-alkoxycarbonyl, bis-[(C₁-C₅)-alkyl]aminocarbonyl-(C₁-C₅)-alkyl, (C₁-C₅)-alkyl-aminocarbonyl-(C₁-C₅)-alkyl, aryl-(C₁-C₅)-alkyl-aminocarbonyl-(C₁-C₅)-alkyl, aryl-(C₁-C₅)-alkoxycarbonyl, heteroaryl-(C₁-C₅)-alkoxycarbonyl, (C₂-C₅)-alkenyloxycarbonyl, (C₂-C₅)-alkynyloxycarbonyl, or heterocyclyl-(C₁-C₅)-alkyl, or R¹¹ and R¹² together with the nitrogen atom to which they are bonded form a fully saturated or partly saturated 3—to 10-membered monocyclic or bicyclic ring optionally interrupted by heteroatoms and optionally having further substitution,
R¹³ represents hydrogen, (C₁-C₅)-alkyl, (C₂-C₅)-alkenyl, (C₂-C₅)-alkynyl, (C₁-C₅)-cyanoalkyl, (C₁-C₁₀)-haloalkyl, (C₂-C₅)-haloalkenyl, (C₃-C₅)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₅)-alkoxy-(C₁-C₅)-alkyl, (C₁-C₅)-haloalkoxy-(C₁-C₅)-alkyl, (C₁-C₅)-alkoxy-(C₁-C₅)-haloalkyl, (C₁-C₅)-alkoxy-(C₁-C₅)-alkoxy-(C₁-C₅)-alkyl, (C₁-C₅)-alkoxy-(C₁-C₅)-alkoxy-(C₁-C₅)-alkoxy-(C₁-C₅)-alkyl, (C₁-C₅)-alkoxy-(C₁-C₅)-alkoxy-(C₁-C₅)-alkoxy-(C₁-C₅)-alkoxy-(C₁-C₅)-alkyl, aryl, aryl-(C₁-C₅)-alkyl, aryl-(C₁-C₅)-alkoxy-(C₁-C₅)-alkyl, heteroaryl, heteroaryl-(C₁-C₅)-alkyl, (C₃-C₅)-cycloalkyl-(C₁-C₅)-alkyl, (C₄-C₁₀)-cycloalkenyl-(C₁-C₅)-alkyl, bis-[(C₁-C₅)-alkyl]aminocarbonyl-(C₁-C₅)-alkyl, (C₁-C₅)-alkyl-aminocarbonyl-(C₁-C₅)-alkyl, aryl-(C₁-C₅)-alkyl-aminocarbonyl-(C₁-C₅)-alkyl, bis-[(C₁-C₅)-alkyl]amino-(C₂-C₆)-alkyl, (C₁-C₅)-alkyl-amino-(C₂-C₆)-alkyl, aryl-(C₁-C₅)-alkyl-amino-(C₂-C₆)-alkyl, R¹⁴S-(C₁-C₅)-alkyl, R¹⁴(O)S-(C₁-C₅)-alkyl, R¹⁴(OS-(C₁-C₅)-alkyl, hydroxycarbonyl-(C₁-C₅)-alkyl, heterocyclyl, heterocyclyl-(C₁-C₅)-alkyl, tris-[(C₁-C₅)-alkyl]silyl-(C₁-C₅)-alkyl, bis-[(C₁-C₅)-alkyl](aryl)silyl(C₁-C₅)-alkyl, [(C₁-C₈)-Alkyl]-bis-(aryl)silyl-(C₁-C₅)-alkyl, (C₁-C₅)-alkylcarbonyloxy-(C₁-C₅)-alkyl, (C₃-C₅)-cycloalkylcarbonyloxy-(C₁-C₅)-alkyl, arylcarbonyloxy-(C₁-C₅)-alkyl, heteroarylcarbonyloxy-(C₁-C₅)-alkyl, heterocyclylcarbonyloxy-(C₁-C₅)-alkyl, aryloxy-(C₁-C₅)-alkyl, heteroaryloxy-(C₁-C₅)-alkyl, or (C₁-C₅)-alkoxycarbonyl,
R¹⁴ represents hydrogen, (C₁-C₅)-alkyl, (C₂-C₅)-alkenyl, (C₂-C₅)-alkynyl, (C₁-C₅)-cyanoalkyl, (C₁-C₁₀)-haloalkyl, (C₂-C₅)-haloalkenyl, (C₃-C₅)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₅)-alkoxy-(C₁-C₅)-alkyl, (C₁-C₅)-alkoxy-(C₁-C₅)-haloalkyl, aryl, aryl-(C₁-C₅)-alkyl, heteroaryl, heteroaryl-(C₁-C₅)-alkyl, heterocyclyl-(C₁-C₅)-alkyl, (C₃-C₅)-cycloalkyl-(C₁-C₅)-alkyl, (C₄-C₁₀)-cycloalkenyl-(C₁-C₅)-alkyl, bis-[(C₁-C₅)-alkyl]amino, (C₁-C₅)-alkylamino, aryl-(C₁-C₅)-amino, aryl-(C₁-C₆)-alkylamino, aryl-[(C₁-C₅)-alkyl]amino; (C₃-C₈)-cycloalkylamino, (C₃-C₅)-cycloalkyl-[(C₁-C₅)-alkyl]amino, N-azetidinyl, N-pyrrolidinyl, N-piperidinyl, or N-morpholinyl, and R¹⁵ and R¹⁶ independently of each other represent (C₁-C₅)-alkyl, (C₃-C₅)-cycloalkyl, aryl, heteroaryl, or heterocyclyl.

Examples of such herbicidally active compounds within the scope of formula (II) include methyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, methyl (2R)-2-{[(E)-({2-chloro-4-fluoro-5-[3-methyl-2,6-dioxo-4-(trifluoromethyl)-3,6-dihydropyrimidin-1(2H)-yl]phenyl}methylidene)amino]oxy}propanoate (also known as methyl (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate or flufenoximacil), methyl (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, methyl 2-{[(Z)-{2-chloro-4-fluoro-5-[3-methyl-2,6-dioxo-4-(trifluoromethyl)-3,6-dihydropyrimidin-1(2H)-yl]benzylidene}amino]oxy}propanoate, 2-{[(Z)-{2-chloro-4-fluoro-5-[3-methyl-2,6-dioxo-4-(trifluoromethyl)-3,6-dihydropyrimidin-1(2H)-yl]benzylidene}amino]oxy}propanoic acid, ethyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, ethyl (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, ethyl (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoic acid, (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoic acid, (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoic acid, methyl 2-{1[(E)-{2-chloro-4-fluoro-5-[3-methyl-2,6-dioxo-4-(trifluoromethyl)-3,6-dihydropyrimidin-1(2H)-yl]benzylidene}amino]oxy}-2-methylpropanoate, ethyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}-2-methylpropanoate, methyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoate, methyl (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoate, methyl (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoate, 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoic acid, (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoic acid, (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoic acid, ethyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoate, methyl 2-({(E)-[2-chloro-5-(3,5-dimethyl-2,6-dioxo-4-sulfanylidene-1,3,5-triazinan-1-yl)-4-fluorobenzylidene]amino}oxy)propanoate, methyl (2R)-2-({(E)-[2-chloro-5-(3,5-dimethyl-2,6-dioxo-4-sulfanylidene-1,3,5-triazinan-1-yl)-4-fluorobenzylidene]amino}oxy)propanoate, methyl (2S)-2-({(E)-[2-chloro-5-(3,5-dimethyl-2,6-dioxo-4-sulfanylidene-1,3,5-triazinan-1-yl)-4-fluorobenzylidene]amino}oxy)propanoate, 2-({(E)-[2-chloro-5-(3,5-dimethyl-2,6-dioxo-4-sulfanylidene-1,3,5-triazinan-1-yl)-4-fluorobenzylidene]amino}oxy)propanoic acid, (2R)-2-({(E)-[2-chloro-5-(3,5-dimethyl-2,6-dioxo-4-sulfanylidene-1,3,5-triazinan-1-yl)-4-fluorobenzylidene]amino}oxy)propanoic acid, (2S)-2-({(E)-[2-chloro-5-(3,5-dimethyl-2,6-dioxo-4-sulfanylidene-1,3,5-triazinan-1-yl)-4-fluorobenzylidene]amino}oxy)propanoic acid, ethyl 2-({(E)-[2-chloro-5-(3,5-dimethyl-2,6-dioxo-4-sulfanylidene-1,3,5-triazinan-1-yl)-4-fluorobenzylidene]amino}oxy)propanoate, ethyl (2R)-2-({(E)-[2-chloro-5-(3,5-dimethyl-2,6-dioxo-4-sulfanylidene-1,3,5-triazinan-1-yl)-4-fluorobenzylidene]amino}oxy)propanoate, ethyl (2S)-2-({(E)-[2-chloro-5-(3,5-dimethyl-2,6-dioxo-4-sulfanylidene-1,3,5-triazinan-1-yl)-4-fluorobenzylidene]amino}oxy)propanoate, methyl 2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoate, methyl (2R)-2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoate, methyl (2S)-2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoate, 2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoic acid, (2R)-2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoic acid, and (2S)-2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoic acid.
3) an herbicidally active compound of the general formula (III) or an agrochemically acceptable salt thereof

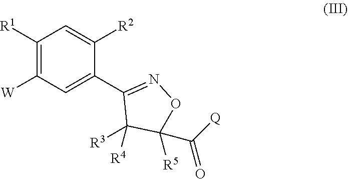

in which:
W represents a group W-1 to W-3

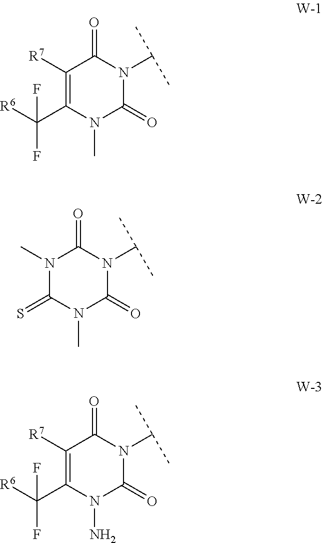

R¹ represents hydrogen, fluorine, chlorine, bromine, methoxy, ethoxy, prop-1-yloxy, prop-2-yloxy, but-1-yloxy, but-2-yloxy, 2-methylprop-1-yloxy, or 1,1-dimethyleth-1-yloxy,
R² represents fluorine, chlorine, bromine, cyano, nitro, C(O)NH₂, C(S)NH₂, trifluoromethyl, difluoromethyl, pentafluoroethyl, ethynyl, propyn-1-yl, 1-butyn-1-yl, pentyn-1-yl, or hexyn-1-yl, R³ and R⁴ independently of each other represent hydrogen, (C₁-C₅)-alkyl,
R⁵ represents hydrogen, (C₁-C₈)-alkyl, (C₁-C₈)-haloalkyl, R¹³O-(C₁-C₅)-alkyl, (C₂-C₈)-alkenyl, aryl-(C₁-C₈)-alkyl, heteroaryl-(C₁-C₅)-alkyl, or heterocyclyl-(C₁-C₅)-alkyl, or
R³ and R⁵ together with the carbon atom to which they are bonded form a fully saturated or partly saturated 3—to 10-membered monocyclic or bicyclic ring optionally interrupted by heteroatoms and optionally having further substitution,
R⁶ represents hydrogen, fluorine, chlorine, bromine, trifluoromethyl, difluoromethyl, methoxy, ethoxy, prop-1-yloxy, or but-1-yloxy,
R⁷ represents hydrogen, methyl,
Q represents hydroxy or a group Q-1, Q-2

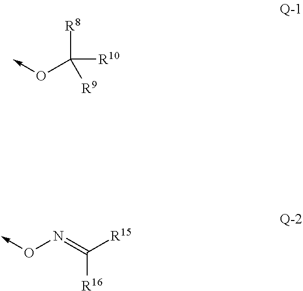

R⁸ represents hydrogen, (C₁-C₅)-alkyl, (C₁-C₅)-haloalkyl, aryl, aryl-(C₁-C₅)-alkyl, heteroaryl, (C₂-C₅)-alkynyl, (C₂-C₅)-alkenyl, C(O)R¹³, C(O)OR¹³, or (C₁-C₈)-alkoxy-(C₁-C₅)-alkyl,
R⁹ represents hydrogen, (C₁-C₅)-alkyl,
R¹⁰ represents hydrogen, halogen, cyano, nitro, (C₁-C₅)-alkyl, (C₁-C₈)-aaloalkyl, (C₃-C₅)-cycloalkyl, (C₃-C₅)-cycloalkyl-(C₁-C₅)-alkyl, (C₃-C₅)-halocycloalkyl, (C₃-C₅)-halocycloalkyl-(C₁-C₅)-alkyl, (C₂-C₅)-alkenyl, (C₂-C₅)-alkynyl, aryl, aryl-(C₁-C₅)-alkyl, heteroaryl, heteroaryl-(C₁-C₅)-alkyl, heterocyclyl, heterocyclyl-(C₁-C₅)-alkyl, R¹¹R¹²N-(C₁-C₅)-alkyl, R¹³O-(C₁-C₈)-alkyl, cyano-(C₁-C₈)-alkyl, (C₁-C₈)-alkylcarbonyloxy-(C₁-C₅)-alkyl, (C₃-C₅)-cycloalkylcarbonyloxy-(C₁-C₅)-alkyl, arylcarbonyloxy-(C₁-C₅)-alkyl, heteroarylcarbonyloxy-(C₁-C₅)-alkyl, heterocyclylcarbonyloxy-(C₁-C₅)-alkyl, OR¹³, NR¹¹R¹², SR¹⁴, S(O)R¹⁴, SO₂R¹⁴, R¹⁴S-(C₁-C₈)-alkyl, R¹⁴(O)S-(C₁-C₈)-alkyl, R¹⁴(OS-(C₁-C₅)-alkyl, tris-[(C₁-C₅)-alkyl]silyl-(C₁-C₅)-alkyl, bis-[(C₁-C₅)-alkyl](aryl)silyl(C₁-C₅)-alkyl, [(C₁-C₅)-alkyl]-bis-(aryl)silyl-(C₁-C₅)-alkyl, tris-[(C₁-C₅)-alkyl]silyl, bis-hydroxyboryl-(C₁-C₅)-alkyl, bis-[(C₁-C₅)-alkoxy]boryl-(C₁-C₅)-alkyl, tetramethyl-1,3,2-Dioxaborolan-2-yl, tetramethyl-1,3,2-dioxaborolan-2-yl-(C₁-C₅)-alkyl, nitro-(C₁-C₅)-alkyl, C(O)OR¹³, C(O)R¹³, C(O)NR¹¹R¹², R¹³O(O)C-(C₁-C₈)-alkyl, R¹¹R¹²N(O)C-(C₁-C₅)-alkyl, or bis-(C₁-C₅)-alkoxy-(C₁-C₅)-alkyl, or
R⁸ and R¹⁰ together with the carbon atom to which they are bonded form a fully saturated or partly saturated 3—to 10-membered monocyclic or bicyclic ring optionally interrupted by heteroatoms and optionally having further substitution, R¹¹ and R¹² independently of each other represent hydrogen, (C₁-C₅)-alkyl, (C₂-C₅)-alkenyl, (C₂-C₅)-alkynyl, (C₁-C₅)-cyanoalkyl, (C₁-C₁₀)-haloalkyl, (C₂-C₅)-haloalkenyl, (C₃-C₅)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₅)-alkoxy-(C₁-C₅)-alkyl, (C₁-C₅)-haloalkoxy-(C₁-C₅)-alkyl, (C₁-C₅)-alkylthio-(C₁-C₅)-alkyl, (C₁-C₅)-haloalkylthio-(C₁-C₅)-alkyl, (C₁-C₅)-alkoxy-(C₁-C₅)-haloalkyl, aryl, aryl-(C₁-C₅)-alkyl, heteroaryl, heteroaryl-(C₁-C₅)-alkyl, (C₃-C₅)-cycloalkyl-(C₁-C₅)-alkyl, (C₄-C₁₀)-cycloalkenyl-(C₁-C₅)-alkyl, C(O)R¹³, SO₂R¹⁴, heterocyclyl, (C₁-C₅)-alkoxycarbonyl, bis-[(C₁-C₅)-alkyl]aminocarbonyl-(C₁-C₅)-alkyl, (C₁-C₅)-alkyl-aminocarbonyl-(C₁-C₅)-alkyl, aryl-(C₁-C₅)-alkyl-aminocarbonyl-(C₁-C₅)-alkyl, aryl-(C₁-C₅)-alkoxycarbonyl, heteroaryl-(C₁-C₅)-alkoxycarbonyl, (C₂-C₅)-alkenyloxycarbonyl, (C₂-C₈)-alkynyloxycarbonyl, or heterocyclyl-(C₁-C₅)-alkyl, or R¹¹ and R¹² together with the nitrogen atom to which they are bonded form a fully saturated or partly saturated 3—to 10-membered monocyclic or bicyclic ring optionally interrupted by heteroatoms and optionally having further substitution,
R¹³ represents hydrogen, (C₁-C₅)-alkyl, (C₂-C₅)-alkenyl, (C₂-C₅)-alkynyl, (C₁-C₅)-cyanoalkyl, (C₁-C₁₀)-haloalkyl, (C₂-C₅)-haloalkenyl, (C₃-C₅)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₅)-alkoxy-(C₁-C₅)-alkyl, (C₁-C₅)-haloalkoxy-(C₁-C₅)-alkyl, (C₁-C₅)-alkoxy-(C₁-C₅)-haloalkyl, (C₁-C₅)-alkoxy-(C₁-C₅)-alkoxy-(C₁-C₅)-alkyl, (C₁-C₅)-alkoxy-(C₁-C₅)-alkoxy-(C₁-C₅)-alkoxy-(C₁-C₅)-alkyl, (C₁-C₅)-alkoxy-(C₁-C₅)-alkoxy-(C₁-C₅)-alkoxy-(C₁-C₅)-alkoxy-(C₁-C₅)-alkyl, aryl, aryl-(C₁-C₅)-alkyl, aryl-(C₁-C₅)-alkoxy-(C₁-C₅)-alkyl, heteroaryl, heteroaryl-(C₁-C₅)-alkyl, (C₃-C₅)-cycloalkyl-(C₁-C₅)-alkyl, (C₄-C₁₀)-cycloalkenyl-(C₁-C₅)-alkyl, bis-[(C₁-C₅)-alkyl]aminocarbonyl-(C₁-C₅)-alkyl, (C₁-C₅)-alkyl-aminocarbonyl-(C₁-C₅)-alkyl, aryl-(C₁-C₅)-alkyl-aminocarbonyl-(C₁-C₅)-alkyl, bis-[(C₁-C₅)-alkyl]amino-(C₂-C₆)-alkyl, (C₁-C₅)-alkyl-amino-(C₂-C₆)-alkyl, aryl-(C₁-C₅)-alkyl-amino-(C₂-C₆)-alkyl, R¹⁴S-(C₁-C₅)-alkyl, R¹⁴(O)S-(C₁-C₅)-alkyl, R¹⁴(OS-(C₁-C₅)-alkyl, hydroxycarbonyl-(C₁-C₅)-alkyl, heterocyclyl, heterocyclyl-(C₁-C₅)-alkyl, tris-[(C₁-C₅)-alkyl]silyl-(C₁-C₅)-alkyl, bis-[(C₁-C₅)-alkyl](aryl)silyl(C₁-C₅)-alkyl, [(C₁-C₈)-Alkyl]-bis-(aryl)silyl-(C₁-C₅)-alkyl, (C₁-C₅)-alkylcarbonyloxy-(C₁-C₅)-alkyl, (C₃-C₅)-cycloalkylcarbonyloxy-(C₁-C₅)-alkyl, arylcarbonyloxy-(C₁-C₅)-alkyl, heteroarylcarbonyloxy-(C₁-C₅)-alkyl, heterocyclylcarbonyloxy-(C₁-C₅)-alkyl, aryloxy-(C₁-C₅)-alkyl, heteroaryloxy-(C₁-C₅)-alkyl, or (C₁-C₅)-alkoxycarbonyl,
R¹⁴ represents hydrogen, (C₁-C₅)-alkyl, (C₂-C₅)-alkenyl, (C₂-C₅)-alkynyl, (C₁-C₅)-cyanoalkyl, (C₁-C₁₀)-haloalkyl, (C₂-C₅)-haloalkenyl, (C₃-C₅)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₅)-alkoxy-(C₁-C₅)-alkyl, (C₁-C₅)-alkoxy-(C₁-C₅)-haloalkyl, aryl, aryl-(C₁-C₅)-alkyl, heteroaryl, heteroaryl-(C₁-C₅)-alkyl, heterocyclyl-(C₁-C₅)-alkyl, (C₃-C₅)-cycloalkyl-(C₁-C₅)-alkyl, (C₄-C₁₀)-cycloalkenyl-(C₁-C₅)-alkyl, bis-[(C₁-C₅)-alkyl]amino, (C₁-C₅)-alkylamino, aryl-(C₁-C₅)-amino, aryl-(C₁-C₆)-alkylamino, aryl-[(C₁-C₅)-alkyl]amino; (C₃-C₈)-cycloalkylamino, (C₃-C₅)-cycloalkyl-[(C₁-C₅)-alkyl]amino, N-azetidinyl, N-pyrrolidinyl, N-piperidinyl, or N-morpholinyl, and R¹⁵ and R¹⁶ independently of each other represent (C₁-C₅)-alkyl, (C₃-C₅)-cycloalkyl, aryl, heteroaryl, or heterocyclyl.

Examples of the herbicidally active compounds within the scope of formula (III) include ethyl 3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylate, methyl 3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylate, 3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylic acid, (5R)-3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylic acid, (5S)-3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylic acid, ethyl (5S)-3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylate, ethyl (5R)-3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylate, ethyl 3-{2-chloro-4-fluoro-5-[3-methyl-2,6-dioxo-4-(trifluoromethyl)-3,6-dihydropyrimidin-1(2H)-yl]phenyl}-5-propyl-4,5-dihydro-1,2-oxazole-5-carboxylate, ethyl 3-{2-chloro-4-fluoro-5-[3-methyl-2,6-dioxo-4-(trifluoromethyl)-3,6-dihydropyrimidin-1(2H)-yl]phenyl}-5-ethyl-4,5-dihydro-1,2-oxazole-5-carboxylate, 3-[4-chloro-2-fluoro-5-(5-{[(isopropylideneamino)oxy]carbonyl}-5-methyl-4,5-dihydro-1,2-oxazol-3-yl)phenyl]-1-methyl-6-(trifluoromethyl)pyrimidine-2,4(1H,3H)-dione, ethyl 3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylate, methyl 3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylate, 3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylic acid, (5R)-3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylic acid, (5S)-3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylic acid, 3-[4-chloro-2-fluoro-5-(5-{[(isopropylideneamino)oxy]carbonyl}-5-methyl-4,5-dihydro-1,2-oxazol-3-yl)phenyl]-1,5-dimethyl-6-sulfanylidene-1,3,5-triazinane-2,4-dione, ethyl 3-{5-[3-amino-2,6-dioxo-4-(trifluoromethyl)-3,6-dihydropyrimidin-1(2H)-yl]-2-chloro-4-fluorophenyl}-5-methyl-4,5-dihydro-1,2-oxazole-5-carboxylate, 3-{5-[3-amino-2,6-dioxo-4-(trifluoromethyl)-3,6-dihydropyrimidin-1(2H)-yl]-2-chloro-4-fluorophenyl}-5-methyl-4,5-dihydro-1,2-oxazole-5-carboxylic acid, methyl 3-{2-chloro-4-fluoro-5-[3-methyl-2,6-dioxo-4-(trifluoromethyl)-3,6-dihydropyrimidin-1(2H)-yl]phenyl}-3a,4,5,6-tetrahydro-6aH-cyclopenta[d][1,2]oxazole-6a-carboxylate, ethyl 3-{2-chloro-4-fluoro-5-[3-methyl-2,6-dioxo-4-(trifluoromethyl)-3,6-dihydropyrimidin-1(2H)-yl]phenyl}-3a,4,5,6-tetrahydro-6aH-cyclopenta[d][1,2]oxazole-6a-carboxylate, and methyl 3-{2-bromo-4-fluoro-5-[3-methyl-2,6-dioxo-4-(trifluoromethyl)-3,6-dihydropyrimidin-1(2H)-yl]phenyl}-3a,4,5,6-tetrahydro-6aH-cyclopenta[d][1,2]oxazole-6a-carboxylate.
4) an herbicidally active compound corresponding to a compound selected from the group consisting of A1, A2, and A3, or an agrochemically acceptable salt thereof, wherein: A1 corresponds to:

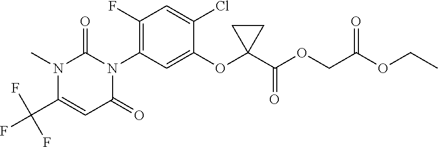 [SMILES: CCOC(=O)COC(=O)C1(Oc2cc(-n3c(=O)cc(C(F)(F)F)n(C)c3=O)c(F)cc2Cl)CC1]

2-ethoxy-2-oxoethyl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate; A2 corresponds to:

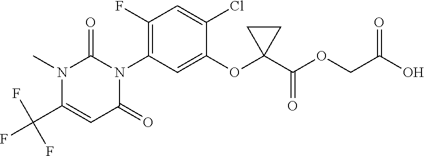 [SMILES: Cn1c(C(F)(F)F)cc(=O)n(-c2cc(OC3(C(=O)OCC(=O)O)CC3)c(Cl)cc2F)c1=O]

{[(1-{2-chloro-4-fluoro-5-[3-methyl-2,6-dioxo-4-(trifluoromethyl)-3,6-dihydropyrimidin-1(2H)-yl]phenoxy}cyclopropyl)carbonyl]oxy}acetic acid; and A3 corresponds to:

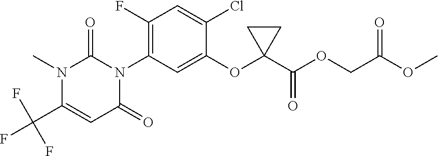 [SMILES: COC(=O)COC(=O)C1(Oc2cc(-n3c(=O)cc(C(F)(F)F)n(C)c3=O)c(F)cc2Cl)CC1]

2-methoxy-2-oxoethyl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate
5) an herbicidally active compound of the general formula (IV) or an agrochemically acceptable salt thereof

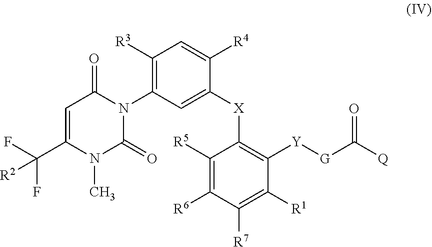

wherein
R¹ is hydrogen, fluoro, chloro, bromo, iodo, cyano, methyl, ethyl, prop-1-yl, 1-methylethyl, but-1-yl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-di-methylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, trifluormethyl, difluormethyl, pentafluorethyl, 2,2-difluorethyl, 2,2,2-trifluorethyl, methoxy, ethoxy, prop-1-yloxy, prop-2-yloxy, but-1-yloxy, but-2-yloxy, 2-methylprop-1-yloxy, 1,1-dimethyleth-1-yloxy, difluormethoxy, trifluormethoxy, pentafluorethoxy, 2,2-difluorethoxy, or 2,2,2-trifluorethoxy,
R² is hydrogen, fluoro, chloro, bromo, methyl, trifluormethyl, methoxy, ethoxy, prop-1-yloxy, or but-1-yloxy,
R³ is hydrogen, fluoro, chloro, bromo, methoxy, ethoxy, prop-1-yloxy, prop-2-yloxy, but-1-yloxy, but-2-yloxy, 2-methylprop-1-yloxy, or 1,1-dimethyleth-1-yloxy,
R⁴ is fluoro, chloro, bromo, cyano, NO₂, C(O)NH₂, C(S)NH₂, trifluormethyl, difluormethyl, pentafluorethyl, ethinyl, propin-1-yl, 1-butin-1-yl, pentin-1-yl, or hexin-1-yl,
R⁵, R⁶ and R⁷ are independently from each other hydrogen, fluoro, chloro, bromo, iodo, cyano, methyl, ethyl, prop-1-yl, 1-methylethyl, but-1-yl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-di-methylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, trifluormethyl, difluormethyl, pentafluorethyl, 2,2-difluorethyl, 2,2,2-trifluorethyl, methoxy, ethoxy, prop-1-yloxy, prop-2-yloxy, but-1-yloxy, but-2-yloxy, 2-methylprop-1-yloxy, 1,1-dimethyleth-1-yloxy, difluormethoxy, trifluormethoxy, pentafluorethoxy, 2,2-difluorethoxy, or 2,2,2-trifluorethoxy,
G is methylene, (methyl)methylene, (ethyl)methylene, (prop-1-yl)methylene, (prop-2-yl)methylene, (but-1-yl)methylene, (but-2-yl)methylene, (pent-1-yl)methylene, (pent-2-yl)methylene, (pent-3-yl)methylene, (dimethyl)methylene, (diethyl)methylene, ethylene, n-propylen, (1-methyl)ethyl-1-en, (2-methyl)ethyl-1-en, n-butylen, 1-methylpropyl-1-en, 2-methylpropyl-1-en, 3-methylpropyl-1-en, 1,1-dimethylethyl-1-en, 2,2-dimethylethyl-1-en, 1-ethylethyl-1-en, 2-ethylethyl-1-en, 1-(prop-1-yl)ethyl-1-en, 2-(prop-1-yl)ethyl-1-en, 1-(prop-2-yl)ethyl-1-en, 2-(prop-2-yl)ethyl-1-en, 1,1,2-trimethylethyl-1-en, 1,2,2-trimethylethyl-1-en, 1,1,2,2-tetramethylethyl-1-en, n-pentylen, 1-methylbutyl-1-en, 2-methylbutyl-1-en, 3-methylbutyl-1-en, 4-methylbutyl-1-en, 1,1-dimethylpropyl-1-en, 2,2-dimethylpropyl-1-en, 3,3-dimethylpropyl-1-en, 1,2-dimethylpropyl-1-en, 1,3-dimethylpropyl-1-en, 1-ethylpropyl-1-en, n-hexylen, 1-methylpentyl-1-en, 2-methylpentyl-1-en, 3-methylpentyl-1-en, 4-methylpentyl-1-en, 1,1-dimethylbutyl-1-en, 1,2-dimethylbutyl-1-en, 1,3-di-methylbutyl-1-en, 2,2-dimethylbutyl-1-en, 2,3-dimethylbutyl-1-en, 3,3-dimethylbutyl-1-en, 1-ethylbutyl-1-en, 2-ethylbutyl-1-en, 1,1,2-trimethylpropyl-1-en, 1,2,2-trimethylpropyl-1-en, 1-ethyl-1-methylpropyl-1-en, or 1-ethyl-2-methylpropyl-1-en,
X and Y are independently from each other O (oxygen) or S (sulfur) and
Q is one of the groups Q-1 to Q-25, wherein in the formulae of the following table the arrow stands for a bond of the respective group Q to the carbonyl group in the general formula (I):

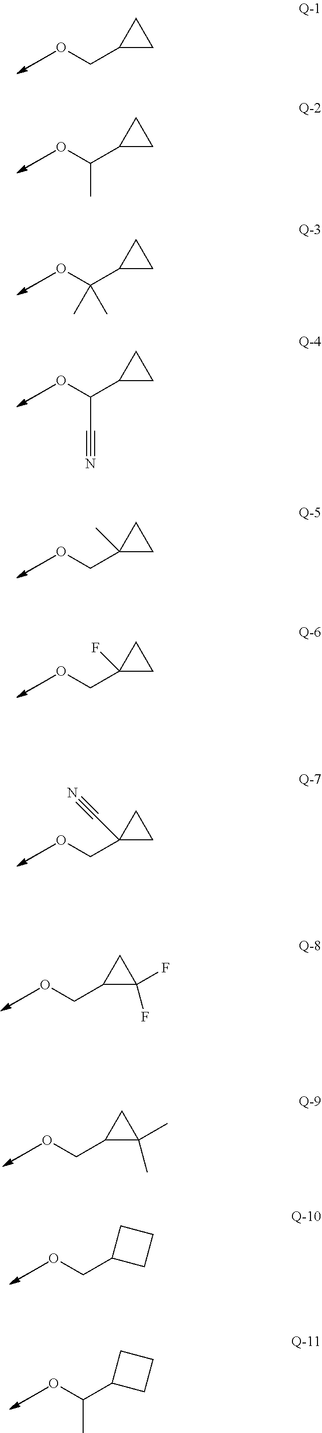

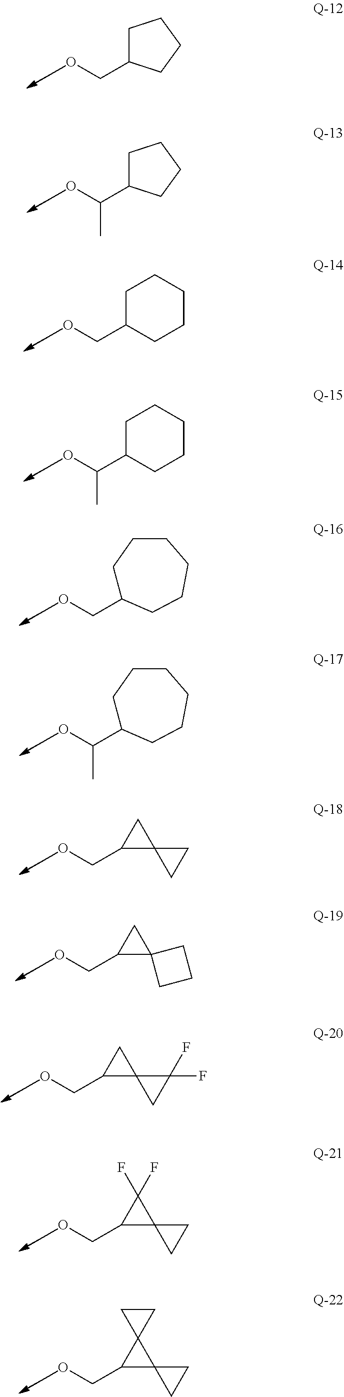

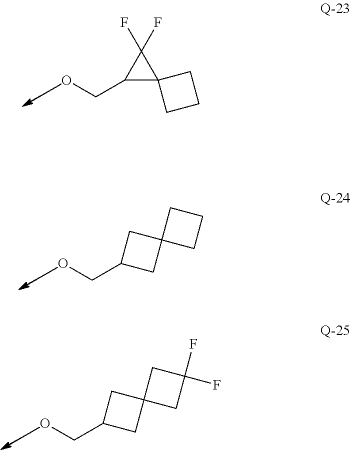

An example of an herbicidally active compound within the scope of formula (IV) is cyclopropylmethyl-(2-{2-chlor-4-fluor-5-[3-methyl-2,6-dioxo-4-(trifluorinethyl)-3,6-dihydropyrimidin-1(2H)-yl]phenoxy}phenoxy)acetate, which has the following structure:

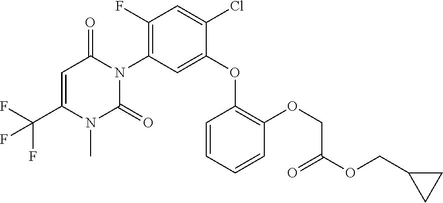 [SMILES: Cn1c(C(F)(F)F)cc(=O)n(-c2cc(Oc3ccccc3OCC(=O)OCC3CC3)c(Cl)cc2F)c1=O]

As used herein, inhibitors of acetyl CoA carboxylase (ACCase) in the aryloxyphenoxy propionate (FOP) group (referred to as “FOP herbicide(s)”) include, but are not limited to, chlorazifop, clodinafop, clodinafop-ethyl, clodinafop-propargyl, clofop, cyhalofop, cyhalofop-butyl, diclofop, diclofop-methyl, diclofop-P, diclofop-P-methyl, fenoxaprop, fenoxaprop-P, fenoxaprop-P-ethyl, fenthiaprop, fluazifop, fluazifop-butyl, fluazifop-P, fluazifop-P-butyl, haloxyfop, haloxyfop.etotyl, haloxyfop-methyl, haloxyfop-P, haloxyfop-P-methyl, isoxapyrifop, metamifop, propaquizafop, quizalofop, quizalafop-ethyl, quizalofop-P, quizalafop-P-ethyl, quizalafop-P-tefuryl, trifop, and combinations of any thereof.

As used herein, synthetic auxins include, but are not limited to, benzoic acid herbicides, phenoxy acid herbicides, arylpicolinate herbicides, and pyridinyloxy acid herbicides. Examples of a benzoic acid herbicides include, but are not limited to, dicamba (3,6-dichloro-2-methoxybenzoic acid), dicamba salts, dicamba-butotyl, dicamba-diglycolamine salt, dicamba-dimethylammonium, dicamba-diethanolammonium, dicamba-isopropylammonium, dicamba-potassium, dicamba-sodium, and dicamba-trolamine. Examples of phenoxy acid herbicides include, but are not limited to, 2,4-D (2,4-dichlorophenoxyacetic acid), 2,4-D-butotyl, 2,4-D-butyl, 2,4-D-choline, 2,4-D-dimethylammonium, 2,4-D-diolamine, 2,4-D-ethyl, 2,4-D-2-ethylhexyl, 2,4-D-isobutyl, 2,4-D-isoctyl, 2,4-D-isopropyl, 2,4-D-isopropylammonium, 2,4-D-potassium, 2,4-D-sodium, 2,4-D-triisopropanolammonium, 2,4-D-trolamine, 2,4,5-T (2,4,5-trichlorophenoxyacetic acid), clomeprop, dichlorprop, fenoprop, MCPA (2-methyl-4-chlorophenoxyacetic acid), MCPA-butotyl, MCPA-dimethylammonium, MCPA-2-ethylhexyl. MCPA-isopropylammonium, MCPA-potassium, MCPA-sodium, MCPA-thioethyl, 2,4-DB, MCPB (4-(4-chloro-2-methylphenoxy)butanoic acid), MCPB-methyl, MCPB-ethyl-sodium, and mecoprop. Examples of arylpicolinate herbicides include, but are not limited to, halauxifen, halauxifen-methyl, and florpyrauxifen-benzyl. Examples of pyridinyloxy acid herbicides include, but are not limited to, triclopyr, fluroxypyr, aminopyralid, and picloram.

As used herein, inhibitors of glutamine synthetase include, but are not limited to, phosphinothricin, glufosinate, glufosinate salts, glufosinate-ammonium, glufosinate-sodium, glufosinate-P, L-glufosinate-ammonium, and L-glufosinate-sodium.

As used herein, inhibitors of 5-enolpyruvylshikimate-3-phosphate synthase (EPSPS) include, but are not limited to, glyphosate, glyphosate salts, glyphosate-isopropylammonium, glyphosate-ammonium, glyphosate-dimethylammonium, glyphosate-trimesium (=sulfosate), glyphosate-diammonium, glyphosate-potassium, and glyphosate-sodium.

As used herein, “herbicide tolerant” or “herbicide tolerance” or “tolerance” means the ability to be wholly or partially unaffected by the presence or application of one of more herbicide(s), for example to resist the toxic effects of an herbicide when applied. A cell, seed, or plant is “herbicide tolerant” or has “improved tolerance” if it can maintain at least some normal growth or phenotype in the presence of one or more herbicide(s). A trait is an herbicide tolerance trait if its presence can confer improved tolerance to an herbicide upon a cell, plant, or seed as compared to the wildtype or control cell, plant, or seed. Crops comprising an herbicide tolerance trait can continue to grow in the presence of the herbicide and may be minimally affected by the presence of the herbicide. A protein confers “herbicide tolerance” if expression of the protein can confer improved tolerance to an herbicide upon a cell, plant, or seed as compared to the wildtype or control cell, plant, or seed. Examples of herbicide tolerance proteins are protoporphyrinogen oxidase, dicamba monooxygenase, phosphinothricin N-acetyltransferase, the alpha-ketoglutarate-dependent non-heme iron dioxygenase, and 5-enolpyruvylshikimate-3-phosphate synthase. Herbicide tolerance may be complete or partial insensitivity to a particular herbicide and may be expressed as a percent (%) tolerance or insensitivity to a particular herbicide.

As used herein “herbicide injury” or “injury” refers to injury to a plant because of the application of one or more herbicides. The “injury rate” or “percent injury” refers to the percentage of leaf area of a plant exhibiting damage such as necrosis (brown or dead tissue), chlorosis (yellow tissue or yellow spotting) and malformation (misshapen leaves or plant structures, epinasty or twisting of stem, cupping of leaves) caused by herbicide application based on visual evaluation. It is measured on a scale of 0 to 100, where “0” representing no crop injury and “100” denoting complete crop injury (death).

For corn plants containing or comprising corn event Zm_CSM63715, the plant will have decreased injury after application of one or more PPO inhibitors. For example, corn plants containing or comprising corn event Zm_CSM63715 will have less than about 5% injury, less than about 10% injury, less than about 15% injury, or less than about 20% injury following application of a PPO herbicide such as flumioxazin, epyrifenacil, lactofen, acifluorfen, pyraflufen, pyraflufen-ethyl, oxadiazon, butafenacil, pyridin-2-ylmethyl[(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl[(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl[(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate, cyanomethyl[(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate, methyl (2R)-2-{[(E)-({2-chloro-4-fluoro-5-[3-methyl-2,6-dioxo-4-(trifluoromethyl)-3,6-dihydropyrimidin-1(2H)-yl]phenyl}methylidene)amino]oxy}propanoate (flufenoximacil), cyclopropylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate, fomesafen, saflufenacil, sulfentrazone, tiafenacil, and trifludimoxazin, or a combination of any thereof, as compared to otherwise identical corn plants that do not contain corn event Zm_CSM63715.

As used herein, a “weed” is any undesired plant. A plant may be considered generally undesirable for agriculture or horticulture purposes (for example, Amaranthus species) or may be considered undesirable in a particular situation (for example, a crop plant of one species in a field of a different species, also known as a volunteer plant). Weeds are commonly known in the art and vary by geography, season, growing environment, and time. Lists of weed species are available from agricultural and scientific societies and efforts (such as the Weed Science Society of America, the Canadian Weed Science Society, the Brazilian Weed Science Society, the International Weed Science Society, and the International Survey of Herbicide Resistant Weeds), government agencies (such as the United States Department of Agriculture and the Australia Department of the Environment and Energy), and industry and farmer associations. Major troublesome weeds in corn production include waterhemp (Amaranthus tuberculatus), giant ragweed (Ambrosia trifida), common ragweed (Ambrosia artemissifolia) common lambsquarters (Chenopodium album), horseweed (Conyza canadensis), marestail (Erigeron canadensis), palmer amaranth (Amaranthus palmeri), redroot pigweed (Amaranthus retrofle), Italian ryegrass (Lolium perenne ssp. multiflorum), velvetleaf (Abutilon theophrasti Medik.), Kochia (Kochia scoparia), common cocklebur (Xanthium strumarium), foxtail (Setaria spp.), barnyard grass (Echinochloa crus-galli), and Johnsongrass (Sorghum halepense) (Heap 2021; Shoup et al, 2016)

Methods for controlling or preventing weed growth in an area are provided. One example of such a method comprises corn comprising event Zm_CSM63715 in the area and applying an effective amount of a PPO herbicide to control weeds in the area without injury to the corn or with less than about 10% injury to the corn. The methods comprise applying one or more PPO herbicides, where seeds or plants comprising corn event Zm_CSM63715 are planted in the area before, at the time of, or after applying the herbicide and the herbicide application prevents or inhibits weed growth and does not injure the corn plants comprising event Zm_CSM63715, or has about less than about 5-20% injury. The plant growth area may or may not comprise weed seeds or plants at the time of herbicide application. The herbicide(s) used in the methods described herein can be applied alone, sequentially with or in combination with one or more herbicide(s) during the growing season. The herbicide(s) used in the methods described herein can be applied in combination with one or more herbicide(s) temporally (for example, as a tank mixture or in sequential applications), spatially (for example, at different times during the growing season including before and after corn seed planting), or both. For example, a method for controlling weeds is provided that consists of planting seed comprising corn event Zm_CSM63715 in an area and applying an herbicidally effective amount over the growing season of one or more PPO herbicides alone or in any combination with another herbicide, for the purpose of controlling weeds in the area with no injury or less than about 5-20% injury to the plants containing corn event Zm_CSM63715. Such application of herbicide(s) may be pre-planting (any time prior to planting seed comprising corn event Zm_CSM63715, including for burn-down purposes, that is application to emerging or existing weeds prior to seed plant), pre-emergence (any time after seed comprising corn event Zm_CSM63715 is planted and before plants comprising corn event Zm_CSM63715 emerge), or post-emergence (any time after plants comprising corn event Zm_CSM63715 emerge). Multiple applications of one or more herbicides, or a combination of herbicides together or individually, may be used over a growing season, for example, two applications (such as a pre-planting application and a post-emergence application, or a pre-emergence application and a post-emergence application) or three or more applications (such as a pre-planting application and two post-emergence applications).

Herbicide application in practicing the methods described herein may be at the recommended commercial rate or any fraction or multiple thereof, such as twice the recommended commercial rate. Herbicide rates may be expressed as pounds acid equivalent per acre (lb ae/acre), pounds active ingredient per acre (lb ai/acre) or pounds active ingredient per hectare (lb ai/ha), depending on the herbicide and the formulation. One gram per hectare is equal to 0.000892179 pound per acre. The use of acres or hectare in the herbicide application rates as provided herein is merely instructive; herbicide application rates in the equivalent dosages to any rate provided herein may be used for areas larger or smaller than an acre. The herbicide application can comprise at least one PPO herbicide including, but not limited to flumioxazin, epyrifenacil, lactofen, acifluorfen, pyraflufen, pyraflufen-ethyl, oxadiazon, butafenacil, pyridin-2-ylmethyl[(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl[(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl[(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate, cyanomethyl[(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate, methyl (2R)-2-{[(E)-({2-chloro-4-fluoro-5-[3-methyl-2,6-dioxo-4-(trifluoromethyl)-3,6-dihydropyrimidin-1(2H)-yl]phenyl}methylidene)amino]oxy}propanoate (flufenoximacil), cyclopropylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate, fomesafen, saflufenacil, sulfentrazone, tiafenacil, and trifludimoxazin, another PPO herbicide, or a combination of any thereof. The plant growth area may or may not comprise weed plants at the time of herbicide application.

The effective amount of a PPO herbicide can be about 0.0009 lb/acre to about 1.5 lb/acre over a growing season. Table 2 provides examples of various PPO herbicides and the application rates that can be used for controlling weeds in a corn crop growing area where Zm_CSM63715 is planted.

TABLE 2
Examples of PPO herbicides and application rates.

| | Broad | Intermediate | Labeled |
|---|---|---|---|
| | Rate Range | Rate Range | Rates |
| | (gram/ | (gram/ | (gram/ |
| PPO Herbicide | hectare) | hectare) | hectare) |
| Epyrifencacil (S3100) | 1-200 | 5-100 | 20-40 |
| Flumioxazin | 1-448 | 5-140 | 70-224 |
| Fomesafen | 1-1680 | 5-840 | 280-420 |
| Lactofen | 1-880 | 5-440 | 140-220 |
| Saflufenacil | 1-400 | 5-200 | 22-88 |
| Sulfentrazone | 1-1680 | 5-840 | 112-420 |
| Trifludimoxazin | 1-75 | 10-50 | 25-50 |
| Tiafenacil | 1-75 | 10-50 | 25-50 |
| Acifluorfen | 1-1680 | 5-840 | 280-560 |
| Pyraflufen | 1-50 | 10-25 | 25-50 |
| Oxadiazon | 1-400 | 50-200 | 200-400 |
| Butafenacil | 1-100 | 10-100 | 25-50 |
| pyridin-2-ylmethyl [(3-{2- | 1-200 | 10-100 | 50-100 |
| chloro-4-fluoro-5-[3-methyl- | | | |
| 2,6-dioxo-4- | | | |
| (trifluoromethyl)-3,6- | | | |
| dihydropyrimidin-1(2H)- | | | |
| yl]phenoxy}pyridin-2- | | | |
| yl)oxy]acetate | | | |
| Flufenoximacil | 1-200 | 5-75 | 25-50 |
| 2-methoxyethyl [(3-{2- | 1-200 | 5-75 | 25-50 |
| chloro-4-fluoro-5-[3-methyl- | | | |
| 2,6-dioxo-4- | | | |
| (trifluoromethyl)-3,6- | | | |
| dihydropyrimidin-1(2H)- | | | |
| yl]phenoxy}pyridin-2- | | | |
| yl)oxy]acetate | | | |
| cyanomethyl [(3-{2-bromo-4- | 1-200 | 5-75 | 25-50 |
| fluoro-5-[3-methyl-2,6-dioxo- | | | |
| 4-(trifluoromethyl)-3,6- | | | |
| dihydropyrimidin-1(2H)- | | | |
| yl]phenoxy}pyridin-2- | | | |
| yl)oxy]acetate | | | |
| cyclopropylmethyl (2-{2- | 1-200 | 5-100 | 50-100 |
| chloro-4-fluoro-5-[3-methyl- | | | |
| 2,6-dioxo-4- | | | |
| (trifluoromethyl)-3,6- | | | |
| dihydropyrimidin-1(2H)- | | | |
| yl]phenoxy}phenoxy)acetate | | | |

Methods for controlling volunteer corn comprising corn event Zm_CSM63715 in an area are provided. The methods comprise applying an herbicidally effective amount of at least one herbicide other than a PPO herbicide, wherein the herbicide application prevents growth of corn comprising event Zm_CSM63715. Illustrative examples of such herbicides are pyrithiobac, trifluralin, fluometuron, trifloxysulfuron, FOP herbicides (e.g., quizalofop or fluazifop), DIM herbicides (e.g., clethodim or sethoxydim), fenoxaprop, glyphosate, glufosinate, and combinations of any thereof, wherein the herbicide application prevents growth of volunteer corn comprising event Zm_CSM63715. For example, to control volunteer corn comprising event Zm_CSM63715 in a cotton cultivation field, fluometuron can be applied preemergence, and FOP herbicides (e.g., quizalofop or fluazifop), trifloxysulfuron, pyrithiobac, DIM herbicides (e.g., clethodim or sethoxydim), glyphosate, or glufosinate can be applied postemergence. To control volunteer corn comprising both event Zm_CSM63715 and event MON87429, the DIM herbicides such as clethodim or sethoxydim can be used.

Methods for producing plants and seeds comprising corn event Zm_CSM63715 are provided. Plants may be bred using any method known in the art. A progeny corn plant comprising the event Zm_CSM63715 may be produced, for example, by selfing a parent plant or line comprising the event Zm_CSM63715, wherein such parent plant or line is homozygous or hemizygous for the event Zm_CSM63715, or by crossing a first parent plant or line comprising the event Zm_CSM63715, wherein such parent plant or line is homozygous or hemizygous for the event Zm_CSM63715, with a second parent plant or line having a different genotype or germplasm than the first parent line, wherein the second parent plant or line may or may not contain or comprise the event Zm_CSM63715. As described further herein, corn event Zm_CSM63715 comprises a PPO expression cassette or transgene encoding a protoporphyrinogen oxidase. According to some embodiments, the transgenic corn plant(s) comprising the event Zm_CSM63715 is/are tolerant to PPO inhibitors, relative to a non-transgenic control plant. Transgenic corn plants used in these methods may be homozygous or heterozygous for the transgene. Progeny plants produced by these methods may be varietal or hybrid plants; may be grown from seeds produced by corn event Zm_CSM63715 containing plant and/or from seeds produced by a plant fertilized with pollen from a corn event Zm_CSM63715 containing plant; and may be homozygous or heterozygous for the transgenes and/or event Zm_CSM63715. Progeny plants may be subsequently self-pollinated to generate a true breeding line of plants, i.e., plants homozygous for the transgene, or alternatively may be out-crossed, e.g., bred with another unrelated plant, to produce a varietal or a hybrid seed or plant.

A method of obtaining a seed of a corn plant or a corn plant that is tolerant to PPO herbicides, is provided. The method comprises: (a) obtaining a population of progeny seed or plants grown therefrom, at least one of which comprises corn event Zm_CSM63715; and (b) identifying at least a first progeny seed or plant grown therefrom that comprises corn event Zm_CSM63715. Identifying the progeny seed or plant grown therefrom that comprises corn event Zm_CSM63715 can comprise: (a) growing the progeny seed or plant to produce progeny plants; (b) treating the progeny plants with an effective amount of a PPO herbicide; and (c) selecting a progeny plant that is tolerant to the PPO herbicide. Alternatively, or in addition, identifying the progeny seed or plant grown therefrom that comprises corn event Zm_CSM63715 comprises detecting the presence of corn event Zm_CSM63715 in a sample derived from the progeny seed or plant grown therefrom. Alternatively, or in addition, identifying the progeny seed or plant grown therefrom that comprises corn event Zm_CSM63715 comprises detecting the presence of the PPO protein encoded by corn event Zm_CSM63715 in a sample derived from the progeny seed or plant grown therefrom.

As used herein, the terms “line”, “breeding line”, “genotype” or “germplasm” are used interchangeably to refers to a group of plants that show little or no genetic variation between individuals for at least one trait. Such “line”, “breeding line”, “genotype” or “germplasm” can be created by self-pollination for several generations, selection, or vegetative propagation from a single parent using tissue or cell culture techniques. As used herein, the terms “cultivar” and “variety” are synonymous and refer to a line used for commercial production.

The production of double haploids may also be used to produce corn plants and seeds homozygous for event Zm_CSM63715 DNA in a breeding program. Double haploids are produced by the doubling of a set of chromosomes (1 N) from a heterozygous plant to produce a completely homozygous individual. For example, see Wan, et al., (1989) and U.S. Pat. No. 7,135,615. This can be advantageous because the process omits the generations of selfing needed to obtain a homozygous plant from a heterozygous source. One way of producing haploid and double haploid corn plant comprising event Zm_CSM63715 is through anther culture of flowers comprising event Zm_CSM63715 (Khan et al., 2010). Other methods such as natural polyembryony, induction with irradiated pollen, crosses with polyploid plants or wild species, unfertilized ovule and microspore culture can also be applied to produce haploid and double haploid corn plants comprising event Zm_CSM63715.

Seed and progeny plants made by the methods described herein comprise corn event Zm_CSM63715. Application of one or more herbicide for which corn event Zm_CSM63715 confers tolerance may be used to select progeny that comprise corn event Zm_CSM63715. Alternatively, progeny may be analyzed using diagnostic methods to select for plants or seeds comprising corn event Zm_CSM63715.

Corn transgenic events are known to one of skill in the art; for example, a list of such traits is provided by the United States Department of Agriculture's (USDA) Animal and Plant Health Inspection Service (APHIS) and can be found on their website at www.aphis.usda.gov. Two or more transgenic events may thus be combined in a progeny seed or plant by crossing two parent plants each comprising one or more transgenic event(s), collecting progeny seed, and selecting for progeny seed or plants that contain the two or more transgenic events; these steps may then be repeated until the desired combination of transgenic events in a progeny is achieved. Back-crossing to a parental plant and out-crossing with a non-transgenic plant are also contemplated, as is vegetative propagation.

Methods of detecting the presence of corn event Zm_CSM63715 in a sample of DNA derived from a corn seed, plant, plant part, plant cell, progeny plant, or commodity product are provided. One method comprises: (i) contacting the sample with at least one primer that is capable of producing DNA sequence specific to event Zm_CSM63715 DNA under conditions appropriate for DNA sequencing; (ii) performing a DNA sequencing reaction; and (iii) confirming that the nucleotide sequence comprises a nucleotide sequence specific for event Zm_CSM63715, of the transgenic insert comprised therein, such as one selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10.

Another method of detecting the presence of corn event Zm_CSM63715 in a sample of DNA derived from a corn seed, plant, plant part, plant cell, progeny plant, or commodity product is provided. The method comprises: (a) contacting the sample with a DNA probe specific for event Zm_CSM63715 DNA; and (b) performing a sequencing reaction to produce a target sequence. The target sequence comprises a nucleotide sequence selected from the group consisting of SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; a complete complement of any thereof; and a fragment of any of SEQ ID NO:1, SEQ ID NO:2, SEQ ID NO:3, SEQ ID NO:4, SEQ ID NO:5, SEQ ID NO:6, SEQ ID NO:7, SEQ ID NO:8, and SEQ ID NO:10 that is at least 10 nucleotides long and comprises nucleotides 1,000-1,001 or 4,552-4,553 of SEQ ID NO:10.

Another method of detecting the presence of corn event Zm_CSM63715 in a sample derived from a corn seed, plant, plant part or plant cell, progeny plant or commodity product comprises: (a) contacting the sample with a primer pair that is capable of producing an amplicon from event Zm_CSM63715 DNA under conditions appropriate for DNA amplification; (b) performing a DNA amplification reaction to produce a DNA amplicon; and (c) detecting the presence of the DNA amplicon. The DNA amplicon comprises a nucleotide sequence specific for event Zm_CSM63715, for example, at least one of (i) a 5′ junction sequence between flanking corn genomic DNA and the transgenic insert of corn event Zm_CSM63715, (b) a 3′ junction sequence between flanking corn genomic DNA and the transgenic insert of corn event Zm_CSM63715, (c) SEQ ID NO: 9, and (d) a fragment of SEQ ID NO: 9 comprising a sufficient length of contiguous nucleotides of SEQ ID NO: 9 to identify the sequence as a fragment of the transgenic insert of Zm_CSM63715. The presence of the DNA amplicon indicates the presence of corn event Zm_CSM63715 in the sample. The amplicon should be one that is specific for event Zm_CSM63715, and comprises the junction at nucleotide positions 1000-1001, and/or nucleotide positions 4,552-4,553 of SEQ ID NO:10. Thus, for example, the amplicon can comprise a nucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10; and a fragment of any of SEQ ID NO:10, SEQ ID NO:8, SEQ ID NO:7, SEQ ID NO:6, SEQ ID NO:5, SEQ ID NO:4, SEQ ID NO:3, SEQ ID NO:2, and SEQ ID NO:1 that is at least 10 nucleotides in length and comprises nucleotides 1,000-1,001 or 4,552-4,553 of SEQ ID NO:10. The amplicon can be at least 10 nucleotides in length, at least 11 nucleotides in length, at least 12 nucleotides in length, at least 13 nucleotides in length, at least 14 nucleotides in length, at least 15 nucleotides in length, at least 16 nucleotides in length, at least 17 nucleotides in length, at least 18 nucleotides in length, at least 19 nucleotides in length, at least 20 nucleotides in length, at least 25 nucleotides in length, at least 30 nucleotides in length, at least 35 nucleotides in length, at least 40 nucleotides in length, at least 45 nucleotides in length, at least 50 nucleotides in length, at least 60 nucleotides in length, at least 70 nucleotides in length, at least 80 nucleotides in length, at least 90 nucleotides in length, or at least 100 nucleotides in length.

The detection of a nucleotide sequence specific for event Zm_CSM63715 in the amplicon is determinative and/or diagnostic for the presence of the corn event Zm_CSM63715 specific DNA in the sample. An illustrative primer pair that is capable of producing an amplicon from event Zm_CSM63715 DNA under conditions appropriate for DNA amplification is provided as SEQ ID NO:14 and SEQ ID NO:15. Other primer pairs may be readily designed by one of skill in the art to produce an amplicon diagnostic for corn event Zm_CSM63715, wherein such a primer pair comprises at least one primer within the genomic region flanking the insert and a second primer within the insert, provided that any primer pair could be designed and used that produces an amplicon comprising a junction sequence and/or all or part of the insert or transgene sequence. Detection of an amplicon could be based on any suitable method, such as sequencing, determining fragment size or migration of the amplicon in a matrix or gel, or a hybridization-based method.

Another method of detecting the presence of corn event Zm_CSM63715 in a sample derived from a corn plant, plant part, plant cell, seed, progeny plant, or commodity product comprises: (i) contacting the sample with a DNA probe specific for event Zm_CSM63715 DNA; (ii) subjecting the sample and the DNA probe to stringent hybridization conditions; and (iii) detecting hybridization between the probe and the target DNA in the sample. An example of the sequence of a DNA probe that is specific for event Zm_CSM63715 is provided as SEQ ID NO:16. Other probes may be readily designed by one of skill in the art. Detection of probe hybridization to the DNA in the sample is diagnostic for the presence of corn event Zm_CSM63715 specific DNA in the sample. Absence of hybridization is alternatively diagnostic of the absence of corn event Zm_CSM63715 specific DNA in the sample.

Another method of detecting the presence of corn event Zm_CSM63715 in a sample derived from a corn plant, plant part, plant cell, seed, progeny plant, or commodity product comprises: (a) contacting the sample with an antibody specific for the PPO (protoporphyrinogen oxidase) protein encoded by corn event Zm_CSM63715; and (b) detecting binding of the antibody to the protein in the sample. The binding of the antibody indicates the presence of corn event Zm_CSM63715 in the sample.

An alternative to antibodies for protein detection is the aptamer-based detection method for detecting proteins or molecules of interest in a sample. As used herein, the term “aptamer(s)” or “aptamer sequences(s)” refers to short synthetic single-stranded oligonucleotide molecules with high-affinity and specificity binding to a target molecule such as a protein, polypeptide, lipid, glycoprotein, glycolipid, glycopeptide, saccharide, or polysaccharide by forming distinct tertiary structures (Ellington and Szostak, 1990; Robertson and Joyce, 1990; Tuerk and Gold, 1990; Wang et al., 2019). The single-stranded nucleic acid can be ssDNA, RNA, or derivatives of either thereof. The aptamer comprises a three-dimensional structure held in certain conformation(s) that provide intermolecular contacts to specifically bind its given target. Although aptamers are nucleic acid-based molecules, the binding to the target molecule is not entirely dependent on a linear base sequence, but rather a particular secondary/tertiary/quaternary structure. The term aptamer also covers next generation aptamers such as X aptamers that cannot typically be amplified by PCR, but can be adapted by adding a link primer. Such aptamers can specifically bind to proteins of interest but can also be easily amplified, sequenced etc. in a downstream process. The term aptamer also covers aptamers that include modified bases. It is envisaged that the aptamers may include traditional aptamers of 15 to 120 bases in length, as well as longer aptamers of approx. 200 bases in length (e.g., Ultramers® by Integrated DNA Technologies, Inc. Coralville, Iowa, USA). To detect the PPO protein in a sample, aptamers specific to the PPO protein are obtained and are then incubated with the sample. If the PPO is present in the sample, protein-aptamer conjugates are formed. Methods for detecting the aptamer/protein complex are known in the art, such as aptablotting or South-Western blot (Li et al., 2017; Sekhon et al., 2017), aptamer-based Western blot (Wang at al., 2020), aptamer sandwich assay (Svobodova et al., 2021). Chemical modifications, additional functional groups, and/or linkers can be added to the nucleic acid aptamer to provide increased binding affinity to a target protein, and to provide a convenient means to detect the target molecule. Such tags and or labels can comprise fluorescent, luminescent, absorbance, or radioactive-based chemical groups, or can comprise an enzyme or substrate that provides a detectable response such as a precipitate, either alone or in the presence of other factors.

Methods for determining the zygosity of the event and transgene with genomic DNA derived from at least one corn plant, plant part, plant cell or seed comprising corn event Zm_CSM63715 in a sample are provided. In one such method of determining the zygosity of a corn plant, plant part, plant seed, or plant cell comprising corn event Zm_CSM63715, the method comprises: (a) contacting a sample comprising DNA derived from the corn plant, plant part, plant seed, or plant cell with a first primer set capable of producing a first amplicon diagnostic for the presence of corn event Zm_CSM63715, and a second primer set capable of producing a second amplicon diagnostic for the wildtype corn genomic DNA not comprising corn event Zm_CSM63715; (b) performing a nucleic acid amplification reaction; and (c) detecting the first amplicon and the second amplicon, wherein the presence of both amplicons indicates that the plant, plant part, seed or cell is heterozygous for corn event Zm_CSM63715, and the presence of only the first amplicon indicates that the plant, plant part, seed, or cell is homozygous for corn event Zm_CSM63715. The presence of only the second amplicon is diagnostic for the absence of event Zm_CSM63715 DNA in the sample. Illustrative sets of primer pairs are SEQ ID NO:14 and SEQ ID NO:15, which produce an amplicon diagnostic for event Zm_CSM63715; and SEQ ID NO:20 and SEQ ID NO:21 or SEQ ID NO: 15 and SEQ ID NO: 21, which produce an amplicon diagnostic for wildtype corn genomic DNA not comprising event Zm_CSM63715. A set of probes can also be incorporated into such an amplification method to be used in a real-time PCR format using the primer pair sets described above. An illustrative set of probes are presented as SEQ ID NO:16 (diagnostic for the amplicon for the event Zm_CSM63715) and SEQ ID NO:22 (diagnostic for the amplicon for wildtype corn genomic DNA not comprising event Zm_CSM63715).

Another method for determining the zygosity of a corn plant, plant part, plant seed, or plant cell comprising corn event Zm_CSM63715 comprises (a) contacting a sample comprising DNA derived from the corn plant, plant part, plant seed, or plant cell with a probe set comprising at least a first probe that specifically hybridizes to corn event Zm_CSM63715, and at least a second probe that specifically hybridizes to corn genomic DNA that was disrupted by insertion of the heterologous DNA of corn event Zm_CSM63715 but does not hybridize to corn event Zm_CSM63715; and (b) hybridizing the probe set with the sample under stringent hybridization conditions. Detecting hybridization of only the first probe under the hybridization conditions is diagnostic for a corn plant, plant part, seed or plant cell homozygous for corn event Zm_CSM63715, and detecting hybridization of both the first probe and the second probe under the hybridization conditions is diagnostic for a corn plant, plant part, seed, or plant cell heterozygous for corn event Zm_CSM63715. Detecting hybridization of only the second probe under the hybridization conditions is diagnostic for the absence of event Zm_CSM63715 DNA in the sample. An illustrative probe set that can be used in the method is SEQ ID NO:16 and SEQ ID NO:20.

Yet another method for determining zygosity comprises (i) extracting a sample comprising DNA from at least one corn plant, plant part, plant cell or seed; (ii) contacting the sample with a first primer pair that is capable of producing a first amplicon diagnostic for event Zm_CSM63715; (iii) contacting the sample with a second primer pair that is capable of producing a second amplicon of an internal standard known to be single-copy and homozygous in the corn plant; (iv) contacting the sample with a probe set which contains at least a first probe that specifically hybridizes to the first amplicon, and at least a second probe that specifically hybridizes to the second amplicon; (v) performing a DNA amplification reaction using real-time PCR and determining the cycle thresholds (Ct values) of the first and second amplicons; (vi) calculating the difference (ΔCt) between the Ct value of the first amplicon and the second amplicon; and (vii) determining zygosity, wherein a ΔCt of about zero (0) indicates homozygosity of the event or inserted T-DNA, and a ΔCt of about one (1) indicates heterozygosity of the event or inserted T-DNA. Heterozygous and homozygous events are differentiated by a ΔCt value unit of approximately one (1). Given the normal variability observed in real-time PCR due to multiple factors such as amplification efficiency and ideal annealing temperatures, the range of “about one (1)” is defined as a ΔCt of 0.75 to 1.25, and the range of “about zero (0)” is defined as a ΔCt of −0.25 to 0.25 (or of 0.0 to 0.25 if the ΔCt is measured as an absolute value). Primer pairs and probes for the above method for determining zygosity can amplify and detect amplicons from the transgene or event DNA and the internal DNA standard.

A DNA construct is provided. The DNA construct comprises an expression cassette, wherein the expression cassette comprises, in operable linkage, i) a ubiquitin (UBQ) promoter, a leader sequence, and an intron sequence from Andropogon gerardi, ii) a chloroplast transit peptide coding sequence of APG6 (Albino and Pale Green 6) from Arabidopsis thaliana, iii) a codon-optimized protoporphyrinogen oxidase (PPO) coding sequence from Enterobacter cloacae for conferring to PPO herbicides, and iv) a 3′ UTR sequence of an alpha tubulin protein from Arundo donax. For example, the DNA construct can comprise SEQ ID NO: 9.

Expression of the PPO in transgenic plants confers tolerance to PPO herbicides. For example, plants, plant parts, plant cells or seeds containing or comprising corn event Zm_CSM63715 are tolerant to PPO herbicides flumioxazin, epyrifenacil, lactofen, acifluorfen, pyraflufen, pyraflufen-ethyl, oxadiazon, butafenacil, pyridin-2-ylmethyl[(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl[(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl[(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate, cyanomethyl[(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate, methyl (2R)-2-{[(E)-({2-chloro-4-fluoro-5-[3-methyl-2,6-dioxo-4-(trifluoromethyl)-3,6-dihydropyrimidin-1(2H)-yl]phenyl}methylidene)amino]oxy}propanoate (flufenoximacil), cyclopropylmethyl (2-{2-chloro-4-fluoro-5-1[3-methyl-2,6-dioxo-4-(trifluoromethyl)-3,6-dihydropyrimidin-1(2H)-yl]phenoxy}phenoxy)acetate, fomesafen, saflufenacil, sulfentrazone, tiafenacil, and trifludimoxazin, and combinations of any thereof.

Any of the DNA constructs or transgenic inserts described herein can further comprise at its 5′ or 3′ end at least 50 contiguous nucleotides of SEQ ID NO:11 or SEQ ID NO:164, or at least 50 contiguous nucleotides of SEQ ID NO:12 or SEQ ID NO:165. Alternatively, any of the DNA constructs or transgenic inserts described herein can further comprise at its 5′ or 3′ end at least 100 contiguous nucleotides, at least 150 contiguous nucleotides, at least 200 contiguous nucleotides, at least 250 contiguous nucleotides, at least 300 contiguous nucleotides, at least 350 contiguous nucleotides, at least 400 contiguous nucleotides, at least 450 contiguous nucleotides, or at least 500 nucleotides of SEQ ID NO:11 or SEQ ID NO:164, or at least 100 contiguous nucleotides, at least 150 contiguous nucleotides, at least 200 contiguous nucleotides, at least 250 contiguous nucleotides, at least 300 contiguous nucleotides, at least 350 contiguous nucleotides, at least 400 contiguous nucleotides, at least 450 contiguous nucleotides, or at least 500 nucleotides of SEQ ID NO:12 or SEQ ID NO:165.

SEQ ID NOs:11 and 12 are 1,000 nucleotide sequences representing corn genomic DNA that flanks the transgenic insert at the 5′ and 3′ ends of the insert in corn event Zm_CSM63715, respectively. SEQ ID NO:11 and SEQ ID NO:12 have been validated by sequencing, as further described in Example 5 hereinbelow. SEQ ID NOs:164 and 165 are 5,000 nucleotide sequences representing corn genomic DNA that flanks the transgenic insert at the 5′ and 3′ ends of the insert, respectively. Nucleotides 4,001-5,000 of SEQ ID NO:164 are identical to nucleotides 1-1,000 of SEQ ID NO:11. The remaining nucleotides of SEQ ID NO:164 (nucleotides 1-4,000) are based on the genomic sequence of the B73 corn cultivar (Zm-B73-REFERENCE-GRAMENE-4.0, NCBI). Similarly, nucleotides 1-1000 of SEQ ID NO:165 are identical to nucleotides 1-1,000 of SEQ NO:12. The remaining nucleotides of SEQ ID NO:165 (nucleotides 1,001-5,000) are based on the genomic sequence of the B73 corn cultivar.

The at least 50 contiguous nucleotides of SEQ ID NO:11 or SEQ ID NO:164 at the 5′ end of the DNA construct or transgenic insertion may be immediately adjacent to and upstream (on the 5′ end) of the transgenic insertion, or may not be immediately adjacent to, but further upstream (on the 5′ end) and within about 5000 nucleotides, within about 3000 nucleotides, or within about 1000 nucleotides of the transgenic insertion. Likewise, the at least 50 contiguous nucleotides of SEQ ID NO:12 or SEQ ID NO:165 at the 3′ end of the DNA construct or transgenic insertion may be immediately adjacent to and downstream (on the 3′ end) of the transgenic insertion, or may not be immediately adjacent to but further downstream (on the 3′ end) and within about 5000 nucleotides, within about 3000 nucleotides, or within about 1000 nucleotides of the transgenic insertion. Illustrative examples of sequences comprising 50 contiguous nucleotides of SEQ ID NO:11 are provided in SEQ ID NOs:44-63. Illustrative examples of 50 contiguous nucleotides of SEQ ID NO:12 are provided in SEQ ID NOs:104-123. Illustrative examples of 50 contiguous nucleotides of SEQ ID NO:164 are provided in SEQ ID NOs:64-103. Illustrative examples of 50 contiguous nucleotides of SEQ ID NO:165 are provided in SEQ ID NOs:124-163. However, any sequence comprising at least 50 contiguous nucleotides of SEQ ID NO:11 or SEQ ID NO:165, or at least 50 contiguous nucleotides of SEQ ID NO:12 or SEQ ID NO:165 is within the scope of the present disclosure.

In addition, a DNA construct comprising a PPO expression cassette is provided. The DNA construct further comprises at the 5′ and/or 3′ end of the construct (i) at least 50 contiguous nucleotides of SEQ ID NO:11 or SEQ ID NO:164; and/or (ii) at least 50 contiguous nucleotides of SEQ ID NO:12 or SEQ ID NO:165.

A further DNA construct is provided. The DNA construct comprises a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO: 9. The DNA construct further comprises at the 5′ and/or 3′ end of the construct (i) at least 50 contiguous nucleotides of SEQ ID NO:11 or 164; and/or (ii) at least 50 contiguous nucleotides of SEQ ID NO:12 or 165.

For example, any of the DNA constructs can comprise at the 5′ end of said construct one or more nucleotide sequences selected from SEQ ID NOs:44-103. Alternatively, or in addition, any of the DNA constructs can comprise at the 3′ end of said construct one or more nucleotide sequences selected from SEQ ID NOs:104-163.

Corn plants, plant cells, plant parts, and plant seeds comprising any of the DNA constructs described herein are also provided.

Also provided are corn plants, plant cells, plant parts, and plant seeds comprising a recombinant DNA construct integrated in chromosome 8, wherein the recombinant DNA construct confers tolerance to at least one PPO herbicide. The recombinant DNA construct is integrated in a position of said chromosome flanked by at least 50 contiguous nucleotides of SEQ ID NO:11 or 164 and at least 50 contiguous nucleotides of SEQ ID NO:12 or 165. The at least 50 contiguous nucleotides of SEQ ID NO:11 or 164 can comprise one or more nucleotide sequences selected from SEQ ID NOs:44-103, and the at least 50 contiguous nucleotides of SEQ ID NO:12 or 165 can comprise one or more nucleotide sequences selected from SEQ ID NOs:104-163.

Methods of improving tolerance to herbicides are provided. The methods comprise: i) inserting a DNA construct comprising a PPO expression cassette, as described herein, into the genome of a corn cell, ii) generating a corn plant from the corn cell; and iii) selecting a corn plant comprising the DNA construct. The selecting can comprise treating the corn cell or plant with an effective amount of a PPO herbicide.

Transgenic plants produced by the methods comprise a unique combination of expression elements for optimal expression of the transgene. Furthermore, transgenic plants produced by the methods as described herein acquire tolerance to PPO herbicides. Selecting the regenerated plant comprising the DNA construct may be done using DNA or protein detection methods as described herein. Alternatively or additionally, selecting may comprise treating the transgenic plant or plant cell with an effective amount of at least one PPO herbicide selected from the group consisting of flumioxazin, epyrifenacil, lactofen, acifluorfen, pyraflufen, pyraflufen-ethyl, oxadiazon, butafenacil, pyridin-2-ylmethyl[(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl[(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl[(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate, cyanomethyl[(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate, methyl (2R)-2-{[(E)-({2-chloro-4-fluoro-5-[3-methyl-2,6-dioxo-4-(trifluoromethyl)-3,6-dihydropyrimidin-1(2H)-yl]phenyl}methylidene)amino]oxy}propanoate (flufenoximacil), cyclopropylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate, fomesafen, saflufenacil, sulfentrazone, tiafenacil, and trifludimoxazin, or a combination of any thereof.

Methods of controlling, preventing, or reducing the development of herbicide-tolerant weeds are provided. The methods comprise: a) cultivating in a crop growing environment a corn plant comprising a DNA construct or transgene of the present disclosure or event Zm_CSM63715 that provides tolerance to PPO herbicides, and b) applying to the crop growing environment at least one PPO herbicide selected from the group consisting of flumioxazin, epyrifenacil, lactofen, acifluorfen, pyraflufen, pyraflufen-ethyl, oxadiazon, butafenacil, pyridin-2-ylmethyl[(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl[(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl[(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate, cyanomethyl[(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate, methyl (2R)-2-{[(E)-({2-chloro-4-fluoro-5-[3-methyl-2,6-dioxo-4-(trifluoromethyl)-3,6-dihydropyrimidin-1(2H)-yl]phenyl}methylidene)amino]oxy}propanoate (flufenoximacil), cyclopropylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate, fomesafen, saflufenacil, sulfentrazone, tiafenacil, and trifludimoxazin, or a combination of any thereof, wherein the corn plant is tolerant to the at least one PPO herbicide.

Methods of controlling, preventing, or reducing the development of herbicide-tolerant weeds are provided. One such method comprises cultivating in a crop growing environment a corn plant comprising transgenes that provide tolerance to (i) a PPO herbicide and (ii) herbicides with at least three additional herbicide modes of action, the three additional herbicide modes of action each being different from one another. The at least three additional herbicide modes of action can be selected from the group consisting of inhibition of glutamine synthetase, inhibition of acetyl CoA carboxylase (ACCase) in the aryloxyphenoxy propionate (FOP) group, inhibition of EPSPS, and synthetic auxins.

Another method of controlling, preventing, or reducing the development of herbicide-tolerant weeds is provided. The method comprises: (a) cultivating in a crop growing environment a corn plant comprising any of the DNA constructs comprising a PPO tolerance gene described herein, and at least three additional transgenes for providing tolerance to herbicides with at least three additional herbicide modes of action, the three additional herbicide modes of action each being different from one another; and (b) applying to the crop growing environment at least one herbicide selected from the group consisting of dicamba, glufosinate, 2,4-D, PPO inhibitor, glyphosate, and any combination thereof, wherein the corn plant is tolerant to the at least one herbicide.

In any of the methods of controlling, preventing, or reducing the development of herbicide-tolerant weeds, the transgenes that provide tolerance to the herbicides with at least three additional herbicide modes of action can be present at a single genomic location in the corn plant. The transgenes that provide tolerance to the herbicides with at least three additional herbicide modes of action can be selected from the group consisting of DMO, PAT, FT_T, EPSPS, and combinations of any thereof. For example, the corn plant, plant seed, plant part, or plant cell used in these methods can further comprise corn event MON87429.

An illustrative PAT coding sequence and its corresponding amino acid sequence from Streptomyces viridochromogenes are provided as SEQ ID NO:166 and SEQ ID NO:167, respectively. An illustrative DMO coding sequence and its corresponding amino acid sequence from Pseudomonas maltophilia are provided as SEQ ID NO:168 and SEQ ID NO:169, respectively. An illustrative FT_T coding sequence and its corresponding amino acid sequence from Sphingobium herbicidovorans are provided as SEQ ID NO:170 and SEQ ID NO:171, respectively. An illustrative EPSPS coding sequence and its corresponding amino acid sequence from Agrobacterium CP4 strain are provided as SEQ ID NO:172 and SEQ ID NO:173, respectively.

The corn plants, plant seeds, plant parts, or plant cells produced by the preceding methods are tolerant to at least one additional herbicide besides PPO inhibitors in a field, selected from the group consisting of inhibitors of acetyl CoA carboxylase (ACCase) in the aryloxyphenoxy propionate (FOP) group, inhibitors of EPSPS, synthetic auxins and inhibitors of glutamine synthetase. Examples of the inhibitor of acetyl CoA carboxylase (ACCase) in the aryloxyphenoxy propionate (FOP) group include, but are not limited to, chlorazifop, clodinafop, clodinafop-ethyl, clodinafop-propargyl, clofop, cyhalofop, cyhalofop-butyl, diclofop, diclofop-methyl, diclofop-P, diclofop-P-methyl, fenoxaprop, fenoxaprop-P, fenoxaprop-P-ethyl, fenthiaprop, fluazifop, fluazifop-butyl, fluazifop-P, fluazifop-P-butyl, haloxyfop, haloxyfop.etotyl, haloxyfop-methyl, haloxyfop-P, haloxyfop-P-methyl, isoxapyrifop, metamifop, propaquizafop, quizalofop, quizalafop-ethyl, quizalofop-P, quizalafop-P-ethyl, quizalafop-P-tefuryl, trifop, and combinations of any thereof. Examples of the synthetic auxin include, but are not limited to, dicamba, 2,4-D, dichlorprop, mecoprop, 2A5 (2,4,5-trichlorophenoxyacetic acid), and combinations of any thereof. An example of the inhibitor of glutamine synthetase is glufosinate. An example of the inhibitor of 5-enolpyruvylshikimate-3-phosphate synthase (EPSPS) is glyphosate.

The herbicide(s) used in the methods described herein can be applied alone, sequentially with or in combination with one or more herbicide(s) during the growing season. The herbicide(s) used in the methods described herein can be applied in combination with one or more herbicide(s) temporally (for example, as a tank mixture or in sequential applications), spatially (for example, at different times during the growing season including before and after corn seed planting), or both. For example, a method for controlling the development of herbicide resistance in weeds is provided that consists of planting seed comprising corn event Zm_CSM63715 in an area and applying an herbicidally effective amount over the growing season of one or more PPO herbicides alone or in any combination, for the purpose of controlling the development of herbicide resistance in weeds in the area. Such application of herbicide(s) may be pre-planting (any time prior to planting seed comprising corn event Zm_CSM63715, including for burn-down purposes, that is application to emerging or existing weeds prior to seed plant), pre-emergence (any time after seed comprising corn event Zm_CSM63715 is planted and before plants comprising corn event Zm_CSM63715 emerge), or post-emergence (any time after plants comprising corn event Zm_CSM63715 emerge). Multiple applications of one or more herbicides, or a combination of herbicides together or individually, may be used over a growing season, for example, two applications (such as a pre-planting application and a post-emergence application, or a pre-emergence application and a post-emergence application) or three or more applications (such as a pre-planting application and two post-emergence applications).

Also provided are methods of reducing loci for corn breeding by site-directed insertion of a transgene that provides tolerance to a PPO herbicide at a genomic location in a corn plant that is within about 3-8 cM of a locus in the genome of the corn plant that comprises transgenes for tolerance to at least three additional herbicide modes of action, the three additional herbicide modes of action each being different from one another. The transgenes for tolerance to the at least three additional herbicide modes of action can be selected from the group consisting of PAT, DMO, FT_T, EPSPS, and combinations of any thereof. These transgenes provide a commercial level of tolerance to at least one herbicide in a field, such as dicamba, glufosinate, quizalofop or haloxyfop, 2,4-D, glyphosate, and combinations of any thereof. Illustrative nucleotide and amino acid sequences of the PAT gene are provided as SEQ ID NOs:166-167; illustrative nucleotide and amino acid sequences of the DMO gene are provided as SEQ ID NOs:168-169; illustrative nucleotide and amino acid sequences of the FT_T gene are provided as SEQ ID NOs:170-171; and illustrative nucleotide and amino acid sequences of the EPSPS gene are provided as SEQ ID NOs:172-173. Crossing of event Zm_CSM63715 with a corn plant that comprises these transgenes (e.g., with a plant comprising corn event MON87429) produces progeny plants comprising PPO, PAT, DMO, FT_T and EPSPS, which segregate as a single locus. As used herein, the term “commercial level” in reference to an herbicide refers to the recommended commercial rate (1X) for herbicide application for a specific herbicide. For example, a 1X rate of dicamba is 0.5 lb/acre; a 1X rate of 2,4-D is 0.75 lb/acre post-emergence; a 1X rate of glufosinate is 0.4 lb/acre; and a 1X rate of quizalofop is 0.08 lb/acre pre-emergence, and 0.08 lb ai/acre post-emergence. As used herein, “commercial level tolerance” refers to tolerance to one or more herbicides at the recommended commercial rates or higher as a result of transgene expression from one or more expression cassettes in plants comprising event Zm_CSM63715.

Corn event Zm_CSM63715 with the unique characteristics such as stable integration and expression of the PPO transgene, consistent and superior combinations of efficacy, including herbicide tolerance and agronomic performance, in and across multiple environment conditions in different geographies can be bred or introgressed into elite lines or varieties by conventional breeding methods, and maintained over subsequent generations. Furthermore, since the event was integrated near the transgenic insertion site of a corn plant comprising the transgenes of PAT, DMO, FT_T and EPSPS, the methods of the present disclosure allow for PPO along with PAT, DMO, FT_T and EPSPS to segregate as a single locus if bred together, which will allow for rapid trait integration of the multiple transgenes on segregating material, saving time and resources in a breeding program and enabling rapid development of lines, compared to cases where the individual transgenes are inserted into two or more loci, necessitating tedious and laborious multiple crosses over multiple generations to select for plants comprising the multiple genes. The newly introgressed or integrated DNA molecule or polynucleotide of event Zm_CSM63715 comprising SEQ ID NO:9 and/or SEQ ID NO:10 will maintain the expression characteristics of the transgene, and the genomic flanking sequences and chromosomal location, where it will confer tolerance to the PPO herbicides.

### Deposit Information

A deposit of a representative sample of corn seed comprising event Zm_CSM63715 has been made on Sep. 9, 2022, according to the Budapest Treaty with the American Type Culture Collection (ATCC) Patent Repository having an address at 10801 University Boulevard, Manassas, Virginia, 20110, USA. The ATCC Patent Deposit Designation (accession number) for seeds comprising corn event Zm_CSM63715 is Accession No. PTA-127361. Access to the deposits will be available during the pendency of the application to the Commissioner of Patents and Trademarks and persons determined by the Commissioner to be entitled thereto upon request. Upon issuance of the patent, all restrictions upon availability to the public will be irrevocably removed. The deposit will be maintained in the depository for a period of thirty (30) years, or five (5) years after the last request, or for the effective life of the patent, whichever is longer, and will be replaced as necessary during that period.

### Examples

The following examples are included to more fully describe the invention. Summarized are the construction and testing of 247 initial proof-of-concept transformation constructs containing various combinations of expression elements and PPO genes/variants, identification, testing and selection of gRNA target sites for site-directed integration of the PPO transgene, leading to the construction and testing of five commercial transformation constructs, the production and testing of over 18,000 unique transformation events, and the analysis of thousands of individual plants over multiple seasons through rigorous molecular characterization, efficacy and agronomic testing both in a controlled environment and in field trials, leading to the creation, identification, and ultimate selection of corn event Zm_CSM63715 (FIG. 3).

The Examples illustrate certain embodiments of the present disclosure. It should be appreciated by those of skilled in the art that many modifications can be made in the specific examples which are disclosed and still obtain a similar result. Certain agents which are both chemically and physiologically related may be substituted for the agents described herein while achieving the same or similar results. All such substitutions and modifications apparent to those skilled in the art are deemed to be within the scope of the invention.

### Example 1: Testing and Selection of PPO Genes and Expression Elements

This example describes design and testing of different protoporphyrinogen oxidase (PPO) genes and variants, promoters, chloroplast targeting peptides, and 3′ UTRs in many different proof-of-concept constructs to select combinations for commercial transformation to achieve tolerance to PPO herbicides.

Transgene expression and performance in transgenic plants may be influenced by many factors. These include but are not limited to: 1) the expression elements used to drive the expression of the transgene in the expression cassette, and their interactions among themselves and with the transgene; 2) efficient targeting of the transgenic protein to the site of action, such as in the plastids; 3) the relative position and orientation of different expression cassettes when the transgenic insert comprises multiple expression cassettes, each carrying a different transgene conferring a distinct trait; 4) the genomic location of the transgenic insertion, also known as positional effect; and 5) faithful integration of the transgene cassette(s) at the intended genomic location on the chromosome for site directed integration. A commercially useful transgenic event requires that the transgene(s) in the transgenic insert expresses in the manner necessary for that trait to be successful, e.g., transgene expression and performance across different tissues and developmental stages, in various germplasms, and under different growth conditions.

For these reasons, it is often necessary to create and screen a large number of constructs and transformation events in order to identify combinations of expression elements for constructs (the lead constructs), and then an event (the lead event), which demonstrates optimal expression and performance of the transgenes without phenotypic and agronomic off-types such as yield drag. Prior to such studies, it is not possible to determine whether a particular beneficial event phenotype or performance can be obtained.

In an initial proof-of-concept and early development stage study, conducted over four years and seven growing seasons, a total of 247 different constructs containing 48 different PPO genes/variants from 25 diverse source of organisms, 22 different 5′ UTRs, 34 different chloroplast transit peptide sequences, and 22 different 3′UTRs were designed, constructed and transformed into corn seed-derived embryo explants through Agrobacterium-mediated transformation using methods known in the art. Each construct also contained a CP4 cassette as a selectable marker. Thousands of transformed plants were regenerated and tested in the greenhouse and in field trials for protein expression through measurement of protein levels by ELISA, for trait efficacy through herbicide spray treatment, and for agronomic performance. Table 3 provides a summary of the proof-of-concept experiments. A total of 18,520R0 transgenic events were generated from 247 constructs, 18,224 of which were subjected to quantitative Taqman PCR assay for transgene copy number. Of these, 8,727 of the events contained a single copy of the transgene. 17,970 of the events were sprayed with PPO herbicides, and about one-fourth (4,561) of these events passed the spray test, showing <10% herbicide injury. A total of 850 events were advanced to R₁ nursery for seed increase and field testing for construct selection.

TABLE 3
Summary of proof-of-construct testing.

| | |
|---|---|
| # Constructs | 247 |
| # PPO variants | 48 |
| # PPO source organisms | 25 |
| # 5′ UTRs | 22 |
| # Chloroplast transit peptides | 34 |
| # 3′ UTRs | 22 |
| # R0 transgenic events generated | 18520 |
| # R0 events with molecular data | 18224 |
| # R0 events passed molecular criteria | 8727 |
| # Events sprayed with PPO herbicides | 17970 |
| # Events passing spray with ≤10% injury | 4561 |
| # Events advanced to field testing | 850 |

R0 events were generated and screened over a period of time. As they passed the molecular screening and greenhouse spray test, they were advanced to field trials over 7 field trial seasons in the US and/or South America. Some events were tested in more than one field trial, while others in one and dropped due to lack of efficacy or/and agronomic performance. Table 4 provides a summary of the field trials for the proof-of-concept constructs.

TABLE 4
Summary of field trials on the proof-of-concept constructs.

| Field | | | | | |
|---|---|---|---|---|---|
| Trial | | # | | # Location/ | Herbicide Application |
| Season | Location | Construct | Field Trials Type | # Replication | Stage |
| 1 | US | 4 | Hybrid efficacy | 2/2 | V2, V4, V8 |
| 2 | US | 11 | Hybrid efficacy | 2/2 | V2, V6 |
| | | 4 | Inbred efficacy | 2/2 | V2, V6 |
| | | 1 | Inbred agronomics | 8/3 | |
| 3 | South | 10 | Hybrid efficacy | 4/2 | V2, V6 |
| | America | 6 | Inbred efficacy | 2/2 | V2, V6 |
| | | 10 | Inbred agronomics | 8/3 | |
| 4 | US | 66 | Hybrid efficacy | 2/2 | Pre-emergence, V2, V6 |
| | | 51 | Inbred efficacy | 2/2 | Pre-emergence, V2, V6 |
| | | 48 | Inbred agronomics | 8/3 | |
| 5 | South | 30 | Hybrid efficacy | 4/2 | Pre-emergence, V2, V6 |
| | America | 28 | Inbred efficacy | 2/2 | Pre-emergence, V2, V6 |
| | | 30 | Inbred agronomics | 8/3 | |
| 6 | US | 51 | Hybrid efficacy | 2/2 | Pre-emergence, V2, V6 |
| | | 48 | Inbred efficacy | 2/2 | Pre-emergence, V2, V6 |
| 7 | South | 2 | Hybrid efficacy | 4/8 | Pre-emergence, V2, V6 |
| | America | 1 | Hybrid agronomics | 6/8 | |
| | | 2 | Inbred efficacy | 2/4 | Pre-emergence, V2, V6 |
| | | 1 | Inbred agronomics | 6/8 | |

Herbicide treatments were applied using a CO₂ backpack or tractor mounted sprayer calibrated to deliver 15 gallons per acre (GPA) using air-inducted Teejet® TTI nozzles with water as the herbicide carrier. Plots were rated for visual crop injury 10-14 days after herbicide treatment on a scale of 0-100 with “0” being none and “100” being complete. Plant height (PHT), ear height (EHT), days to 50% silk (S50D), days to 50% pollen (P50D), shell weight (SHW), test weight (TWT), moisture (MST), and grain yield (YLD) were also collected. All data were subjected to analysis of variance and means separation at p<0.05.

Based on the results of greenhouse spray test, transgene copy number, and the seven season field trials, top performing combinations of expression elements, chloroplast transit peptide and PPO gene were identified, and were used for designing commercial transformation vectors.

### Example 2: Selection of Genomic Target Sites and Guide RNAs

This example describes the bioinformatic analysis and identification of genomic target sites for site directed integration (SDI) of transgenes; screening of LbCas12a (also known as LbCpf1) guide RNAs targeting the genomic sites and identification of unique, high-quality gRNAs to facilitate SDI of transgenes.

In order to identify potential genomic sites for site directed integration of the PPO transgene cassette at a site close to the location of the MON87429 event, and generate a transgene stack to reduce transgenic loci to facilitate trait introgression and breeding, the MON87429 event was first mapped onto the Corn B73 reference genome assembly. The genome sequence 5 centimorgan (cM) up and downstream of the event MON87429 insertion site (referred to as the 10 cM window) was identified and the genetic coordinates were linked to physical positions on the B73 chromosome. The 10 cM window was interrogated for regions that were more than 1 kb from a gene, more than 1 kb from a repressive chromatin mark (e.g., H3K27me3 peak), more than 200 nucleotides (nt) from a small RNA hotspot, more than 1 kb from a long repeat region, had low DNA methylation (less than or equal to 10% of genome-wide population average) and had a low redundancy score (less than or equal to 30%). A suite of genomic loci was identified from this analysis.

CRISPR Cas12a-based cleavage of DNA sequences requires the use of gRNAs that can target specific sequences (target sites) comprising a Cas12a-recognizable protospacer adjacent motif (PAM) sequence so as to facilitate Cas12a enzymatic activity and DNA cleavage. The genomic loci identified as described above were therefore scanned for the presence of LbCas12a preferred PAM (TTTV, where “V” is A, C, or G), and 17 gRNA target sites were chosen for testing based on additional factors like GC content, uniqueness in the genome, and absence of potential off-targets. Table 5 summarizes the identified target sites that are represented by SEQ IDs:174-190. PAM sites are italicized and shown in bold.

TABLE 5
Cas 12a gRNA target sites on Zea mays
B73 chromosome 8.

| | | | Corre- |
|---|---|---|---|
| | | Tar- | spond- |
| gRNA | | get | ing |
| Tar- | | Site | gRNA |
| get | | SEQ | Spacer |
| Site | | ID | SEQ |
| Name | gRNA Target Sequence | NO | ID NO |
| ZmTS1 | TTTAACCGACTTGGGCATATATTATTT | 174 | 195 |
| ZmTS2 | TTTAACTCTAAATTTAGTTGGAGACAA | 175 | 196 |
| ZmTS3 | TTTACGTTTCGACAACAGTAATATATC | 176 | 197 |
| ZmTS4 | TTTCAACCTGCTGAAGTTGATAACTCT | 177 | 198 |
| ZmTS5 | TTTGGCAGTACGTATAGACCCACTTAA | 178 | 199 |
| ZmTS6 | TTTCATCGTCTAGGGAGCCTGACGAAA | 179 | 200 |
| ZmTS7 | TTTCCTTTAGGCTCCCTTGGAAATGCA | 180 | 201 |
| ZmTS8 | TTTAAGCCTATAGTCAAGAATACAATA | 181 | 202 |
| ZmTS9 | TTTGAGCAAATATCACATCATTGGGCC | 182 | 203 |
| ZmTS10 | TTTGCCACAAGGCTATTCACTCCCTAT | 183 | 204 |
| ZmTS11 | TTTCGTTAACTATTCTATCAACTAGAG | 184 | 205 |
| ZmTS12 | TTTATTGTTTGCTACAGATGACATATC | 185 | 206 |
| ZmTS13 | TTTGCCTCGAGGACTACATTCATAGGA | 186 | 207 |
| ZmTS14 | TTTGAGAGGAACCACCATTTCATGTGA | 187 | 208 |
| ZmTS15 | TTTCGACAACAGTAATATATCGTTCAA | 188 | 209 |
| ZmTS16 | TTTGGATAACCAAACCAGGGCCTATAG | 189 | 210 |
| ZmTS17 | TTTAAGGCGGACAGAGGGAGACTATCC | 190 | 211 |

Agrobacterium T-DNA vectors were designed to evaluate gRNA and LbCas12a nuclease mediated cleavage at the selected target sites. Each T-DNA vector comprised 3 cassettes. The first cassette comprised a plant codon optimized LbCas12a (SEQ ID NO:33) flanked by the same nuclear localization signal sequence (NLS) at the 5′ and 3′ ends (SEQ ID:191), and was operably linked to Zea mays Ubiquitin promoter (SEQ ID NO:192) and a 3′UTR sequence from a rice lipid transfer protein (LTP) gene (SEQ ID NO:193). The second cassette was a unique gRNA cassette targeting one of the 17 targeting sites described in Table 5. The cassette comprised a Zea mays Pol III promoter operably linked to a Cas12a gRNA unit. Each gRNA unit comprised a 36 bp upstream pre-crRNA scaffold (also called direct repeat) sequence (SEQ ID NO:194) compatible with LbCas12a, a 23 bp unique spacer sequence (SEQ ID NOs. 195-211) that when transcribed resulted in a sequence that was complementary to and hybridized with a genomic target site listed in Table 5, a 21 bp downstream mature crRNA scaffold (SEQ ID NO: 23) and a 7 bp poly T sequence. The third cassette was an EPSPS expression cassette encoding a 5-enolpyruvylshikimate-3-phosphate synthase as a selectable marker for selecting transformants in the present of glyphosate.

Corn embryo explants were transformed with the vectors described above by Agrobacterium-mediated transformation. Transformed plants were selected on glyphosate. Leaf samples from regenerated plantlets were harvested, and genomic DNA was extracted for Fragment Length Analysis (FLA). FLA is a PCR-based molecular assay that can be used to identify indel (insertion or deletion) mutations introduced at the target site by NHEJ-mediated (Non-Homologous End Joining) DNA repair following dsDNA cleavage by the LbCas12a-guide complex. Genomic DNA was subjected to a PCR reaction with primers flanking each target site to generate amplicons. The amplicon fragment lengths were subsequently analyzed using capillary electrophoresis, and compared to a wildtype amplicon to identify mutants that had larger or smaller amplicons due to the presence of indels. PCR reactions were carried out using a 5′ fluorescein amidite (FAM)-labeled primer, a standard primer and Phusion™ polymerase (New England Biolabs, MA) according to the manufacturer's instructions to generate 200 to 500 bp PCR fragments. One μl of the PCR product was combined with 0.5 μl of the GeneScan 1200 LIZ Size Standard (Thermo Fisher, MA), 8.5 μl of formamide, and ran on ABI3730 sequencer (Thermo Fisher, MA) to detect amplicon size and analyzed for fragment length variation to identify plants with mutations at the target sites. Table 6 summarizes the results and shows the mutation rate detected at each site in stably transformed corn plants. NA denotes that a transformation was performed, but was not successful. gRNA activity was determined by calculating the percentage of plants that contained a target site indel edit out of the total number of plants examined. As shown in Table 6, the target site editing rate for the tested gRNAs ranged from 4% to 95%.

TABLE 6
gRNA target site editing rate.

| gRNA Target | Number of Plants | Number of Plants with | Target |
|---|---|---|---|
| Site Name | Examined | Target Site Edits | Editing Rate |
| ZmTS1 | 151 | 16 | 11% |
| ZmTS2 | NA | NA | NA |
| ZmTS3 | NA | NA | NA |
| ZmTS4 | 174 | 7 | 4% |
| ZmTS5 | 35 | 12 | 34.29% |
| ZmTS6 | 129 | 26 | 20% |
| ZmTS7 | 37 | 15 | 41% |
| ZmTS8 | 205 | 40 | 20% |
| ZmTS9 | 65 | 5 | 8% |
| ZmTS10 | 55 | 32 | 58% |
| ZmTS11 | 201 | 25 | 12% |
| ZmTS12 | 144 | 19 | 13% |
| ZmTS13 | 78 | 4 | 5% |
| ZmTS14 | 161 | 54 | 33.5% |
| ZmTS15 | 163 | 28 | 17% |
| ZmTS16 | 132 | 125 | 95% |
| ZmTS17 | NA | NA | NA |

gRNAs targeting three sites ZmTS10, ZmTS14, and ZmTS16 had greater than 30% editing activity. The top performing gRNAs, i.e., the gRNAs targeting ZmTS10 comprising spacer SEQ ID NO:204 (58% editing rate), the gRNA targeting ZmTS14 comprising spacer SEQ ID NO:208 (33.5% editing rate), and the gRNA targeting ZmTS16 comprising spacer SEQ ID NO:210 (95% Editing rate), were advanced for site directed integration of the PPO trait cassette.

### Example 3: Construct Design, Molecular Analysis, and Event Selection

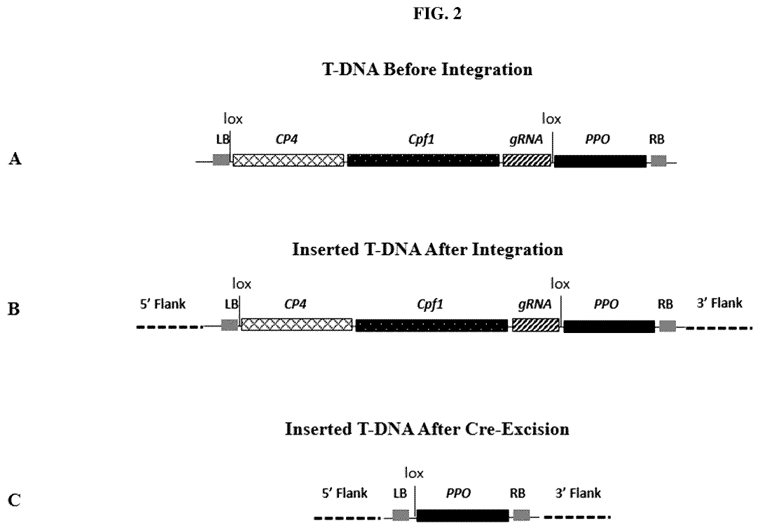

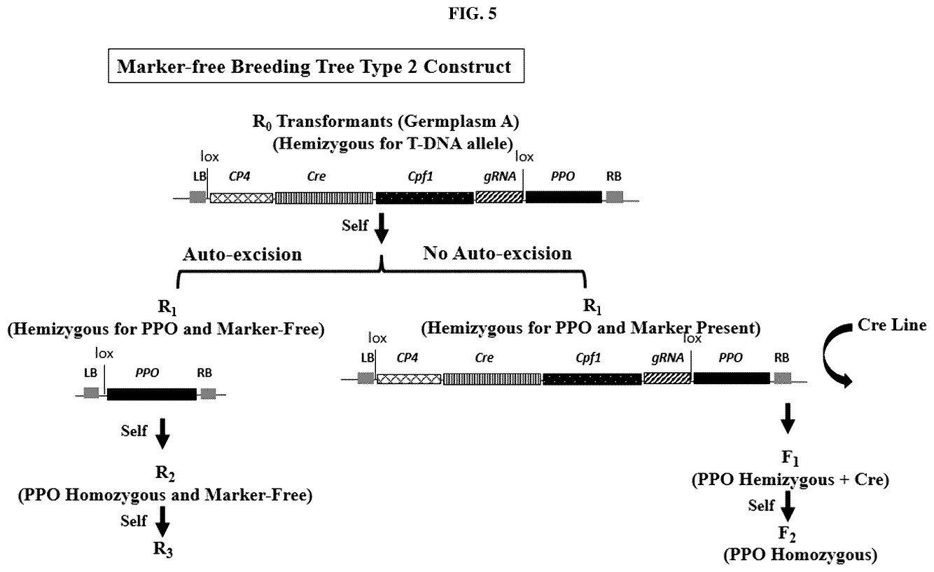

Based on the test results of individual expression cassettes from Example 1, and the results of guide RNA target site testing from Example 2, two types of commercial transformation constructs were designed. The three Type 1 constructs each contained four cassettes between the left and right borders on the T-DNA: a CP4 cassette, a Cpf1 cassette, a gRNA cassette, and a PPO cassette. Two lox sites flanked the CP4 and the gRNA cassettes. Upon crossing of the transgenic events with a line expressing the Cre enzyme, the CP4, Cpf1 and gRNA cassettes were excised out, leading to progenies containing only the PPO cassette (FIG. 2). The three Type 1 constructs contained identical CP4, Cpf1 and PPO cassettes, but differed in the gRNA spacers in the gRNA cassette for targeting the PPO cassette into different sites on the same chromosome. The two Type 2 constructs were similar to Type 1 constructs in organization, but contained a Cre cassette between the CP4 and the Cpf1 cassettes, for removal of the CP4, Cpf1, Cre and gRNA cassettes upon selfing (see FIG. 5). The CP4, Cre, Cpf1 and PPO cassettes were identical in the two Type 2 constructs. The gRNA targeted to two of the three target sites used in the Type 1 constructs. In addition, the PPO cassette in the Type 2 constructs contained different expression elements from the PPO cassette in the Type 1 constructs.

The five different constructs were cloned into plant transformation vectors and introduced into corn seed-derived dry excised explants through Agrobacterium-mediated transformation using methods known in the art. The CP4 expression cassette contained the aroA gene (also known as 5-enolpyruvylshikimate-3-phosphate synthase (EPSPS)) from Agrobacterium CP4 strain under the control of a constitutive promoter, and targeted the EPSPS to the chloroplast for selection of transgenic events resistant to glyphosate. The Cpf1 expression cassette encoded a nuclear targeted Cpf1 protein from Lachnospiraceae bacterium ND2006 codon-optimized for corn expression and under the control of a constitutive promoter for site-directed integration of the transgenes. The gRNA in the gRNA cassettes was under the control of a Pol III constitutive promoter and targeted the Cpf1 enzyme to three different genomic locations of the corn genome, respectively, for site-directed integration of the transgenes. The PPO expression cassette encoded a chloroplast targeted PPO protein from Enterobacter cloacae for conferring tolerance to the PPO herbicides under the control of a constitutive promoter, and differed between the Type 1 and Type 2 constructs in that the promoter for Type 1 constructs was derived from a monocot species, whereas the promoter for the Type 2 constructs was synthetic. The Type 2 constructs also contained a Cre expression cassette, which encoded a Cre recombinase from Enterobacteria Phage P1 under the control of a cell division specific promoter, for removal of CP4, Cpf1, Cre and gRNA cassettes in the progeny. FIG. 2 shows the organization of the Type 1 constructs before and after T-TDNA insertion into the genome (Panels A and B, respectively), and the inserted T-DNA after excision of the CP4, Cpf1, and gRNA cassettes following cross with a Cre line (Panel C). FIG. 5 shows the organization of the Type 2 constructs before insertion into the genome (top) and the inserted T-DNA after excision of the CP4, Cpf1, and gRNA cassettes (bottom left). Transformation was performed in two stages. The first stage of transformation included the three Type 1 constructs, and the second stage included the two Type 2 constructs. A total of 11,844 unique transformation events were produced, 7,230 from the Type 1 constructs, and 4614 from the Type 2 constructs. R0 plants were subsequently regenerated from the transgenic events. Rooted plants with normal phenotype were transferred to soil for growth and further assessment.

Molecular analyses were conducted concurrently with the controlled environment testing and field trials on events that were advanced. DNA amplification and sequencing were used to determine the composition and intactness of the insert sequence, insert copy number, the presence/absence of Agrobacterium Ti-plasmid backbone sequence, the insertion site properties (e.g., proximity to endogenous genes or repeated regions), and targeted insertion. Specifically, the 11,844 unique R0 events were analyzed for the following desirable molecular characteristics: 1) The PPO transgene was single copy and full length; 2) The PPO transgene was inserted at the target site defined by the gRNA (targeted insert event); 3) In case the PPO transgene was inserted at a non-target site, it was inserted on the same chromosome and less than 30 centimorgan (cM) away from the event MON87429 locus (linked event); 4) The transgene was not inserted in an endogenous gene or a repeated region, and has no SNPs (single nucleotide polymorphisms), to ensure that the sequence in planta matches the sequence in the construct; 5) If an R0 event had multiple PPO inserts, one insert met the criteria that it was full-length, not linked to other inserts, and was inserted on the target chromosome. Events meeting criteria 3) and 5) served as back-ups in case none of the transgenic events were PPO single copy and inserted at the target site defined by the gRNA.

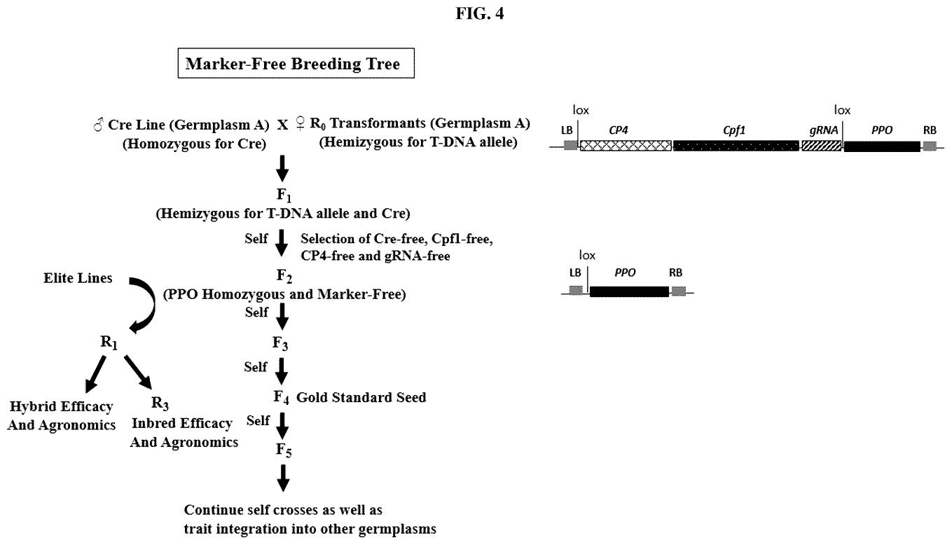

As shown in Table 7, from a total of 11,844 unique R0 events generated from the five constructs, 21 unique R0 events from the Type 1 constructs, and 14 from the Type 2 constructs met the criteria, and were advanced to crossing with a Cre line (events from the Type 1 constructs, FIG. 4) or selfing (events from the Type 2 constructs, FIG. 5), respectively to produce F1 or R1 seeds. Events from the Type 2 constructs were also crossed with a Cre line to generate F1 progeny as a back-up in case the auto-excision failed. A total of 19 F1/R1 events hemizygous for the PPO transgene, and absent of the CP4, Cpf1 and gRNA, were self-pollinated to produce F2/R2 seeds. The numbers in parentheses in Table 7 represent events that were randomly inserted near the target loci (designated as linked events), but not targeted as defined by the gRNAs. The seeds were analyzed to select for events that were homozygous for the PPO transgene, and did not contain Cre, or CP4, Cpf1 or the gRNA. As a result, one event from each of constructs ZmHT5-1 and ZmHT5-2 were selected as the targeted events for further testing. Based on the molecular analyses and field performance of the events as described in Example 4, the lead event was selected from construct ZmHT5-2.

Northern analysis was done to detect and measure mRNA transcript of the PPO transgene in the transgenic events. Protein analysis of plants comprising each event was conducted using techniques known in the art. N-terminal protein sequencing of the PPO proteins purified from transgenic plants containing each event was done to confirm the recombinant protein sequence. Western blot analysis was conducted on protein extracts to confirm the production of the PPO protein from each transgenic event.

TABLE 7
Summary of events selection from the five transformation constructs.

| | ZmHT5-1 | ZmHT5-2 | ZmHT5-3 | ZmHT5-4 | ZmHT5-5 |
|---|---|---|---|---|---|
| Construct | Type 1 | | | Type 2 | |
| Target Site | ZmTS16 | ZmTS10 | ZmTS14 | ZmTS16 | ZmTS10 |
| Total # R0 Event | 2092 | 3360 | 1778 | 2171 | 2443 |
| # R0 Event for Crossing | 8 (3) | 6 (4) | 7 (4) | 8 (6) | 6 (5) |
| with Cre Line or Selfing | | | | | |
| # F1/R1 Event | 3 (1) | 4 (3) | 0 | 8 (6) | 4 (4) |
| # F2/R2 Targeted Event | 2 | 1 | 0 | 2 (1) | 2 (2) |
| # F3/R3 Targeted Event | 2 | 1 | 0 | 0 | 0 |
| # F4/R4 Targeted Event | 1 | 1 | 0 | 0 | 0 |
| Lead event | — | 1 | — | — | — |

### Example 4: Field Trials

Field trials were conducted over multiple seasons/years and across many locations in different geographies to evaluate the performance of the constructs/events, and for selecting the best performing construct/event. The field trials comprised efficacy trials for herbicide tolerance, and agronomic trials for yield performance of the events. The performance of many individual plants for each event in each field trial was analyzed as a set. Each event was thus represented by many individual plants. This allowed the performance of each event to be analyzed under many different conditions, in different locations and geographies, and for a variety of properties. Field trials were conducted on homozygous plants to assess trait efficacy for tolerance to commercial rates of PPO herbicides, and agronomic performance.

### First Season Field Trials

The first season efficacy and agronomic field trials were conducted at 2-6 locations in South America using a randomized complete block design (RCBD), and included 3 events from construct ZmHT5-1, and 4 events from ZmHT5-2. In the agronomy trials, the events were evaluated for plant height (PHT), ear height (EHT), final stand count (FNSC), final stand % (FNSP), days to 50% pollen (P50D), days to 50% silk (S50D), grain moisture (MST), test weight (TWT), shell weight (SHW), and grain yield (YLD, presented as bushel per acre). Fields were maintained weed-free by hand hoeing or by use of conventional herbicides.

In the inbred efficacy trials, herbicide treatments included fomesafen at 1.5 lbs ai/acre plus flumioxazin at 0.375 lbs ai/acre at pre-emergence (PRE), followed by epyrifenacil at 0.144 lbs ai/acre at V2 and V6, respectively. Table 8 lists the PPO herbicides and application rates in the trials.

TABLE 8
PPO herbicides and application rates.

| | | 1 × Rate | 1 × Rate | 4 × Rate | 4 × Rate |
|---|---|---|---|---|---|
| Product | Common Name | (lb/acre) | (kg/hectare) | (lb/acre) | (kg/hectare) |
| Reflex | fomesafen (4×) | 0.375 | 0.42 | 1.5 | 1.68 |
| Valor SX | flumioxazin (4×) | 0.094 | 0.105 | 0.375 | 0.42 |
| Rapidicil | epyrifenacil (4×) | 0.018 | 0.02 | 0.072 | 0.08 |
| COC | surfactant | 1% v/v | 1% v/v | 1% v/v | 1% v/v |

In the hybrid efficacy trials, herbicide treatments included: Treatment 1: non treated control; Treatment 2: fomesafen at 0.75 lbs ai/acre plus flumioxazin at 0.1875 lbs ai/acre applied PRE, followed by epyrifenacil at 0.072 lbs ai/acre applied to V2 and V6; Treatment 3: fomesafen at 1.5 lbs ai/acre plus flumioxazin at 0.375 lbs ai/acre applied PRE, followed by epyrifenacil at 0.144 lbs ai/acre applied to V2 and V6 (Table 9). Each treatment contained 4-8 replications per location.

TABLE 9
First season hybrid efficacy trial treatments.

| Treatment | Product | Common Name | Rate (lb/acre) | | Timing |
|---|---|---|---|---|---|
| 1 | Non-treated | Non-treated | NA | | NA |
| 2 | Reflex | fomesafen (2×) | 0.75 lbs acre | (0.84 kg/ha) | PRE |
| 2 | Valor SX | flumioxazin (2×) | 0.1875 lbs ai/acre | (0.21 kg/ha) | PRE |
| 2 | Rapidicil | epyrifenacil (4×) | 0.072 lb/acre | (0.08 kg/ha) | V2 and V6 |
| 2 | COC | surfactant | 1% v/v | | V2 |
| 3 | Reflex | fomesafen (4×) | 1.5 lbs acre | (1.68 kg/ha) | PRE |
| 3 | Valor SX | flumioxazin (4×) | 0.375 lbs ai/acre | (0.42 kg/ha) | PRE |
| 3 | Rapidicil | epyrifenacil (8×) | 0.144 lb/acre | (0.16 kg/ha) | V2 and V6 |

| | | | | |
|---|---|---|---|---|
| 3 | COC | surfactant | 1% v/v | V2 and V6 |

Herbicides were applied using a tractor-mounted or backpack sprayer calibrated to deliver 15 gallons per acre (GPA) with water as the herbicide carrier using Teejet Air Induction (TTI) nozzles. Plots were visually rated for overall crop injury 10-14 days after each herbicide application on a scale of 0 to 100 with “0” being no visible crop injury and “100” being complete crop injury or death. Data collection consisted of crop injury 10 to 14 days after herbicide applications each at preemergence (CIPVE); at V2 stage (CIPV2), at V6 stage (CIPV6), plus a final end-of season rating at VT (CIPVT). Plant height (PHT), ear height (EHT), days to 50% pollen (P50D), days to 50% silk (S50D), 50% pollen shed—50% silking interval in days after planting (ASI50D), the number of susceptible plants per plot after the first herbicide spray (V4S), shell weight (SHW), test weight (TWT), moisture (MST), and grain yield (YLD) were also collected.

All data from both agronomy and efficacy trials were subjected to analysis of variance and means separated at p<0.05. Statistical analysis was automated using ASRem1 software for mixed model fitting. Events were prioritized for further testing based on molecular assessment, yield performance, and crop injury. In the efficacy trials, yield of herbicide treated events was compared to that of the untreated events. In the agronomy trials, yield of events was compared to that of the untransformed (wildtype) control plants.

Results of the inbred and hybrid agronomy trials for the individual events are summarized in Table 10. Italic font represents events that were randomly inserted into chromosome 8 (linked events), whereas regular font represents events that were inserted at the target sites.

TABLE 10
Summary of first season agronomic trial results.

| | | | | | | | | | | | |
|---|---|---|---|---|---|---|---|---|---|---|---|
| Event ID | ASI50D | EHT | FNSC | FNSP | MST | P50D | PHT | S50D | SHW | TWT | YLD |

Inbred

| | | | | | | | | | | | |
|---|---|---|---|---|---|---|---|---|---|---|---|
| ZmHT5-1-1 | 0.75 | 27.09 | 66.55 | 99.57 | 16.22 | 73.82 | 60.92 | 74.52 | 14.65 | 54.97 | 125.03 |
| ZmHT5-1-2 | 0.60 | 25.87 | 68.08 | 101.65 | 16.19 | 74.00 | 60.36 | 74.57 | 15.07 | 53.88 | 128.69 |
| ZmHT5-1-3 | 0.58 | 27.53 | 66.16 | 98.63 | 16.66 | 73.99 | 61.95 | 74.49 | 14.61 | 53.69 | 124.16 |
| ZmHT5-2-1 | 0.63 | 25.99 | 67.05 | 100.09 | 15.88 | 73.64 | 60.65 | 74.30 | 14.58 | 54.63 | 124.93 |
| (Zm_CSM63715) | | | | | | | | | | | |
| ZmHT5-2-2 | 0.58 | 27.16 | 66.39 | 99.08 | 16.19 | 73.59 | 60.60 | 74.15 | 14.11 | 54.23 | 120.42 |
| ZmHT5-2-3 | 0.49 | 26.47 | 67.28 | 100.42 | 16.68 | 74.37 | 61.12 | 74.85 | 14.93 | 55.51 | 126.83 |
| ZmHT5-2-4 | 0.58 | 25.92 | 67.19 | 100.29 | 16.12 | 73.48 | 61.55 | 74.04 | 14.77 | 54.77 | 126.29 |
| wildtype | 0.60 | 26.52 | 66.94 | 99.91 | 16.07 | 73.74 | 61.80 | 74.36 | 15.12 | 55.02 | 129.34 |
| LSD_{(0.05)} | 0.53 | 3.01 | 1.89 | 2.82 | 0.54 | 0.77 | 2.65 | 0.84 | 0.93 | 2.72 | 7.79 |

Hybrid

| | | | | | | | | | | | |
|---|---|---|---|---|---|---|---|---|---|---|---|
| ZmHT5-1-1 | 0.67 | 37.47 | 69.22 | 103.37 | 17.35 | 71.14 | 75.78 | 71.78 | 28.67 | 55.98 | 240.91 |
| ZmHT5-1-2 | 0.87 | 37.38 | 68.23 | 101.96 | 17.28 | 71.01 | 76.16 | 71.85 | 28.89 | 54.30 | 242.88 |
| ZmHT5-1-3 | 0.69 | 37.52 | 68.17 | 101.85 | 17.35 | 71.04 | 74.27 | 71.68 | 28.49 | 54.40 | 239.34 |
| ZmHT5-2-1 | 0.88 | 38.64 | 67.14 | 100.56 | 17.24 | 70.40 | 75.58 | 71.25 | 28.25 | 54.79 | 237.68 |
| ZmHT5-2-2 | 0.88 | 38.16 | 66.79 | 99.79 | 17.03 | 70.67 | 75.93 | 71.52 | 28.30 | 54.32 | 238.62 |
| ZmHT5-2-3 | 1.03 | 39.16 | 67.91 | 101.43 | 17.24 | 70.90 | 74.52 | 71.90 | 28.31 | 54.60 | 238.04 |
| ZmHT5-2-4 | 0.67 | 39.92 | 68.99 | 103.01 | 17.19 | 70.64 | 76.65 | 71.28 | 29.16 | 54.01 | 245.49 |
| wildtype | 0.98 | 38.39 | 69.40 | 103.65 | 17.14 | 70.92 | 75.34 | 71.87 | 28.56 | 55.04 | 240.54 |
| LSD_{(0.05)} | 0.71 | 2.94 | 1.57 | 2.29 | 0.26 | 0.93 | 2.94 | 0.82 | 0.78 | 1.34 | 6.40 |

Results of the inbred and hybrid efficacy trials for the individual events are summarized in Table 11. All events performed well in both inbred and hybrid efficacy trials, with event ZmHT5-2-1 (Zm_CSM63715) showing the highest numerical yield in the inbred efficacy trials.

TABLE 11
Summary of first season efficacy trial results.
Inbred
Treatment: fomesafen 1.5 lbs + flumioxazin 0.375 lbs PRE, followed by epyrifenacil 0.144 lbs V2 and V6

| Event ID | ASI50D | CIPVE | CIPV2 | CIPV6 | CIPVT | EHT | FNSC | MST | P50D | PHT | S50D | SHW | TWT | V4S | YLD |
|---|---|---|---|---|---|---|---|---|---|---|---|---|---|---|---|
| ZmHT5-1-1 | −0.6 | 0 | 0.63 | 0.13 | 0 | 27.81 | 64.25 | 15.96 | 75.75 | 60.71 | 75.53 | 16.12 | 55.81 | 0.00 | 137.98 |
| ZmHT5-1-2 | −0.2 | 0 | 0.25 | 0.63 | 0.38 | 27.10 | 64.38 | 16.49 | 74.95 | 61.34 | 75.02 | 15.56 | 57.16 | 0.00 | 132.28 |
| ZmHT5-1-3 | −0.8 | 0 | 0 | 0.25 | 1.25 | 26.71 | 66.13 | 16.53 | 74.95 | 60.56 | 74.14 | 16.21 | 58.11 | 0.00 | 137.71 |
| ZmHT5-2-1 | −1 | 0.125 | 0.25 | 0.38 | 0 | 26.00 | 67.25 | 15.77 | 74.95 | 58.80 | 74.18 | 16.56 | 57.63 | 0.13 | 141.95 |
| ZmHT5-2-2 | −0.8 | 0 | 0.25 | 0.00 | 0 | 27.42 | 65.38 | 15.78 | 75.35 | 60.01 | 75.05 | 15.71 | 57.57 | 0.25 | 134.65 |
| ZmHT5-2-3 | −0.6 | 0.5 | 0 | 0.75 | 0.25 | 25.53 | 66.50 | 16.39 | 75.55 | 60.45 | 74.98 | 15.17 | 57.78 | 0.13 | 129.13 |
| ZmHT5-2-4 | −1.8 | 0 | 0 | 0.13 | 1 | 25.53 | 62.88 | 16.00 | 74.75 | 61.29 | 73.18 | 15.54 | 56.86 | 0.13 | 132.84 |
| LSD_{(0.05)} | 1.03 | 0.32 | 0.74 | 0.83 | 1.59 | 2.98 | 3.29 | 0.97 | 1.1 | 4.2 | 2.04 | 1.43 | 3.02 | 0.31 | 12.26 |

Hybrid
Treatment 1: Non-treated

| Event ID | ASI50D | CIPV2 | CIPVT | EHT | FNSC | MST | P50D | PHT | S50D | SHW | TWT | V4S | YLD |
|---|---|---|---|---|---|---|---|---|---|---|---|---|---|
| ZmHT5-1-1 | 0.75 | 0.03 | 0.00 | 38.72 | 68.88 | 16.87 | 66.64 | 77.02 | 67.30 | 27.72 | 55.04 | 0.03 | 234.40 |
| ZmHT5-1-2 | 0.64 | 0.00 | 0.00 | 38.47 | 65.63 | 17.16 | 66.64 | 79.10 | 67.19 | 27.78 | 55.54 | 0.13 | 234.20 |
| ZmHT5-1-3 | 0.47 | 0.00 | 0.00 | 38.32 | 67.84 | 17.18 | 66.81 | 78.96 | 67.19 | 27.90 | 55.11 | 0.13 | 235.12 |
| ZmHT5-2-1 | 0.92 | 0.00 | 0.13 | 38.32 | 64.75 | 16.83 | 66.42 | 78.13 | 67.24 | 26.67 | 54.49 | 0.22 | 225.69 |
| ZmHT5-2-2 | 0.64 | 0.00 | 0.00 | 39.33 | 66.63 | 17.32 | 66.31 | 78.07 | 66.86 | 27.52 | 55.27 | 0.03 | 231.55 |
| ZmHT5-2-3 | 0.86 | 0.03 | 0.00 | 39.61 | 67.22 | 16.68 | 66.37 | 77.99 | 67.13 | 27.15 | 55.05 | 0.16 | 230.14 |
| ZmHT5-2-4 | 0.64 | 0.00 | 0.00 | 38.72 | 69.13 | 17.09 | 66.09 | 75.59 | 66.63 | 27.55 | 55.44 | 0.03 | 232.46 |
| Wildtype | 0.58 | 0.00 | 0.00 | 37.61 | 69.88 | 17.08 | 66.75 | 78.98 | 67.24 | 28.13 | 55.33 | 0.06 | 237.42 |
| LSD_{(0.05)} | 0.51 | 0.04 | 0.10 | 2.50 | 1.62 | 0.38 | 0.52 | 2.47 | 0.49 | 0.88 | 1.12 | 0.17 | 7.19 |

| | | | | | | | | | | | | | | | |
|---|---|---|---|---|---|---|---|---|---|---|---|---|---|---|---|
| Event ID | ASI50D | CIPVE | CIPV2 | CIPV6 | CIPVT | EHT | FNSC | MST | P50D | PHT | S50D | SHW | TWT | V4S | YLD |

Treatment 2: fomesafen 0.75 lbs + flumioxazin 0.1875 lbs PRE, followed by epyrifenacil 0.072 lbs V2 and V6

| | | | | | | | | | | | | | | | |
|---|---|---|---|---|---|---|---|---|---|---|---|---|---|---|---|
| ZmHT5-1-1 | 0.74 | 0.17 | 0.53 | 1.13 | 0.19 | 40.44 | 68.16 | 17.11 | 66.73 | 80.34 | 67.41 | 27.79 | 55.31 | 0.63 | 233.82 |
| ZmHT5-1-2 | 0.68 | 0.04 | 0.47 | 1.00 | 0.25 | 37.79 | 65.94 | 17.26 | 66.96 | 77.98 | 67.58 | 26.85 | 55.36 | 0.56 | 226.06 |
| ZmHT5-1-3 | 0.40 | 0.17 | 0.31 | 1.44 | 0.63 | 38.07 | 67.69 | 17.10 | 66.90 | 78.16 | 67.26 | 27.09 | 55.76 | 0.41 | 228.56 |
| ZmHT5-2-1 | 0.35 | 0.04 | 0.38 | 2.37 | 0.50 | 38.11 | 64.28 | 17.15 | 66.79 | 78.27 | 67.08 | 26.17 | 56.11 | 0.56 | 220.67 |
| ZmHT5-2-2 | 0.79 | 0.04 | 0.41 | 1.91 | 0.87 | 37.79 | 66.25 | 17.15 | 66.84 | 78.62 | 67.57 | 27.10 | 56.11 | 0.38 | 228.47 |
| ZmHT5-2-3 | 0.68 | 0.08 | 0.63 | 1.69 | 0.36 | 38.52 | 66.53 | 16.91 | 67.12 | 81.14 | 67.75 | 26.22 | 55.41 | 0.59 | 221.72 |
| ZmHT5-2-4 | 0.68 | 0.17 | 0.50 | 1.31 | 0.94 | 39.65 | 68.81 | 16.85 | 66.68 | 79.32 | 67.34 | 27.43 | 55.54 | 0.56 | 232.32 |
| Wildtype | NA | NA | NA | NA | NA | NA | NA | NA | NA | NA | NA | NA | NA | NA | NA |
| LSD_{(0.05)} | 0.55 | 0.73 | 0.81 | 1.20 | 0.99 | 3.14 | 2.16 | 0.54 | 0.54 | 5.23 | 0.68 | 1.32 | 1.40 | 0.86 | 10.22 |

Treatment 3: fomesafen 1.5 lbs + flumioxazin 0.375 lbs PRE, followed by epyrifenacil 0.144 lbs V2 and V6

| | | | | | | | | | | | | | | | |
|---|---|---|---|---|---|---|---|---|---|---|---|---|---|---|---|
| ZmHT5-1-1 | 0.65 | 0.08 | 0.53 | 1.34 | 1.13 | 38.45 | 68.00 | 16.95 | 66.99 | 80.14 | 67.62 | 27.41 | 55.36 | 0.66 | 231.75 |
| ZmHT5-1-2 | 0.87 | 0.13 | 0.41 | 1.88 | 0.88 | 37.77 | 65.84 | 17.00 | 66.77 | 81.19 | 67.60 | 26.79 | 55.24 | 0.50 | 226.20 |
| ZmHT5-1-3 | 0.81 | 0.08 | 0.44 | 1.34 | 0.78 | 38.74 | 66.72 | 16.86 | 66.55 | 79.67 | 67.34 | 26.86 | 56.06 | 0.53 | 227.23 |
| ZmHT5-2-1 | 0.76 | 0.29 | 0.63 | 2.16 | 0.81 | 37.52 | 66.41 | 16.75 | 67.16 | 79.59 | 67.87 | 26.20 | 54.70 | 0.97 | 222.09 |
| ZmHT5-2-2 | 0.69 | 0.17 | 0.75 | 1.34 | 0.78 | 39.20 | 66.56 | 16.97 | 67.05 | 78.07 | 67.66 | 27.08 | 54.87 | 0.88 | 228.84 |
| ZmHT5-2-3 | 0.93 | 0.17 | 0.47 | 1.81 | 0.66 | 38.84 | 68.56 | 17.03 | 66.66 | 78.42 | 67.57 | 26.82 | 56.39 | 0.63 | 226.56 |
| ZmHT5-2-4 | 0.64 | 0.08 | 0.34 | 1.06 | 0.38 | 39.20 | 67.00 | 16.93 | 66.72 | 76.13 | 67.32 | 27.89 | 55.16 | 0.38 | 235.76 |
| Wildtype | NA | NA | NA | NA | NA | NA | NA | NA | NA | NA | NA | NA | NA | NA | NA |
| LSD_{(0.05)} | 0.48 | 0.26 | 0.49 | 0.90 | 0.69 | 2.10 | 1.80 | 0.41 | 0.48 | 4.77 | 0.63 | 1.13 | 1.35 | 0.50 | 9.16 |

Since ZmHT5-1-3, ZmHT5-2-2, ZmHT5-2-3 and ZmHT5-2-4 were randomly inserted events, not at the targeted sites, they were dropped for further testing. Events ZmHT5-1-1, ZmHT5-1-2 and ZmHT5-2-1 were advanced as priority events to the next season trials.

### Second Season Field Trials

The second season field trials were conducted in North America. Inbred and hybrid agronomic trials included events ZmHT5-1-1, ZmHT5-1-2, and ZmHT5-2-1 at 16 locations for the inbred trials and 29 locations for the hybrid trials, using a randomized complete block design with 6 replications per location. Standard agronomic practices for conventional inbred corn were performed. Data collection included final stand counts (FNSC), plant height (PHT), ear height (EHT), days to 50% silk (S50D), days to 50% pollen (P50D), shell weight (SHW), test weight (TWT), moisture (MST), and grain yield (YLD).

Inbred efficacy trials included four targeted events (ZmHT5-1-1, ZmHT5-1-2, ZmHT5-2-1 and ZmHT5-4-1) at 2 locations. Herbicide treatments were fomesafen at 1.5 pounds of active ingredient per acre (lbs ai/a) plus flumioxazin at 0.375 lbs ai/a applied preemergence (PRE) followed by epyrifenacil at 0.144 lbs ai/a plus crop oil concentrate (COC) applied at V2 and V6.

Hybrid efficacy trials included three events (ZmHT5-1-1, ZmHT5-1-2, and ZmHT5-2-1) at 12 locations. Herbicide treatments were the same as shown in Table 9. Herbicides were applied using the same methods as described for the first season efficacy trials.

All data from both agronomy and efficacy trials were subjected to analysis of variance and means separated at p<0.05. Statistical analysis was automated using ASRem1 software for mixed model fitting. Events were prioritized for further testing based on molecular assessment, yield performance, and crop injury. In the efficacy trials, yield of herbicide-treated events was compared to that of the non-treated and each other within a treatment block. In the agronomy trials, yield of events was compared to that of the non-transformed (wildtype) control plots.

The results of the second season inbred and hybrid agronomic trials are shown in Table 12.

TABLE 12
Summary of the second season agronomic trial results.

| | | | | | | | | | |
|---|---|---|---|---|---|---|---|---|---|
| Event ID | EHT | FNSC | MST | P50D | PHT | S50D | SHW | TWT | YLD |

Inbred

| | | | | | | | | | |
|---|---|---|---|---|---|---|---|---|---|
| ZmHT5-1-1 | 30.69 | 118.58 | 24.01 | 68.77 | 80.09 | 69.04 | 22.43 | 55.15 | 92.82 |
| ZmHT5-1-2 | 31.17 | 122.08 | 23.92 | 68.89 | 78.68 | 69.32 | 23.09 | 54.44 | 95.30 |
| ZmHT5-2-1 | 31.11 | 122.62 | 23.00 | 68.28 | 77.60 | 68.82 | 21.87 | 55.25 | 91.98 |
| (Zm_CSM63715) | | | | | | | | | |
| Wildtype | 31.67 | 124.51 | 22.33 | 68.52 | 79.79 | 68.91 | 23.57 | 54.79 | 99.46 |
| LSD_{(0.05)} | 1.21 | 2.53 | 1.21 | 0.28 | 1.94 | 0.35 | 1.07 | 1.59 | 4.35 |

Hybrid

| | | | | | | | | | |
|---|---|---|---|---|---|---|---|---|---|
| ZmHT5-1-1 | 46.95 | 59.00 | 20.35 | 62.34 | 99.85 | 62.82 | 23.48 | 55.69 | 208.83 |
| ZmHT5-1-2 | 45.97 | 59.38 | 20.65 | 62.24 | 100.62 | 62.78 | 24.15 | 55.61 | 213.87 |
| ZmHT5-2-1 | 46.63 | 59.97 | 20.24 | 62.09 | 99.78 | 62.50 | 24.38 | 55.33 | 217.66 |
| (Zm_CSM63715) | | | | | | | | | |
| Wildtype | 45.35 | 58.25 | 20.58 | 62.47 | 99.65 | 62.84 | 23.86 | 55.37 | 211.70 |
| LSD_{(0.05)} | 1.75 | 0.92 | 0.29 | 0.18 | 0.87 | 0.24 | 0.57 | 0.19 | 4.90 |

The results of second season inbred and hybrid efficacy trials are summarized in Table 13 and Table 14.

TABLE 13
Summary of second season inbred efficacy trial results.

| Event ID | CIPVE | CIPV2 | CIPV6 | CIPVT | EHT | PHT | FNSC | P50D | S50D |
|---|---|---|---|---|---|---|---|---|---|
| ZmHT5-2-1 | 0.25 | 0.50 | 0.00 | 0.00 | 31.75 | 77.30 | 43.75 | 65.75 | 66.25 |
| ZmHT5-1-2 | 2.50 | 2.00 | 1.25 | 0.00 | 31.25 | 78.97 | 52.00 | 66.00 | 66.75 |
| ZmHT5-1-1 | 3.25 | 1.75 | 1.50 | 0.00 | 34.50 | 77.55 | 49.25 | 66.75 | 67.25 |
| ZmHT5-4-1 | 0.25 | 1.75 | 1.25 | 1.25 | 37.00 | 80.19 | 45.25 | 68.75 | 68.75 |
| LSD_{(0.05)} | 3.79 | 2.01 | 2.43 | 1.68 | 6.00 | 2.97 | 15.07 | 1.15 | 1.55 |

TABLE 14
Summary of second season hybrid efficacy trial results.

| Trt | Event ID | CIPVE | CIPV2 | CIPV6 | CIPVT | EHT | PHT | FNSC | MST | P50D | S50D | SHW | TWT | YLD |
|---|---|---|---|---|---|---|---|---|---|---|---|---|---|---|
| 1 | ZmHT5-2-1 | 0.00 | 0.00 | 0.00 | 0.00 | 45.41 | 96.08 | 60.08 | 20.21 | 61.45 | 61.52 | 24.04 | 54.29 | 212.92 |
| | ZmHT5-1-2 | 0.00 | 0.00 | 0.00 | 0.00 | 46.06 | 96.39 | 59.57 | 20.65 | 61.92 | 62.27 | 24.49 | 54.58 | 215.44 |
| | ZmHT5-1-1 | 0.00 | 0.00 | 0.00 | 0.00 | 45.66 | 95.86 | 59.84 | 20.55 | 61.74 | 62.18 | 24.05 | 54.47 | 211.89 |
| | Wildtype | 0.00 | 0.00 | 0.00 | 0.00 | 45.01 | 95.74 | 59.77 | 20.50 | 61.60 | 61.95 | 24.23 | 54.28 | 213.77 |
| | LSD_{(0.05)} | NS | NS | NS | NS | 1.15 | 1.08 | 1.19 | 0.36 | 0.36 | 0.43 | 0.67 | 0.20 | 6.04 |
| 2 | ZmHT5-2-1 | 0.15 | 0.24 | 0.23 | 0.00 | 45.73 | 95.35 | 59.13 | 20.81 | 61.57 | 61.82 | 24.91 | 54.39 | 219.21 |
| | ZmHT5-1-2 | 0.31 | 0.26 | 0.64 | 0.00 | 45.09 | 96.33 | 59.24 | 20.91 | 61.86 | 62.29 | 24.78 | 54.53 | 217.43 |
| | ZmHT5-1-1 | 0.52 | 0.40 | 0.39 | 0.00 | 45.09 | 96.50 | 59.48 | 20.96 | 61.81 | 62.14 | 24.60 | 54.54 | 215.73 |
| | LSD_{(0.05)} | 0.70 | 0.32 | 0.44 | NS | 1.24 | 0.97 | 1.24 | 0.27 | 0.29 | 0.34 | 0.84 | 0.23 | 7.26 |
| 3 | ZmHT5-2-1 | 0.82 | 0.71 | 0.33 | 0.00 | 45.63 | 95.32 | 58.81 | 20.77 | 61.99 | 62.03 | 25.05 | 54.30 | 219.76 |
| | ZmHT5-1-2 | 0.84 | 0.40 | 0.36 | 0.00 | 45.25 | 96.06 | 58.61 | 21.10 | 62.26 | 62.39 | 24.92 | 54.12 | 217.69 |
| | ZmHT5-1-1 | 0.71 | 0.31 | 0.61 | 0.02 | 45.66 | 95.90 | 57.89 | 20.93 | 62.21 | 62.53 | 24.68 | 54.14 | 215.84 |
| | LSD_{(0.05)} | 0.82 | 0.51 | 0.46 | 0.02 | 1.24 | 0.97 | 1.49 | 0.29 | 0.25 | 0.31 | 0.68 | 0.20 | 5.76 |

In addition, a two-location hybrid efficacy screen was conducted including three events for construct ZmHT5-1, four events for ZmHT5-2, four events for ZmHT5-4, and three events for ZmHT5-5. Results are shown in Table 15. Italic font represents events that were randomly inserted into chromosome 8 (linked events), whereas regular font represents events that were inserted at the target sites. Herbicide treatments were the same as shown in Table 9. Herbicides were applied using the same methods as described for the first season efficacy trials.

As shown in Table 15, events from both Type 2 constructs yielded less in general for all three treatments. Event ZmHT5-2-1 (ZmCSM63715) performed consistently better than or as well when compared to other events.

TABLE 15
Summary of second season 2-location
hybrid efficacy screen results.

| Treat- | | | | | | | |
|---|---|---|---|---|---|---|---|
| ment | Event ID | EHT | PHT | MST | TWT | SHW | YLD |
| 1 | ZmHT5-1-3 | 47.94 | 106.84 | 22.11 | 53.89 | 27.58 | 235.31 |
| | ZmHT5-1-2 | 47.14 | 107.50 | 22.03 | 53.76 | 26.96 | 230.25 |
| | ZmHT5-1-1 | 51.54 | 105.86 | 21.69 | 54.16 | 26.92 | 231.18 |
| | ZmHT5-2-4 | 46.54 | 106.11 | 23.24 | 53.80 | 26.07 | 219.95 |
| | ZmHT5-2-1 | 51.94 | 106.00 | 22.22 | 53.87 | 27.46 | 233.98 |
| | ZmHT5-2-3 | 49.34 | 106.49 | 22.52 | 53.98 | 27.48 | 233.69 |
| | ZmHT5-2-2 | 50.74 | 105.10 | 22.71 | 53.66 | 26.61 | 225.51 |
| | ZmHT5-4-3 | 49.14 | 105.24 | 22.82 | 53.45 | 25.04 | 212.13 |
| | ZmHT5-4-4 | 48.94 | 105.23 | 22.79 | 53.08 | 26.17 | 222.91 |
| | ZmHT5-4-5 | 48.74 | 107.61 | 22.76 | 53.16 | 26.03 | 219.86 |
| | ZmHT5-4-6 | 48.98 | 106.91 | 22.81 | 53.51 | 25.27 | 215.07 |
| | ZmHT5-5-6 | 48.54 | 107.83 | 22.59 | 53.48 | 25.94 | 220.17 |
| | ZmHT5-5-7 | 50.98 | 106.73 | 23.07 | 53.24 | 25.13 | 212.74 |
| | ZmHT5-5-8 | 48.14 | 107.56 | 23.29 | 53.48 | 26.33 | 221.53 |
| | Wildtype | 48.15 | 106.08 | 22.02 | 53.95 | 26.86 | 228.02 |
| | LSD_{(0.05)} | 4.20 | 2.31 | 1.14 | 0.65 | 2.20 | 17.64 |
| 2 | ZmHT5-1-3 | 47.88 | 22.00 | 103.94 | 28.27 | 53.95 | 242.61 |
| | ZmHT5-1-2 | 49.34 | 22.41 | 108.09 | 27.60 | 53.70 | 234.50 |
| | ZmHT5-1-1 | 49.54 | 22.24 | 106.45 | 25.81 | 53.72 | 219.83 |
| | ZmHT5-2-4 | 45.74 | 22.16 | 107.61 | 27.88 | 53.73 | 237.90 |
| | ZmHT5-2-1 | 50.54 | 22.65 | 106.55 | 27.31 | 53.32 | 231.90 |
| | ZmHT5-2-3 | 47.74 | 21.89 | 107.09 | 27.12 | 54.16 | 232.23 |
| | ZmHT5-2-2 | 46.34 | 21.94 | 106.28 | 27.36 | 54.06 | 234.23 |
| | ZmHT5-4-3 | 47.13 | 23.40 | 104.50 | 24.61 | 53.40 | 206.34 |
| | ZmHT5-4-4 | 47.05 | 23.10 | 108.00 | 23.07 | 52.75 | 195.03 |
| | ZmHT5-4-5 | 48.94 | 23.25 | 107.47 | 24.19 | 52.88 | 203.49 |
| | ZmHT5-4-6 | 47.94 | 22.59 | 106.42 | 24.19 | 53.47 | 205.27 |
| | ZmHT5-5-6 | 49.74 | 23.31 | 106.70 | 25.46 | 53.16 | 213.14 |
| | ZmHT5-5-7 | 47.54 | 23.47 | 106.35 | 26.71 | 53.12 | 223.90 |
| | ZmHT5-5-8 | 46.54 | 23.41 | 108.39 | 26.18 | 53.26 | 219.85 |
| | LSD_{(0.05)} | 3.58 | 1.11 | 2.19 | 1.98 | 0.57 | 16.84 |
| 3 | ZmHT5-1-3 | 48.88 | 104.26 | 22.97 | 53.50 | 24.81 | 208.87 |
| | ZmHT5-1-2 | 48.67 | 105.64 | 22.65 | 53.64 | 25.58 | 216.16 |
| | ZmHT5-1-1 | 45.42 | 103.55 | 22.74 | 53.74 | 26.43 | 223.03 |
| | ZmHT5-2-4 | 45.27 | 104.85 | 22.85 | 53.64 | 25.78 | 217.73 |
| | ZmHT5-2-1 | 49.38 | 103.73 | 23.10 | 53.40 | 26.04 | 219.11 |
| | ZmHT5-2-3 | 49.42 | 105.68 | 23.12 | 53.81 | 25.85 | 226.24 |
| | ZmHT5-2-2 | 46.14 | 105.59 | 23.04 | 53.88 | 26.80 | 225.48 |
| | ZmHT5-4-3 | 51.20 | 104.26 | 23.56 | 53.51 | 21.56 | 177.87 |
| | ZmHT5-4-4 | 48.27 | 104.20 | 23.00 | 52.50 | 25.09 | 210.80 |
| | ZmHT5-4-5 | 48.69 | 105.93 | 23.92 | 52.64 | 24.85 | 206.34 |
| | ZmHT5-4-6 | 48.87 | 104.31 | 23.69 | 52.81 | 24.34 | 202.65 |
| | ZmHT5-5-6 | 45.86 | 106.49 | 23.73 | 53.43 | 24.43 | 202.71 |
| | ZmHT5-5-7 | 45.21 | 104.41 | 24.49 | 53.13 | 24.06 | 198.87 |
| | ZmHT5-5-8 | 51.36 | 107.17 | 23.92 | 52.59 | 25.48 | 211.65 |
| | LSD_{(0.05)} | 6.23 | 2.03 | 1.01 | 0.71 | 2.20 | 18.21 |

### Third, Fourth and Fifth Season Field Trials

The third, fourth and fifth season field trials were conducted in South America, North America, and South America, respectively. Details of the third season hybrid and inbred efficacy trials are presented in Table 16. The third season agronomic trials included 4 events at 8 locations, using randomized complete block design with 8 replications per location. Herbicide application, data collection and analysis were as described in the proceeding sections.

TABLE 16
Third season efficacy trial details.

| | | | | |
|---|---|---|---|---|
| | | | Rate | |
| Treatment | Product | Common name | (lb/acre) | Timing |

Hybrid efficacy trials: 4 events, 8 locations, randomized complete
block design with 8 replications per treatment per location.

| | | | | |
|---|---|---|---|---|
| 1 | Non-treated | Non-treated | NA | NA |
| 2 | Flex | fomesafen (1×) | 0.375 | V2 |
| | Flumizin SC | flumioxazin (1×) | 0.094 | V2 |
| | Rapidicil | epyrifenacil (1×) | 0.018 | V2 and V6 |
| | COC | surfactant | 1% v/v | V2 and V6 |
| 3 | Flex | fomesafen (2×) | 0.75 | V2 |
| | Flumizin SC | flumioxazin (2×) | 0.1875 | V2 |
| | Rapidicil | epyrifenacil (2×) | 0.036 | V2 and V6 |
| | COC | Surfactant | 1% v/v | V2 and V6 |
| 4 | Flex | fomesafen (4×) | 1.5 | V2 |
| | Flumizin SC | flumioxazin (4×) | 0.375 | V2 |
| | Rapidicil | epyrifenacil (4×) | 0.072 | V2 and V6 |
| | COC | Surfactant | 1% v/v | V2 and V6 |

Inbred efficacy trials: 4 events, two locations, randomized
complete block design with 4 replications per location.

| | | | | |
|---|---|---|---|---|
| 1 | Flex | fomesafen (4×) | 1.5 | V2 |
| | Flumizin SC | flumioxazin (4×) | 0.375 | V2 |
| | Rapidicil | epyrifenacil (4×) | 0.072 | V2 and V6 |
| | COC | surfactant | 1% v/v | V2 and V6 |

Table 17 provides a summary of the third season hybrid agronomic trial results. Event ZmHT5-2-1 (Zm_CSM63715) outperformed ZmHT5-1-2 and the wildtype control in yield. Similarly, event ZmHT5-2-1 (Zm_CSM63715) outperformed ZmHT5-1-2 in yield in both inbred efficacy and hybrid efficacy trial (Table 18).

TABLE 17
Summary of the third season hybrid agronomic trial results.

| Event ID | ASI50D | EHT | FNSC | P50D | PHT | S50D | MST | SHW | TWT | YLD |
|---|---|---|---|---|---|---|---|---|---|---|
| ZmHT5-1-2 | 0.45 | 26.39 | 68.85 | 73.54 | 61.75 | 73.81 | 16.57 | 12.82 | 52.23 | 109.44 |
| ZmHT5-2-1 | 0.32 | 25.42 | 68.28 | 72.76 | 59.81 | 72.93 | 15.50 | 14.07 | 53.27 | 122.70 |
| Wildtype | 0.41 | 25.68 | 69.23 | 72.50 | 60.77 | 72.75 | 15.71 | 12.79 | 52.43 | 110.19 |
| LSD_{(0.05)} | 0.41 | 0.76 | 1.68 | 0.47 | 0.86 | 0.51 | 0.27 | 0.83 | 2.02 | 7.84 |

TABLE 18
Summary of the third season efficacy trial results.

| Trial Type | Event ID | Treatment | MST | SHW | TWT | YLD |
|---|---|---|---|---|---|---|
| Hybrid efficacy | ZmHT5-1-2 | 1 | 16.04 | 26.26 | 51.71 | 226.01 |
| | | 2 | 16.05 | 26.34 | 50.91 | 226.39 |
| | | 3 | 16.11 | 26.02 | 51.34 | 222.33 |
| | | 4 | 16.06 | 25.86 | 51.18 | 221.15 |
| | ZmHT5-2-1 | 1 | 15.89 | 27.86 | 50.79 | 238.35 |
| | | 2 | 15.72 | 27.72 | 52.12 | 237.91 |
| | | 3 | 15.80 | 28.02 | 51.95 | 240.29 |
| | | 4 | 15.81 | 27.00 | 50.97 | 232.59 |
| | | LSD_{(0.05)} | 0.15 | 0.88 | 1.57 | 7.66 |

| | | | | | |
|---|---|---|---|---|---|
| Inbred efficacy | ZmHT5-1-2 | 16.40 | 10.94 | 50.34 | 93.16 |
| | ZmHT5-2-1 | 15.66 | 11.62 | 49.86 | 100.05 |
| | LSD_{(0.05)} | 1.35 | 2.76 | 6.13 | 23.47 |

### Meta Analysis

To compare the field trial data and obtain more precise estimate on the performance of the lead transgenic events, a statistical meta-analysis was performed using the aggregate of all plants for the lead events ZmHT5-1-2 and ZmHT5-2-1 in the multi-season, multi-location field trial data.

Table 19 provides the meta-analysis results for yield for the two events from two constructs in the agronomy and efficacy trials. Yield is presented in bushel per acre for the event, or for the untransformed control (wildtype). Based on the results of meta-analysis and molecular assessment, ZmHT5-2-1 was selected as the commercial event and was named Zm_CSM63715.

TABLE 19
Meta-analysis of yield from agronomic and efficacy field trials.

| Trial Type | Event ID | Yield (bu/acre) | LSD05 | Yield bars |
|---|---|---|---|---|
| Hybrid agronomic | Wildtype | 218.47 | 2.48 | 1.24 |
| Hybrid agronomic | ZmHT5-1-2 | 219.89 | 2.56 | 1.28 |
| Hybrid agronomic | ZmHT5-2-1 | 221.25 | 2.55 | 1.28 |
| Hybrid efficacy | ZmHT5-1-2 | 219.47 | 3.27 | 1.63 |
| Hybrid efficacy | ZmHT5-2-1 | 225.44 | 3.27 | 1.63 |
| Inbred agronomic | Wildtype | 108.46 | 4.43 | 2.21 |
| Inbred agronomic | ZmHT5-1-2 | 106.33 | 4.49 | 2.25 |
| Inbred agronomic | ZmHT5-2-1 | 107.39 | 4.50 | 2.25 |
| Inbred efficacy | ZmHT5-1-2 | 114.71 | 20.55 | 10.28 |
| Inbred efficacy | ZmHT5-2-1 | 111.30 | 20.55 | 10.28 |

Field Trials of Breeding Stack with MON87429

Inbred efficacy and agronomic trials, and hybrid agronomic trials were conducted in South America to evaluate agronomic performance and plant tolerance to PPO herbicides. Plants comprising event Zm_CSM63715 were crossed with plants comprising event MON87429 to produce inbred and hybrid progenies comprising both event Zm_CSM63715 and event MON87429.

The inbred efficacy trials were conducted in one inbred germplasm tester at 2 locations using a randomized complete block design with 4 replications per location. Herbicide treatments consisted of flumioxazin at 1 lb ai/acre (1.12 kg/ha) plus epyrifencacil at 0.072 lb/acre (0.08 kg/ha) plus crop oil concentrate (COC) at 1% v/v applied pre-emergence (PRE) followed by flumioxazin at 0.5 lbs ai/acre plus epyrifencacil at 0.072 lb/acre (0.08 kg/ha) plus COC at 1% v/v applied to V2 followed by V6 corn.

Data collection included, but was not limited to, crop injury 10 to 14 days after herbicide applications and a final rating at VT, days to 50% pollen (P50D), days to 50% silk (S50D), plant height (PHT), ear height (EHT), shell weight (SHW), test weight (TWT), moisture (MST), and grain yield (YLD). All data were subjected to analysis of variance and means separated at p<0.05.

Herbicide tolerance of plants containing MON87429xZm_CSM63715 was excellent (<10% crop injury in most cases) over all rates of PPO herbicides tested. PPO herbicide treatment rates did not produce differences with respect to visual crop injury among most treatments evaluated.

The results of the inbred efficacy trial are summarized in Table 20.

TABLE 20
Inbred efficacy trial results of the breeding stack.

| | | Plant | Days | Days | Ear | Shell | Test | |
|---|---|---|---|---|---|---|---|---|
| | Moisture | Height | to 50% | to 50% | Height | Weight | Weight | Yield |
| Event | (%) | (inches) | Pollen | Silk | (inches) | (lb/plot) | (lb/bushel) | (bushel/acre) |
| Zm_CSM63715 | 15.90 | 61.24 | 75.38 | 76.75 | 30.33 | 8.03 | 50.94 | 68.59 |
| Stack | | | | | | | | |
| Zm_CSM63715 | 15.47 | 63.22 | 74.21 | 75.46 | 28.18 | 8.89 | 51.67 | 76.40 |
| LSD (0.05) | 1.71 | 3.05 | 0.92 | 1.86 | 2.02 | 2.78 | 16.76 | 23.55 |

In the inbred agronomic trials, the Zm_CSM63715/MON87429 stacked plants were tested in one inbred germplasm tester at 8 locations, using a randomized complete block design with 4 replications per location. Standard agronomic practices for conventional inbred corn were performed. The hybrid agronomic trials were similar to the inbred trials except that they contained 5 hybrid testers. The results of the inbred and the one hybrid agronomic trial containing MON87429xZm_CSM63715 are summarized in Table 21, and show that the Zm_CSM63715 event and the stack performed similarly to each other, and were comparable to the wildtype controls in grain moisture, plant height, timing of pollen shed and silking, ear height, shell weight, test weight, and yield. Wildtype 1 and Wildtype 2 represent two seed lots of wildtype corn.

TABLE 21
Summary of inbred and hybrid agronomic trial results of the breeding stack.

| | | | | | | | | |
|---|---|---|---|---|---|---|---|---|
| | | | Days | Days | | | | |
| | | Plant | to | to | Ear | Shell | Test | |
| | Moisture | Height | 50% | 50% | Height | Weight | Weight | Yield |
| Event | (%) | (inches) | Pollen | Silk | (inches) | (lb/plot) | (lb/bushel) | (bushel/acre) |

Hybrid

| | | | | | | | | |
|---|---|---|---|---|---|---|---|---|
| Zm_CSM63715 | 15.02 | 82.05 | 69.60 | 69.76 | 43.01 | 24.09 | 55.44 | 207.41 |
| stack | | | | | | | | |
| Zm_CSM63715 | 14.80 | 82.27 | 68.81 | 69.30 | 41.43 | 23.61 | 56.19 | 203.94 |
| Wildtype 1 | 14.77 | 81.77 | 69.18 | 69.52 | 41.99 | 22.86 | 57.06 | 197.47 |
| Wildtype 2 | 14.66 | 81.15 | 69.14 | 69.56 | 41.06 | 23.16 | 55.94 | 200.15 |
| LSD (0.05) | 0.31 | 1.59 | 0.71 | 0.62 | 1.67 | 2.17 | 1.33 | 19.10 |

Inbred

| | | | | | | | | |
|---|---|---|---|---|---|---|---|---|
| Zm_CSM63715 | 12.26 | 62.01 | 73.40 | 73.73 | 26.85 | 6.98 | 38.18 | 65.05 |
| stack | | | | | | | | |
| Zm_CSM63715 | 12.85 | 59.32 | 74.56 | 74.86 | 28.52 | 8.28 | 41.56 | 71.37 |
| Wildtype 1 | 12.17 | 61.24 | 73.90 | 74.15 | 27.16 | 5.87 | 36.21 | 51.13 |
| Wildtype 2 | 12.75 | 61.60 | 73.68 | 73.88 | 28.43 | 6.73 | 40.22 | 58.68 |
| LSD (0.05) | 1.04 | 2.07 | 0.55 | 0.78 | 1.91 | 1.49 | 5.47 | 12.58 |

### Example 5: Molecular Characterization of Corn Event Zm_CSM63715

As described above, corn event Zm_CSM63715 was identified through comprehensive and rigorous molecular characterization and event selection processes carried out from R0 to R4 inbred generations, coupled with field performance testing including trait efficacy and yield. This example describes the extensive molecular characterization upon selection of Zm_CSM63715 as the commercial event, including confirmation of one copy of intact PPO at a targeted locus, absence of Agrobacterium Ti plasmid backbone DNA, CP4, Cpf1, gRNA and Cre sequences; confirmation of the chromosomal location of the PPO transgene, confirmation that the T-DNA did not interrupt any known endogenous gene and did not insert into any repeat regions; and identification of the transgene 5′ and 3′ genomic flanking sequences and the wildtype allele sequence. The transgenic insert of corn event Zm_CSM63715 contains the elements and sequences described in Table 1A.

DNA sequence analysis of the corn event Zm_CSM63715 was conducted. Southern hybridization analysis was conducted to confirm that plants of corn event Zm_CSM63715 contained a single and intact copy of the entire PPO cassette without any transformation vector backbone sequence, or the CP4, Cpf1, gRNA and Cre sequences. The in planta transgenic insert was isolated and sequenced using methods known in the art. Mapping of the sequence reads of the event and the nontransgenic O1DKD2 inbred line against construct ZmHT5-2, respectively, confirmed the absence of Agrobacterium Ti plasmid backbone and marker sequences including Cpf1, CP4 and gRNA, and showed that the inserted in planta T-DNA sequence was inserted at the target site on chromosome 8, and perfectly matched the expected T-DNA sequence from the transformation vector ZmHT5-2 after the removal of the CP4, Cpf1 and gRNA cassettes.

Amplicons covering transgenic insert and the 5′ or the 3′ flanking sequence were generated by PCR. The transgenic insertion site, and the respective 5′ and 3′ junction sequences were subsequently determined by amplicon sequencing. The resulting event locus comprised a 1000-nucleotide left flanking genomic sequence, a 3-nucleotide filler sequence, a 3549-nucleotide PPO expression cassette sequence, and a 1000-nucleotide right flanking genomic sequence (see FIG. 1 and Table 1A). The WT allele sequence corresponding to the event Zm_CSM63715 transgenic insertion site was also generated from 01DKD2 genomic DNA by PCR and amplicon sequencing. Comparison of the WT allele sequence with that of the event Zm_CSM63715 at the insertion site indicated that the transgenic insertion resulted in a 19-nucleotide genomic sequence deletion in the event. Sequence information for the transgenic insert, the 5′ and 3′ flanking sequences, and the 5′ and 3′ junctions are provided herein as SEQ ID NOs:1-10.

RNA analysis of plants comprising the corn event Zm_CSM63715 was conducted. Northern hybridization was performed on total RNA and mRNA isolated from immature kernel and leaf. The results confirmed RNA transcripts corresponding in size to the PPO mRNA products in corn event Zm_CSM63715.

Protein analyses of plant comprising corn event Zm_CSM63715 were also conducted. The N-terminal amino acid sequence of the expressed PPO protein was determined by Edman sequencing and mass spectrometry using immunopurified protein extracts from mature seed and leaf to confirm the authentic N-terminal amino acid sequence. Western blot analysis was conducted on protein extracts from mature seed and leaf of corn event Zm_CSM63715 to confirm the cleavage of the chloroplast transit peptide and the production of a single expected-sized protein for PPO. In addition, ELISA was used to determine PPO protein level in different tissues in corn event Zm_CSM63715 including root, silk, seed, and mature leaves during different developmental stages (V3, V6, VT and R1), under different growth conditions (greenhouse, growth chamber, and field), over multiple generations (F1 to F8) and in multiple germplasms. The results showed that the PPO protein remained stable across different growth conditions over multiple generations and in multiple germplasms tested.

### Example 6: Detection of Corn Event Zm_CSM63715

This example describes methods useful in identifying or detecting the presence of corn event Zm_CSM63715. Detection of the event in a sample can be achieved using DNA, RNA, or protein detection techniques. Illustrative detection methods and materials are provided below. 1) Corn Event Zm_CSM63715 Event-Specific Endpoint Taqman™ Assays An event-specific endpoint Applied Biosystems™ TaqMan thermal amplification method (Thermo Fisher Scientific) was developed to identify corn event Zm_CSM63715 in a sample. The DNA primers and probe used in the endpoint assay for this example are shown in Table 22, although it will be appreciated by those of skill in the art that that other primers and probes may also be used.

TABLE 22
Primers and probe for corn Zm_CSM63715 event-specific assay.

| SEQ ID NO. | Name | Type |
|---|---|---|
| 14 | Primer SQ21524 | Event-specific |
| 15 | Primer SQ51880 | Event-specific |
| 16 | 6FAM ™ probe PB10269 | Event-specific |
| 17 | Primer SQ20222 | Internal control |
| 18 | Primer SQ20221 | Internal control |
| 19 | VIC ™ Probe PB50298 | Internal control |

6-FAM™ is a fluorescent dye product of Applied Biosystems (Foster City, Calif.) and is attached to the DNA probe. For TaqMan MGB (Minor Grove Binder) probes, the 5′ exonuclease activity of Taq DNA polymerase cleaves the probe from the 5′-end, between the fluorophore and quencher. When hybridized to the target DNA strand, quencher and fluorophore are separated enough to produce a fluorescent signal, thus releasing fluorescence. The pair of primers when used with these reaction methods and the probe produce a DNA amplicon that is diagnostic for corn event Zm_CSM63715. The controls for this analysis should include a positive control containing corn event Zm_CSM63715, a negative control from a non-transgenic plant, and a negative control that contains no template DNA. Additionally, a control for the PCR reaction should optimally include internal control primers and an internal control probe, specific to a single copy gene in the corn genome. These assays are optimized for use with the Applied Biosystems GeneAmp® PCR System 9700 (Thermo Fisher Scientific) run at maximum speed, but other equipment may be used.

Examples of PCR reaction components and cycling conditions useful for the event-specific qualitative endpoint TaqMan PCR assay for corn event Zm_CSM63715 are presented in Table 23 and Table 24. The extracted DNA template was a leaf DNA sample to be analyzed, a negative control (non-transgenic corn DNA), no template (water) control, or a positive control containing corn event Zm_CSM63715 DNA.

TABLE 23
Zm_CSM63715 event-specific endpoint TaqMan ™ PCR
reaction components.

| | Stock | | Final |
|---|---|---|---|
| | Concentration | Volume | Concen- |
| Reagent | (μM) | (μl) | tration |
| Reaction Volume | | 5 | |
| 2× Master Mix | | 2.28 | 1× |
| Event Specific Primer | 100 | 0.05 | 0.9 μM |
| SQ21524 | | | |
| Event Specific Primer | 100 | 0.05 | 0.9 μM |
| SQ51880 | | | |
| Event Specific 6FAM ™ | 100 | 0.01 | 0.2 μM |
| probe PB 10269 | | | |
| Internal Control Primer | 100 | 0.05 | 0.9 μM |
| SQ20222 | | | |
| Internal Control Primer | 100 | 0.05 | 0.9 μM |
| SQ20221 | | | |
| Internal Control VIC ™ | 100 | 0.01 | 0.2 μM |
| Probe PB50298 | | | |
| Extracted DNA template | | 2.5 | |

TABLE 24
Endpoint TaqMan ™ thermocycler conditions.

| Step No. | Cycle No. | Settings |
|---|---|---|
| 1 | 1 | 95° C., 20 seconds |
| 2 | 35 | 95° C., 3 seconds |
| | | 60° C., 20 seconds |

### 2) Detection of Corn Event Zm_CSM63715 Using Antibody

Another example of detection of corn event Zm_CSM63715 involves the use of antibody specific for the PPO protein encoded by corn event Zm_CSM63715. For example, a detection kit comprising such antibody can be used. Such a kit may utilize a lateral flow strip comprising reagents activated when the tip of the strip is contacted with an aqueous solution. Illustrative protein sufficient for use in antibody production is the PPO encoded by the sequence provided as SEQ ID NO:10, or any fragment thereof.

A protein detection method is developed to determine whether a sample is from a plant, seed, cell, or plant part comprising corn event Zm_CSM63715. An antibody specific for the PPO protein encoded by corn event Zm_CSM63715 is used to detect a protein encoded by corn event Zm_CSM63715 in a sample. A detection kit comprising an antibody specific for the PPO protein encoded by corn event Zm_CSM63715 may utilize a lateral flow strip containing reagent activated when the tip of the strip is contacted with an aqueous solution. Samples of corn tissues may be ground up and protein extracted for analysis using water or an aqueous buffer (for example, phosphate buffered saline containing detergent and bovine serum albumin). Following centrifugation, the aqueous supernatant is analyzed using the ELISA method in a sandwich format on a lateral flow strip containing an absorbent pad. Detection is activated by dipping the tip of the strip into the aqueous solution containing the sample to be tested.

The aqueous solution is carried up the strip by capillary action and solubilizes gold labeled antibody on the strip. The gold-labeled antibody is specific for the PPO protein encoded by corn event Zm_CSM63715 and will bind to an epitope on the protein in the sample to form an antibody-antigen complex. The gold labeled antibody-antigen complex is then carried up the strip to a nitrocellulose membrane. The membrane comprises a test line of immobilized antibody that binds to a second, separate epitope on the PPO protein encoded by corn event Zm_CSM63715, causing a visible line to appear across the test strip if the protein encoded by corn event Zm_CSM63715 is present in the sample.

### 3) Detection of Corn Event Zm_CSM63715 by Southern Analysis

Another method to detect the presence of corn event Zm_CSM63715 in a plant sample is Southern analysis as generally understood in the art. One of skill in art, based on the present disclosure and description of corn event Zm_CSM63715, would understand how to design Southern hybridization probe(s) specific for the event and a second Southern hybridization probe specific for a plant which is null for the event (wildtype). With Southern analysis, a signal detected only from the first Southern hybridization probe will be indicative of a plant positive for corn event Zm_CSM63715; a signal detected only from the second Southern hybridization probe will be indicative that the DNA was extracted from a plant that is null for the event (wildtype).

### Example 7: Zygosity Assays for Corn Event Zm_CSM63715

This example describes methods useful in determining the zygosity of event Zm_CSM63715. The zygosity assay determines whether a plant comprising corn event Zm_CSM63715 is heterozygous or homozygous for the event or the wildtype allele. Illustrative detection methods and materials are provided below.

A zygosity assay was developed to determine whether a plant comprising corn event Zm_CSM63715 is heterozygous or homozygous for the event allele. An amplification reaction assay can be designed using the sequence information provided herein. For example, such a PCR assay would include design of at least three primers: primer-1, primer-2 and primer-3, where primer-1 is specific to corn genomic DNA on the 5′ flanking DNA of corn event Zm_CSM63715 (for example, SEQ ID NO:15); primer-2 is specific to corn event Zm_CSM63715 transgenic insert (for example, SEQ ID NO:14); and primer-3 is specific to the wildtype allele (for example, SEQ ID NO:21). Alternatively, primer-3 (SEQ ID NO:21) and primer-4 (SEQ ID NO:20) are both specific to the wildtype allele. When used as a primer pair in an amplification reaction, primer-1 with primer-2 will produce a PCR amplicon specific for corn event Zm_CSM63715. When used as a primer pair in an amplification reaction, primer-1 with primer-3, or primer-3 with primer-4 will produce a PCR amplicon specific for wildtype allele. In a PCR reaction performed on corn event Zm_CSM63715, the respective PCR amplicons generated from primer-1+primer-2 and those generated from primer-1 or primer-4+primer-3 will differ in sequence and size of the amplicon. When the three or four primers are included in a PCR reaction with DNA extracted from a plant homozygous for corn event Zm_CSM63715, only the primer-1+primer-2 amplicon (specific for the corn event Zm_CSM63715) will be generated. When the three or four primers are included in a PCR reaction with DNA extracted from a plant heterozygous for corn event Zm_CSM63715, both the primer-1+primer-2 amplicon (specific for the corn event Zm_CSM63715 insert) and the primer-1 or primer-4+primer-3 amplicon (specific for the wildtype allele or absence of the corn event Zm_CSM63715 insert) will be generated. When the three or four primers are mixed together in a PCR reaction with DNA extracted from a plant that is null for corn event Zm_CSM63715 (i.e., wildtype), only the primer-1 or primer-4+primer-3 amplicon (specific for the wildtype allele) will be generated. The amplicons produced using the PCR reaction may be identified or distinguished using any method known in the art.

Another zygosity assay for corn event Zm_CSM63715 is a Tagman™ thermal amplification method. Two fluorescently labeled probes are included in addition to the primers as described in the proceeding section. Probe-1, containing a fluorescent label (for example, the 6-FAM™-labeled SEQ ID NO:16), is specific for corn event Zm_CSM63715; whereas Probe-2, containing a different fluorescent label (for example, the VIC™-labeled SEQ ID NO:22), is specific for a wildtype corn plant that is null for corn event Zm_CSM63715.

When the three (SEQ ID NO:14, SEQ ID NO:15, and SEQ ID NO:21) or four primers (SEQ ID NO:14, SEQ ID NO:15, SEQ ID NO:20 and SEQ ID NO:21) and two probes (SEQ ID NO:16 and SEQ ID NO:22) are mixed together in a PCR reaction with DNA extracted from a plant homozygous for corn event Zm_CSM63715, a fluorescent signal from the 6FAM™_labeledprobe PB10269 (SEQ ID NO:16) is released, which is indicative of and diagnostic for a plant homozygous for corn event Zm_CSM63715. When the three or four primers and two probes are mixed together in a PCR reaction with DNA extracted from a plant heterozygous for corn event Zm_CSM63715, two distinct fluorescent signals are generated, one from the 6FAM™_labeled probe PB10269 (SEQ ID NO:16), and one from the VIC™-labeled probe PB50707 (SEQ ID NO:22). When the three or four primers and the two probes are mixed together in a PCR reaction with DNA extracted from a wildtype plant which is null for corn event Zm_CSM63715, a fluorescent signal from the VIC™-labeled probe PB50707 (SEQ ID NO:22) is generated.

Examples of PCR reaction components and cycling conditions useful for TaqMan™ PCR zygosity assay for corn event Zm_CSM63715 are presented in Table 25 (for an assay where four primers and two probes are used), Table 26 (for an assay where three primers and two probes are used), and Table 27. The extracted DNA template was a DNA sample to be analyzed, a negative control (non-transgenic corn DNA), no template (water) control, or a positive control containing corn event Zm_CSM63715 DNA.

TABLE 25
Zm_CSM63715 zygosity TaqMan ™ PCR
reaction components (four primers).

| | Stock | | Final |
|---|---|---|---|
| | Concentration | Volume | Concen- |
| Reagent | (μM) | (μl) | tration |
| Reaction Volume | | 5 | |
| 2× Master Mix | | 2.3 | 1× |
| Event Specific Primer SQ21524 | 100 | 0.045 | 0.9 μM |
| (SEQ ID NO: 14) | | | |
| Event Specific Primer SQ51880 | 100 | 0.045 | 0.9 μM |
| (SEQ ID NO: 15) | | | |
| Event Specific 6FAM ™ probe | 100 | 0.01 | 0.2 μM |
| PB10269 (SEQ ID NO: 16) | | | |
| Wildtype allele primer SQ52146 | 100 | 0.045 | 0.9 μM |
| (SEQ ID NO: 20) | | | |
| Wildtype allele primer SQ5214 | 100 | 0.045 | 0.9 μM |
| (SEQ ID NO: 21)7 | | | |
| Wildtype allele VIC ™ probe | 100 | 0.01 | 0.2 μM |
| PB50707 (SEQ ID NO: 22) | | | |
| Extracted DNA template | | 2.5 | |

TABLE 26
Zm_CSM63715 zygosity TaqMan ™ PCR
reaction components (three primers).

| | Stock | | Final |
|---|---|---|---|
| | Concentration | Volume | Concen- |
| Reagent | (μM) | (μl) | tration |
| Reaction Volume | | 5 | |
| 2× Master Mix | | 2.345 | 1× |
| Event Specific Primer SQ21524 | 100 | 0.045 | 0.9 μM |
| (SEQ ID NO: 14) | | | |
| Event Specific Primer SQ51880 | 100 | 0.045 | 0.9 μM |
| (SEQ ID NO: 15) | | | |
| Event Specific 6FAM ™ probe | 100 | 0.01 | 0.2 μM |
| PB10269 (SEQ ID NO: 16) | | | |
| Wildtype allele primer SQ5214 | 100 | 0.045 | 0.9 μM |
| (SEQ ID NO: 21)7 | | | |
| Wildtype allele VIC ™ probe | 100 | 0.01 | 0.2 μM |
| PB50707 (SEQ ID NO: 22) | | | |
| Extracted DNA template | | 2.5 | |

TABLE 27
Zygosity TaqMan ™ thermocycler conditions.

| Step No. | Cycle No. | Settings |
|---|---|---|
| 1 | 1 | 95° C., 20 seconds |
| 2 | 35 | 95° C., 3 seconds |
| | | 60° C., 20 seconds |

Another method to detect the presence and zygosity of corn event Zm_CSM63715 in a plant sample is Southern blot analysis. One of skill in art would understand how to design a first Southern hybridization probe(s) specific for corn event Zm_CSM63715 and a second Southern hybridization probe specific for a corn plant which is null for the corn event Zm_CMS63715 (wildtype). With Southern blot analysis, a signal detected only from the first Southern hybridization probe is indicative a plant homozygous for corn event Zm_CSM63715; a signal detected from both the first and the second hybridization probes is indicative of a plant heterozygous for corn event Zm_CSM63715; and a signal detected only from the second Southern hybridization probe indicates that the DNA was extracted from a plant that is null for corn event Zm_CSM63715 (wildtype).

### Example 8: Modification of Corn Event Zm_CSM63715 with Genome Editing Techniques Using a Single Guide RNA

This example describes how one may alter or excise all or a part of the transgenic insertion present in corn event Zm_CSM63715, as well as flanking genomic DNA segments, such as by making one or more insertions, deletions, substitutions, or transpositions using genomic editing techniques. For example, such alterations can be made using Clustered Regularly Interspersed Short Palindromic Repeats (CRISPR) editing systems comprising a single guide RNA by genome editing methods. Sequences useful in excision of the event Zm_CSM63715 transgenic insertion or the expression cassette within SEQ ID NO:9 or SEQ ID NO:10 can be introduced through genome editing using a variety of methods. In one embodiment, Clustered Regularly Interspersed Short Palindromic Repeats (CRISPR) editing systems comprising a CRISPR associated protein and cognate guide RNAs may be used for targeted excision. The CRISPR-associated protein is an RNA guided nuclease and can be selected from a Type I CRISPR-associated protein, a Type II CRISPR-associated protein, a Type III CRISPR-associated protein, a Type IV CRISPR-associated protein, a Type V CRISPR-associated protein, or a Type VI CRISPR-associated protein, such as, but not limited to, Cas1, Cas1B, Cas2, Cas3, Cas4, Cas5, Cas6, Cas7, Cas8, Cas9 (also known as Csn1 and Csx12), Cas10, Cas12a (also known as Cpf1), Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, CasX, CasY, and Mad7. The CRISPR-associated protein and one or more guide RNAs (gRNAs) can be introduced into a plant cell corresponding to corn event Zm_CSM63715 to target a specific sequence within the transgene insertion locus. In one embodiment, the CRISPR nuclease system cleaves at two identical guide RNA hybridization sites thereby permitting the excision of the intervening sequence. Following DNA cleavage, the genomic sequence can be repaired via a double strand break repair pathway, which may include, for example, non-homologous end-joining (NHEJ), microhomology-mediated end joining (MMEJ), homologous recombination, synthesis-dependent strand annealing (SDSA), single-strand annealing (SSA), or a combination of any thereof, at the genomic target site. One or more guide RNA hybridization sequences can be inserted within the event Zm_CSM63715 transgene insertion locus which can subsequently allow for the excision of the transgene insertion from event Zm_CSM63715 or the expression cassette within SEQ ID NO:9 or SEQ ID NO:10.

Sequences corresponding to the 5′ and 3′ flanking genomic sequences of event Zm_CSM63715 (presented as SEQ ID NOs:11, 12, 164 and 165), the 5′ and 3′ junction regions (presented as SEQ ID NOs:1-6) and the transgenic insertion (presented as SEQ ID NO:9) were scanned for potential originator guide RNA recognition sites (OgRRS). As used herein, the term “originator guide RNA recognition site” or “OgRRS” refers to an endogenous DNA polynucleotide in the flanking genomic sequence or the integrated transgenic polynucleotide comprising a protospacer adjacent motif (PAM) site operably linked to a guide RNA hybridization site (i.e., protospacer sequence). In some embodiments, an OgRRS can be located in the flanking 5′ or 3′ genomic sequence (i.e., in non-transgenic DNA of a junction polynucleotide). In some embodiments, an OgRRS can be located in the 5′ or 3′ junction region (i.e., in both transgenic DNA and non-transgenic DNA of a junction polynucleotide, or spanning transgenic and non-transgenic DNA in a DNA junction polynucleotide). In some embodiments, an OgRRS can be located in the transgenic insert. The OgRRS can be determined based upon the specific CRISPR editing system chosen. For example, Cas9 recognizes a G-rich protospacer-adjacent motif (PAM) that is 3′ to its guide RNA hybridization site whereas Cas12a systems recognize a T-rich protospacer-adjacent motif (PAM) that is 5′ to its guide RNA hybridization site.

The OgRRS sequence is then used to define a cognate guide RNA recognition site (CgRRS) which is inserted into the transgenic insertion locus of event Zm_CSM63715 using a CRISPR editing system. As used herein the term “cognate guide R NA recognition site” or “CgRRS” refers to a DNA polynucleotide comprising a PAM site operably linked to a guide RNA hybridization site (i.e., protospacer sequence), wherer the CgRRS is absent from event Zm_CSM63715 comprising the original transgenic locus that is unmodified and where the CgRRS and its corresponding OgRRS can hybridize to a single gRNA, A CgRRS can be located in the flanking 5′ or 3′ genomic sequence (i.e., in non-transgenic DNA of a junction polynucleotide), in the 5′ or 3′ junction region (i.e., in both transgenic DNA and non-transgenic DNA of a junction polynucleotide, or spanning transgenic and non-transgenic DNA in a DNA junction polynucleotide), or in the transgenic insert. A CgRRS comprises the same gRNA target sequence as the corresponding OgRRS. The CgRRS is inserted in a region within the transgenic insertion locus of event Zm_CSM63715 that is on the opposite side of the transgenic insertion, relative to the OgRRS in a manner that will permit the excision of a fragment of DNA corresponding to either the entire transgenic insertion of event Zm_CSM63715, or a fragment within the transgene insert of event Zm_CSM63715 such as an expression cassette or genetic element within the transgene cassette, using a single gRNA. For example, if the OgRRS is located within the 3′ flanking genomic sequence or the 3′ junction region, then the CgRRS can be inserted within the 5′ flanking genomic sequence, or the 5′ junction region, or within the transgene insert such as between genetic elements within the expression cassette. Insertion of the CgRRS on the opposite side of the transgenic insertion or within the transgenic insert, relative to the OgRRS allows for excision of the transgenic insertion or specific expression element(s) or expression cassette to be excised using a single gRNA. An OgRRS located in the expression cassette of event Zm_CSM63715 can be used to design a CgRRS that can be inserted in either the 5′ or 3′-flanking genomic sequence to permit excision of one or more expression elements or the expression cassette using a single gRNA.

Table 28 shows OgRRS sequences located within the 5′ and 3′-flanking genomic sequences, spanning the 5′ and 3′ junction sequences, and in the transgenic insertion of event Zm_CSM63715 that can be used in a CRISPR editing system employing Cas12a, a Type V CRISPR-associated protein. The analysis was performed for four Cas12a endonucleases. FnCas12a (SEQ ID NO:37) refers to Francisella novicida U112 Cas12a (also known as FnCpf1), and requires the PAM sequence of 5′-TTN, where N is A, C, G or T (Zetsche et al., 2015). LbCas12a (SEQ ID NO:34) refers to the Cas12a from Lachnospiraceae bacterium ND2006 (also known as LbCpf1), and requires the PAM sequence of 5′-TTTV, where V is A, C, or G. LbCas12a-TYC and LbCas12a-TAT refer to engineered variants of Lachnospiraceae bacterium ND2006 Cas12a (Gao et al., 2017). The LbCas12a-TYC variant (SEQ ID NO:35) contains the G532R/K595R mutations and recognizes 5′-TYCV PAM; whereas the LbCas12a-TAT variant (SEQ ID NO:36) contains the G532R/K538V/Y542R mutations and recognizes 5′-TATV PAM, where Y is C or T, and V is A, C or G. The PAM sequence, the coordinates of the gRNA hybridization site (also known as OgRRS) relative to SEQ ID NO:10, and the corresponding Casl12a endonuclease are shown under the headings of “PAM”, “Casl12a Nuclease”, and “Start..End of gRNA Hybridization Site in SED ID NO:10”, respectively. “Strand of SEQ ID NO: 10” indicates whether the identified gRNA hybridization site along with its PAM sequence is on the forward strand (+) or the complementary strand (−). “Target Site” refers to the location of the gRNA hybridization site in the corn event CSM63715 locus.

TABLE 28
OgRRS sequences within event Zm_CSM63715.

| | | | Start . . . End | | |
|---|---|---|---|---|---|
| | | | of gRNA | | |
| Name of gRNA | | | Hybridization | Strand | |
| Hybridization | | | Site | of | |
| Site | | Cas12a | in SEQ ID | SEQ ID | |
| (OgRRS) | PAM | Nuclease | NO: 10 | NO: 10 | Target Site |
| 5F-1 | ATTA | FnCas12a | 627 . . . 649 | - | 5′ Flanking genomic |
| | | | | | DNA |
| 5F-2 | ATTA | FnCas12a | 649 . . . 671 | − | 5′ Flanking genomic |
| | | | | | DNA |
| 5F-3 | ATTA | FnCas12a | 815 . . . 837 | − | 5′ Flanking genomic |
| | | | | | DNA |
| 5F-4 | ATTA | FnCas12a | 827 . . . 849 | − | 5′ Flanking genomic |
| | | | | | DNA |
| 5F-5 | ATTA | FnCas12a | 912 . . . 934 | + | 5′ Flanking genomic |
| | | | | | DNA |
| 5F-6 | ATTA | FnCas12a | 906 . . . 928 | − | 5′ Flanking genomic |
| | | | | | DNA |
| 5F-7 | ATTA | FnCas12a | 918 . . . 940 | − | 5′ Flanking genomic |
| | | | | | DNA |
| 5F-8 | ATTA | FnCas12a | 941 . . . 963 | + | 5′ Flanking genomic |
| | | | | | DNA |
| 5F-9 | ATTA | FnCas12a | 947 . . . 969 | − | 5′ Flanking genomic |
| | | | | | DNA |
| 5F-10 | ATTA | FnCas12a | 962 . . . 984 | − | 5′ Flanking genomic |
| | | | | | DNA |
| 5F-11 | ATTC | FnCas12a | 349 . . . 371 | + | 5′ Flanking genomic |
| | | | | | DNA |
| 5F-12 | ATTC | FnCas12a | 369 . . . 391 | + | 5′ Flanking genomic |
| | | | | | DNA |
| 5F-13 | ATTC | FnCas12a | 609 . . . 631 | + | 5′ Flanking genomic |
| | | | | | DNA |
| 5F-14 | ATTC | FnCas12a | 895 . . . 917 | − | 5′ Flanking genomic |
| | | | | | DNA |
| 5F-15 | ATTG | FnCas12a | 11 . . . 33 | − | 5′ Flanking genomic |
| | | | | | DNA |
| 5F-16 | ATTG | FnCas12a | 24 . . . 46 | − | 5′ Flanking genomic |
| | | | | | DNA |
| 5F-17 | ATTG | FnCas12a | 211 . . . 233 | − | 5′ Flanking genomic |
| | | | | | DNA |
| 5F-18 | ATTG | FnCas12a | 286 . . . 308 | − | 5′ Flanking genomic |
| | | | | | DNA |
| 5F-19 | ATTG | FnCas12a | 346 . . . 368 | − | 5′ Flanking genomic |
| | | | | | DNA |
| 5F-20 | ATTG | FnCas12a | 447 . . . 469 | − | 5′ Flanking genomic |
| | | | | | DNA |
| 5F-21 | ATTG | FnCas12a | 531 . . . 553 | − | 5′ Flanking genomic |
| | | | | | DNA |
| 5F-22 | ATTG | FnCas12a | 614 . . . 636 | + | 5′ Flanking genomic |
| | | | | | DNA |
| 5F-23 | ATTG | FnCas12a | 620 . . . 642 | + | 5′ Flanking genomic |
| | | | | | DNA |
| 5F-24 | ATTG | FnCas12a | 721 . . . 743 | + | 5′ Flanking genomic |
| | | | | | DNA |
| 5F-25 | CTTA | FnCas12a | 125 . . . 147 | + | 5′ Flanking genomic |
| | | | | | DNA |
| 5F-26 | CTTA | FnCas12a | 545 . . . 567 | − | 5′ Flanking genomic |
| | | | | | DNA |
| 5F-27 | CTTA | FnCas12a | 908 . . . 930 | + | 5′ Flanking genomic |
| | | | | | DNA |
| 5F-28 | CTTC | FnCas12a | 12 . . . 34 | + | 5′ Flanking genomic |
| | | | | | DNA |
| 5F-29 | CTTC | FnCas12a | 25 . . . 47 | + | 5′ Flanking genomic |
| | | | | | DNA |
| 5F-30 | CTTC | FnCas12a | 170 . . . 192 | + | 5′ Flanking genomic |
| | | | | | DNA |
| 5F-31 | CTTC | FnCas12a | 203 . . . 225 | + | 5′ Flanking genomic |
| | | | | | DNA |
| 5F-32 | CTTC | FnCas12a | 189 . . . 211 | − | 5′ Flanking genomic |
| | | | | | DNA |
| 5F-33 | CTTC | FnCas12a | 273 . . . 295 | + | 5′ Flanking genomic |
| | | | | | DNA |
| 5F-34 | CTTC | FnCas12a | 437 . . . 459 | − | 5′ Flanking genomic |
| | | | | | DNA |
| 5F-35 | CTTC | FnCas12a | 475 . . . 497 | − | 5′ Flanking genomic |
| | | | | | DNA |
| 5F-36 | CTTC | FnCas12a | 630 . . . 652 | + | 5′ Flanking genomic |
| | | | | | DNA |
| 5F-37 | CTTC | FnCas12a | 653 . . . 675 | − | 5′ Flanking genomic |
| | | | | | DNA |
| 5F-38 | CTTC | FnCas12a | 662 . . . 684 | − | 5′ Flanking genomic |
| | | | | | DNA |
| 5F-39 | CTTC | FnCas12a | 744 . . . 766 | + | 5′ Flanking genomic |
| | | | | | DNA |
| 5F-40 | CTTG | FnCas12a | 18 . . . 40 | + | 5′ Flanking genomic |
| | | | | | DNA |
| 5F-41 | CTTG | FnCas12a | 59 . . . 81 | − | 5′ Flanking genomic |
| | | | | | DNA |
| 5F-42 | CTTG | FnCas12a | 492 . . . 514 | − | 5′ Flanking genomic |
| | | | | | DNA |
| 5F-43 | CTTG | FnCas12a | 552 . . . 574 | − | 5′ Flanking genomic |
| | | | | | DNA |
| 5F-44 | CTTG | FnCas12a | 637 . . . 659 | − | 5′ Flanking genomic |
| | | | | | DNA |
| 5F-45 | GTTA | FnCas12a | 530 . . . 552 | + | 5′ Flanking genomic |
| | | | | | DNA |
| 5F-46 | GTTA | FnCas12a | 534 . . . 556 | − | 5′ Flanking genomic |
| | | | | | DNA |
| 5F-47 | GTTA | FnCas12a | 538 . . . 560 | − | 5′ Flanking genomic |
| | | | | | DNA |
| 5F-48 | GTTA | FnCas12a | 575 . . . 597 | − | 5′ Flanking genomic |
| | | | | | DNA |
| 5F-49 | GTTA | FnCas12a | 652 . . . 674 | + | 5′ Flanking genomic |
| | | | | | DNA |
| 5F-50 | GTTA | FnCas12a | 823 . . . 845 | − | 5′ Flanking genomic |
| | | | | | DNA |
| 5F-51 | GTTA | FnCas12a | 860 . . . 882 | − | 5′ Flanking genomic |
| | | | | | DNA |
| 5F-52 | GTTC | FnCas12a | 31 . . . 53 | − | 5′ Flanking genomic |
| | | | | | DNA |
| 5F-53 | GTTC | FnCas12a | 212 . . . 234 | + | 5′ Flanking genomic |
| | | | | | DNA |
| 5F-54 | GTTC | FnCas12a | 253 . . . 275 | − | 5′ Flanking genomic |
| | | | | | DNA |
| 5F-55 | GTTC | FnCas12a | 575 . . . 597 | + | 5′ Flanking genomic |
| | | | | | DNA |
| 5F-56 | GTTC | FnCas12a | 582 . . . 604 | + | 5′ Flanking genomic |
| | | | | | DNA |
| 5F-57 | GTTC | FnCas12a | 596 . . . 618 | + | 5′ Flanking genomic |
| | | | | | DNA |
| 5F-58 | GTTG | FnCas12a | 5 . . . 27 | + | 5′ Flanking genomic |
| | | | | | DNA |
| 5F-59 | GTTG | FnCas12a | 18 . . . 40 | − | 5′ Flanking genomic |
| | | | | | DNA |
| 5F-60 | GTTG | FnCas12a | 149 . . . 171 | + | 5′ Flanking genomic |
| | | | | | DNA |
| 5F-61 | GTTG | FnCas12a | 220 . . . 242 | − | 5′ Flanking genomic |
| | | | | | DNA |
| 5F-62 | GTTG | FnCas12a | 499 . . . 521 | + | 5′ Flanking genomic |
| | | | | | DNA |
| 5F-63 | GTTG | FnCas12a | 550 . . . 572 | + | 5′ Flanking genomic |
| | | | | | DNA |
| 5F-64 | GTTG | FnCas12a | 692 . . . 714 | + | 5′ Flanking genomic |
| | | | | | DNA |
| 5F-65 | TATA | LbCas12a- | 573 . . . 595 | − | 5′ Flanking genomic |
| | | TAT | | | DNA |
| 5F-66 | TATA | LbCas12a- | 600 . . . 622 | + | 5′ Flanking genomic |
| | | TAT | | | DNA |
| 5F-67 | TATA | LbCas12a- | 643 . . . 665 | − | 5′ Flanking genomic |
| | | TAT | | | DNA |
| 5F-68 | TATA | LbCas12a- | 670 . . . 692 | + | 5′ Flanking genomic |
| | | TAT | | | DNA |
| 5F-69 | TATA | LbCas12a- | 688 . . . 710 | − | 5′ Flanking genomic |
| | | TAT | | | DNA |
| 5F-70 | TATA | LbCas12a- | 715 . . . 737 | + | 5′ Flanking genomic |
| | | TAT | | | DNA |
| 5F-71 | TATA | LbCas12a- | 821 . . . 843 | − | 5′ Flanking genomic |
| | | TAT | | | DNA |
| 5F-72 | TATA | LbCas12a- | 848 . . . 870 | + | 5′ Flanking genomic |
| | | TAT | | | DNA |
| 5F-73 | TATA | LbCas12a- | 916 . . . 938 | − | 5′ Flanking genomic |
| | | TAT | | | DNA |
| 5F-74 | TATC | LbCas12a- | 513 . . . 535 | + | 5′ Flanking genomic |
| | | TAT | | | DNA |
| 5F-75 | TATC | LbCas12a- | 749 . . . 771 | + | 5′ Flanking genomic |
| | | TAT | | | DNA |
| 5F-76 | TATC | LbCas12a- | 819 . . . 841 | − | 5′ Flanking genomic |
| | | TAT | | | DNA |
| 5F-77 | TATC | LbCas12a- | 925 . . . 947 | − | 5′ Flanking genomic |
| | | TAT | | | DNA |
| 5F-78 | TATG | LbCas12a- | 720 . . . 742 | − | 5′ Flanking genomic |
| | | TAT | | | DNA |
| 5F-79 | TATG | LbCas12a- | 813 . . . 835 | − | 5′ Flanking genomic |
| | | TAT | | | DNA |
| 5F-80 | TATG | LbCas12a- | 960 . . . 982 | − | 5′ Flanking genomic |
| | | TAT | | | DNA |
| 5F-81 | TCCA | LbCas12a- | 29 . . . 51 | − | 5′ Flanking genomic |
| | | TYC | | | DNA |
| 5F-82 | TCCA | LbCas12a- | 184 . . . 206 | + | 5′ Flanking genomic |
| | | TYC | | | DNA |
| 5F-83 | TCCA | LbCas12a- | 203 . . . 225 | − | 5′ Flanking genomic |
| | | TYC | | | DNA |
| 5F-84 | TCCA | LbCas12a- | 245 . . . 267 | + | 5′ Flanking genomic |
| | | TYC | | | DNA |
| 5F-85 | TCCA | LbCas12a- | 275 . . . 297 | + | 5′ Flanking genomic |
| | | TYC | | | DNA |
| 5F-86 | TCCA | LbCas12a- | 371 . . . 393 | + | 5′ Flanking genomic |
| | | TYC | | | DNA |
| 5F-87 | TCCA | LbCas12a- | 409 . . . 431 | − | 5′ Flanking genomic |
| | | TYC | | | DNA |
| 5F-88 | TCCA | LbCas12a- | 414 . . . 436 | − | 5′ Flanking genomic |
| | | TYC | | | DNA |
| 5F-89 | TCCA | LbCas12a- | 445 . . . 467 | + | 5′ Flanking genomic |
| | | TYC | | | DNA |
| 5F-90 | TCCA | LbCas12a- | 424 . . . 446 | − | 5′ Flanking genomic |
| | | TYC | | | DNA |
| 5F-91 | TCCA | LbCas12a- | 456 . . . 478 | − | 5′ Flanking genomic |
| | | TYC | | | DNA |
| 5F-92 | TCCA | LbCas12a- | 460 . . . 482 | − | 5′ Flanking genomic |
| | | TYC | | | DNA |
| 5F-93 | TCCA | LbCas12a- | 467 . . . 489 | − | 5′ Flanking genomic |
| | | TYC | | | DNA |
| 5F-94 | TCCA | LbCas12a- | 577 . . . 599 | + | 5′ Flanking genomic |
| | | TYC | | | DNA |
| 5F-95 | TCCA | LbCas12a- | 751 . . . 773 | + | 5′ Flanking genomic |
| | | TYC | | | DNA |
| 5F-96 | TCCA | LbCas12a- | 861 . . . 883 | + | 5′ Flanking genomic |
| | | TYC | | | DNA |
| 5F-97 | TCCC | LbCas12a- | 64 . . . 86 | + | 5′ Flanking genomic |
| | | TYC | | | DNA |
| 5F-98 | TCCC | LbCas12a- | 118 . . . 140 | + | 5′ Flanking genomic |
| | | TYC | | | DNA |
| 5F-99 | TCCC | LbCas12a- | 177 . . . 199 | + | 5′ Flanking genomic |
| | | TYC | | | DNA |
| 5F-100 | TCCC | LbCas12a- | 241 . . . 263 | + | 5′ Flanking genomic |
| | | TYC | | | DNA |
| 5F-101 | TCCC | LbCas12a- | 284 . . . 306 | + | 5′ Flanking genomic |
| | | TYC | | | DNA |
| 5F-102 | TCCC | LbCas12a- | 293 . . . 315 | + | 5′ Flanking genomic |
| | | TYC | | | DNA |
| 5F-103 | TCCC | LbCas12a- | 351 . . . 373 | + | 5′ Flanking genomic |
| | | TYC | | | DNA |
| 5F-104 | TCCC | LbCas12a- | 627 . . . 649 | + | 5′ Flanking genomic |
| | | TYC | | | DNA |
| 5F-105 | TCCG | LbCas12a- | 135 . . . 157 | − | 5′ Flanking genomic |
| | | TYC | | | DNA |
| 5F-106 | TCCG | LbCas12a- | 209 . . . 231 | + | 5′ Flanking genomic |
| | | TYC | | | DNA |
| 5F-107 | TCCG | LbCas12a- | 224 . . . 246 | + | 5′ Flanking genomic |
| | | TYC | | | DNA |
| 5F-108 | TCCG | LbCas12a- | 350 . . . 372 | − | 5′ Flanking genomic |
| | | TYC | | | DNA |
| 5F-109 | TCCG | LbCas12a- | 430 . . . 452 | + | 5′ Flanking genomic |
| | | TYC | | | DNA |
| 5F-110 | TTCA | LbCas12a- | 26 . . . 48 | + | 5′ Flanking genomic |
| | | TYC | | | DNA |
| 5F-111 | TTCA | LbCas12a- | 278 . . . 300 | − | 5′ Flanking genomic |
| | | TYC | | | DNA |
| 5F-112 | TTCA | LbCas12a- | 436 . . . 458 | − | 5′ Flanking genomic |
| | | TYC | | | DNA |
| 5F-113 | TTCA | LbCas12a- | 478 . . . 500 | + | 5′ Flanking genomic |
| | | TYC | | | DNA |
| 5F-114 | TTCA | LbCas12a- | 474 . . . 496 | − | 5′ Flanking genomic |
| | | TYC | | | DNA |
| 5F-115 | TTCA | LbCas12a- | 583 . . . 605 | + | 5′ Flanking genomic |
| | | TYC | | | DNA |
| 5F-116 | TTCA | LbCas12a- | 589 . . . 611 | − | 5′ Flanking genomic |
| | | TYC | | | DNA |
| 5F-117 | TTCA | LbCas12a- | 652 . . . 674 | − | 5′ Flanking genomic |
| | | TYC | | | DNA |
| 5F-118 | TTCA | LbCas12a- | 661 . . . 683 | − | 5′ Flanking genomic |
| | | TYC | | | DNA |
| 5F-119 | TTCA | LbCas12a- | 745 . . . 767 | + | 5′ Flanking genomic |
| | | TYC | | | DNA |
| 5F-120 | TTCA | LbCas12a- | 745 . . . 767 | − | 5′ Flanking genomic |
| | | TYC | | | DNA |
| 5F-121 | TTCC | LbCas12a- | 30 . . . 52 | − | 5′ Flanking genomic |
| | | TYC | | | DNA |
| 5F-122 | TTCC | LbCas12a- | 171 . . . 193 | + | 5′ Flanking genomic |
| | | TYC | | | DNA |
| 5F-123 | TTCC | LbCas12a- | 252 . . . 274 | − | 5′ Flanking genomic |
| | | TYC | | | DNA |
| 5F-124 | TTCC | LbCas12a- | 274 . . . 296 | + | 5′ Flanking genomic |
| | | TYC | | | DNA |
| 5F-125 | TTCC | LbCas12a- | 350 . . . 372 | + | 5′ Flanking genomic |
| | | TYC | | | DNA |
| 5F-126 | TTCC | LbCas12a- | 370 . . . 392 | + | 5′ Flanking genomic |
| | | TYC | | | DNA |
| 5F-127 | TTCC | LbCas12a- | 576 . . . 598 | + | 5′ Flanking genomic |
| | | TYC | | | DNA |
| 5F-128 | TTCC | LbCas12a- | 610 . . . 632 | + | 5′ Flanking genomic |
| | | TYC | | | DNA |
| 5F-129 | TTCC | LbCas12a- | 626 . . . 648 | + | 5′ Flanking genomic |
| | | TYC | | | DNA |
| 5F-130 | TTCC | LbCas12a- | 860 . . . 882 | + | 5′ Flanking genomic |
| | | TYC | | | DNA |
| 5F-131 | TTCG | LbCas12a- | 188 . . . 210 | − | 5′ Flanking genomic |
| | | TYC | | | DNA |
| 5F-132 | TTCG | LbCas12a- | 213 . . . 235 | + | 5′ Flanking genomic |
| | | TYC | | | DNA |
| 5F-133 | TTCG | LbCas12a- | 732 . . . 754 | + | 5′ Flanking genomic |
| | | TYC | | | DNA |
| 5F-134 | TTTA | LbCas12a | 33 . . . 55 | + | 5′ Flanking genomic |
| | | | | | DNA |
| 5F-135 | TTTG | LbCas12a | 54 . . . 76 | + | 5′ Flanking genomic |
| | | | | | DNA |
| 5F-136 | TTTG | LbCas12a | 163 . . . 185 | − | 5′ Flanking genomic |
| | | | | | DNA |
| 5F-137 | TTTC | LbCas12a | 279 . . . 301 | − | 5′ Flanking genomic |
| | | | | | DNA |
| 5F-138 | TTTG | LbCas12a | 426 . . . 448 | + | 5′ Flanking genomic |
| | | | | | DNA |
| 5F-139 | TTTC | LbCas12a | 477 . . . 499 | + | 5′ Flanking genomic |
| | | | | | DNA |
| 5F-140 | TTTC | LbCas12a | 497 . . . 519 | − | 5′ Flanking genomic |
| | | | | | DNA |
| 5F-141 | TTTC | LbCas12a | 590 . . . 612 | − | 5′ Flanking genomic |
| | | | | | DNA |
| 5F-142 | TTTC | LbCas12a | 625 . . . 647 | + | 5′ Flanking genomic |
| | | | | | DNA |
| 5F-143 | TTTC | LbCas12a | 635 . . . 657 | + | 5′ Flanking genomic |
| | | | | | DNA |
| 5F-144 | TTTG | LbCas12a | 641 . . . 663 | + | 5′ Flanking genomic |
| | | | | | DNA |
| 5F-145 | TTTA | LbCas12a | 645 . . . 667 | − | 5′ Flanking genomic |
| | | | | | DNA |
| 5F-146 | TTTA | LbCas12a | 668 . . . 690 | + | 5′ Flanking genomic |
| | | | | | DNA |
| 5F-147 | TTTC | LbCas12a | 670 . . . 692 | − | 5′ Flanking genomic |
| | | | | | DNA |
| 5F-148 | TTTA | LbCas12a | 713 . . . 735 | + | 5′ Flanking genomic |
| | | | | | DNA |
| 5F-149 | TTTC | LbCas12a | 731 . . . 753 | + | 5′ Flanking genomic |
| | | | | | DNA |
| 5F-150 | TTTC | LbCas12a | 741 . . . 763 | + | 5′ Flanking genomic |
| | | | | | DNA |
| 5F-151 | TTTG | LbCas12a | 738 . . . 760 | − | 5′ Flanking genomic |
| | | | | | DNA |
| 5F-152 | TTTC | LbCas12a | 746 . . . 768 | 1 | 5′ Flanking genomic |
| | | | | | DNA |
| 5F-153 | TTTG | LbCas12a | 786 . . . 808 | + | 5′ Flanking genomic |
| | | | | | DNA |
| 5F-154 | TTTG | LbCas12a | 792 . . . 814 | + | 5′ Flanking genomic |
| | | | | | DNA |
| 5F-155 | TTTA | LbCas12a | 779 . . . 801 | − | 5′ Flanking genomic |
| | | | | | DNA |
| 5F-156 | TTTG | LbCas12a | 785 . . . 807 | − | 5′ Flanking genomic |
| | | | | | DNA |
| 5F-157 | TTTG | LbCas12a | 792 . . . 814 | − | 5′ Flanking genomic |
| | | | | | DNA |
| 5F-158 | TTTC | LbCas12a | 796 . . . 818 | − | 5′ Flanking genomic |
| | | | | | DNA |
| 5F-159 | TTTG | LbCas12a | 836 . . . 858 | − | 5′ Flanking genomic |
| | | | | | DNA |
| 5F-160 | TTTC | LbCas12a | 859 . . . 881 | + | 5′ Flanking genomic |
| | | | | | DNA |
| 5F-161 | TTTG | LbCas12a | 849 . . . 871 | − | 5′ Flanking genomic |
| | | | | | DNA |
| 5F-162 | TTTC | LbCas12a | 867 . . . 889 | − | 5′ Flanking genomic |
| | | | | | DNA |
| 5F-163 | TTTA | LbCas12a | 901 . . . 923 | − | 5′ Flanking genomic |
| | | | | | DNA |
| 5F-164 | TTTG | LbCas12a | 911 . . . 933 | − | 5′ Flanking genomic |
| | | | | | DNA |
| 5F-165 | TTTG | LbCas12a | 949 . . . 971 | + | 5′ Flanking genomic |
| | | | | | DNA |
| 5F-166 | TTTA | LbCas12a | 927 . . . 949 | − | 5′ Flanking genomic |
| | | | | | DNA |
| 5F-167 | TTTG | LbCas12a | 931 . . . 953 | − | 5′ Flanking genomic |
| | | | | | DNA |
| 5F-168 | TTTA | LbCas12a | 939 . . . 961 | − | 5′ Flanking genomic |
| | | | | | DNA |
| 5J-1 | ATTG | FnCas12a | 985 . . . 1007 | − | 5′ Junction |
| TI-1 | ATTA | FnCas12a | 1053 . . . 1075 | + | Transgenic insert |
| TI-2 | ATTA | FnCas12a | 1089 . . . 1111 | + | Transgenic insert |
| TI-3 | ATTA | FnCas12a | 1129 . . . 1151 | + | Transgenic insert |
| TI-4 | ATTA | FnCas12a | 1109 . . . 1131 | − | Transgenic insert |
| TI-5 | ATTA | FnCas12a | 1187 . . . 1209 | − | Transgenic insert |
| TI-6 | ATTA | FnCas12a | 1440 . . . 1462 | − | Transgenic insert |
| TI-7 | ATTA | FnCas12a | 1497 . . . 1519 | − | Transgenic insert |
| TI-8 | ATTA | FnCas12a | 1717 . . . 1739 | + | Transgenic insert |
| TI-9 | ATTA | FnCas12a | 1799 . . . 1821 | + | Transgenic insert |
| TI-10 | ATTA | FnCas12a | 2436 . . . 2458 | − | Transgenic insert |
| TI-11 | ATTA | FnCas12a | 2570 . . . 2592 | + | Transgenic insert |
| TI-12 | ATTA | FnCas12a | 2620 . . . 2642 | + | Transgenic insert |
| TI-13 | ATTA | FnCas12a | 2634 . . . 2656 | − | Transgenic insert |
| TI-14 | ATTA | FnCas12a | 2682 . . . 2704 | + | Transgenic insert |
| TI-15 | ATTA | FnCas12a | 2788 . . . 2810 | + | Transgenic insert |
| TI-16 | ATTA | FnCas12a | 2778 . . . 2800 | − | Transgenic insert |
| TI-17 | ATTA | FnCas12a | 2833 . . . 2855 | + | Transgenic insert |
| TI-18 | ATTA | FnCas12a | 2841 . . . 2863 | + | Transgenic insert |
| TI-19 | ATTA | FnCas12a | 2901 . . . 2923 | + | Transgenic insert |
| TI-20 | ATTA | FnCas12a | 2993 . . . 3015 | − | Transgenic insert |
| TI-21 | ATTA | FnCas12a | 3005 . . . 3027 | − | Transgenic insert |
| TI-22 | ATTA | FnCas12a | 3034 . . . 3056 | + | Transgenic insert |
| TI-23 | ATTA | FnCas12a | 3041 . . . 3063 | − | Transgenic insert |
| TI-24 | ATTA | FnCas12a | 3074 . . . 3096 | − | Transgenic insert |
| TI-25 | ATTA | FnCas12a | 3206 . . . 3228 | + | Transgenic insert |
| TI-26 | ATTA | FnCas12a | 3398 . . . 3420 | + | Transgenic insert |
| TI-27 | ATTA | FnCas12a | 3580 . . . 3602 | − | Transgenic insert |
| TI-28 | ATTA | FnCas12a | 3847 . . . 3869 | − | Transgenic insert |
| TI-29 | ATTA | FnCas12a | 3850 . . . 3872 | − | Transgenic insert |
| TI-30 | ATTA | FnCas12a | 3970 . . . 3992 | − | Transgenic insert |
| TI-31 | ATTA | FnCas12a | 3999 . . . 4021 | + | Transgenic insert |
| TI-32 | ATTA | FnCas12a | 4143 . . . 4165 | − | Transgenic insert |
| TI-33 | ATTA | FnCas12a | 4194 . . . 4216 | + | Transgenic insert |
| TI-34 | ATTA | FnCas12a | 4360 . . . 4382 | + | Transgenic insert |
| TI-35 | ATTA | FnCas12a | 4383 . . . 4405 | − | Transgenic insert |
| TI-36 | ATTA | FnCas12a | 4445 . . . 4467 | − | Transgenic insert |
| TI-37 | ATTC | FnCas12a | 1132 . . . 1154 | + | Transgenic insert |
| TI-38 | ATTC | FnCas12a | 1138 . . . 1160 | + | Transgenic insert |
| TI-39 | ATTC | FnCas12a | 1921 . . . 1943 | − | Transgenic insert |
| TI-40 | ATTC | FnCas12a | 2263 . . . 2285 | + | Transgenic insert |
| TI-41 | ATTC | FnCas12a | 2301 . . . 2323 | + | Transgenic insert |
| TI-42 | ATTC | FnCas12a | 2317 . . . 2339 | − | Transgenic insert |
| TI-43 | ATTC | FnCas12a | 2352 . . . 2374 | + | Transgenic insert |
| TI-44 | ATTC | FnCas12a | 2374 . . . 2396 | − | Transgenic insert |
| TI-45 | ATTC | FnCas12a | 2416 . . . 2438 | + | Transgenic insert |
| TI-46 | ATTC | FnCas12a | 2469 . . . 2491 | + | Transgenic insert |
| TI-47 | ATTC | FnCas12a | 2590 . . . 2612 | + | Transgenic insert |
| TI-48 | ATTC | FnCas12a | 2653 . . . 2675 | − | Transgenic insert |
| TI-49 | ATTC | FnCas12a | 2664 . . . 2686 | − | Transgenic insert |
| TI-50 | ATTC | FnCas12a | 2693 . . . 2715 | + | Transgenic insert |
| TI-51 | ATTC | FnCas12a | 2759 . . . 2781 | − | Transgenic insert |
| TI-52 | ATTC | FnCas12a | 2981 . . . 3003 | − | Transgenic insert |
| TI-53 | ATTC | FnCas12a | 3141 . . . 3163 | − | Transgenic insert |
| TI-54 | ATTC | FnCas12a | 3331 . . . 3353 | + | Transgenic insert |
| TI-55 | ATTC | FnCas12a | 3516 . . . 3538 | − | Transgenic insert |
| TI-56 | ATTC | FnCas12a | 3621 . . . 3643 | + | Transgenic insert |
| TI-57 | ATTC | FnCas12a | 4351 . . . 4373 | + | Transgenic insert |
| TI-58 | ATTC | FnCas12a | 4437 . . . 4459 | + | Transgenic insert |
| TI-59 | ATTG | FnCas12a | 1092 . . . 1114 | − | Transgenic insert |
| TI-60 | ATTG | FnCas12a | 1494 . . . 1516 | − | Transgenic insert |
| TI-61 | ATTG | FnCas12a | 1921 . . . 1943 | + | Transgenic insert |
| TI-62 | ATTG | FnCas12a | 2044 . . . 2066 | + | Transgenic insert |
| TI-63 | ATTG | FnCas12a | 2127 . . . 2149 | − | Transgenic insert |
| TI-64 | ATTG | FnCas12a | 2151 . . . 2173 | − | Transgenic insert |
| TI-65 | ATTG | FnCas12a | 2377 . . . 2399 | + | Transgenic insert |
| TI-66 | ATTG | FnCas12a | 2499 . . . 2521 | + | Transgenic insert |
| TI-67 | ATTG | FnCas12a | 2503 . . . 2525 | + | Transgenic insert |
| TI-68 | ATTG | FnCas12a | 2561 . . . 2583 | − | Transgenic insert |
| TI-69 | ATTG | FnCas12a | 2669 . . . 2691 | − | Transgenic insert |
| TI-70 | ATTG | FnCas12a | 2702 . . . 2724 | + | Transgenic insert |
| TI-71 | ATTG | FnCas12a | 2735 . . . 2757 | − | Transgenic insert |
| TI-72 | ATTG | FnCas12a | 2829 . . . 2851 | + | Transgenic insert |
| TI-73 | ATTG | FnCas12a | 2844 . . . 2866 | + | Transgenic insert |
| TI-74 | ATTG | FnCas12a | 2852 . . . 2874 | + | Transgenic insert |
| TI-75 | ATTG | FnCas12a | 3022 . . . 3044 | + | Transgenic insert |
| TI-76 | ATTG | FnCas12a | 3042 . . . 3064 | + | Transgenic insert |
| TI-77 | ATTG | FnCas12a | 3475 . . . 3497 | − | Transgenic insert |
| TI-78 | ATTG | FnCas12a | 3504 . . . 3526 | + | Transgenic insert |
| TI-79 | ATTG | FnCas12a | 3564 . . . 3586 | − | Transgenic insert |
| TI-80 | ATTG | FnCas12a | 3931 . . . 3953 | + | Transgenic insert |
| TI-81 | ATTG | FnCas12a | 4481 . . . 4503 | + | Transgenic insert |
| TI-82 | ATTG | FnCas12a | 4516 . . . 4538 | + | Transgenic insert |
| TI-83 | CTTA | FnCas12a | 1187 . . . 1209 | + | Transgenic insert |
| TI-84 | CTTA | FnCas12a | 1261 . . . 1283 | + | Transgenic insert |
| TI-85 | CTTA | FnCas12a | 1938 . . . 1960 | + | Transgenic insert |
| TI-86 | CTTA | FnCas12a | 2295 . . . 2317 | − | Transgenic insert |
| TI-87 | CTTA | FnCas12a | 2437 . . . 2459 | + | Transgenic insert |
| TI-88 | CTTA | FnCas12a | 2461 . . . 2483 | + | Transgenic insert |
| TI-89 | CTTA | FnCas12a | 2488 . . . 2510 | + | Transgenic insert |
| TI-90 | CTTA | FnCas12a | 2777 . . . 2799 | + | Transgenic insert |
| TI-91 | CTTA | FnCas12a | 2782 . . . 2804 | + | Transgenic insert |
| TI-92 | CTTA | FnCas12a | 2763 . . . 2785 | − | Transgenic insert |
| TI-93 | CTTA | FnCas12a | 2937 . . . 2959 | + | Transgenic insert |
| TI-94 | CTTA | FnCas12a | 3216 . . . 3238 | + | Transgenic insert |
| TI-95 | CTTA | FnCas12a | 3431 . . . 3453 | − | Transgenic insert |
| TI-96 | CTTA | FnCas12a | 3760 . . . 3782 | + | Transgenic insert |
| TI-97 | CTTA | FnCas12a | 3917 . . . 3939 | − | Transgenic insert |
| TI-98 | CTTA | FnCas12a | 3974 . . . 3996 | − | Transgenic insert |
| TI-99 | CTTA | FnCas12a | 4127 . . . 4149 | − | Transgenic insert |
| TI-100 | CTTA | FnCas12a | 4470 . . . 4492 | + | Transgenic insert |
| TI-101 | CTTC | FnCas12a | 1207 . . . 1229 | + | Transgenic insert |
| TI-102 | CTTC | FnCas12a | 1202 . . . 1224 | − | Transgenic insert |
| TI-103 | CTTC | FnCas12a | 1258 . . . 1280 | + | Transgenic insert |
| TI-104 | CTTC | FnCas12a | 1565 . . . 1587 | − | Transgenic insert |
| TI-105 | CTTC | FnCas12a | 1608 . . . 1630 | + | Transgenic insert |
| TI-106 | CTTC | FnCas12a | 1766 . . . 1788 | + | Transgenic insert |
| TI-107 | CTTC | FnCas12a | 1870 . . . 1892 | − | Transgenic insert |
| TI-108 | CTTC | FnCas12a | 2134 . . . 2156 | + | Transgenic insert |
| TI-109 | CTTC | FnCas12a | 2186 . . . 2208 | − | Transgenic insert |
| TI-110 | CTTC | FnCas12a | 2190 . . . 2212 | − | Transgenic insert |
| TI-111 | CTTC | FnCas12a | 2217 . . . 2239 | − | Transgenic insert |
| TI-112 | CTTC | FnCas12a | 2255 . . . 2277 | + | Transgenic insert |
| TI-113 | CTTC | FnCas12a | 2293 . . . 2315 | + | Transgenic insert |
| TI-114 | CTTC | FnCas12a | 2426 . . . 2448 | + | Transgenic insert |
| TI-115 | CTTC | FnCas12a | 2743 . . . 2765 | + | Transgenic insert |
| TI-116 | CTTC | FnCas12a | 2830 . . . 2852 | − | Transgenic insert |
| TI-117 | CTTC | FnCas12a | 2882 . . . 2904 | − | Transgenic insert |
| TI-118 | CTTC | FnCas12a | 2926 . . . 2948 | + | Transgenic insert |
| TI-119 | CTTC | FnCas12a | 3063 . . . 3085 | + | Transgenic insert |
| TI-120 | CTTC | FnCas12a | 3295 . . . 3317 | − | Transgenic insert |
| TI-121 | CTTC | FnCas12a | 3345 . . . 3367 | + | Transgenic insert |
| TI-122 | CTTC | FnCas12a | 3352 . . . 3374 | + | Transgenic insert |
| TI-123 | CTTC | FnCas12a | 3344 . . . 3366 | − | Transgenic insert |
| TI-124 | CTTC | FnCas12a | 3383 . . . 3405 | + | Transgenic insert |
| TI-125 | CTTC | FnCas12a | 3508 . . . 3530 | + | Transgenic insert |
| TI-126 | CTTC | FnCas12a | 3503 . . . 3525 | − | Transgenic insert |
| TI-127 | CTTC | FnCas12a | 3509 . . . 3531 | − | Transgenic insert |
| TI-128 | CTTC | FnCas12a | 3635 . . . 3657 | + | Transgenic insert |
| TI-129 | CTTC | FnCas12a | 3704 . . . 3726 | + | Transgenic insert |
| TI-130 | CTTC | FnCas12a | 3701 . . . 3723 | − | Transgenic insert |
| TI-131 | CTTC | FnCas12a | 3710 . . . 3732 | − | Transgenic insert |
| TI-132 | CTTC | FnCas12a | 3828 . . . 3850 | + | Transgenic insert |
| TI-133 | CTTC | FnCas12a | 3920 . . . 3942 | − | Transgenic insert |
| TI-134 | CTTC | FnCas12a | 3956 . . . 3978 | − | Transgenic insert |
| TI-135 | CTTC | FnCas12a | 4013 . . . 4035 | + | Transgenic insert |
| TI-136 | CTTC | FnCas12a | 4451 . . . 4473 | + | Transgenic insert |
| TI-137 | CTTG | FnCas12a | 1218 . . . 1240 | − | Transgenic insert |
| TI-138 | CTTG | FnCas12a | 1251 . . . 1273 | + | Transgenic insert |
| TI-139 | CTTG | FnCas12a | 1876 . . . 1898 | − | Transgenic insert |
| TI-140 | CTTG | FnCas12a | 1917 . . . 1939 | − | Transgenic insert |
| TI-141 | CTTG | FnCas12a | 2086 . . . 2108 | + | Transgenic insert |
| TI-142 | CTTG | FnCas12a | 2194 . . . 2216 | − | Transgenic insert |
| TI-143 | CTTG | FnCas12a | 2241 . . . 2263 | + | Transgenic insert |
| TI-144 | CTTG | FnCas12a | 2511 . . . 2533 | + | Transgenic insert |
| TI-145 | CTTG | FnCas12a | 2698 . . . 2720 | − | Transgenic insert |
| TI-146 | CTTG | FnCas12a | 2996 . . . 3018 | + | Transgenic insert |
| TI-147 | CTTG | FnCas12a | 3165 . . . 3187 | + | Transgenic insert |
| TI-148 | CTTG | FnCas12a | 3429 . . . 3451 | + | Transgenic insert |
| TI-149 | CTTG | FnCas12a | 3464 . . . 3486 | + | Transgenic insert |
| TI-150 | CTTG | FnCas12a | 3626 . . . 3648 | − | Transgenic insert |
| TI-151 | CTTG | FnCas12a | 3648 . . . 3670 | + | Transgenic insert |
| TI-152 | CTTG | FnCas12a | 3635 . . . 3657 | − | Transgenic insert |
| TI-153 | CTTG | FnCas12a | 3667 . . . 3689 | − | Transgenic insert |
| TI-154 | CTTG | FnCas12a | 3697 . . . 3719 | − | Transgenic insert |
| TI-155 | CTTG | FnCas12a | 3830 . . . 3852 | − | Transgenic insert |
| TI-156 | CTTG | FnCas12a | 3953 . . . 3975 | − | Transgenic insert |
| TI-157 | CTTG | FnCas12a | 4096 . . . 4118 | + | Transgenic insert |
| TI-158 | CTTG | FnCas12a | 4159 . . . 4181 | + | Transgenic insert |
| TI-159 | CTTG | FnCas12a | 4273 . . . 4295 | + | Transgenic insert |
| TI-160 | GTTA | FnCas12a | 1050 . . . 1072 | + | Transgenic insert |
| TI-161 | GTTA | FnCas12a | 1232 . . . 1254 | + | Transgenic insert |
| TI-162 | GTTA | FnCas12a | 1444 . . . 1466 | + | Transgenic insert |
| TI-163 | GTTA | FnCas12a | 1708 . . . 1730 | − | Transgenic insert |
| TI-164 | GTTA | FnCas12a | 1776 . . . 1798 | − | Transgenic insert |
| TI-165 | GTTA | FnCas12a | 1780 . . . 1802 | − | Transgenic insert |
| TI-166 | GTTA | FnCas12a | 1987 . . . 2009 | + | Transgenic insert |
| TI-167 | GTTA | FnCas12a | 2484 . . . 2506 | + | Transgenic insert |
| TI-168 | GTTA | FnCas12a | 2578 . . . 2600 | + | Transgenic insert |
| TI-169 | GTTA | FnCas12a | 2861 . . . 2883 | + | Transgenic insert |
| TI-170 | GTTA | FnCas12a | 3625 . . . 3647 | + | Transgenic insert |
| TI-171 | GTTA | FnCas12a | 3942 . . . 3964 | + | Transgenic insert |
| TI-172 | GTTA | FnCas12a | 3995 . . . 4017 | + | Transgenic insert |
| TI-173 | GTTA | FnCas12a | 4080 . . . 4102 | + | Transgenic insert |
| TI-174 | GTTA | FnCas12a | 4162 . . . 4184 | + | Transgenic insert |
| TI-175 | GTTA | FnCas12a | 4191 . . . 4213 | + | Transgenic insert |
| TI-176 | GTTA | FnCas12a | 4206 . . . 4228 | + | Transgenic insert |
| TI-177 | GTTA | FnCas12a | 4189 . . . 4211 | − | Transgenic insert |
| TI-178 | GTTA | FnCas12a | 4252 . . . 4274 | + | Transgenic insert |
| TI-179 | GTTA | FnCas12a | 4304 . . . 4326 | + | Transgenic insert |
| TI-180 | GTTA | FnCas12a | 4335 . . . 4357 | − | Transgenic insert |
| TI-181 | GTTA | FnCas12a | 4359 . . . 4381 | − | Transgenic insert |
| TI-182 | GTTC | FnCas12a | 1241 . . . 1263 | + | Transgenic insert |
| TI-183 | GTTC | FnCas12a | 1248 . . . 1270 | + | Transgenic insert |
| TI-184 | GTTC | FnCas12a | 1947 . . . 1969 | − | Transgenic insert |
| TI-185 | GTTC | FnCas12a | 2017 . . . 2039 | + | Transgenic insert |
| TI-186 | GTTC | FnCas12a | 2062 . . . 2084 | + | Transgenic insert |
| TI-187 | GTTC | FnCas12a | 2187 . . . 2209 | + | Transgenic insert |
| TI-188 | GTTC | FnCas12a | 2338 . . . 2360 | + | Transgenic insert |
| TI-189 | GTTC | FnCas12a | 2353 . . . 2375 | − | Transgenic insert |
| TI-190 | GTTC | FnCas12a | 2519 . . . 2541 | + | Transgenic insert |
| TI-191 | GTTC | FnCas12a | 2546 . . . 2568 | + | Transgenic insert |
| TI-192 | GTTC | FnCas12a | 2525 . . . 2547 | − | Transgenic insert |
| TI-193 | GTTC | FnCas12a | 2584 . . . 2606 | + | Transgenic insert |
| TI-194 | GTTC | FnCas12a | 2637 . . . 2659 | + | Transgenic insert |
| TI-195 | GTTC | FnCas12a | 3097 . . . 3119 | + | Transgenic insert |
| TI-196 | GTTC | FnCas12a | 3110 . . . 3132 | + | Transgenic insert |
| TI-197 | GTTC | FnCas12a | 3150 . . . 3172 | + | Transgenic insert |
| TI-198 | GTTC | FnCas12a | 3283 . . . 3305 | − | Transgenic insert |
| TI-199 | GTTC | FnCas12a | 3379 . . . 3401 | + | Transgenic insert |
| TI-200 | GTTC | FnCas12a | 3443 . . . 3465 | + | Transgenic insert |
| TI-201 | GTTC | FnCas12a | 3554 . . . 3576 | − | Transgenic insert |
| TI-202 | GTTC | FnCas12a | 3656 . . . 3678 | + | Transgenic insert |
| TI-203 | GTTC | FnCas12a | 3656 . . . 3678 | − | Transgenic insert |
| TI-204 | GTTC | FnCas12a | 3701 . . . 3723 | + | Transgenic insert |
| TI-205 | GTTC | FnCas12a | 3815 . . . 3837 | + | Transgenic insert |
| TI-206 | GTTC | FnCas12a | 3950 . . . 3972 | + | Transgenic insert |
| TI-207 | GTTC | FnCas12a | 4072 . . . 4094 | − | Transgenic insert |
| TI-208 | GTTC | FnCas12a | 4172 . . . 4194 | − | Transgenic insert |
| TI-209 | GTTC | FnCas12a | 4493 . . . 4515 | + | Transgenic insert |
| TI-210 | GTTG | FnCas12a | 1556 . . . 1578 | − | Transgenic insert |
| TI-211 | GTTG | FnCas12a | 1800 . . . 1822 | − | Transgenic insert |
| TI-212 | GTTG | FnCas12a | 1996 . . . 2018 | + | Transgenic insert |
| TI-213 | GTTG | FnCas12a | 2081 . . . 2103 | + | Transgenic insert |
| TI-214 | GTTG | FnCas12a | 2113 . . . 2135 | + | Transgenic insert |
| TI-215 | GTTG | FnCas12a | 2242 . . . 2264 | − | Transgenic insert |
| TI-216 | GTTG | FnCas12a | 2280 . . . 2302 | − | Transgenic insert |
| TI-217 | GTTG | FnCas12a | 2507 . . . 2529 | + | Transgenic insert |
| TI-218 | GTTG | FnCas12a | 2514 . . . 2536 | + | Transgenic insert |
| TI-219 | GTTG | FnCas12a | 2648 . . . 2670 | + | Transgenic insert |
| TI-220 | GTTG | FnCas12a | 2748 . . . 2770 | + | Transgenic insert |
| TI-221 | GTTG | FnCas12a | 2880 . . . 2902 | + | Transgenic insert |
| TI-222 | GTTG | FnCas12a | 2970 . . . 2992 | + | Transgenic insert |
| TI-223 | GTTG | FnCas12a | 3005 . . . 3027 | + | Transgenic insert |
| TI-224 | GTTG | FnCas12a | 3049 . . . 3071 | + | Transgenic insert |
| TI-225 | GTTG | FnCas12a | 3087 . . . 3109 | + | Transgenic insert |
| TI-226 | GTTG | FnCas12a | 3141 . . . 3163 | + | Transgenic insert |
| TI-227 | GTTG | FnCas12a | 3158 . . . 3180 | + | Transgenic insert |
| TI-228 | GTTG | FnCas12a | 3321 . . . 3343 | − | Transgenic insert |
| TI-229 | GTTG | FnCas12a | 3447 . . . 3469 | + | Transgenic insert |
| TI-230 | GTTG | FnCas12a | 3611 . . . 3633 | − | Transgenic insert |
| TI-231 | GTTG | FnCas12a | 3659 . . . 3681 | − | Transgenic insert |
| TI-232 | GTTG | FnCas12a | 3803 . . . 3825 | + | Transgenic insert |
| TI-233 | GTTG | FnCas12a | 4111 . . . 4133 | + | Transgenic insert |
| TI-234 | GTTG | FnCas12a | 4112 . . . 4134 | − | Transgenic insert |
| TI-235 | GTTG | FnCas12a | 4150 . . . 4172 | + | Transgenic insert |
| TI-236 | GTTG | FnCas12a | 4258 . . . 4280 | + | Transgenic insert |
| TI-237 | GTTG | FnCas12a | 4428 . . . 4450 | + | Transgenic insert |
| TI-238 | GTTG | FnCas12a | 4443 . . . 4465 | + | Transgenic insert |
| TI-239 | GTTG | FnCas12a | 4447 . . . 4469 | + | Transgenic insert |
| TI-240 | TATA | LbCas12a- | 1028 . . . 1050 | − | Transgenic insert |
| | | TAT | | | |
| TI-241 | TATA | LbCas12a- | 1055 . . . 1077 | + | Transgenic insert |
| | | TAT | | | |
| TI-242 | TATA | LbCas12a- | 1080 . . . 1102 | − | Transgenic insert |
| | | TAT | | | |
| TI-243 | TATA | LbCas12a- | 1107 . . . 1129 | + | Transgenic insert |
| | | TAT | | | |
| TI-244 | TATA | LbCas12a- | 1185 . . . 1207 | − | Transgenic insert |
| | | TAT | | | |
| TI-245 | TATA | LbCas12a- | 1212 . . . 1234 | + | Transgenic insert |
| | | TAT | | | |
| TI-246 | TATA | LbCas12a- | 1197 . . . 1219 | − | Transgenic insert |
| | | TAT | | | |
| TI-247 | TATA | LbCas12a- | 1224 . . . 1246 | + | Transgenic insert |
| | | TAT | | | |
| TI-248 | TATA | LbCas12a- | 1207 . . . 1229 | − | Transgenic insert |
| | | TAT | | | |
| TI-249 | TATA | LbCas12a- | 1234 . . . 1256 | + | Transgenic insert |
| | | TAT | | | |
| TI-250 | TATA | LbCas12a- | 1362 . . . 1384 | − | Transgenic insert |
| | | TAT | | | |
| TI-251 | TATA | LbCas12a- | 1419 . . . 1441 | − | Transgenic insert |
| | | TAT | | | |
| TI-252 | TATA | LbCas12a- | 1446 . . . 1468 | + | Transgenic insert |
| | | TAT | | | |
| TI-253 | TATA | LbCas12a- | 1681 . . . 1703 | + | Transgenic insert |
| | | TAT | | | |
| TI-254 | TATA | LbCas12a- | 1774 . . . 1796 | − | Transgenic insert |
| | | TAT | | | |
| TI-255 | TATA | LbCas12a- | 1801 . . . 1823 | + | Transgenic insert |
| | | TAT | | | |
| TI-256 | TATA | LbCas12a- | 1903 . . . 1925 | − | Transgenic insert |
| | | TAT | | | |
| TI-257 | TATA | LbCas12a- | 1930 . . . 1952 | + | Transgenic insert |
| | | TAT | | | |
| TI-258 | TATA | LbCas12a- | 2545 . . . 2567 | − | Transgenic insert |
| | | TAT | | | |
| TI-259 | TATA | LbCas12a- | 2547 . . . 2569 | 1 | Transgenic insert |
| | | TAT | | | |
| TI-260 | TATA | LbCas12a- | 2572 . . . 2594 | + | Transgenic insert |
| | | TAT | | | |
| TI-261 | TATA | LbCas12a- | 2574 . . . 2596 | + | Transgenic insert |
| | | TAT | | | |
| TI-262 | TATA | LbCas12a- | 2586 . . . 2608 | − | Transgenic insert |
| | | TAT | | | |
| TI-263 | TATA | LbCas12a- | 2838 . . . 2860 | + | Transgenic insert |
| | | TAT | | | |
| TI-264 | TATA | LbCas12a- | 2985 . . . 3007 | + | Transgenic insert |
| | | TAT | | | |
| TI-265 | TATA | LbCas12a- | 2973 . . . 2995 | − | Transgenic insert |
| | | TAT | | | |
| TI-266 | TATA | LbCas12a- | 3000 . . . 3022 | + | Transgenic insert |
| | | TAT | | | |
| TI-267 | TATA | LbCas12a- | 3027 . . . 3049 | − | Transgenic insert |
| | | TAT | | | |
| TI-268 | TATA | LbCas12a- | 3054 . . . 3076 | + | Transgenic insert |
| | | TAT | | | |
| TI-269 | TATC | LbCas12a- | 1049 . . . 1071 | − | Transgenic insert |
| | | TAT | | | |
| TI-270 | TATC | LbCas12a- | 1410 . . . 1432 | + | Transgenic insert |
| | | TAT | | | |
| TI-271 | TATC | LbCas12a- | 1433 . . . 1455 | + | Transgenic insert |
| | | TAT | | | |
| TI-272 | TATC | LbCas12a- | 1577 . . . 1599 | − | Transgenic insert |
| | | TAT | | | |
| TI-273 | TATC | LbCas12a- | 1666 . . . 1688 | − | Transgenic insert |
| | | TAT | | | |
| TI-274 | TATC | LbCas12a- | 1989 . . . 2011 | + | Transgenic insert |
| | | TAT | | | |
| TI-275 | TATC | LbCas12a- | 2095 . . . 2117 | − | Transgenic insert |
| | | TAT | | | |
| TI-276 | TATC | LbCas12a- | 2451 . . . 2473 | + | Transgenic insert |
| | | TAT | | | |
| TI-277 | TATC | LbCas12a- | 2540 . . . 2562 | I | Transgenic insert |
| | | TAT | | | |
| TI-278 | TATC | LbCas12a- | 2642 . . . 2664 | + | Transgenic insert |
| | | TAT | | | |
| TI-279 | TATC | LbCas12a- | 2673 . . . 2695 | + | Transgenic insert |
| | | TAT | | | |
| TI-280 | TATC | LbCas12a- | 2713 . . . 2735 | + | Transgenic insert |
| | | TAT | | | |
| TI-281 | TATC | LbCas12a- | 2774 . . . 2796 | + | Transgenic insert |
| | | TAT | | | |
| TI-282 | TATC | LbCas12a- | 2779 . . . 2801 | + | Transgenic insert |
| | | TAT | | | |
| TI-283 | TATC | LbCas12a- | 2771 . . . 2793 | − | Transgenic insert |
| | | TAT | | | |
| TI-284 | TATC | LbCas12a- | 2886 . . . 2908 | − | Transgenic insert |
| | | TAT | | | |
| TI-285 | TATC | LbCas12a- | 3814 . . . 3836 | − | Transgenic insert |
| | | TAT | | | |
| TI-286 | TATC | LbCas12a- | 3973 . . . 3995 | + | Transgenic insert |
| | | TAT | | | |
| TI-287 | TATC | LbCas12a- | 4048 . . . 4070 | + | Transgenic insert |
| | | TAT | | | |
| TI-288 | TATC | LbCas12a- | 4075 . . . 4097 | + | Transgenic insert |
| | | TAT | | | |
| TI-289 | TATC | LbCas12a- | 4103 . . . 4125 | + | Transgenic insert |
| | | TAT | | | |
| TI-290 | TATC | LbCas12a- | 4256 . . . 4278 | − | Transgenic insert |
| | | TAT | | | |
| TI-291 | TATC | LbCas12a- | 4306 . . . 4328 | + | Transgenic insert |
| | | TAT | | | |
| TI-292 | TATG | LbCas12a- | 1219 . . . 1241 | + | Transgenic insert |
| | | TAT | | | |
| TI-293 | TATG | LbCas12a- | 1623 . . . 1645 | − | Transgenic insert |
| | | TAT | | | |
| TI-294 | TATG | LbCas12a- | 2330 . . . 2352 | + | Transgenic insert |
| | | TAT | | | |
| TI-295 | TATG | LbCas12a- | 2333 . . . 2355 | − | Transgenic insert |
| | | TAT | | | |
| TI-296 | TATG | LbCas12a- | 2369 . . . 2391 | + | Transgenic insert |
| | | TAT | | | |
| TI-297 | TATG | LbCas12a- | 2387 . . . 2409 | + | Transgenic insert |
| | | TAT | | | |
| TI-298 | TATG | LbCas12a- | 2439 . . . 2461 | + | Transgenic insert |
| | | TAT | | | |
| TI-299 | TATG | LbCas12a- | 2429 . . . 2451 | − | Transgenic insert |
| | | TAT | | | |
| TI-300 | TATG | LbCas12a- | 2649 . . . 2671 | − | Transgenic insert |
| | | TAT | | | |
| TI-301 | TATG | LbCas12a- | 2684 . . . 2706 | + | Transgenic insert |
| | | TAT | | | |
| TI-302 | TATG | LbCas12a- | 2871 . . . 2893 | − | Transgenic insert |
| | | TAT | | | |
| TI-303 | TATG | LbCas12a- | 2892 . . . 2914 | − | Transgenic insert |
| | | TAT | | | |
| TI-304 | TATG | LbCas12a- | 2915 . . . 2937 | + | Transgenic insert |
| | | TAT | | | |
| TI-305 | TATG | LbCas12a- | 2967 . . . 2989 | + | Transgenic insert |
| | | TAT | | | |
| TI-306 | TATG | LbCas12a- | 3002 . . . 3024 | + | Transgenic insert |
| | | TAT | | | |
| TI-307 | TATG | LbCas12a- | 3012 . . . 3034 | − | Transgenic insert |
| | | TAT | | | |
| TI-308 | TATG | LbCas12a- | 3056 . . . 3078 | + | Transgenic insert |
| | | TAT | | | |
| TI-309 | TATG | LbCas12a- | 3094 . . . 3116 | + | Transgenic insert |
| | | TAT | | | |
| TI-310 | TATG | LbCas12a- | 3208 . . . 3230 | + | Transgenic insert |
| | | TAT | | | |
| TI-311 | TATG | LbCas12a- | 3484 . . . 3506 | − | Transgenic insert |
| | | TAT | | | |
| TI-312 | TATG | LbCas12a- | 3762 . . . 3784 | + | Transgenic insert |
| | | TAT | | | |
| TI-313 | TATG | LbCas12a- | 4053 . . . 4075 | + | Transgenic insert |
| | | TAT | | | |
| TI-314 | TATG | LbCas12a- | 4082 . . . 4104 | + | Transgenic insert |
| | | TAT | | | |
| TI-315 | TATG | LbCas12a- | 4122 . . . 4144 | + | Transgenic insert |
| | | TAT | | | |
| TI-316 | TATG | LbCas12a- | 4130 . . . 4152 | + | Transgenic insert |
| | | TAT | | | |
| TI-317 | TATG | LbCas12a- | 4164 . . . 4186 | + | Transgenic insert |
| | | TAT | | | |
| TI-318 | TATG | LbCas12a- | 4196 . . . 4218 | + | Transgenic insert |
| | | TAT | | | |
| TI-319 | TATG | LbCas12a- | 4208 . . . 4230 | + | Transgenic insert |
| | | TAT | | | |
| TI-320 | TCCA | LbCas12a- | 1243 . . . 1265 | + | Transgenic insert |
| | | TYC | | | |
| TI-321 | TCCA | LbCas12a- | 1397 . . . 1419 | − | Transgenic insert |
| | | TYC | | | |
| TI-322 | TCCA | LbCas12a- | 1435 . . . 1457 | + | Transgenic insert |
| | | TYC | | | |
| TI-323 | TCCA | LbCas12a- | 1455 . . . 1477 | + | Transgenic insert |
| | | TYC | | | |
| TI-324 | TCCA | LbCas12a- | 1499 . . . 1521 | + | Transgenic insert |
| | | TYC | | | |
| TI-325 | TCCA | LbCas12a- | 1562 . . . 1584 | + | Transgenic insert |
| | | TYC | | | |
| TI-326 | TCCA | LbCas12a- | 1563 . . . 1585 | | Transgenic insert |
| | | TYC | | | |
| TI-327 | TCCA | LbCas12a- | 1610 . . . 1632 | + | Transgenic insert |
| | | TYC | | | |
| TI-328 | TCCA | LbCas12a- | 1633 . . . 1655 | + | Transgenic insert |
| | | TYC | | | |
| TI-329 | TCCA | LbCas12a- | 1758 . . . 1780 | − | Transgenic insert |
| | | TYC | | | |
| TI-330 | TCCA | LbCas12a- | 1971 . . . 1993 | − | Transgenic insert |
| | | TYC | | | |
| TI-331 | TCCA | LbCas12a- | 2009 . . . 2031 | + | Transgenic insert |
| | | TYC | | | |
| TI-332 | TCCA | LbCas12a- | 2019 . . . 2041 | + | Transgenic insert |
| | | TYC | | | |
| TI-333 | TCCA | LbCas12a- | 2014 . . . 2036 | − | Transgenic insert |
| | | TYC | | | |
| TI-334 | TCCA | LbCas12a- | 2130 . . . 2152 | + | Transgenic insert |
| | | TYC | | | |
| TI-335 | TCCA | LbCas12a- | 2157 . . . 2179 | + | Transgenic insert |
| | | TYC | | | |
| TI-336 | TCCA | LbCas12a- | 2386 . . . 2408 | − | Transgenic insert |
| | | TYC | | | |
| TI-337 | TCCA | LbCas12a- | 2521 . . . 2543 | + | Transgenic insert |
| | | TYC | | | |
| TI-338 | TCCA | LbCas12a- | 2523 . . . 2545 | − | Transgenic insert |
| | | TYC | | | |
| TI-339 | TCCA | LbCas12a- | 2586 . . . 2608 | + | Transgenic insert |
| | | TYC | | | |
| TI-340 | TCCA | LbCas12a- | 3046 . . . 3068 | − | Transgenic insert |
| | | TYC | | | |
| TI-341 | TCCA | LbCas12a- | 3050 . . . 3072 | − | Transgenic insert |
| | | TYC | | | |
| TI-342 | TCCA | LbCas12a- | 3152 . . . 3174 | + | Transgenic insert |
| | | TYC | | | |
| TI-343 | TCCA | LbCas12a- | 3646 . . . 3668 | − | Transgenic insert |
| | | TYC | | | |
| TI-344 | TCCA | LbCas12a- | 3822 . . . 3844 | − | Transgenic insert |
| | | TYC | | | |
| TI-345 | TCCA | LbCas12a- | 3868 . . . 3890 | + | Transgenic insert |
| | | TYC | | | |
| TI-346 | TCCA | LbCas12a- | 4415 . . . 4437 | + | Transgenic insert |
| | | TYC | | | |
| TI-347 | TCCA | LbCas12a- | 4439 . . . 4461 | + | Transgenic insert |
| | | TYC | | | |
| TI-348 | TCCA | LbCas12a- | 4453 . . . 4475 | + | Transgenic insert |
| | | TYC | | | |
| TI-349 | TCCC | LbCas12a- | 2150 . . . 2172 | + | Transgenic insert |
| | | TYC | | | |
| TI-350 | TCCC | LbCas12a- | 2175 . . . 2197 | + | Transgenic insert |
| | | TYC | | | |
| TI-351 | TCCC | LbCas12a- | 2354 . . . 2376 | + | Transgenic insert |
| | | TYC | | | |
| TI-352 | TCCC | LbCas12a- | 2428 . . . 2450 | + | Transgenic insert |
| | | TYC | | | |
| TI-353 | TCCC | LbCas12a- | 3457 . . . 3479 | − | Transgenic insert |
| | | TYC | | | |
| TI-354 | TCCC | LbCas12a- | 3544 . . . 3566 | − | Transgenic insert |
| | | TYC | | | |
| TI-355 | TCCC | LbCas12a- | 3746 . . . 3768 | + | Transgenic insert |
| | | TYC | | | |
| TI-356 | TCCC | LbCas12a- | 3907 . . . 3929 | − | Transgenic insert |
| | | TYC | | | |
| TI-357 | TCCC | LbCas12a- | 4173 . . . 4195 | + | Transgenic insert |
| | | TYC | | | |
| TI-358 | TCCC | LbCas12a- | 4331 . . . 4353 | + | Transgenic insert |
| | | TYC | | | |
| TI-359 | TCCC | LbCas12a- | 4316 . . . 4338 | − | Transgenic insert |
| | | TYC | | | |
| TI-360 | TCCC | LbCas12a- | 4495 . . . 4517 | + | Transgenic insert |
| | | TYC | | | |
| TI-361 | TCCC | LbCas12a- | 4525 . . . 4547 | + | Transgenic insert |
| | | TYC | | | |
| TI-362 | TCCG | LbCas12a- | 1042 . . . 1064 | + | Transgenic insert |
| | | TYC | | | |
| TI-363 | TCCG | LbCas12a- | 1096 . . . 1118 | + | Transgenic insert |
| | | TYC | | | |
| TI-364 | TCCG | LbCas12a- | 1114 . . . 1136 | − | Transgenic insert |
| | | TYC | | | |
| TI-365 | TCCG | LbCas12a- | 1925 . . . 1947 | − | Transgenic insert |
| | | TYC | | | |
| TI-366 | TCCG | LbCas12a- | 1945 . . . 1967 | + | Transgenic insert |
| | | TYC | | | |
| TI-367 | TCCG | LbCas12a- | 2110 . . . 2132 | + | Transgenic insert |
| | | TYC | | | |
| TI-368 | TCCG | LbCas12a- | 2136 . . . 2158 | + | Transgenic insert |
| | | TYC | | | |
| TI-369 | TCCG | LbCas12a- | 2143 . . . 2165 | + | Transgenic insert |
| | | TYC | | | |
| TI-370 | TCCG | LbCas12a- | 2191 . . . 2213 | + | Transgenic insert |
| | | TYC | | | |
| TI-371 | TCCG | LbCas12a- | 2886 . . . 2908 | + | Transgenic insert |
| | | TYC | | | |
| TI-372 | TCCG | LbCas12a- | 3286 . . . 3308 | − | Transgenic insert |
| | | TYC | | | |
| TI-373 | TCCG | LbCas12a- | 3365 . . . 3387 | + | Transgenic insert |
| | | TYC | | | |
| TI-374 | TCCG | LbCas12a- | 3451 . . . 3473 | + | Transgenic insert |
| | | TYC | | | |
| TI-375 | TCCG | LbCas12a- | 3855 . . . 3877 | − | Transgenic insert |
| | | TYC | | | |
| TI-376 | TCCG | LbCas12a- | 3901 . . . 3923 | − | Transgenic insert |
| | | TYC | | | |
| TI-377 | TCCG | LbCas12a- | 3928 . . . 3950 | − | Transgenic insert |
| | | TYC | | | |
| TI-378 | TCCG | LbCas12a- | 4024 . . . 4046 | + | Transgenic insert |
| | | TYC | | | |
| TI-379 | TCCG | LbCas12a- | 4077 . . . 4099 | + | Transgenic insert |
| | | TYC | | | |
| TI-380 | TTCA | LbCas12a- | 1035 . . . 1057 | − | Transgenic insert |
| | | TYC | | | |
| TI-381 | TTCA | LbCas12a- | 1259 . . . 1281 | − | Transgenic insert |
| | | TYC | | | |
| TI-382 | TTCA | LbCas12a- | 1639 . . . 1661 | + | Transgenic insert |
| | | TYC | | | |
| TI-383 | TTCA | LbCas12a- | 1869 . . . 1891 | − | Transgenic insert |
| | | TYC | | | |
| TI-384 | TTCA | LbCas12a- | 2216 . . . 2238 | − | Transgenic insert |
| | | TYC | | | |
| TI-385 | TTCA | LbCas12a- | 2352 . . . 2374 | − | Transgenic insert |
| | | TYC | | | |
| TI-386 | TTCA | LbCas12a- | 2591 . . . 2613 | + | Transgenic insert |
| | | TYC | | | |
| TI-387 | TTCA | LbCas12a- | 2663 . . . 2685 | − | Transgenic insert |
| | | TYC | | | |
| TI-388 | TTCA | LbCas12a- | 2694 . . . 2716 | + | Transgenic insert |
| | | TYC | | | |
| TI-389 | TTCA | LbCas12a- | 2980 . . . 3002 | − | Transgenic insert |
| | | TYC | | | |
| TI-390 | TTCA | LbCas12a- | 3111 . . . 3133 | + | Transgenic insert |
| | | TYC | | | |
| TI-391 | TTCA | LbCas12a- | 3140 . . . 3162 | − | Transgenic insert |
| | | TYC | | | |
| TI-392 | TTCA | LbCas12a- | 3346 . . . 3368 | + | Transgenic insert |
| | | TYC | | | |
| TI-393 | TTCA | LbCas12a- | 3384 . . . 3406 | + | Transgenic insert |
| | | TYC | | | |
| TI-394 | TTCA | LbCas12a- | 3509 . . . 3531 | + | Transgenic insert |
| | | TYC | | | |
| TI-395 | TTCA | LbCas12a- | 3502 . . . 3524 | − | Transgenic insert |
| | | TYC | | | |
| TI-396 | TTCA | LbCas12a- | 3553 . . . 3575 | − | Transgenic insert |
| | | TYC | | | |
| TI-397 | TTCA | LbCas12a- | 3636 . . . 3658 | + | Transgenic insert |
| | | TYC | | | |
| TI-398 | TTCA | LbCas12a- | 3657 . . . 3679 | + | Transgenic insert |
| | | TYC | | | |
| TI-399 | TTCA | LbCas12a- | 3655 . . . 3677 | − | Transgenic insert |
| | | TYC | | | |
| TI-400 | TTCA | LbCas12a- | 4071 . . . 4093 | − | Transgenic insert |
| | | TYC | | | |
| TI-401 | TTCA | LbCas12a- | 4171 . . . 4193 | − | Transgenic insert |
| | | TYC | | | |
| TI-402 | TTCC | LbCas12a- | 1115 . . . 1137 | − | Transgenic insert |
| | | TYC | | | |
| TI-403 | TTCC | LbCas12a- | 1242 . . . 1264 | + | Transgenic insert |
| | | TYC | | | |
| TI-404 | TTCC | LbCas12a- | 1454 . . . 1476 | + | Transgenic insert |
| | | TYC | | | |
| TI-405 | TTCC | LbCas12a- | 1462 . . . 1484 | − | Transgenic insert |
| | | TYC | | | |
| TI-406 | TTCC | LbCas12a- | 1498 . . . 1520 | + | Transgenic insert |
| | | TYC | | | |
| TI-407 | TTCC | LbCas12a- | 1561 . . . 1583 | + | Transgenic insert |
| | | TYC | | | |
| TI-408 | TTCC | LbCas12a- | 1567 . . . 1589 | + | Transgenic insert |
| | | TYC | | | |
| TI-409 | TTCC | LbCas12a- | 1564 . . . 1586 | − | Transgenic insert |
| | | TYC | | | |
| TI-410 | TTCC | LbCas12a- | 1609 . . . 1631 | + | Transgenic insert |
| | | TYC | | | |
| TI-411 | TTCC | LbCas12a- | 1632 . . . 1654 | + | Transgenic insert |
| | | TYC | | | |
| TI-412 | TTCC | LbCas12a- | 1920 . . . 1942 | − | Transgenic insert |
| | | TYC | | | |
| TI-413 | TTCC | LbCas12a- | 1946 . . . 1968 | − | Transgenic insert |
| | | TYC | | | |
| TI-414 | TTCC | LbCas12a- | 2018 . . . 2040 | + | Transgenic insert |
| | | TYC | | | |
| TI-415 | TTCC | LbCas12a- | 2135 . . . 2157 | + | Transgenic insert |
| | | TYC | | | |
| TI-416 | TTCC | LbCas12a- | 2149 . . . 2171 | + | Transgenic insert |
| | | TYC | | | |
| TI-417 | TTCC | LbCas12a- | 2189 . . . 2211 | − | Transgenic insert |
| | | TYC | | | |
| TI-418 | TTCC | LbCas12a- | 2256 . . . 2278 | + | Transgenic insert |
| | | TYC | | | |
| TI-419 | TTCC | LbCas12a- | 2294 . . . 2316 | + | Transgenic insert |
| | | TYC | | | |
| TI-420 | TTCC | LbCas12a- | 2353 . . . 2375 | + | Transgenic insert |
| | | TYC | | | |
| TI-421 | TTCC | LbCas12a- | 2427 . . . 2449 | + | Transgenic insert |
| | | TYC | | | |
| TI-422 | TTCC | LbCas12a- | 2520 . . . 2542 | + | Transgenic insert |
| | | TYC | | | |
| TI-423 | TTCC | LbCas12a- | 2524 . . . 2546 | − | Transgenic insert |
| | | TYC | | | |
| TI-424 | TTCC | LbCas12a- | 2585 . . . 2607 | + | Transgenic insert |
| | | TYC | | | |
| TI-425 | TTCC | LbCas12a- | 2638 . . . 2660 | + | Transgenic insert |
| | | TYC | | | |
| TI-426 | TTCC | LbCas12a- | 3064 . . . 3086 | + | Transgenic insert |
| | | TYC | | | |
| TI-427 | TTCC | LbCas12a- | 3151 . . . 3173 | + | Transgenic insert |
| | | TYC | | | |
| TI-428 | TTCC | LbCas12a- | 3282 . . . 3304 | − | Transgenic insert |
| | | TYC | | | |
| TI-429 | TTCC | LbCas12a- | 3364 . . . 3386 | + | Transgenic insert |
| | | TYC | | | |
| TI-430 | TTCC | LbCas12a- | 3380 . . . 3402 | + | Transgenic insert |
| | | TYC | | | |
| TI-431 | TTCC | LbCas12a- | 3700 . . . 3722 | − | Transgenic insert |
| | | TYC | | | |
| TI-432 | TTCC | LbCas12a- | 4014 . . . 4036 | + | Transgenic insert |
| | | TYC | | | |
| TI-433 | TTCC | LbCas12a- | 4438 . . . 4460 | + | Transgenic insert |
| | | TYC | | | |
| TI-434 | TTCC | LbCas12a- | 4452 . . . 4474 | + | Transgenic insert |
| | | TYC | | | |
| TI-435 | TTCC | LbCas12a- | 4494 . . . 4516 | + | Transgenic insert |
| | | TYC | | | |
| TI-436 | TTCC | LbCas12a- | 4524 . . . 4546 | + | Transgenic insert |
| | | TYC | | | |
| TI-437 | TTCG | LbCas12a- | 1086 . . . 1108 | − | Transgenic insert |
| | | TYC | | | |
| TI-438 | TTCG | LbCas12a- | 1139 . . . 1161 | + | Transgenic insert |
| | | TYC | | | |
| TI-439 | TTCG | LbCas12a- | 1154 . . . 1176 | + | Transgenic insert |
| | | TYC | | | |
| TI-440 | TTCG | LbCas12a- | 1208 . . . 1230 | + | Transgenic insert |
| | | TYC | | | |
| TI-441 | TTCG | LbCas12a- | 1201 . . . 1223 | − | Transgenic insert |
| | | TYC | | | |
| TI-442 | TTCG | LbCas12a- | 1915 . . . 1937 | + | Transgenic insert |
| | | TYC | | | |
| TI-443 | TTCG | LbCas12a- | 2063 . . . 2085 | + | Transgenic insert |
| | | TYC | | | |
| TI-444 | TTCG | LbCas12a- | 2185 . . . 2207 | − | Transgenic insert |
| | | TYC | | | |
| TI-445 | TTCG | LbCas12a- | 2264 . . . 2286 | + | Transgenic insert |
| | | TYC | | | |
| TI-446 | TTCG | LbCas12a- | 2302 . . . 2324 | + | Transgenic insert |
| | | TYC | | | |
| TI-447 | TTCG | LbCas12a- | 2316 . . . 2338 | − | Transgenic insert |
| | | TYC | | | |
| TI-448 | TTCG | LbCas12a- | 2373 . . . 2395 | − | Transgenic insert |
| | | TYC | | | |
| TI-449 | TTCG | LbCas12a- | 2398 . . . 2420 | + | Transgenic insert |
| | | TYC | | | |
| TI-450 | TTCG | LbCas12a- | 3294 . . . 3316 | − | Transgenic insert |
| | | TYC | | | |
| TI-451 | TTCG | LbCas12a- | 3332 . . . 3354 | + | Transgenic insert |
| | | TYC | | | |
| TI-452 | TTCG | LbCas12a- | 3343 . . . 3365 | − | Transgenic insert |
| | | TYC | | | |
| TI-453 | TTCG | LbCas12a- | 3444 . . . 3466 | + | Transgenic insert |
| | | TYC | | | |
| TI-454 | TTCG | LbCas12a- | 3515 . . . 3537 | − | Transgenic insert |
| | | TYC | | | |
| TI-455 | TTCG | LbCas12a- | 3622 . . . 3644 | + | Transgenic insert |
| | | TYC | | | |
| TI-456 | TTCG | LbCas12a- | 3741 . . . 3763 | − | Transgenic insert |
| | | TYC | | | |
| TI-457 | TTCG | LbCas12a- | 3816 . . . 3838 | + | Transgenic insert |
| | | TYC | | | |
| TI-458 | TTCG | LbCas12a- | 3829 . . . 3851 | + | Transgenic insert |
| | | TYC | | | |
| TI-459 | TTCG | LbCas12a- | 3951 . . . 3973 | + | Transgenic insert |
| | | TYC | | | |
| TI-460 | TTCG | LbCas12a- | 4352 . . . 4374 | + | Transgenic insert |
| | | TYC | | | |
| TI-461 | TTTA | LbCas12a | 1030 . . . 1052 | − | Transgenic insert |
| TI-462 | TTTC | LbCas12a | 1036 . . . 1058 | − | Transgenic insert |
| TI-463 | TTTG | LbCas12a | 1073 . . . 1095 | + | Transgenic insert |
| TI-464 | TTTA | LbCas12a | 1051 . . . 1073 | − | Transgenic insert |
| TI-465 | TTTG | LbCas12a | 1059 . . . 1081 | − | Transgenic insert |
| TI-466 | TTTA | LbCas12a | 1105 . . . 1127 | + | Transgenic insert |
| TI-467 | TTTC | LbCas12a | 1087 . . . 1109 | − | Transgenic insert |
| TI-468 | TTTA | LbCas12a | 1095 . . . 1117 | − | Transgenic insert |
| TI-469 | TTTG | LbCas12a | 1099 . . . 1121 | − | Transgenic insert |
| TI-470 | TTTC | LbCas12a | 1116 . . . 1138 | − | Transgenic insert |
| TI-471 | TTTA | LbCas12a | 1149 . . . 1171 | + | Transgenic insert |
| TI-472 | TTTC | LbCas12a | 1153 . . . 1175 | + | Transgenic insert |
| TI-473 | TTTG | LbCas12a | 1150 . . . 1172 | − | Transgenic insert |
| TI-474 | TTTA | LbCas12a | 1245 . . . 1267 | − | Transgenic insert |
| TI-475 | TTTA | LbCas12a | 1253 . . . 1275 | − | Transgenic insert |
| TI-476 | TTTC | LbCas12a | 1260 . . . 1282 | − | Transgenic insert |
| TI-477 | TTTG | LbCas12a | 1314 . . . 1336 | + | Transgenic insert |
| TI-478 | TTTA | LbCas12a | 1368 . . . 1390 | + | Transgenic insert |
| TI-479 | TTTA | LbCas12a | 1364 . . . 1386 | − | Transgenic insert |
| TI-480 | TTTA | LbCas12a | 1372 . . . 1394 | − | Transgenic insert |
| TI-481 | TTTA | LbCas12a | 1408 . . . 1430 | + | Transgenic insert |
| TI-482 | TTTC | LbCas12a | 1412 . . . 1434 | − | Transgenic insert |
| TI-483 | TTTA | LbCas12a | 1421 . . . 1443 | − | Transgenic insert |
| TI-484 | TTTC | LbCas12a | 1453 . . . 1475 | + | Transgenic insert |
| TI-485 | TTTG | LbCas12a | 1432 . . . 1454 | − | Transgenic insert |
| TI-486 | TTTA | LbCas12a | 1465 . . . 1487 | + | Transgenic insert |
| TI-487 | TTTA | LbCas12a | 1451 . . . 1473 | − | Transgenic insert |
| TI-488 | TTTC | LbCas12a | 1463 . . . 1485 | − | Transgenic insert |
| TI-489 | TTTC | LbCas12a | 1497 . . . 1519 | + | Transgenic insert |
| TI-490 | TTTC | LbCas12a | 1560 . . . 1582 | + | Transgenic insert |
| TI-491 | TTTC | LbCas12a | 1566 . . . 1588 | + | Transgenic insert |
| TI-492 | TTTG | LbCas12a | 1588 . . . 1610 | + | Transgenic insert |
| TI-493 | TTTA | LbCas12a | 1598 . . . 1620 | + | Transgenic insert |
| TI-494 | TTTA | LbCas12a | 1594 . . . 1616 | − | Transgenic insert |
| TI-495 | TTTA | LbCas12a | 1619 . . . 1641 | + | Transgenic insert |
| TI-496 | TTTA | LbCas12a | 1627 . . . 1649 | + | Transgenic insert |
| TI-497 | TTTC | LbCas12a | 1631 . . . 1653 | + | Transgenic insert |
| TI-498 | TTTC | LbCas12a | 1638 . . . 1660 | + | Transgenic insert |
| TI-499 | TTTC | LbCas12a | 1667 . . . 1689 | + | Transgenic insert |
| TI-500 | TTTA | LbCas12a | 1679 . . . 1701 | + | Transgenic insert |
| TI-501 | TTTA | LbCas12a | 1706 . . . 1728 | + | Transgenic insert |
| TI-502 | TTTA | LbCas12a | 1712 . . . 1734 | − | Transgenic insert |
| TI-503 | TTTA | LbCas12a | 1769 . . . 1791 | − | Transgenic insert |
| TI-504 | TTTA | LbCas12a | 1794 . . . 1816 | + | Transgenic insert |
| TI-505 | TTTA | LbCas12a | 1869 . . . 1891 | + | Transgenic insert |
| TI-506 | TTTC | LbCas12a | 1914 . . . 1936 | + | Transgenic insert |
| TI-507 | TTTA | LbCas12a | 1913 . . . 1935 | − | Transgenic insert |
| TI-508 | TTTG | LbCas12a | 1932 . . . 1954 | − | Transgenic insert |
| TI-509 | TTTG | LbCas12a | 1984 . . . 2006 | − | Transgenic insert |
| TI-510 | TTTC | LbCas12a | 2075 . . . 2097 | + | Transgenic insert |
| TI-511 | TTTA | LbCas12a | 2097 . . . 2119 | − | Transgenic insert |
| TI-512 | TTTC | LbCas12a | 2148 . . . 2170 | + | Transgenic insert |
| TI-513 | TTTA | LbCas12a | 2278 . . . 2300 | + | Transgenic insert |
| TI-514 | TTTG | LbCas12a | 2316 . . . 2338 | + | Transgenic insert |
| TI-515 | TTTC | LbCas12a | 2397 . . . 2419 | + | Transgenic insert |
| TI-516 | TTTG | LbCas12a | 2411 . . . 2433 | + | Transgenic insert |
| TI-517 | TTTA | LbCas12a | 2433 . . . 2455 | + | Transgenic insert |
| TI-518 | TTTG | LbCas12a | 2444 . . . 2466 | + | Transgenic insert |
| TI-519 | TTTG | LbCas12a | 2495 . . . 2517 | + | Transgenic insert |
| TI-520 | TTTG | LbCas12a | 2498 . . . 2520 | − | Transgenic insert |
| TI-521 | TTTC | LbCas12a | 2541 . . . 2563 | + | Transgenic insert |
| TI-522 | TTTG | LbCas12a | 2566 . . . 2588 | − | Transgenic insert |
| TI-523 | TTTA | LbCas12a | 2588 . . . 2610 | − | Transgenic insert |
| TI-524 | TTTG | LbCas12a | 2688 . . . 2710 | + | Transgenic insert |
| TI-525 | TTTA | LbCas12a | 2693 . . . 2715 | − | Transgenic insert |
| TI-526 | TTTA | LbCas12a | 2718 . . . 2740 | + | Transgenic insert |
| TI-527 | TTTA | LbCas12a | 2704 . . . 2726 | − | Transgenic insert |
| TI-528 | TTTA | LbCas12a | 2819 . . . 2841 | + | Transgenic insert |
| TI-529 | TTTG | LbCas12a | 2822 . . . 2844 | − | Transgenic insert |
| TI-530 | TTTA | LbCas12a | 2836 . . . 2858 | − | Transgenic insert |
| TI-531 | TTTG | LbCas12a | 2890 . . . 2912 | + | Transgenic insert |
| TI-532 | TTTA | LbCas12a | 2894 . . . 2916 | − | Transgenic insert |
| TI-533 | TTTA | LbCas12a | 2906 . . . 2928 | − | Transgenic insert |
| TI-534 | TTTA | LbCas12a | 2965 . . . 2987 | + | Transgenic insert |
| TI-535 | TTTC | LbCas12a | 2979 . . . 3001 | + | Transgenic insert |
| TI-536 | TTTG | LbCas12a | 2989 . . . 3011 | + | Transgenic insert |
| TI-537 | TTTG | LbCas12a | 2999 . . . 3021 | − | Transgenic insert |
| TI-538 | TTTG | LbCas12a | 3089 . . . 3111 | − | Transgenic insert |
| TI-539 | TTTG | LbCas12a | 3138 . . . 3160 | + | Transgenic insert |
| TI-540 | TTTC | LbCas12a | 3195 . . . 3217 | + | Transgenic insert |
| TI-541 | TTTG | LbCas12a | 3230 . . . 3252 | + | Transgenic insert |
| TI-542 | TTTA | LbCas12a | 3288 . . . 3310 | + | Transgenic insert |
| TI-543 | TTTA | LbCas12a | 3328 . . . 3350 | + | Transgenic insert |
| TI-544 | TTTC | LbCas12a | 3337 . . . 3359 | + | Transgenic insert |
| TI-545 | TTTC | LbCas12a | 3363 . . . 3385 | + | Transgenic insert |
| TI-546 | TTTG | LbCas12a | 3359 . . . 3381 | − | Transgenic insert |
| TI-547 | TTTG | LbCas12a | 3368 . . . 3390 | − | Transgenic insert |
| TI-548 | TTTA | LbCas12a | 3373 . . . 3395 | − | Transgenic insert |
| TI-549 | TTTC | LbCas12a | 3640 . . . 3662 | − | Transgenic insert |
| TI-550 | TTTA | LbCas12a | 3685 . . . 3707 | − | Transgenic insert |
| TI-551 | TTTG | LbCas12a | 3727 . . . 3749 | − | Transgenic insert |
| TI-552 | TTTC | LbCas12a | 3742 . . . 3764 | − | Transgenic insert |
| TI-553 | TTTG | LbCas12a | 3940 . . . 3962 | − | Transgenic insert |
| TI-554 | TTTG | LbCas12a | 3963 . . . 3985 | + | Transgenic insert |
| TI-555 | TTTG | LbCas12a | 4092 . . . 4114 | + | Transgenic insert |
| TI-556 | TTTA | LbCas12a | 4135 . . . 4157 | + | Transgenic insert |
| TI-557 | TTTA | LbCas12a | 4168 . . . 4190 | + | Transgenic insert |
| TI-558 | TTTG | LbCas12a | 4180 . . . 4202 | + | Transgenic insert |
| TI-559 | TTTA | LbCas12a | 4237 . . . 4259 | + | Transgenic insert |
| TI-560 | TTTC | LbCas12a | 4242 . . . 4264 | + | Transgenic insert |
| TI-561 | TTTA | LbCas12a | 4258 . . . 4280 | − | Transgenic insert |
| TI-562 | TTTC | LbCas12a | 4290 . . . 4312 | + | Transgenic insert |
| TI-563 | TTTG | LbCas12a | 4315 . . . 4337 | + | Transgenic insert |
| TI-564 | TTTG | LbCas12a | 4327 . . . 4349 | + | Transgenic insert |
| TI-565 | TTTC | LbCas12a | 4373 . . . 4395 | + | Transgenic insert |
| TI-566 | TTTC | LbCas12a | 4397 . . . 4419 | + | Transgenic insert |
| TI-567 | TTTG | LbCas12a | 4460 . . . 4482 | + | Transgenic insert |
| TI-568 | TTTG | LbCas12a | 4459 . . . 4481 | − | Transgenic insert |
| TI-569 | TTTG | LbCas12a | 4503 . . . 4525 | + | Transgenic insert |
| TI-570 | TTTC | LbCas12a | 4523 . . . 4545 | + | Transgenic insert |
| TI-571 | TTTA | LbCas12a | 4512 . . . 4534 | − | Transgenic insert |
| 3J-1 | CTTC | FnCas12a | 4531 . . . 4553 | + | 3′ Junction |
| 3J-2 | GTTG | FnCas12a | 4536 . . . 4558 | − | 3′ Junction |
| 3J-3 | TATC | LbCas12a- | 4544 . . . 4566 | + | 3′ Junction |
| | | TAT | | | |
| 3J-4 | TTCA | LbCas12a- | 4532 . . . 4554 | + | 3′ Junction |
| | | TYC | | | |
| 3J-5 | TTTA | LbCas12a | 4537 . . . 4559 | + | 3′ Junction |
| 3J-6 | TTTG | LbCas12a | 4552 . . . 4574 | + | 3′ Junction |
| 3J-7 | TATC | LbCas12a- | 4552 . . . 4574 | − | 3′ Flanking genomic |
| | | TAT | | | DNA |
| 3F-1 | ATTA | FnCas12a | 4657 . . . 4679 | + | 3′ Flanking genomic |
| | | | | | DNA |
| 3F-2 | ATTA | FnCas12a | 4740 . . . 4762 | + | 3′ Flanking genomic |
| | | | | | DNA |
| 3F-3 | ATTA | FnCas12a | 4793 . . . 4815 | + | 3′ Flanking genomic |
| | | | | | DNA |
| 3F-4 | ATTA | FnCas12a | 4854 . . . 4876 | + | 3′ Flanking genomic |
| | | | | | DNA |
| 3F-5 | ATTA | FnCas12a | 4888 . . . 4910 | − | 3′ Flanking genomic |
| | | | | | DNA |
| 3F-6 | ATTA | FnCas12a | 5041 . . . 5063 | + | 3′ Flanking genomic |
| | | | | | DNA |
| 3F-7 | ATTA | FnCas12a | 5054 . . . 5076 | + | 3′ Flanking genomic |
| | | | | | DNA |
| 3F-8 | ATTA | FnCas12a | 5107 . . . 5129 | + | 3′ Flanking genomic |
| | | | | | DNA |
| 3F-9 | ATTA | FnCas12a | 5098 . . . 5120 | − | 3′ Flanking genomic |
| | | | | | DNA |
| 3F-10 | ATTA | FnCas12a | 5116 . . . 5138 | − | 3′ Flanking genomic |
| | | | | | DNA |
| 3F-11 | ATTA | FnCas12a | 5130 . . . 5152 | − | 3′ Flanking genomic |
| | | | | | DNA |
| 3F-12 | ATTA | FnCas12a | 5155 . . . 5177 | + | 3′ Flanking genomic |
| | | | | | DNA |
| 3F-13 | ATTA | FnCas12a | 5162 . . . 5184 | + | 3′ Flanking genomic |
| | | | | | DNA |
| 3F-14 | ATTA | FnCas12a | 5145 . . . 5167 | − | 3′ Flanking genomic |
| | | | | | DNA |
| 3F-15 | ATTA | FnCas12a | 5278 . . . 5300 | + | 3′ Flanking genomic |
| | | | | | DNA |
| 3F-16 | ATTC | FnCas12a | 4654 . . . 4676 | − | 3′ Flanking genomic |
| | | | | | DNA |
| 3F-17 | ATTC | FnCas12a | 4718 . . . 4740 | + | 3′ Flanking genomic |
| | | | | | DNA |
| 3F-18 | ATTC | FnCas12a | 4743 . . . 4765 | − | 3′ Flanking genomic |
| | | | | | DNA |
| 3F-19 | ATTC | FnCas12a | 4812 . . . 4834 | + | 3′ Flanking genomic |
| | | | | | DNA |
| 3F-20 | ATTC | FnCas12a | 5167 . . . 5189 | + | 3′ Flanking genomic |
| | | | | | DNA |
| 3F-21 | ATTC | FnCas12a | 5251 . . . 5273 | + | 3′ Flanking genomic |
| | | | | | DNA |
| 3F-22 | ATTG | FnCas12a | 4601 . . . 4623 | + | 3′ Flanking genomic |
| | | | | | DNA |
| 3F-23 | ATTG | FnCas12a | 4586 . . . 4608 | − | 3′ Flanking genomic |
| | | | | | DNA |
| 3F-24 | ATTG | FnCas12a | 4635 . . . 4657 | − | 3′ Flanking genomic |
| | | | | | DNA |
| 3F-25 | ATTG | FnCas12a | 4689 . . . 4711 | − | 3′ Flanking genomic |
| | | | | | DNA |
| 3F-26 | ATTG | FnCas12a | 4751 . . . 4773 | − | 3′ Flanking genomic |
| | | | | | DNA |
| 3F-27 | ATTG | FnCas12a | 4771 . . . 4793 | − | 3′ Flanking genomic |
| | | | | | DNA |
| 3F-28 | ATTG | FnCas12a | 4813 . . . 4835 | − | 3′ Flanking genomic |
| | | | | | DNA |
| 3F-29 | ATTG | FnCas12a | 4899 . . . 4921 | + | 3′ Flanking genomic |
| | | | | | DNA |
| 3F-30 | ATTG | FnCas12a | 5112 . . . 5134 | + | 3′ Flanking genomic |
| | | | | | DNA |
| 3F-31 | ATTG | FnCas12a | 5118 . . . 5140 | + | 3′ Flanking genomic |
| | | | | | DNA |
| 3F-32 | ATTG | FnCas12a | 5126 . . . 5148 | − | 3′ Flanking genomic |
| | | | | | DNA |
| 3F-33 | ATTG | FnCas12a | 5243 . . . 5265 | − | 3′ Flanking genomic |
| | | | | | DNA |
| 3F-34 | ATTG | FnCas12a | 5259 . . . 5281 | − | 3′ Flanking genomic |
| | | | | | DNA |
| 3F-35 | ATTG | FnCas12a | 5335 . . . 5357 | + | 3′ Flanking genomic |
| | | | | | DNA |
| 3F-36 | ATTG | FnCas12a | 5362 . . . 5384 | + | 3′ Flanking genomic |
| | | | | | DNA |
| 3F-37 | ATTG | FnCas12a | 5467 . . . 5489 | + | 3′ Flanking genomic |
| | | | | | DNA |
| 3F-38 | ATTG | FnCas12a | 5506 . . . 5528 | + | 3′ Flanking genomic |
| | | | | | DNA |
| 3F-39 | ATTG | FnCas12a | 5489 . . . 5511 | − | 3′ Flanking genomic |
| | | | | | DNA |
| 3F-40 | CTTA | FnCas12a | 4575 . . . 4597 | + | 3′ Flanking genomic |
| | | | | | DNA |
| 3F-41 | CTTA | FnCas12a | 4642 . . . 4664 | − | 3′ Flanking genomic |
| | | | | | DNA |
| 3F-42 | CTTA | FnCas12a | 4809 . . . 4831 | + | 3′ Flanking genomic |
| | | | | | DNA |
| 3F-43 | CTTA | FnCas12a | 4829 . . . 4851 | − | 3′ Flanking genomic |
| | | | | | DNA |
| 3F-44 | CTTA | FnCas12a | 5048 . . . 5070 | − | 3′ Flanking genomic |
| | | | | | DNA |
| 3F-45 | CTTA | FnCas12a | 5086 . . . 5108 | + | 3′ Flanking genomic |
| | | | | | DNA |
| 3F-46 | CTTA | FnCas12a | 5101 . . . 5123 | + | 3′ Flanking genomic |
| | | | | | DNA |
| 3F-47 | CTTA | FnCas12a | 5170 . . . 5192 | + | 3′ Flanking genomic |
| | | | | | DNA |
| 3F-48 | CTTA | FnCas12a | 5254 . . . 5276 | + | 3′ Flanking genomic |
| | | | | | DNA |
| 3F-49 | CTTC | FnCas12a | 4882 . . . 4904 | − | 3′ Flanking genomic |
| | | | | | DNA |
| 3F-50 | CTTC | FnCas12a | 5233 . . . 5255 | + | 3′ Flanking genomic |
| | | | | | DNA |
| 3F-51 | CTTC | FnCas12a | 5353 . . . 5375 | + | 3′ Flanking genomic |
| | | | | | DNA |
| 3F-52 | CTTC | FnCas12a | 5481 . . . 5503 | + | 3′ Flanking genomic |
| | | | | | DNA |
| 3F-53 | CTTG | FnCas12a | 4614 . . . 4636 | − | 3′ Flanking genomic |
| | | | | | DNA |
| 3F-54 | CTTG | FnCas12a | 4728 . . . 4750 | + | 3′ Flanking genomic |
| | | | | | DNA |
| 3F-55 | CTTG | FnCas12a | 4740 . . . 4762 | − | 3′ Flanking genomic |
| | | | | | DNA |
| 3F-56 | CTTG | FnCas12a | 5341 . . . 5363 | + | 3′ Flanking genomic |
| | | | | | DNA |
| 3F-57 | CTTG | FnCas12a | 5399 . . . 5421 | + | 3′ Flanking genomic |
| | | | | | DNA |
| 3F-58 | CTTG | FnCas12a | 5428 . . . 5450 | + | 3′ Flanking genomic |
| | | | | | DNA |
| 3F-59 | CTTG | FnCas12a | 5408 . . . 5430 | − | 3′ Flanking genomic |
| | | | | | DNA |
| 3F-60 | GTTA | FnCas12a | 4562 . . . 4584 | − | 3′ Flanking genomic |
| | | | | | DNA |
| 3F-61 | GTTA | FnCas12a | 4589 . . . 4611 | − | 3′ Flanking genomic |
| | | | | | DNA |
| 3F-62 | GTTA | FnCas12a | 4598 . . . 4620 | − | 3′ Flanking genomic |
| | | | | | DNA |
| 3F-63 | GTTA | FnCas12a | 4632 . . . 4654 | − | 3′ Flanking genomic |
| | | | | | DNA |
| 3F-64 | GTTA | FnCas12a | 5035 . . . 5057 | + | 3′ Flanking genomic |
| | | | | | DNA |
| 3F-65 | GTTA | FnCas12a | 5130 . . . 5152 | + | 3′ Flanking genomic |
| | | | | | DNA |
| 3F-66 | GTTC | FnCas12a | 4572 . . . 4594 | + | 3′ Flanking genomic |
| | | | | | DNA |
| 3F-67 | GTTC | FnCas12a | 4644 . . . 4666 | + | 3′ Flanking genomic |
| | | | | | DNA |
| 3F-68 | GTTC | FnCas12a | 4710 . . . 4732 | + | 3′ Flanking genomic |
| | | | | | DNA |
| 3F-69 | GTTC | FnCas12a | 4737 . . . 4759 | − | 3′ Flanking genomic |
| | | | | | DNA |
| 3F-70 | GTTC | FnCas12a | 5204 . . . 5226 | + | 3′ Flanking genomic |
| | | | | | DNA |
| 3F-71 | GTTC | FnCas12a | 5247 . . . 5269 | I | 3′ Flanking genomic |
| | | | | | DNA |
| 3F-72 | GTTC | FnCas12a | 5408 . . . 5430 | + | 3′ Flanking genomic |
| | | | | | DNA |
| 3F-73 | GTTC | FnCas12a | 5463 . . . 5485 | − | 3′ Flanking genomic |
| | | | | | DNA |
| 3F-74 | GTTG | FnCas12a | 4719 . . . 4741 | − | 3′ Flanking genomic |
| | | | | | DNA |
| 3F-75 | GTTG | FnCas12a | 5068 . . . 5090 | − | 3′ Flanking genomic |
| | | | | | DNA |
| 3F-76 | GTTG | FnCas12a | 5365 . . . 5387 | + | 3′ Flanking genomic |
| | | | | | DNA |
| 3F-77 | GTTG | FnCas12a | 5346 . . . 5368 | − | 3′ Flanking genomic |
| | | | | | DNA |
| 3F-78 | TATA | LbCas12a- | 4678 . . . 4700 | − | 3′ Flanking genomic |
| | | TAT | | | DNA |
| 3F-79 | TATA | LbCas12a- | 4705 . . . 4727 | + | 3′ Flanking genomic |
| | | TAT | | | DNA |
| 3F-80 | TATA | LbCas12a- | 4715 . . . 4737 | − | 3′ Flanking genomic |
| | | TAT | | | DNA |
| 3F-81 | TATA | LbCas12a- | 4742 . . . 4764 | + | 3′ Flanking genomic |
| | | TAT | | | DNA |
| 3F-82 | TATA | LbCas12a- | 4839 . . . 4861 | − | 3′ Flanking genomic |
| | | TAT | | | DNA |
| 3F-83 | TATA | LbCas12a- | 4866 . . . 4888 | + | 3′ Flanking genomic |
| | | TAT | | | DNA |
| 3F-84 | TATA | LbCas12a- | 4855 . . . 4877 | − | 3′ Flanking genomic |
| | | TAT | | | DNA |
| 3F-85 | TATA | LbCas12a- | 4882 . . . 4904 | + | 3′ Flanking genomic |
| | | TAT | | | DNA |
| 3F-86 | TATA | LbCas12a- | 4865 . . . 4887 | − | 3′ Flanking genomic |
| | | TAT | | | DNA |
| 3F-87 | TATA | LbCas12a- | 4867 . . . 4889 | − | 3′ Flanking genomic |
| | | TAT | | | DNA |
| 3F-88 | TATA | LbCas12a- | 4892 . . . 4914 | + | 3′ Flanking genomic |
| | | TAT | | | DNA |
| 3F-89 | TATA | LbCas12a- | 4894 . . . 4916 | + | 3′ Flanking genomic |
| | | TAT | | | DNA |
| 3F-90 | TATA | LbCas12a- | 4895 . . . 4917 | − | 3′ Flanking genomic |
| | | TAT | | | DNA |
| 3F-91 | TATA | LbCas12a- | 5114 . . . 5136 | − | 3′ Flanking genomic |
| | | TAT | | | DNA |
| 3F-92 | TATA | LbCas12a- | 5141 . . . 5163 | + | 3′ Flanking genomic |
| | | TAT | | | DNA |
| 3F-93 | TATA | LbCas12a- | 5137 . . . 5159 | − | 3′ Flanking genomic |
| | | TAT | | | DNA |
| 3F-94 | TATA | LbCas12a- | 5164 . . . 5186 | + | 3′ Flanking genomic |
| | | TAT | | | DNA |
| 3F-95 | TATA | LbCas12a- | 5280 . . . 5302 | − | 3′ Flanking genomic |
| | | TAT | | | DNA |
| 3F-96 | TATA | LbCas12a- | 5446 . . . 5468 | − | 3′ Flanking genomic |
| | | TAT | | | DNA |
| 3F-97 | TATA | LbCas12a- | 5448 . . . 5470 | − | 3′ Flanking genomic |
| | | TAT | | | DNA |
| 3F-98 | TATA | LbCas12a- | 5450 . . . 5472 | − | 3′ Flanking genomic |
| | | TAT | | | DNA |
| 3F-99 | TATA | LbCas12a- | 5473 . . . 5495 | + | 3′ Flanking genomic |
| | | TAT | | | DNA |
| 3F-100 | TATA | LbCas12a- | 5475 . . . 5497 | + | 3′ Flanking genomic |
| | | TAT | | | DNA |
| 3F-101 | TATA | LbCas12a- | 5477 . . . 5499 | + | 3′ Flanking genomic |
| | | TAT | | | DNA |
| 3F-102 | TATA | LbCas12a- | 5474 . . . 5496 | − | 3′ Flanking genomic |
| | | TAT | | | DNA |
| 3F-103 | TATA | LbCas12a- | 5476 . . . 5498 | − | 3′ Flanking genomic |
| | | TAT | | | DNA |
| 3F-104 | TATA | LbCas12a- | 5501 . . . 5523 | + | 3′ Flanking genomic |
| | | TAT | | | DNA |
| 3F-105 | TATA | LbCas12a- | 5503 . . . 5525 | + | 3′ Flanking genomic |
| | | TAT | | | DNA |
| 3F-106 | TATA | LbCas12a- | 5492 . . . 5514 | − | 3′ Flanking genomic |
| | | TAT | | | DNA |
| 3F-107 | TATA | LbCas12a- | 5519 . . . 5541 | + | 3′ Flanking genomic |
| | | TAT | | | DNA |
| 3F-108 | TATC | LbCas12a- | 4560 . . . 4582 | + | 3′ Flanking genomic |
| | | TAT | | | DNA |
| 3F-109 | TATC | LbCas12a- | 4691 . . . 4713 | + | 3′ Flanking genomic |
| | | TAT | | | DNA |
| 3F-110 | TATC | LbCas12a- | 4863 . . . 4885 | − | 3′ Flanking genomic |
| | | TAT | | | DNA |
| 3F-111 | TATC | LbCas12a- | 5280 . . . 5302 | + | 3′ Flanking genomic |
| | | TAT | | | DNA |
| 3F-112 | TATG | LbCas12a- | 4707 . . . 4729 | + | 3′ Flanking genomic |
| | | TAT | | | DNA |
| 3F-113 | TATG | LbCas12a- | 4850 . . . 4872 | + | 3′ Flanking genomic |
| | | TAT | | | DNA |
| 3F-114 | TATG | LbCas12a- | 4859 . . . 4881 | − | 3′ Flanking genomic |
| | | TAT | | | DNA |
| 3F-115 | TATG | LbCas12a- | 5088 . . . 5110 | + | 3′ Flanking genomic |
| | | TAT | | | DNA |
| 3F-116 | TATG | LbCas12a- | 5168 . . . 5190 | − | 3′ Flanking genomic |
| | | TAT | | | DNA |
| 3F-117 | TATG | LbCas12a- | 5221 . . . 5243 | + | 3′ Flanking genomic |
| | | TAT | | | DNA |
| 3F-118 | TATG | LbCas12a- | 5271 . . . 5293 | − | 3′ Flanking genomic |
| | | TAT | | | DNA |
| 3F-119 | TATG | LbCas12a- | 5328 . . . 5350 | + | 3′ Flanking genomic |
| | | TAT | | | DNA |
| 3F-120 | TATG | LbCas12a- | 5485 . . . 5507 | + | 3′ Flanking genomic |
| | | TAT | | | DNA |
| 3F-121 | TATG | LbCas12a- | 5472 . . . 5494 | − | 3′ Flanking genomic |
| | | TAT | | | DNA |
| 3F-122 | TCCA | LbCas12a- | 4652 . . . 4674 | − | 3′ Flanking genomic |
| | | TYC | | | DNA |
| 3F-123 | TCCA | LbCas12a- | 4730 . . . 4752 | − | 3′ Flanking genomic |
| | | TYC | | | DNA |
| 3F-124 | TCCA | LbCas12a- | 4874 . . . 4896 | − | 3′ Flanking genomic |
| | | TYC | | | DNA |
| 3F-125 | TCCA | LbCas12a- | 5340 . . . 5362 | − | 3′ Flanking genomic |
| | | TYC | | | DNA |
| 3F-126 | TCCA | LbCas12a- | 5388 . . . 5410 | + | 3′ Flanking genomic |
| | | TYC | | | DNA |
| 3F-127 | TCCA | LbCas12a- | 5392 . . . 5414 | + | 3′ Flanking genomic |
| | | TYC | | | DNA |
| 3F-128 | TCCA | LbCas12a- | 5497 . . . 5519 | + | 3′ Flanking genomic |
| | | TYC | | | DNA |
| 3F-129 | TCCA | LbCas12a- | 5514 . . . 5536 | + | 3′ Flanking genomic |
| | | TYC | | | DNA |
| 3F-130 | TCCA | LbCas12a- | 5521 . . . 5543 | − | 3′ Flanking genomic |
| | | TYC | | | DNA |
| 3F-131 | TCCG | LbCas12a- | 4693 . . . 4715 | + | 3′ Flanking genomic |
| | | TYC | | | DNA |
| 3F-132 | TTCA | LbCas12a- | 4576 . . . 4598 | − | 3′ Flanking genomic |
| | | TYC | | | DNA |
| 3F-133 | TTCA | LbCas12a- | 4645 . . . 4667 | + | 3′ Flanking genomic |
| | | TYC | | | DNA |
| 3F-134 | TTCA | LbCas12a- | 4675 . . . 4697 | + | 3′ Flanking genomic |
| | | TYC | | | DNA |
| 3F-135 | TTCA | LbCas12a- | 4711 . . . 4733 | + | 3′ Flanking genomic |
| | | TYC | | | DNA |
| 3F-136 | TTCA | LbCas12a- | 4719 . . . 4741 | + | 3′ Flanking genomic |
| | | TYC | | | DNA |
| 3F-137 | TTCA | LbCas12a- | 4776 . . . 4798 | + | 3′ Flanking genomic |
| | | TYC | | | DNA |
| 3F-138 | TTCA | LbCas12a- | 4813 . . . 4835 | + | 3′ Flanking genomic |
| | | TYC | | | DNA |
| 3F-139 | TTCA | LbCas12a- | 4881 . . . 4903 | − | 3′ Flanking genomic |
| | | TYC | | | DNA |
| 3F-140 | TTCA | LbCas12a- | 5205 . . . 5227 | + | 3′ Flanking genomic |
| | | TYC | | | DNA |
| 3F-141 | TTCA | LbCas12a- | 5246 . . . 5268 | − | 3′ Flanking genomic |
| | | TYC | | | DNA |
| 3F-142 | TTCA | LbCas12a- | 5462 . . . 5484 | − | 3′ Flanking genomic |
| | | TYC | | | DNA |
| 3F-143 | TTCC | LbCas12a- | 4653 . . . 4675 | − | 3′ Flanking genomic |
| | | TYC | | | DNA |
| 3F-144 | TTCC | LbCas12a- | 4731 . . . 4753 | − | 3′ Flanking genomic |
| | | TYC | | | DNA |
| 3F-145 | TTCG | LbCas12a- | 4698 . . . 4720 | + | 3′ Flanking genomic |
| | | TYC | | | DNA |
| 3F-146 | TTCG | LbCas12a- | 5354 . . . 5376 | + | 3′ Flanking genomic |
| | | TYC | | | DNA |
| 3F-147 | TTCG | LbCas12a- | 5409 . . . 5431 | + | 3′ Flanking genomic |
| | | TYC | | | DNA |
| 3F-148 | TTTA | LbCas12a | 4554 . . . 4576 | − | 3′ Flanking genomic |
| | | | | | DNA |
| 3F-149 | TTTC | LbCas12a | 4571 . . . 4593 | − | 3′ Flanking genomic |
| | | | | | DNA |
| 3F-150 | TTTC | LbCas12a | 4577 . . . 4599 | − | 3′ Flanking genomic |
| | | | | | DNA |
| 3F-151 | TTTA | LbCas12a | 4606 . . . 4628 | − | 3′ Flanking genomic |
| | | | | | DNA |
| 3F-152 | TTTA | LbCas12a | 4649 . . . 4671 | + | 3′ Flanking genomic |
| | | | | | DNA |
| 3F-153 | TTTA | LbCas12a | 4638 . . . 4660 | − | 3′ Flanking genomic |
| | | | | | DNA |
| 3F-154 | TTTC | LbCas12a | 4674 . . . 4696 | + | 3′ Flanking genomic |
| | | | | | DNA |
| 3F-155 | TTTA | LbCas12a | 4689 . . . 4711 | + | 3′ Flanking genomic |
| | | | | | DNA |
| 3F-156 | TTTC | LbCas12a | 4697 . . . 4719 | + | 3′ Flanking genomic |
| | | | | | DNA |
| 3F-157 | TTTA | LbCas12a | 4703 . . . 4725 | + | 3′ Flanking genomic |
| | | | | | DNA |
| 3F-158 | TTTA | LbCas12a | 4737 . . . 4759 | + | 3′ Flanking genomic |
| | | | | | DNA |
| 3F-159 | TTTA | LbCas12a | 4726 . . . 4748 | − | 3′ Flanking genomic |
| | | | | | DNA |
| 3F-160 | TTTC | LbCas12a | 4775 . . . 4797 | + | 3′ Flanking genomic |
| | | | | | DNA |
| 3F-161 | TTTC | LbCas12a | 4756 . . . 4778 | − | 3′ Flanking genomic |
| | | | | | DNA |
| 3F-162 | TTTG | LbCas12a | 4799 . . . 4821 | − | 3′ Flanking genomic |
| | | | | | DNA |
| 3F-163 | TTTA | LbCas12a | 4848 . . . 4870 | + | 3′ Flanking genomic |
| | | | | | DNA |
| 3F-164 | TTTG | LbCas12a | 4851 . . . 4873 | − | 3′ Flanking genomic |
| | | | | | DNA |
| 3F-165 | TTTA | LbCas12a | 5170 . . . 5192 | − | 3′ Flanking genomic |
| | | | | | DNA |
| 3F-166 | TTTG | LbCas12a | 5214 . . . 5236 | − | 3′ Flanking genomic |
| | | | | | DNA |
| 3F-167 | TTTA | LbCas12a | 5229 . . . 5251 | − | 3′ Flanking genomic |
| | | | | | DNA |
| 3F-168 | TTTA | LbCas12a | 5234 . . . 5256 | − | 3′ Flanking genomic |
| | | | | | DNA |
| 3F-169 | TTTA | LbCas12a | 5276 . . . 5298 | − | 3′ Flanking genomic |
| | | | | | DNA |
| 3F-170 | TTTA | LbCas12a | 5282 . . . 5304 | − | 3′ Flanking genomic |
| | | | | | DNA |
| 3F-171 | TTTG | LbCas12a | 5387 . . . 5409 | − | 3′ Flanking genomic |
| | | | | | DNA |
| 3F-172 | TTTG | LbCas12a | 5500 . . . 5522 | − | 3′ Flanking genomic |
| | | | | | DNA |
| 3F-173 | TTTA | LbCas12a | 5523 . . . 5545 | + | 3′ Flanking genomic |
| | | | | | DNA |

gRNAs may include a G leader sequence to facilitate transcription start from a poi III promoter positioned 5′ to a gRNA repeat, resulting in GAATTTCTACTAAGTGTAGAT (SEQ ID NO:38) for LbCas2a, or GTAATTTCTACTGTTGTAGAT (SEQ ID NO:39) for FnCas2a and an OgRRS sequence (as shown in the fourth column of Table 28). For Agrobacterium-based plant expression vectors, the cassette further comprises a poly-T transcript termination region (TTTTTTT). These gRNAs can be used to target the Cas12a nuclease to cut within both the OgRRS and CgRRS sequences. Illustrative examples of such gRNAs for FnCas12a expressed in stable plants are shown in Table 29, wherein the gRNA repeat of TAATTTCTACTGTTGTAGAT is underlined, and the poly-T transcript termination sequence of TTTTTTT is shown in italic font.

TABLE 29
Illustrative examples of gRNAs useful in targeting FnCas12a nuclease.

| gRNA | Sequence |
|---|---|
| gRNA_5F-63 | GTAATTTCTACTGTTGTAGATTCGTCAATAACTAACTAGTAAGTTTTTTTT |
| (SEQ ID NO: 40) | |
| gRNA_3F-4 | GTAATTTCTACTGTTGTAGATAGTGGGTCTATACGTACTGCCAATTTTTTT |
| (SEQ ID NO: 41) | |

Any of the OgRRS sequences presented in Table 28 can be used alternatively as a site to insert a CgRRS that is designed using a different OgRRS. For example, a CgRRS can be inserted into a flanking sequence to allow for the excision of the entire transgenic insertion of event Zm_CSM63715. To illustrate this approach, OgRRS 3F-4 is selected as the OgRRS that can be used to design a corresponding CgRRS 3F-4 comprising DNA fragment, and OgRRS 5F-63 is selected as the target site into which the CgRRS 3F-4 comprising DNA fragment is inserted. Using a Cas12a editing system such as with the Fn Cas12a endonuclease, the OgRRS 5F-63 site is targeted using the gRNA, gRNA_5F-63 presented in Table 29 to cut within the OgRRS 5F-63 site. The CgRRS 3F-4 comprising DNA fragment that comprises the OgRRS 3F-4 target site is then inserted within the cut site that was introduced into the OgRRS 5F-63 sequence. After selection of a transgenic event comprising the introduced CgRRS 3F-4 site, the event can be bred into another germplasm. When desired, the transgenic insert of Zm_CSM63715 can be excised from the plant using a Cas12a editing system and the gRNA, gRNA_3F-4 as presented in Table 29.

The CgRRS can be introduced into the transgenic insertion locus through multiple methods using a CRISPR system. For example, a CRISPR system can be utilized for targeting 5′ insertion of a blunt-end double-stranded DNA fragment into a genomic target site of interest such as an OgRRS that is not the OgRRS that has been selected for the design of the CgRRS. The CRISPR-mediated endonuclease activity can introduce a double stand break (DSB) in the selected genomic target site and DNA repair, such as microhomology-driven nonhomologous end-joining DNA repair, results in insertion of the blunt-end double-stranded DNA fragment into the DSB. Blunt-end double-stranded DNA fragments can be designed with 1-10 bp of microhomology, on both the 5′ and 3′ ends of the DNA fragment, that correspond to the 5′ and 3′-flanking sequence at the cut site of the protospacer in the genomic target site.

The CRISPR system can be introduced into event Zm_CSM63715 by several methods, including but not limited to Agrobacterium-mediated transformation, polyethylene glycol-mediated transformation, biolistic transformation, liposome-mediated transfection, viral transduction, the use of one or more delivery particles, microinjection, or electroporation. One or more expression cassettes encoding the gRNA and/or CRISPR associated protein components of a Type I, Type II, Type III, Type IV, Type V, or Type VI CRISPR-Cas system is transiently introduced into a cell. The CRISPR associated protein and guide RNA can be synthesized and assembled in vitro to form a ribonucleoprotein complex (RNP). The ribonucleoprotein along with a DNA fragment encoding the CgRRS can be provided to the plant via polyethylene glycol-mediated transformation, biolistic transformation, liposome-mediated transfection, the use of one or more delivery particles, microinjection, or electroporation. The introduced one or more gRNAs, or expression cassettes encoding the gRNA and/or CRISPR associated protein, along with a DNA fragment comprising the CgRRS is provided in sufficient quantity to modify the cell but does not persist after a contemplated period of time has passed or after one or more cell divisions. In such embodiments, no further steps are needed to remove or segregate the one or more expression cassettes encoding the gRNA and/or CRISPR associated protein from the modified cell. Double-stranded DNA fragments can also be transiently introduced into a cell along with one or more expression cassettes encoding the gRNA and/or CRISPR associated protein. The introduced double-stranded DNA fragments are provided in sufficient quantity to modify the cell but do not persist after a contemplated period of time has passed or after one or more cell divisions.

Alternatively, an expression construct comprising one or more expression cassettes for the expression of one or more gRNAs, and an expression construct encoding a Type I, Type II, Type III, Type IV, Type V, or Type VI CRISPR associated protein is stably transformed into event Zm_CSM63715 to modify the plant cell in the targeted region of the transgene insertion locus, to introduce the CgRRS within the desired target locus.

### Example 9. Modification of Corn Event Zm_CSM63715 with Genome Editing Techniques Using Two Guide RNAs

This example describes how one may alter or excise all or a part of the transgenic insertion present in corn event Zm_CSM63715, as well as flanking genomic DNA segments, such as by making one or more insertions, deletions, substitutions, or transpositions using genomic editing techniques. Excision of the event Zm_CSM63715 transgenic insertion or expression element(s) or cassette within SEQ ID NO:9 or SEQ ID NO:10 can be performed through genome editing using a variety of methods. In one embodiment, Clustered Regularly Interspersed Short Palindromic Repeats (CRISPR) editing systems comprising a CRISPR associated protein and two cognate guide RNAs may be used for targeted excision. The CRISPR-associated protein is an RNA guided nuclease and can be selected from a Type I CRISPR-associated protein, a Type II CRISPR-associated protein, a Type III CRISPR-associated protein, a Type IV CRISPR-associated protein, Type V CRISPR-associated protein, or a Type VI CRISPR-associated protein, such as but not limited to, Cas1, Cas1B, Cas2, Cas3, Cas4, Cas5, Cas6, Cas7, Cas8, Cas9 (also known as Csn1 and Csx12), Cas10, Cas12a (also known as Cpf1), Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, CasX, CasY, and Mad7. The CRISPR-associated protein and two guide RNAs (gRNA) can be introduced into a plant cell comprising the corn event Zm_CSM63715 to target a specific sequence within the transgene insertion locus. In one embodiment, the CRISPR nuclease system cleaves at two distinct guide RNA hybridization sites thereby permitting the excision of the intervening sequence. Following DNA cleavage, the genomic sequence can be repaired via a double strand break repair pathway, which may include, for example, non-homologous end-joining (NHEJ), microhomology-mediated end joining (MMEJ), homologous recombination, synthesis-dependent strand annealing (SDSA), single-strand annealing (SSA), or a combination thereof, at the genomic target site.

Sequences corresponding to the 5′ and 3′ flanking genomic sequences, and the transgenic insert of event Zm_CSM63715 (presented as SEQ ID NOs:11, 12, 164, 165 and 9, respectively) and the 5′ and 3′ junction regions (presented as SEQ ID NOs:1-6) were scanned for potential guide RNA recognition sites which comprise a protospacer adjacent motif (PAM) site that will be recognized by a Cas12a endonuclease, operably linked to a guide RNA hybridization site, and the results are shown in Table 28. The identified gRNA recognition sites are located within the 5′ or 3′ flanking genomic sequence, within the 5′ or 3′ junction regions, or within the transgenic insertion.

Two functional guide RNAs (gRNAs) for an RNA guided nuclease system are created to target the event Zm_CSM63715 transgenic insertion locus in a manner that will permit the excision of a fragment of DNA corresponding to either the entire transgenic insertion of event Zm_CSM63715, or a fragment within the transgenic insertion of event Zm_CSM63715 such as the expression cassette or genetic element within the transgene cassette. Illustrative examples are described below using FnCas12a editing system (see Table 29), in which the gRNAs may include a G leader sequence to facilitate transcription start and a gRNA repeat GTAATTTCTACTGTTGTAGAT (SEQ ID NO:39, gRNA repeat underlined), an OgRRS sequence (as shown in the fourth column of Table 28) and a TTTTTTT (poly-T transcript termination region, italic in Table 29) to target the FnCas12a nuclease to the gRNA recognition sites. Similar methods can be used to excise either the entire transgene insertion or a fragment within the transgene insertion such as the expression cassette by selecting gRNAs targeted to the specific regions. Alternatively, the LbCas12a editing systems can be used, in which the gRNAs include a G leader sequence to facilitate transcription start and a gRNA repeat GAATTTCTACTAAGTGTAGAT (SEQ ID NO:38, gRNA repeat underlined), an OgRRS sequence (as shown in the third column of Table 28), and a TTTTTTT (poly-T transcript termination region).

To excise the entire transgenic insertion of event Zm_CSM63715, the first gRNA targets an area in the 5′ flanking genomic sequence such as 5F-63 (Table 29), and the second gRNA targets a region in the 3′ flanking genomic sequence such as 3F-4 (Table 29). A transfer DNA (T-DNA) construct suitable for use in Agrobacterium-mediated transformation is used. The T-DNA construct comprises several expression cassettes between a left border (LB) sequence and a right border (RB) sequence. The first expression cassette comprises a promoter that is operable in a plant cell operably linked to a polynucleotide encoding a Cas12a RNA guided nuclease. A second expression cassette comprises a promoter that is operable in a plant cell operably linked to a selection marker gene, such as aadA for conferring resistance to spectinomycin and/or streptomycin. The construct also comprises expression cassettes comprising Polymerase III or Polymerase II promoters operable in a plant cell operably linked to polynucleotides encoding the two gRNAs gRNA_5F-63 and gRNA_3F-4 (Table 29).

Following Agrobacterium-mediated transformation of corn comprising event Zm_CSM63715, and upon expression of the integrated polynucleotides, the gRNAs guide the nuclease to each of the two target sites at the transgenic insertion locus, where the nuclease creates a double-stranded break at each target site, resulting in deletion of the region between the target sites, and non-homologous end-joining repair mechanisms joins the flanking regions. Suitable methods known in the art (e.g., PCR, DNA hybridization (Southern) blots, sequencing) are used to identify plants comprising a complete deletion.

### REFERENCES

X. Chen, L. Levine and P.-Y. Kwok (1999). Fluorescence polarization in homogeneous nucleic acid analysis. Genome Res. 9:492-498.
S. Cheng, C. Fockler, W. M. Barnes and R. Higuchi (1994). Effective amplification of long targets from cloned inserts and human genomic DNA. Proc. Natl. Acad. Sci. USA 91:5695-5699.
C. D. Day, E. Lee, J. Kobayashi, L. D. Holappa, H. Albert and D. W. Ow (2000). Transgene integration into the same chromosome location can produce alleles that express at a predictable level, or alleles that are differentially silenced. Genes Dev. 14 (22):2869-2880.
F. B. Dean, S. Hosono, L. Fang, X. Wu, A. F. Faruqi, P. Bray-Ward, Z. Sun, Q. Zong, Y. Du, J. Du, M. Driscoll, W. Song, S. F. Kingsmore, M. Egholm and R. S. Lasken (2002). Proc. Natl. Acad Sci. USA 99:5261-5266.
A. Fire and S. Q. Xu (1995). Rolling replication of short DNA circles. Proc. Natl. Acad Sci. USA 92:4641-4645.
L. Gao, D. B. T. Cox, W. X. Yan, J. C. Manteiga, M. W. Schneider, T. Yamano, H. Nishimasu, O. Nureki, N. Crosetto and F. Zhang (2017). Engineered Cpf1 variants with altered PAM specificities. Nature Biotechnology 35(8):789-792.
J. C. Guatelli, K. M. Whitefield, D. Y. Kwon, K. J. Barringer, D. D. Richman and T. R. Gingeras (1990). Isothermal, in vitro amplification of nucleic acids by a multienzyme reaction modeled after retroviral replication. Proc. Natl. Acad. Sci. USA 87:1874-1878.
I. M. Heap (2021). International Survey of Herbicide Resistant Weeds. http://www.weedscience.org/Home.aspx.
S. Khan, A. Latif, S. Q, Ahmad, F. Ahmad and M. Ghaffar (2010). Double haploid technique: in corn and other species. International Journal of Applied Agricultural Research 5:649-655.
Q. Li, Y. Huo, Y. Guo, X. Zheng, W. Sun and Z. Hao (2017) Generation and applications of a DNA aptamer against gremlin-1. Molecules 22:706.
D. Liu, S. L. Daubendiek, M. Zillman, K. Ryan and E. T. Kool (1996). Rolling circle DNA synthesis: small circular oligonucleotides as efficient templates for DNA polymerases. J. Am. Chem. Soc. 118:1587-1594.
I. Lermontova, E. Kruse, H. P. Mock, and B. Grimm (1997). Cloning and characterization of plastidal and mitochondrial isoform of tobacco protoporphyrinogen IX oxidase. Proc. Natl. Acad. Sci. USA 94:8895-8900.
P. M. Lizardi, X. Huang, Z. Zhu, P. Bray-Ward, D. C. Thomas and D. C. Ward (1998). Mutation detection and single-molecule counting using isothermal rolling-circle amplification. Nature Genetics 19:225-232.
T. T. Nikiforov, R. B. Rendie, P. Goelet, Y.-H. Rogers, M. L. Kotewicz, S. Anderson, G. L. Trainor and M. R. Knapp (1994). Genetic bit analysis: a solid phase method for typing single nucleotide polymorphisms. Nucleic Acids Res. 22:4167-4175.
T. Notomi, H. Okayama, H. Masubuchi, T. Yonekawa, K. Watanabe, N. Amino, T. Flase (2000). Loop-mediated isothermal amplification of DNA”, Nucleic Acids Res. 28 (12): 63e-63.
D. T. Patterson (1995), Effects of environmental stress on weed/crop interactions. Weed Science 43:483-490.
M. D. Pearson, L. Nguyen, Y. Zhao, W. L. McKenna, T. J. Morin and W. B. Dunbar (2019). Past and accurate quantification of insertion-site specific transgene levels from raw seed samples using solid-state nanopore technology. PloS One 14 (12):e0226719.
D. L. Robertson and J. G. Joyce (1990). Selection in vitro of an RNA enzyme that specifically cleaves single-stranded DNA. Nature 344:467-468.
S. S. Sekhon, H. J. Um, W. R. Shin, S. H. Lee, J. Min, J. Y. Ahn and Y. H. Kim. (2017). Aptabody-aptatope interactions in aptablotting assays. Nanoscale 9:7464-7475.
D. Shoup, S. Duncan and I A. Ciampitti (2016). Corn Production Handbook. Kansas State University Agricultural Experiment Station and Cooperative Extension Service.
N. Soltani, J. A. Dille, I. C. Burke, W. J. Everman, M. J. VanGessel, V. M. Davis, and P. H. Sikkema (2016). Potential corn yield losses from weeds in North America. Weed Technology 30 (4): 979-984.
L. E. Steckel and C. L. Sprague (2004). Common waterhemp (Amarwamhus rudis) interference in corn. Weed Science 52:359-364.
M. Svobodova. V. Skouridou, M. Jauset-Rubio, L Viditez. A. Fernmndez-Villar, J. J. C. Alvargonzalez, E. Poveda, C. B. Bofill, T. Sans. A. Bashammakh, A. O. Alyoubi and C. K. O'Sullivan (2021). Aptamer sandwich assay for detection of SA RS-CoV-2-spike protein antigen. ACS Omega 6:35657-35666.
A. D. Taylor, L. Mataseje, C. J. Urfano, L. Schmidt, K. S. Antonation, M. R. Mulvey and C. R. Corbett (2018). Evaluation of Oxford Nanopore's MinION Sequencing Device for Microbial Whole Genome Sequencing Applications. Scientific Reports 8:10931.
C. Tuerk and L. Gold (1990). Systematic evolution of ligands by exponential enrichment: RNA ligands to bacteriophage T4 DNA polymerase. Science 249:505-510.
S. Tyagi and F. R. Kramer (1996). Molecular beacons: probes that fluoresce upon hybridization. Nature Biotechnology 14:303-308.
A. Verkest, S. Bourout, J. Debaveye, K. Reynaert, B. Saey, I. Van den Brande and K. D′Halluin (2019). Impact of differential DNA methylation on transgene expression in cotton (Gossypium hirsutum L.) events generated by targeted sequence insertion. Plant Biotechnology J. 17:1236-1247.
M. Vincent, Y. Xu and H. Kong (2004). Helicase-dependent isothermal DNA amplification. EMBO Reports 5(8):795-800.
Y. Wan, J. F. Petolino and J. M. Widholm (1989). Efficient Production of Doubled Haploid Plants Through Colchicine Treatment of Anther-Derived Maize Callus. Theor. Appl. Genet. 77:889-892.
T. Wang, C. Chen, L. M. Larcher, R. A. Barrero and R. N. Veedu (2019). Three decades of nucleic acid aptamer technologies: Lessons learned, progress and opportunities on aptamer development. Biotechnol. Adv. 37:28-50.
Y. Wang, Z. Li and H. Yu (2020). Aptamer-based Western blot for selective protein recognition. Frontiers in Chem. 8:1-9.
Y. Wang, Y. Zhao, A. Bollas, Y. Wang and K. F. Au (2021). Nanopore sequencing technology, bioinformatics and applications. Nature Biotechnology 39:1348-1365.
M. Winge (2000). Pyrosequencing-a new approach to DNA analysis. Innovation in Pharma. Tech. 00:18-24.
B. Zetsche, J. S. Gootenberg, 0.0. Abudayyeh, I. M. Slaymaker, K. S. Makarova, P. Essletzbichler, S. E. Volz, J. Joung, J. v.d. Oost, A. Regev, E. V. Koonin and F. Zhang (2015). Cpf1 is a single RNA-guided endonuclease of a class 2 CRISPR-Cas system. Cell 163(3):759-771.

1. A recombinant DNA molecule comprising a nucleotide sequence selected from the group consisting of SEQ ID NO:10; SEQ ID NO:1; SEQ ID NO:2; SEQ ID NO:3; SEQ ID NO:4; SEQ ID NO:5; SEQ ID NO:6; SEQ ID NO:7; SEQ ID NO:8;
SEQ ID NO:9; a polynucleotide having a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO: 9; and a complete complement of any of the foregoing.

2. The recombinant DNA molecule of claim 1, wherein;
a) the recombinant DNA molecule is derived from a corn plant, seed, plant part, plant cell, progeny plant, or commodity product comprising corn event Zm_CSM63715, a representative sample of seed comprising the event having been deposited as ATCC Accession No. PTA-127361;
b) the recombinant DNA molecule is comprised in a corn plant, seed, plant part, plant cell, or progeny plant comprising corn event Zm CSM63715, or a commodity product produced therefrom, a representative sample of seed comprising the event having been deposited as ATCC Accession No. PTA-127361;
c) the recombinant DNA molecule is formed by the insertion of a heterologous nucleic acid molecule into the genomic DNA of a corn plant or corn cell; or
d) the recombinant DNA molecule comprises an amplicon diagnostic for the presence of corn event Zm CSM63715.

3-5. (canceled)

6. A DNA molecule comprising a polynucleotide segment of sufficient length to function as a DNA probe, wherein detecting hybridization of the DNA molecule under stringent hybridization conditions is diagnostic for the presence of the recombinant DNA molecule of claim 1 in a sample, and wherein:
a) said DNA molecule hybridizes specifically under the stringent hybridization conditions with corn event Zm_CSM63715 DNA in the sample;
b) said DNA molecule is specific for detecting in the sample:
i) a 5′ junction sequence between flanking corn genomic DNA and the transgenic insert of corn event Zm CSM63715;
ii) a 3′ junction sequence between the transgenic insert of corn event Zm CSM63715 and flanking corn genomic DNA; SEQ ID NO:9; or
iii) a fragment of SEQ ID NO:9 comprising a sufficient length of contiguous nucleotides of SEQ ID NO:9 to identify the sequence as a fragment of the transgenic insert of Zm CSM63715;
c) said DNA molecule comprises SEQ ID NO:16; or
d) said DNA molecule comprises a nucleotide sequence selected from the group consisting of SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; and a complement of any of the foregoing.

7-9. (canceled)

10. The DNA molecule of claim 6, wherein the sample is derived from a corn plant, seed, plant part, plant cell, progeny plant, or commodity product.

11. A pair of DNA molecules comprising a first DNA molecule and a second DNA molecule that function as DNA primers when used together in an amplification reaction with DNA comprising corn event Zm CSM63715 to produce an amplicon diagnostic for the recombinant DNA molecule of claim 1 in a sample, wherein:
a) the first and the second DNA molecules comprise a fragment of SEQ ID NO:10 or a complement thereof;
b) the first and the second DNA molecules comprise SEQ ID NO:14 and SEQ ID NO:15; or
c) the amplicon comprises a nucleotide sequence selected from the group consisting of:
SEQ ID NO:1;
SEQ ID NO:2;
SEQ ID NO:3;
SEQ ID NO:4;
SEQ ID NO:5;
SEQ ID NO:6;
SEQ ID NO:7;
SEQ ID NO:8;
SEQ ID NO:9;
SEQ ID NO:10; and
a fragment of any of SEQ ID NO:1, SEQ ID NO:2, SEQ ID NO:3, SEQ ID NO:4, SEQ ID NO:5, SEQ ID NO:6, SEQ ID NO:7, SEQ ID NO:8, wherein
the fragment is at least 10 nucleotides in length and comprises nucleotides 1,000-1,001 or 4,552-4,553 of SEQ ID NO:10.

12-13. (canceled)

14. A method of detecting the presence of corn event Zm_CSM63715 in a sample derived from a corn seed, plant, plant part, plant cell, progeny plant, or commodity product, the method comprising:
a) contacting the sample with the DNA molecule that functions as a DNA probe of claim 6;
b) subjecting the sample and the DNA molecule that functions as a DNA probe to stringent hybridization conditions; and
c) detecting the hybridization of the DNA molecule that functions as a DNA probe to a DNA molecule in the sample,
wherein the hybridization of the DNA molecule that functions as a DNA probe to the DNA molecule in the sample is diagnostic for the presence of corn event Zm_CSM63715 in the sample.

15. A method of detecting the presence of corn event Zm_CSM63715 in a sample derived from a corn seed, plant, plant part or plant cell, progeny plant or commodity product, the method comprising:
a) contacting the sample with the pair of DNA molecules of claim 11;
b) performing an amplification reaction sufficient to produce a DNA amplicon; and
c) detecting the presence of the DNA amplicon;
wherein the DNA amplicon comprises at least one of:
a 5′ junction sequence between flanking corn genomic DNA and the transgenic insert of corn event Zm_CSM63715,
a 3′ junction sequence between flanking corn genomic DNA and the transgenic insert of corn event Zm_CSM63715, SEQ ID NO: 9, or
a fragment of SEQ ID NO: 9 comprising a sufficient length of contiguous nucleotides of SEQ ID NO: 9 to identify the sequence as a fragment of the transgenic insert of Zm_CSM63715; and
wherein the presence of the DNA amplicon indicates the presence of corn event Zm_CSM63715 in the sample.

16. The method of claim 15, wherein;
a) the DNA amplicon is at least 10 nucleotides in length, at least 11 nucleotides in length, at least 12 nucleotides in length, at least 13 nucleotides in length, at least 14 nucleotides in length, at least 15 nucleotides in length, at least 16 nucleotides in length, at least 17 nucleotides in length, at least 18 nucleotides in length, at least 19 nucleotides in length, at least 20 nucleotides in length, at least 25 nucleotides in length, at least 30 nucleotides in length, at least 35 nucleotides in length, at least 40 nucleotides in length, at least 45 nucleotides in length, at least 50 nucleotides in length, at least 60 nucleotides in length, at least 70 nucleotides in length, at least 80 nucleotides in length, at least 90 nucleotides in length, or at least 100 nucleotides in length, or
b) the DNA amplicon comprises a nucleotide sequence selected from the group consisting of SEQ ID NO:10; SEQ ID NO:9; SEQ ID NO:8; SEQ ID NO:7;
SEQ ID NO:6; SEQ ID NO:5; SEQ ID NO:4; SEQ ID NO:3; SEQ ID NO:2; SEQ ID NO:1; and a fragment of any of SEQ ID NO:10, SEQ ID NO:8, SEQ ID NO:7, SEQ ID NO:6, SEQ ID NO:5, SEQ ID NO:4, SEQ ID NO:3, SEQ ID NO:2, and SEQ ID NO:1 that is at least 10 nucleotides in length and comprises nucleotides 1,000-1,001 or 4,552-4,553 of SEQ ID NO:10.

17. (canceled)

18. A method of detecting the presence of corn event Zm_CSM63715 in a sample of DNA derived from a corn seed, plant, plant part, plant cell, progeny plant or commodity product, the method comprising:
a) contacting the sample with the DNA molecule of claim 6; and
b) performing a sequencing reaction to produce a target sequence,
wherein the target sequence comprises a nucleotide sequence selected from the group consisting of SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; a complete complement of any thereof; and a fragment of any of SEQ ID NO:1, SEQ ID NO:2, SEQ ID NO:3, SEQ ID NO:4, SEQ ID NO:5, SEQ ID NO:6, SEQ ID NO:7, SEQ ID NO:8, and SEQ ID NO:10 that is at least 10 nucleotides long and comprises nucleotides 1,000-1,001 or 4,552-4,553 of SEQ ID NO:10.

19. A method of detecting the presence of corn event Zm_CSM63715 in a sample derived from a corn seed, plant, plant part, cell, progeny plant or commodity product comprising the recombinant DNA molecule of claim 1, the method comprising:
a) contacting the sample with an antibody specific for the PPO (protoporphyrinogen oxidase) protein encoded by corn event Zm_CSM63715; and
b) detecting binding of the antibody to the protein in the sample,
wherein the binding of the antibody indicates the presence of corn event Zm_CSM63715 in the sample.

20. A kit for detecting the presence of corn event Zm_CSM63715 in a sample, wherein the kit comprises:
a) the pair of DNA primers of claim 11;
b) a DNA molecule comprising a polynucleotide segment of sufficient length to function as a DNA probe that hybridizes specifically under stringent hybridization conditions with corn event Zm CSM63715 DNA in a sample, wherein detecting hybridization of the DNA molecule under the stringent hybridization conditions is diagnostic for the presence of corn event Zm CSM63715 in the sample; or
c) an antibody specific for the PPO protein encoded by corn event Zm CSM63715; wherein detecting binding of the antibody to the protein encoded by corn event Zm CSM63715 in a sample is diagnostic for the presence of corn event Zm CSM63715 in the sample.

21. (canceled)

22. A method of determining the zygosity of a corn plant, seed,
plant part, or plant cell comprising the recombinant DNA molecule of claim 2, wherein the recombinant DNA molecule is comprised in the corn plant, seed, plant part, or plant cell comprising corn event Zm CSM63715, the method comprising:
a) contacting a sample comprising DNA derived from the corn plant, plant part, seed, or plant cell with a first primer set capable of producing a first amplicon diagnostic for the presence of corn event Zm_CSM63715, and a second primer set capable of producing a second amplicon diagnostic for the wildtype corn genomic DNA not comprising corn event Zm_CSM63715;
b) performing a nucleic acid amplification reaction; and
c) detecting the first amplicon and the second amplicon, wherein the presence of both amplicons indicates that the plant, plant part, seed or cell is heterozygous for corn event Zm_CSM63715, and the presence of only the first amplicon indicates that the plant, plant part, seed, or cell is homozygous for corn event Zm_CSM63715.

23. The method of claim 22, wherein the first primer set comprises SEQ ID NO:14 and SEQ ID NO:15, and the second primer set comprises SEQ ID NO:20 and SEQ ID NO:21 or SEQ ID NO: 15 and SEQ ID NO: 21.

24. A method of determining the zygosity of a corn plant, seed, plant part, or plant cell comprising the recombinant DNA molecule of claim 2, wherein the recombinant DNA molecule is comprised in the corn plant, seed, plant part, or plant cell comprising corn event Zm CM63715, the method comprising:
a) contacting a sample comprising DNA derived from the corn plant, plant part, seed, or plant cell with a probe set comprising at least a first probe that specifically hybridizes to corn event Zm_CSM63715, and at least a second probe that specifically hybridizes to corn genomic DNA that was disrupted by insertion of the heterologous DNA of corn event Zm_CSM63715 but does not hybridize to corn event Zm_CSM63715; and
b) hybridizing the probe set with the sample under stringent hybridization conditions, wherein detecting hybridization of only the first probe under the hybridization conditions is diagnostic for a corn plant, plant part, seed or plant cell homozygous for corn event Zm_CSM63715, and wherein detecting hybridization of both the first probe and the second probe under the hybridization conditions is diagnostic for a corn plant, plant part, seed, or plant cell heterozygous for corn event Zm_CSM63715.

25. The method of claim 24, wherein the probe set comprises SEQ ID NO:16 and SEQ ID NO:22.

26. A DNA construct comprising an expression cassette, wherein the expression cassette comprises in operable linkage i) a ubiquitin (UBQ) promoter, a leader sequence, and an intron sequence from Andropogon gerardii, ii) a chloroplast transit peptide coding sequence of APG6 (Albino and Pale Green 6) from Arabidopsis thaliana, iii) a codon-optimized protoporphyrinogen oxidase coding sequence from Enterobacter cloacae, and iv) a 3′ UTR sequence of an alpha tubulin gene from Arundo donax.

27. The DNA construct of claim 26, wherein:
a) the DNA construct comprises SEQ ID NO:9;
b) the DNA construct comprises a polynucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO: 9; and wherein the DNA construct comprises at the 5′ or 3′ end of said construct (i) at least 50 contiguous nucleotides of SEQ ID NO: 11 or SEQ ID NO:164; or (ii) at least 50 contiguous nucleotides of SEQ ID NO: 12 or SEQ ID NO:165
c) the DNA construct further comprises at the 5′ or 3′ end of said DNA construct:
i) at least 50 contiguous nucleotides of SEQ ID NO: 11 or SEQ ID NO:164;
or
ii) at least 50 contiguous nucleotides of SEQ ID NO: 12 or SEQ ID NO:165;
d) the DNA construct comprises at the 5′ end of said DNA construct at least one nucleotide sequence selected from SEQ ID NOs:44-103; or
e) the DNA construct comprises at the 3′ end of said DNA construct at least one nucleotide sequence selected from SEQ ID NOs:104-163.

28. (canceled)

29. A corn plant, plant part, seed, or plant cell comprising corn event Zm CSM63715, a representative sample of seed comprising corn event Zm CSM63715 having been deposited under ATCC Accession No. PTA-127361.

30-31. (canceled)

32. A method for controlling or preventing weed growth in an area, the method comprising planting the corn plant or seed of claim 29 in the area and applying an effective amount of a PPO herbicide to control weeds in the area without injury to the corn or with less than about 10% injury to the corn.

33. A method for controlling volunteer corn comprising the corn plant of claim 29 in an area, the method comprising applying an herbicidally effective amount of at least one herbicide other than a PPO herbicide, wherein the herbicide application prevents growth of corn comprising corn event Zm_CSM63715.

34. The method of claim 33, wherein the herbicide other than a PPO herbicide is selected from the group consisting of pyrithiobac, trifluralin, fluometuron, trifloxysulfuron, FOP herbicides such as quizalofop or fluazifop, DIM herbicides such as clethodim or sethoxydim, fenoxaprop, glyphosate, glufosinate, and combinations of any thereof.

35. A method of obtaining a seed of a corn plant or a corn plant that is tolerant to PPO herbicides, the method comprising:
a) obtaining a population of progeny seed or plants grown therefrom derived from the corn plant claim 29, at least one of which comprises corn event Zm_CSM63715; and
b) identifying at least a first progeny seed or plant grown therefrom that comprises corn event Zm_CSM63715.

36. The method of claim 35, wherein identifying the progeny seed or plant grown therefrom that comprises corn event Zm_CSM63715 comprises:
a) growing the progeny seed or plant to produce progeny plants;
b) treating the progeny plants with an effective amount of a PPO herbicide; and
c) selecting a progeny plant that is tolerant to the PPO herbicide.

37. The method of claim 35, wherein identifying the progeny seed or plant grown therefrom that comprises corn event Zm_CSM63715 comprises;
a) detecting the presence of corn event Zm_CSM63715 in a sample derived from the progeny seed or plant grown therefrom, or
b) detecting the presence of the PPO protein encoded by corn event Zm CSM63715 in a sample derived from the progeny seed or plant grown therefrom.

38. (canceled)

39. A method of improving tolerance to PPO herbicides in a corn plant comprising:
a) inserting the DNA construct of claim 26 into the genome of a corn cell;
b) generating a corn plant from the corn cell; and
c) selecting a corn plant comprising the DNA construct.

40. The method of claim 39, wherein the selecting comprises treating the corn cell or plant with an effective amount of a PPO herbicide.

41. A corn plant, plant seed, plant part, or plant cell comprising the recombinant DNA molecule of claim 1.

42. The corn plant, plant seed, plant part, or plant cell of claim 41, wherein;
a) the plant, plant seed, plant part, or plant cell expresses a PPO herbicide tolerance gene;
b) the plant, plant seed, plant part, or plant cell is tolerant to at least one PPO herbicide;
c) the plant, plant seed, plant part, or plant cell further comprises at least one additional transgene for tolerance to at least one additional herbicide;
d) the plant, plant seed, plant part, or plant cell comprises corn event Zm CSM63715, a representative sample of seed comprising the event having been deposited under ATCC Accession No. PTA-127361;
e) the plant, plant seed, plant part, or plant cell is further defined as a progeny plant of any generation of a corn plant comprising corn event Zm CSM63715, or a corn plant part, plant seed, or plant cell derived therefrom;
f) the plant part comprises a microspore, pollen, an anther, silk, spike, an ovule, an ovary, a pod, a flower, a cob, an embryo, a stem, a leaf, a root, or a callus;
g) the plant, plant seed, plant part, or plant cell further comprises corn event MON87429; or
h) the plant, plant seed, plant part, or plant cell further comprises a recombinant DNA molecule comprising a sequence selected from the group consisting of SEQ ID NO:212; SEQ ID NO:213; SEQ ID NO:214; SEQ ID NO:215; SEQ ID NO:216; SEQ ID NO:217; SEQ ID NO:218; SEQ ID NO:219; SEQ ID NO:220; SEQ ID NO:221; a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:212 or the full length of SEQ ID NO: 213; and a complete complement of any of the foregoing..

43-48. (canceled)

49. A corn plant, plant seed, plant part, or plant cell tolerant to one or more PPO herbicides, wherein the corn plant, plant seed, plant part, or plant cell comprises the DNA construct of claim 26.

50. A corn plant or corn seed obtained by the method of claim 35, wherein the corn plant or corn seed comprises a recombinant DNA molecule comprising a nucleotide sequence selected from the group consisting of SEQ ID NO:10; SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; a polynucleotide having a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO: 9; and a complete complement of any of the foregoing.

51. A corn plant, plant cell, plant part, plant seed, or progeny plant comprising:
aa) a recombinant DNA construct integrated in chromosome 8, wherein the recombinant DNA construct confers tolerance to at least one PPO herbicide, and wherein the recombinant DNA construct is integrated in a position of said chromosome flanked by at least 50 contiguous nucleotides of SEQ ID NO:11 or SEQ ID NO:164 and at least 50 contiguous nucleotides of SEQ ID NO:12 or SEQ ID NO:165, or
b) a recombinant nucleic acid molecule, said recombinant nucleic acid molecule comprising a target corn genomic nucleic acid sequence having at least 85% sequence identity, at least 90% sequence identity, or at least 95% sequence identity, to a nucleic acid molecule selected from the group consisting of SEQ ID NOs:174-190; and a DNA sequence of interest, wherein the DNA sequence of interest is inserted into said target corn genomic nucleic acid sequence.

52. The corn plant, plant cell, plant part, plant seed, or progeny plant of claim 51, wherein;
a) the at least 50 contiguous nucleotides of SEQ ID NO:11 or SEQ ID NO:164 comprise-ene-or-more at least one nucleotide sequence selected from SEQ ID NOs:44-103;
b) the at least 50 contiguous nucleotides of SEQ ID NO:12 or SEQ ID NO:165 comprise at least one nucleotide sequence selected from SEQ ID NOs:104-163;
c) the recombinant nucleic acid molecule comprises a target corn genomic nucleic acid sequence having a sequence selected from the group consisting of SEQ ID NOs:174-190;
d) the DNA sequence of interest comprises a gene of agronomic interest;
e) the target corn genomic nucleic acid sequence is at least 1 kb from the MON87429 insertion site;
f) the target corn genomic nucleic acid sequence maps to within 5 cM of the MON87429 insertion site;
g) the target corn genomic nucleic acid sequence is more than 1 kb from a gene, is more than 1 kb from a repressive chromatin mark, is more than 200 nucleotides from a small RNA hotspot, is more than 1 kb from a long repeat region, has DNA methylation less than or equal to 10% of genome-wide population average, and/or has a redundancy score less than or equal to 30%; or
h) the DNA sequence of interest comprises a gene that confers herbicide tolerance in plants.

53. (canceled)

54. The corn plant, plant cell, plant part, or plant seed of claim 42, wherein the PPO herbicide is selected from the group consisting of diphenylethers, N-phenylphthalimides, oxadiazoles, oxazolidinediones, phenylpyrazoles, pyrimidinediones, thiadiazoles, triazolinones, benzoxazinone derivatives, other PPO herbicides, and combinations of any thereof.

55. The corn plant, plant cell, plant part, or plant seed of claim 54, wherein the diphenylether is selected from the group consisting of acifluorfen, bifenox, ethoxyfen, fluorodifen, fluoronitrofen, furyloxyfen, halosafen, chlomethoxyfen, chlornitrofen, ethoxyfen-ethyl, fluoroglycofen, lactofen, nitrofen, oxyfluorfen, fomesafen, a salt of any thereof, and an ester of any thereof; the N-phenylphthalimide is selected from the group consisting of cinidon-ethyl, flumiclorac, flumiclorac-pentyl, and flumioxazin; the oxadiazole is selected from the group consisting of oxadiargyl and oxadiazon; the oxazolidinedione is pentoxazone; the phenylpyrazole is selected from the group consisting of fluazolate, pyraflufen, and pyraflufen-ethyl; the pyrimidinedione is selected from the group consisting of benzfendizone, butafenacil, epyrifencacil (S-3100), flupropacil, flufenoximacil, saflufenacil, and tiafenacil; the thiadiazole is selected from the group consisting of fluthiacet-methyl and thidiazimin; the triazolinone is selected from the group consisting of azafenidin, bencarbazone, carfentrazone, its salts and esters, and sulfentrazone; the benzoxazinone derivative is 1,5-dimethyl-6-thioxo-3-(2,2,7-trifluoro-3,4-dihydro-3-oxo-4-prop-2-ynyl-2H-1,4-benzoxazin-6-yl)-1,3,5-triazinane-2,4-dione (trifludimoxazin)); the other PPO herbicide is selected from the group consisting of chlorphthalim, flufenpyr, flufenpyr-ethyl, flumipropyn, pyraclonil, profluazol, pyridin-2-ylmethyl[(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl[(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl[(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate, cyanomethyl[(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate; methyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, methyl (2R)-2-{1[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate (flufenoximacil), methyl (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, methyl 2-{[(Z)-{2-chloro-4-fluoro-5-[3-methyl-2,6-dioxo-4-(trifluoromethyl)-3,6-dihydropyrimidin-1(2H)-yl]benzylidene}amino]oxy}propanoate, 2-{[(Z)-{2-chloro-4-fluoro-5-[3-methyl-2,6-dioxo-4-(trifluoromethyl)-3,6-dihydropyrimidin-1(2H)-yl]benzylidene}amino]oxy}propanoic acid, ethyl 2-{1[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, ethyl (2R)-2-{1[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, ethyl (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoic acid, (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoic acid, (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoic acid, methyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}-2-methylpropanoate, ethyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}-2-methylpropanoate, methyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoate, methyl (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoate, methyl (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoate, 2-{1[(E)-{2-chloro-4-fluoro-5-[3-methyl-2,6-dioxo-4-(trifluoromethyl)-3,6-dihydropyrimidin-1(2H)-yl]benzylidene}amino]oxy}butanoic acid, (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoic acid, (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoic acid, ethyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoate, methyl 2-({(E)-[2-chloro-5-(3,5-dimethyl-2,6-dioxo-4-sulfanylidene-1,3,5-triazinan-1-yl)-4-fluorobenzylidene]amino}oxy)propanoate methyl (2R)-2-({(E)-[2-chloro-5-(3,5-dimethyl-2,6-dioxo-4-sulfanylidene-1,3,5-triazinan-1-yl)-4-fluorobenzylidene]amino}oxy)propanoate, methyl (2S)-2-({(E)-[2-chloro-5-(3,5-dimethyl-2,6-dioxo-4-sulfanylidene-1,3,5-triazinan-1-yl)-4-fluorobenzylidene]amino}oxy)propanoate, 2-({(E)-[2-chloro-5-(3,5-dimethyl-2,6-dioxo-4-sulfanylidene-1,3,5-triazinan-1-yl)-4-fluorobenzylidene]amino}oxy)propanoic acid, (2R)-2-({(E)-[2-chloro-5-(3,5-dimethyl-2,6-dioxo-4-sulfanylidene-1,3,5-triazinan-1-yl)-4-fluorobenzylidene]amino}oxy)propanoic acid, (2S)-2-({(E)-[2-chloro-5-(3,5-dimethyl-2,6-dioxo-4-sulfanylidene-1,3,5-triazinan-1-yl)-4-fluorobenzylidene]amino}oxy)propanoic acid, ethyl 2-({(E)-[2-chloro-5-(3,5-dimethyl-2,6-dioxo-4-sulfanylidene-1,3,5-triazinan-1-yl)-4-fluorobenzylidene]amino}oxy)propanoate, ethyl (2R)-2-({(E)-[2-chloro-5-(3,5-dimethyl-2,6-dioxo-4-sulfanylidene-1,3,5-triazinan-1-yl)-4-fluorobenzylidene]amino}oxy)propanoate, ethyl (2S)-2-({(E)-[2-chloro-5-(3,5-dimethyl-2,6-dioxo-4-sulfanylidene-1,3,5-triazinan-1-yl)-4-fluorobenzylidene]amino}oxy)propanoate, methyl 2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoate, methyl (2R)-2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoate, methyl (2S)-2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoate, 2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoic acid, (2R)-2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoic acid, (2S)-2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoic acid, ethyl 3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylate, methyl 3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylate, 3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylic acid, (5R)-3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylic acid, (5S)-3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylic acid, ethyl (5S)-3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylate, ethyl (5R)-3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylate, ethyl 3-{2-chloro-4-fluoro-5-[3-methyl-2,6-dioxo-4-(trifluoromethyl)-3,6-dihydropyrimidin-1(2H)-yl]phenyl}-5-propyl-4,5-dihydro-1,2-oxazole-5-carboxylate, ethyl 3-{2-chloro-4-fluoro-5-[3-methyl-2,6-dioxo-4-(trifluoromethyl)-3,6-dihydropyrimidin-1(2H)-yl]phenyl}-5-ethyl-4,5-dihydro-1,2-oxazole-5-carboxylate, 3-[4-chloro-2-fluoro-5-(5-{[(isopropylideneamino)oxy]carbonyl}-5-methyl-4,5-dihydro-1,2-oxazol-3-yl)phenyl]-1-methyl-6-(trifluoromethyl)pyrimidine-2,4(1H,3H)-dione, ethyl 3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylate, methyl 3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylate, 3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylic acid, (5R)-3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylic acid, (5S)-3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylic acid, 3-[4-chloro-2-fluoro-5-(5-{[(isopropylideneamino)oxy]carbonyl}-5-methyl-4,5-dihydro-1,2-oxazol-3-yl)phenyl]-1,5-dimethyl-6-sulfanylidene-1,3,5-triazinane-2,4-dione, ethyl 3-{5-[3-amino-2,6-dioxo-4-(trifluoromethyl)-3,6-dihydropyrimidin-1(2H)-yl]-2-chloro-4-fluorophenyl}-5-methyl-4,5-dihydro-1,2-oxazole-5-carboxylate, 3-{5-[3-amino-2,6-dioxo-4-(trifluoromethyl)-3,6-dihydropyrimidin-1(2H)-yl]-2-chloro-4-fluorophenyl}-5-methyl-4,5-dihydro-1,2-oxazole-5-carboxylic acid, methyl 3-{2-chloro-4-fluoro-5-[3-methyl-2,6-dioxo-4-(trifluoromethyl)-3,6-dihydropyrimidin-1(2H)-yl]phenyl}-3a,4,5,6-tetrahydro-6aH-cyclopenta[d][1,2]oxazole-6a-carboxylate, ethyl 3-{2-chloro-4-fluoro-5-[3-methyl-2,6-dioxo-4-(trifluoromethyl)-3,6-dihydropyrimidin-1(2H)-yl]phenyl}-3a,4,5,6-tetrahydro-6aH-cyclopenta[d][1,2]oxazole-6a-carboxylate, methyl 3-{2-bromo-4-fluoro-5-[3-methyl-2,6-dioxo-4-(trifluoromethyl)-3,6-dihydropyrimidin-1(2H)-yl]phenyl}-3a,4,5,6-tetrahydro-6aH-cyclopenta[d][1,2]oxazole-6a-carboxylate, 2-ethoxy-2-oxoethyl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, {[(1-{2-chloro-4-fluoro-5-[3-methyl-2,6-dioxo-4-(trifluoromethyl)-3,6-dihydropyrimidin-1(2H)-yl]phenoxy}cyclopropyl)carbonyl]oxy}acetic acid, 2-methoxy-2-oxoethyl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, and cyclopropylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate.

56. The method claim 32, and wherein the effective amount of PPO herbicide is about 0.0009 lb/acre to about 1.5 lb/acre over a growing season.

57. A method of producing a progeny corn plant comprising corn event Zm_CSM63715 comprising:
a) sexually crossing the corn plant of claim 29 with itself or a second corn plant;
b) collecting one or more seeds produced from the cross;
c) growing one or more seeds to produce one or more progeny plants; and
d) selecting at least a first progeny plant or seed comprising corn event Zm_CSM63715.

58. An inbred or hybrid corn plant or seed comprising corn event Zm_CSM63715 produced by the method of claim 57.

59. A nonliving or nonregenerable corn plant material or a commodity product comprising;
a) the recombinant DNA molecule of claim 1;
a DNA construct comprising an expression cassette, wherein the expression cassette comprises in operable linkage i) a ubiquitin (UBQ) promoter, a leader sequence, and an intron sequence from Andropogon gerardii, ii) a chloroplast transit peptide coding sequence of APG6 (Albino and Pale Green 6) from Arabidopsis thaliana, iii) a codon-optimized protoporphyrinogen oxidase coding sequence from Enterobacter cloacae, and iv) a 3′ UTR sequence of an alpha tubulin gene from Arundo donax; or
c) corn event Zm CSM63715, a representative sample of seed comprising the corn event corn event Zm CSM63715 having been deposited under ATCC Accession No. PTA-127361.

60-61. (canceled)

62. The commodity product of claim 59 wherein;
a) the commodity product is produced from a transgenic corn plant, plant part, plant seed, or plant cell comprising the corn event Zm_CSM63715, or b) the commodity product comprises whole or processed seeds; viable or nonviable seeds; viable plant parts (such as roots and leaves); viable plant cells;
processed plant parts; processed plant tissues; dehydrated plant tissues; dehydrated plant parts; frozen plant tissues; frozen plant parts; food for human consumption such as corn oil, corn meal, corn flour, corn grits, corn flakes, corn bran, corn starch, sweetener such as high fructose corn syrup (HFCS), glucose and dextrose, beverage alcohol, brewer grits for beer production, fiber; animal feed such as corn, corn biomass; industrial alcohol; fuel ethanol; corn pollen; corn plastic; dried distillers grains (DDGs); or bio-degradable packing material.

63. (canceled)

64. A method of producing a commodity product, the method comprising:
a) obtaining the corn plant, plant part, or seed of claim 29; and
b) producing a commodity product from the corn plant, plant part, or seed.

65. A method of controlling, preventing, or reducing the development of herbicide-tolerant weeds comprising;
a) cultivating in a crop growing environment a corn plant comprising transgenes that provide tolerance to (i) a PPO herbicide and (ii) herbicides with at least three additional herbicide modes of action, the three additional herbicide modes of action each being different from one another, or
b) (i) cultivating in a crop growing environment a corn plant comprising the DNA construct of claim 26 and at least three additional transgenes for providing tolerance to herbicides with at least three additional herbicide modes of action, the three additional herbicide modes of action each being different from one another;
and (ii) applying to the crop growing environment at least one herbicide selected from the group consisting of dicamba, glufosinate, 2,4-D, PPO inhibitor, glyphosate, and any combination thereof, wherein the corn plant is tolerant to the at least one herbicide.

66. (canceled)

67. The method of claim 65, wherein;
a) the transgenes that provide tolerance to the herbicides with the at least three additional herbicide modes of action are present at a single genomic location in the corn plant;
b) the additional transgenes are selected from the group consisting of FT T, dicamba monooxygenase (DMO), phosphinothricin N-acetyltransferase (PAT), 5-enolpyruvylshikimate-3-phosphate synthase (EPSPS), and combinations of any thereof; or
c) the additional transgenes provide tolerance to herbicides having modes of action selected from the group consisting of inhibitors of glutamine synthetase, inhibitors of acetyl CoA carboxylase (ACCase) in the aryloxyphenoxy propionate (FOP) group, inhibitors of EPSPS, synthetic auxins, and combinations of any thereof.

68. A method of reducing loci for corn breeding by site-directed insertion of a transgene that provides tolerance to a PPO herbicide at a genomic location in a corn plant that is within about 3-8 cM of a locus in the genome of the corn plant that comprises transgenes for tolerance to at least three additional herbicide modes of action, the three additional herbicide modes of action each being different from one another.

69. (canceled)

70. The method of claim 67, wherein;
a) the FT_T transgene comprises a polynucleotide sequence encoding a protein having the amino acid sequence of SEQ ID NO:171; the DMO transgene comprises a polynucleotide sequence encoding a protein having the amino acid sequence of SEQ ID NO:169; the PAT transgene comprises a polynucleotide sequence encoding a protein having the amino acid sequence of SEQ ID NO:167; and the EPSPS transgene comprises a polynucleotide sequence encoding a protein having the amino acid sequence of SEQ ID NO:173, or
b) the inhibitor of acetyl CoA carboxylase (ACCase) in the aryloxyphenoxy propionate (FOP) group is selected from the group consisting of chlorazifop, clodinafop, clodinafop-ethyl, clodinafop-propargyl, clofop, cyhalofop, cyhalofop-butyl, diclofop, diclofop-methyl, diclofop-P, diclofop-P-methyl, fenoxaprop, fenoxaprop-P, fenoxaprop-P-ethyl, fenthiaprop, fluazifop, fluazifop-butyl, fluazifop-P, fluazifop-P-butyl, haloxyfop, haloxyfop-etotyl, haloxyfop-methyl, haloxyfop-P, haloxyfop-P-methyl, isoxapyrifop, metamifop, propaquizafop, quizalofop, quizalafop-ethyl, quizalofop-P, quizalafop-P-ethyl, quizalafop-P-tefuryl, trifop, and combinations of any thereof; the synthetic auxin is selected from the group consisting of dicamba, 2,4-D, dichlorprop, mecoprop, 2,4,5-T (2,4,5-trichlorophenoxyacetic acid), and combinations of any thereof; the inhibitor of glutamine synthetase comprises glufosinate; or the inhibitor of 5-enolpyruvylshikimate-3-phosphate synthase (EPSPS) comprises glyphosate

71-81. (canceled)

82. A method of generating a recombinant corn plant cell comprising:
a) obtaining a corn plant, seed, or cell, wherein said plant, seed, or cell comprises a target corn genomic nucleic acid molecule having at least 85% sequence identity, at least 90% sequence identity, or at least 95% sequence identity to a nucleic acid molecule selected from the group consisting of SEQ ID NOs:174-190, or a complement thereof;
b) introducing into the corn plant, seed, or cell a site-specific nuclease that can specifically bind to and cleave the target corn genomic nucleic acid molecule;
c) introducing a DNA sequence of interest into the corn plant, seed, or cell; and
d) selecting recombinant corn plants, seeds or cells comprising the DNA sequence of interest inserted in the target corn genomic nucleic acid molecule.

83. The method of claim 82, wherein:
a) the site-specific nuclease is selected from the group consisting of an RNA-guided nuclease, a zinc finger nuclease, and a TALEN;
b) the RNA-guided nuclease is Cas12a;
c) the target corn genomic nucleic acid sequence is at least 1 kb from the MON87429 insertion site;
d) the target corn genomic nucleic acid sequence maps to within 5 cM of the MON87429 insertion site; or
e) the target corn genomic nucleic acid sequence is more than 1 kb from a gene, is more than 1 kb from a repressive chromatin mark, is more than 200 nucleotides from a small RNA hotspot, is more than 1 kb from a long repeat region, has DNA methylation less than or equal to 10% of genome-wide population average, and/or has a redundancy score less than or equal to 30%.

84. (canceled)

85. The method of claim 83, further comprising introducing into the corn plant, seed, or cell a guide polynucleotide comprising a nucleic acid sequence that is substantially complementary to the target corn genomic nucleic acid, wherein the guide polynucleotide and the RNA-guided nuclease form a complex that can bind to and cleave the corn genomic nucleic acid molecule.

86. The method of claim 85, wherein;
a) the guide polynucleotide comprises a nucleotide sequence having at least 85% sequence identity, at least 90% sequence identity, or at least 95% sequence identity to a nucleic acid molecule selected from the group consisting of SEQ ID NOs:195-211; or
b) the guide polynucleotide further comprises SEQ ID NO: 23.

87-90. (canceled)

91. A recombinant polynucleotide molecule comprising:
a) a recombinant DNA molecule comprising a DNA sequence having at least 85% sequence identity, at least 90% sequence identity, or at least 95% sequence identity to a nucleic acid molecule selected from the group consisting of SEQ ID NOs:195-211; or
b) a recombinant RNA molecule comprising an RNA sequence that is at least 85% complementary, at least 90% complementary, or at least 95% complementary, to a nucleic acid molecule selected from the group consisting of SEQ ID NOs:195-211.

92. The recombinant polynucleotide molecule of claim 91, wherein:
a) said recombinant DNA molecule comprises a nucleic acid molecule selected from the group consisting of SEQ ID NOs:195-211;
b) said DNA sequence is operably linked to a heterologous promoter sequence;
c) said recombinant DNA molecule comprises SEQ ID ON: 23; or
d) said RNA sequence is 100% complementary to a nucleic acid molecule selected from the group consisting of SEQ ID NOs:195-211.

93-96. (canceled)

97. A method for controlling or preventing weed growth in an area, the method comprising planting the corn plant or seed of claim 29, wherein said corn plant or seed further comprises event MON87429 in the area and applying an effective amount of at least one herbicide selected from the group consisting of a PPO herbicide, dicamba, glufosinate, 2,4-D, glyphosate, a FOP herbicide, and combinations of any thereof to control weeds in the area without injury to the corn or with less than about 10% injury to the corn.


SEQUENCE LISTING (221 sequences)

SEQ ID NO: 1 - DNA, 30 bp, synthetic construct
  source 1..30: mol_type = other DNA
aataggttttgccaccccttaccaattttt

SEQ ID NO: 2 - DNA, 30 bp, synthetic construct
  source 1..30: mol_type = other DNA
actatcagtgtttgagcctatcaacatgca

SEQ ID NO: 3 - DNA, 60 bp, synthetic construct
  source 1..60: mol_type = other DNA
aatcacactcagcataataggttttgccaccccttaccaattttttttcaattcaaaaat

SEQ ID NO: 4 - DNA, 60 bp, synthetic construct
  source 1..60: mol_type = other DNA
ccgccttcagtttaaactatcagtgtttgagcctatcaacatgcagttcttagataaaat

SEQ ID NO: 5 - DNA, 100 bp, synthetic construct
  source 1..100: mol_type = other DNA
aaacaaaatagtaaagacctaatcacactcagcataataggttttgccaccccttaccaattttttttcaattcaaaaatgtagatgtccgcagcgttat

SEQ ID NO: 6 - DNA, 100 bp, synthetic construct
  source 1..100: mol_type = other DNA
tacgcgtagattgtcgtttcccgccttcagtttaaactatcagtgtttgagcctatcaacatgcagttcttagataaaatcctaactgtcagaaattgaa

SEQ ID NO: 7 - DNA, 1050 bp, synthetic construct
  source 1..1050: mol_type = other DNA
gttggatcttctacttgcggcttcatcgtttagcaatgctcaactacaatttggaactctcccacgtacctggccacacgacaagtcgcgatgctttgagagcttcttccccctcccctccttaaaccctgacatcgtgctggtgttgcctgtcgctcggagcgtcttcctctccccactccactcaaacatcgacgtcttctgtccgttcgaagcgactccgagtggagcaccaatccctccaaccagacaccgccggcatgacccacttccaggaactccctactctccctcatcctctgaaacgacaatgccgctgtcgtgtcggcgcgggccagagcaccattccctcctctgtgtgctgattccaatcggagctccatccctcccctcccccggccgccctcaagcgccaacacgttttgtccgtgtggagtggatccaggtggagcaccaggtgaaggcccatcaatttcagtggatggacagtggacgttgaaggcccagctatcagcaagagaaacagttagcagacctgcacacgagttgtcgtcaataactaactagtaagttccaagttcactagcacgtgttctataaccttattccattgaaattgatttcccttcttttctgtttgtcacgtggttaatgtgatgcaagtttataaataatgaagatgctgaagttgagaaagctcactaatactttatagcattgtctacatttcgatgcttttcttcatatccagctgtcttttcaaaccttgaaaaaatactactttggttttgatagactacctaaacacaaatcacaaagaaaagatgatgcatgtcataatgatataactaatctttccaaagtagtgtaccaaattttatttaacccagaaaaataccaccccttaattagagaaagaatcctaaactaatgcaaattataattttgataaacaaaatagtaaagacctaatcacactcagcataataggttttgccaccccttaccaattttttttcaattcaaaaatgtagatgtccgcagcgttat

SEQ ID NO: 8 - DNA, 1050 bp, synthetic construct
  source 1..1050: mol_type = other DNA
tacgcgtagattgtcgtttcccgccttcagtttaaactatcagtgtttgagcctatcaacatgcagttcttagataaaatcctaactgtcagaaattgaaaacacacaataactgatgtaacagactaaaagagcaagttcatttaccccattaacaataaataagatttcatggaataggctttatccgtttcgttttatatgttcagcaattcatctagcttgcatcttttattatacaacatgtaaatggaagagaacaagaatatttcaatagaaatgactgattagcaatcagtacacttattcacagtgagtacaaacagtggcggccaatagcatttatgattaagtgggtctatacgtactgccaaatatacatagatatatacattggagactgaagtctaatatttataggttgttttgtttgaggaataagctagttcatcatcttctcactcacttttttgtttggtttgtgaaattgaatgagttgatctatcaccacctcattcttcatagttagttagtattaacatgaagaattagaccatcccactaaatttaaggaatgtacttatgatgcaacaccttacaattacattgacattgtcgtaatagttaccggcactataatttaccacaattaatcattatattcttaatagttatgcaccattgacatcataaaaaggttcaataggataataatatgcacatgggcttctgtacaaaaccatgattcttaaagtaaataacacaatgaacattatctgcaatgttcacatcatattaaatataaaaaaataggtaaaacatatgtctattgtgcttgatcgtagacttcggcccattgttggacacaacatggcccgccatccatccatgtcttgtctcggttcgacaaaatcggctaggcttgtggcaagctcgtgctgagctggcccgacacgacctattgcctatatatacttctatgtgaacctgtccatatatattggtgctccaatatatttacaaaattgatctttgagtcagtggatgcat

SEQ ID NO: 9 - DNA, 3552 bp, synthetic construct
  source 1..3552: mol_type = other DNA
cccttaccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgaggctagcataacttcgtataatgtatgctatacgaagttatacgcgttccaagttcttgtggcttcttattttgtttaaattgttaaaactgaaaaatgtccctaggcctctaaaaactttgagaaaactcatataaaatatttttggtacatgagaaaccctaaaaaaatatttagaacacatgaaagtatctataaaagcttaaaaagaatttatcataagaaacatggagacagtatccagaaaagttataaaatttccacaaacatttaatgaatatgtaaacatgtttggaaaaactttccacaacaaaaccatgagatgcaataatcgtacacatttttcaagatttttttgaatatttttccatttcctagagctaaatacaacttttggaagcttttacagatacttccatgagatttaaatatttatttccattttcatgtatcacatatctttagattttttttctaggagcttttataagcttagagatattttcactttttacaaccttattatagttttcttattttaactaaaaaataaaatatttttaaaaaacacttctaatctgaacgggcatggaggtattttaaattataactaacgagtagaccactactacaactttttccttttttctatagaaatgaggatagttttttttttaaatggaggaatgaggataggtactgaagcccaagtcgtcgttttcgagattgggctgtatagcctcttaaacaaggaatcggacctcaaaagcccatcacggaactgccctgctgttatcggtgttggagagcgtgtccaaatggttccagaggctgtgtggtggggctggattgcgccacggcctcatgttcgctgcgcgatttctggttgccttgatgaggcgcaggtgggcccctccgttgcccaggataaaagtccacttccggcctccggtttccccaatccatcagccgccaccgatcccaatcgtgagttctccgcaccatcggcacgcagacgaaggaagcaaggctctaccgatcgtctcttgaaggtacactcttcctcgattcgtcaacctcgatttaggaggtacactcttcctcgattcgtcaacctcgatttgcctaagcgtgtatggtcggttctcgaatctcgattccccgcatagcgtgtatggtcgattgaacgtgtatggtcgattttcgaatctcgattttggattctgatgtcttcccttttacttatgatttgatgtatcacatagcttaatagattctgcaccgatctgttacttatgatttgattgattggttgcttgttgcgttccacaaaggacggtgctattttctgttctggaacgagatcatgtagatattatatagttagggttccaattcaaaaggttgattagtgtattataaaaattaaaagggctaaatcgttcctatcatgttgtctcgagcgtaatcctgcaggtatcatagaattatgtttgaattcaatagattgatcggtgtatcatttaaatcaaggctaaatcactacacttctgttgctacagagcacaatcctgtaggtatcttatcttagaattaagttttgataaattaattagtgtattttttagaacaaattgattagtatattattgtcaaattgagaaggttaaatcatgcctaccatgttgtctccgtttgtgatcatattagtcagaaggatatgcataaatcttctgctaaacttatttgttgtttagccctttcagatttttatgttgaatcctttctctatatttgctgcttgtatatgttgaatcgtcatgctaattgcaaatgtaattagcatattggcagttggtatatgcatcttcctaatctggatggatgcagtgttgtagtatgttctaatgcatggttcatcaaattttgtctgtattcactttttgttgcctctgttccatagttgagacttgaatcattattttctgagcactatgtttttctacacctattatggtagcttaccatgtgacttttgcctctgcaggtgcagggtaccatggcgacggctacgacgactgctacggcggcgtttagtggtgtagtcagtgtaggaacggagactcgaaggatttattcgttttctcatcttcaaccttctgcggcttttccggcgaagcctagttccttcaaatctctcaaattaaagcagagcgcgaggctcacacggcggcttgatcatcggccgttcgttgtccgatgtaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctagttaattaagggcccatgcttcctggtgctccgtcaggcctgtgtgccgctgctatcctatgatctgcccgagtggctcgtatccgttatgtgtctgtttgcttgaactatcagtcgttgtgtgtggtatggtactatggtttacaacacctgatgttgtaagacttgttatgtttaatccccgctttgctactgggttattatgaacatcgttatggttgtaacatttggtgctctttttttttactttctgctgagttaatgttgctgaggacggccttgacgcctgataaattttctgtgccgtctgttatcaccgctttgcccactgttttgtccctcatgcgagggagactattcgtggtattaaccagtgcttttctgtcgatgctaaccagtgcttttctgtcgatgctaatctccagtgacagtagttgtacccattccagttggttgcttccagtctttgccacctcttaatgtgacattgtcaaagcagttccccgtgtttgtacgcgtagattgtcgtttcccgccttcagtttaaactatcagtgtttga

SEQ ID NO: 10 - DNA, 5552 bp, synthetic construct
  source 1..5552: mol_type = other DNA
gttggatcttctacttgcggcttcatcgtttagcaatgctcaactacaatttggaactctcccacgtacctggccacacgacaagtcgcgatgctttgagagcttcttccccctcccctccttaaaccctgacatcgtgctggtgttgcctgtcgctcggagcgtcttcctctccccactccactcaaacatcgacgtcttctgtccgttcgaagcgactccgagtggagcaccaatccctccaaccagacaccgccggcatgacccacttccaggaactccctactctccctcatcctctgaaacgacaatgccgctgtcgtgtcggcgcgggccagagcaccattccctcctctgtgtgctgattccaatcggagctccatccctcccctcccccggccgccctcaagcgccaacacgttttgtccgtgtggagtggatccaggtggagcaccaggtgaaggcccatcaatttcagtggatggacagtggacgttgaaggcccagctatcagcaagagaaacagttagcagacctgcacacgagttgtcgtcaataactaactagtaagttccaagttcactagcacgtgttctataaccttattccattgaaattgatttcccttcttttctgtttgtcacgtggttaatgtgatgcaagtttataaataatgaagatgctgaagttgagaaagctcactaatactttatagcattgtctacatttcgatgcttttcttcatatccagctgtcttttcaaaccttgaaaaaatactactttggttttgatagactacctaaacacaaatcacaaagaaaagatgatgcatgtcataatgatataactaatctttccaaagtagtgtaccaaattttatttaacccagaaaaataccaccccttaattagagaaagaatcctaaactaatgcaaattataattttgataaacaaaatagtaaagacctaatcacactcagcataataggttttgccaccccttaccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgaggctagcataacttcgtataatgtatgctatacgaagttatacgcgttccaagttcttgtggcttcttattttgtttaaattgttaaaactgaaaaatgtccctaggcctctaaaaactttgagaaaactcatataaaatatttttggtacatgagaaaccctaaaaaaatatttagaacacatgaaagtatctataaaagcttaaaaagaatttatcataagaaacatggagacagtatccagaaaagttataaaatttccacaaacatttaatgaatatgtaaacatgtttggaaaaactttccacaacaaaaccatgagatgcaataatcgtacacatttttcaagatttttttgaatatttttccatttcctagagctaaatacaacttttggaagcttttacagatacttccatgagatttaaatatttatttccattttcatgtatcacatatctttagattttttttctaggagcttttataagcttagagatattttcactttttacaaccttattatagttttcttattttaactaaaaaataaaatatttttaaaaaacacttctaatctgaacgggcatggaggtattttaaattataactaacgagtagaccactactacaactttttccttttttctatagaaatgaggatagttttttttttaaatggaggaatgaggataggtactgaagcccaagtcgtcgttttcgagattgggctgtatagcctcttaaacaaggaatcggacctcaaaagcccatcacggaactgccctgctgttatcggtgttggagagcgtgtccaaatggttccagaggctgtgtggtggggctggattgcgccacggcctcatgttcgctgcgcgatttctggttgccttgatgaggcgcaggtgggcccctccgttgcccaggataaaagtccacttccggcctccggtttccccaatccatcagccgccaccgatcccaatcgtgagttctccgcaccatcggcacgcagacgaaggaagcaaggctctaccgatcgtctcttgaaggtacactcttcctcgattcgtcaacctcgatttaggaggtacactcttcctcgattcgtcaacctcgatttgcctaagcgtgtatggtcggttctcgaatctcgattccccgcatagcgtgtatggtcgattgaacgtgtatggtcgattttcgaatctcgattttggattctgatgtcttcccttttacttatgatttgatgtatcacatagcttaatagattctgcaccgatctgttacttatgatttgattgattggttgcttgttgcgttccacaaaggacggtgctattttctgttctggaacgagatcatgtagatattatatagttagggttccaattcaaaaggttgattagtgtattataaaaattaaaagggctaaatcgttcctatcatgttgtctcgagcgtaatcctgcaggtatcatagaattatgtttgaattcaatagattgatcggtgtatcatttaaatcaaggctaaatcactacacttctgttgctacagagcacaatcctgtaggtatcttatcttagaattaagttttgataaattaattagtgtattttttagaacaaattgattagtatattattgtcaaattgagaaggttaaatcatgcctaccatgttgtctccgtttgtgatcatattagtcagaaggatatgcataaatcttctgctaaacttatttgttgtttagccctttcagatttttatgttgaatcctttctctatatttgctgcttgtatatgttgaatcgtcatgctaattgcaaatgtaattagcatattggcagttggtatatgcatcttcctaatctggatggatgcagtgttgtagtatgttctaatgcatggttcatcaaattttgtctgtattcactttttgttgcctctgttccatagttgagacttgaatcattattttctgagcactatgtttttctacacctattatggtagcttaccatgtgacttttgcctctgcaggtgcagggtaccatggcgacggctacgacgactgctacggcggcgtttagtggtgtagtcagtgtaggaacggagactcgaaggatttattcgttttctcatcttcaaccttctgcggcttttccggcgaagcctagttccttcaaatctctcaaattaaagcagagcgcgaggctcacacggcggcttgatcatcggccgttcgttgtccgatgtaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctagttaattaagggcccatgcttcctggtgctccgtcaggcctgtgtgccgctgctatcctatgatctgcccgagtggctcgtatccgttatgtgtctgtttgcttgaactatcagtcgttgtgtgtggtatggtactatggtttacaacacctgatgttgtaagacttgttatgtttaatccccgctttgctactgggttattatgaacatcgttatggttgtaacatttggtgctctttttttttactttctgctgagttaatgttgctgaggacggccttgacgcctgataaattttctgtgccgtctgttatcaccgctttgcccactgttttgtccctcatgcgagggagactattcgtggtattaaccagtgcttttctgtcgatgctaaccagtgcttttctgtcgatgctaatctccagtgacagtagttgtacccattccagttggttgcttccagtctttgccacctcttaatgtgacattgtcaaagcagttccccgtgtttgtacgcgtagattgtcgtttcccgccttcagtttaaactatcagtgtttgagcctatcaacatgcagttcttagataaaatcctaactgtcagaaattgaaaacacacaataactgatgtaacagactaaaagagcaagttcatttaccccattaacaataaataagatttcatggaataggctttatccgtttcgttttatatgttcagcaattcatctagcttgcatcttttattatacaacatgtaaatggaagagaacaagaatatttcaatagaaatgactgattagcaatcagtacacttattcacagtgagtacaaacagtggcggccaatagcatttatgattaagtgggtctatacgtactgccaaatatacatagatatatacattggagactgaagtctaatatttataggttgttttgtttgaggaataagctagttcatcatcttctcactcacttttttgtttggtttgtgaaattgaatgagttgatctatcaccacctcattcttcatagttagttagtattaacatgaagaattagaccatcccactaaatttaaggaatgtacttatgatgcaacaccttacaattacattgacattgtcgtaatagttaccggcactataatttaccacaattaatcattatattcttaatagttatgcaccattgacatcataaaaaggttcaataggataataatatgcacatgggcttctgtacaaaaccatgattcttaaagtaaataacacaatgaacattatctgcaatgttcacatcatattaaatataaaaaaataggtaaaacatatgtctattgtgcttgatcgtagacttcggcccattgttggacacaacatggcccgccatccatccatgtcttgtctcggttcgacaaaatcggctaggcttgtggcaagctcgtgctgagctggcccgacacgacctattgcctatatatacttctatgtgaacctgtccatatatattggtgctccaatatatttacaaaattgatctttgagtcagtggatgcat

SEQ ID NO: 11 - DNA, 1000 bp, Glycine max
  source 1..1000: mol_type = genomic DNA
gttggatcttctacttgcggcttcatcgtttagcaatgctcaactacaatttggaactctcccacgtacctggccacacgacaagtcgcgatgctttgagagcttcttccccctcccctccttaaaccctgacatcgtgctggtgttgcctgtcgctcggagcgtcttcctctccccactccactcaaacatcgacgtcttctgtccgttcgaagcgactccgagtggagcaccaatccctccaaccagacaccgccggcatgacccacttccaggaactccctactctccctcatcctctgaaacgacaatgccgctgtcgtgtcggcgcgggccagagcaccattccctcctctgtgtgctgattccaatcggagctccatccctcccctcccccggccgccctcaagcgccaacacgttttgtccgtgtggagtggatccaggtggagcaccaggtgaaggcccatcaatttcagtggatggacagtggacgttgaaggcccagctatcagcaagagaaacagttagcagacctgcacacgagttgtcgtcaataactaactagtaagttccaagttcactagcacgtgttctataaccttattccattgaaattgatttcccttcttttctgtttgtcacgtggttaatgtgatgcaagtttataaataatgaagatgctgaagttgagaaagctcactaatactttatagcattgtctacatttcgatgcttttcttcatatccagctgtcttttcaaaccttgaaaaaatactactttggttttgatagactacctaaacacaaatcacaaagaaaagatgatgcatgtcataatgatataactaatctttccaaagtagtgtaccaaattttatttaacccagaaaaataccaccccttaattagagaaagaatcctaaactaatgcaaattataattttgataaacaaaatagtaaagacctaatcacactcagcataataggttttgccac

SEQ ID NO: 12 - DNA, 1000 bp, Glycine max
  source 1..1000: mol_type = genomic DNA
gcctatcaacatgcagttcttagataaaatcctaactgtcagaaattgaaaacacacaataactgatgtaacagactaaaagagcaagttcatttaccccattaacaataaataagatttcatggaataggctttatccgtttcgttttatatgttcagcaattcatctagcttgcatcttttattatacaacatgtaaatggaagagaacaagaatatttcaatagaaatgactgattagcaatcagtacacttattcacagtgagtacaaacagtggcggccaatagcatttatgattaagtgggtctatacgtactgccaaatatacatagatatatacattggagactgaagtctaatatttataggttgttttgtttgaggaataagctagttcatcatcttctcactcacttttttgtttggtttgtgaaattgaatgagttgatctatcaccacctcattcttcatagttagttagtattaacatgaagaattagaccatcccactaaatttaaggaatgtacttatgatgcaacaccttacaattacattgacattgtcgtaatagttaccggcactataatttaccacaattaatcattatattcttaatagttatgcaccattgacatcataaaaaggttcaataggataataatatgcacatgggcttctgtacaaaaccatgattcttaaagtaaataacacaatgaacattatctgcaatgttcacatcatattaaatataaaaaaataggtaaaacatatgtctattgtgcttgatcgtagacttcggcccattgttggacacaacatggcccgccatccatccatgtcttgtctcggttcgacaaaatcggctaggcttgtggcaagctcgtgctgagctggcccgacacgacctattgcctatatatacttctatgtgaacctgtccatatatattggtgctccaatatatttacaaaattgatctttgagtcagtggatgcat

SEQ ID NO: 13 - DNA, 2019 bp, Glycine max
  source 1..2019: mol_type = genomic DNA
gttggatcttctacttgcggcttcatcgtttagcaatgctcaactacaatttggaactctcccacgtacctggccacacgacaagtcgcgatgctttgagagcttcttccccctcccctccttaaaccctgacatcgtgctggtgttgcctgtcgctcggagcgtcttcctctccccactccactcaaacatcgacgtcttctgtccgttcgaagcgactccgagtggagcaccaatccctccaaccagacaccgccggcatgacccacttccaggaactccctactctccctcatcctctgaaacgacaatgccgctgtcgtgtcggcgcgggccagagcaccattccctcctctgtgtgctgattccaatcggagctccatccctcccctcccccggccgccctcaagcgccaacacgttttgtccgtgtggagtggatccaggtggagcaccaggtgaaggcccatcaatttcagtggatggacagtggacgttgaaggcccagctatcagcaagagaaacagttagcagacctgcacacgagttgtcgtcaataactaactagtaagttccaagttcactagcacgtgttctataaccttattccattgaaattgatttcccttcttttctgtttgtcacgtggttaatgtgatgcaagtttataaataatgaagatgctgaagttgagaaagctcactaatactttatagcattgtctacatttcgatgcttttcttcatatccagctgtcttttcaaaccttgaaaaaatactactttggttttgatagactacctaaacacaaatcacaaagaaaagatgatgcatgtcataatgatataactaatctttccaaagtagtgtaccaaattttatttaacccagaaaaataccaccccttaattagagaaagaatcctaaactaatgcaaattataattttgataaacaaaatagtaaagacctaatcacactcagcataataggttttgccacaaggctattcactccctatgcctatcaacatgcagttcttagataaaatcctaactgtcagaaattgaaaacacacaataactgatgtaacagactaaaagagcaagttcatttaccccattaacaataaataagatttcatggaataggctttatccgtttcgttttatatgttcagcaattcatctagcttgcatcttttattatacaacatgtaaatggaagagaacaagaatatttcaatagaaatgactgattagcaatcagtacacttattcacagtgagtacaaacagtggcggccaatagcatttatgattaagtgggtctatacgtactgccaaatatacatagatatatacattggagactgaagtctaatatttataggttgttttgtttgaggaataagctagttcatcatcttctcactcacttttttgtttggtttgtgaaattgaatgagttgatctatcaccacctcattcttcatagttagttagtattaacatgaagaattagaccatcccactaaatttaaggaatgtacttatgatgcaacaccttacaattacattgacattgtcgtaatagttaccggcactataatttaccacaattaatcattatattcttaatagttatgcaccattgacatcataaaaaggttcaataggataataatatgcacatgggcttctgtacaaaaccatgattcttaaagtaaataacacaatgaacattatctgcaatgttcacatcatattaaatataaaaaaataggtaaaacatatgtctattgtgcttgatcgtagacttcggcccattgttggacacaacatggcccgccatccatccatgtcttgtctcggttcgacaaaatcggctaggcttgtggcaagctcgtgctgagctggcccgacacgacctattgcctatatatacttctatgtgaacctgtccatatatattggtgctccaatatatttacaaaattgatctttgagtcagtggatgcat

SEQ ID NO: 14 - DNA, 30 bp, synthetic construct
  source 1..30: mol_type = other DNA
gtcgttttatcaaaatgtactttcatttta

SEQ ID NO: 15 - DNA, 30 bp, synthetic construct
  source 1..30: mol_type = other DNA
aaatagtaaagacctaatcacactcagcat

SEQ ID NO: 16 - DNA, 16 bp, synthetic construct
  source 1..16: mol_type = other DNA
taacgctgcggacatc

SEQ ID NO: 17 - DNA, 24 bp, synthetic construct
  source 1..24: mol_type = other DNA
gccctatgacttaccgagagttca

SEQ ID NO: 18 - DNA, 28 bp, synthetic construct
  source 1..28: mol_type = other DNA
gttgctatgtactaacagaactgcatgt

SEQ ID NO: 19 - DNA, 17 bp, synthetic construct
  source 1..17: mol_type = other DNA
ttgttgtgtggctccat

SEQ ID NO: 20 - DNA, 34 bp, synthetic construct
  source 1..34: mol_type = other DNA
gataaacaaaatagtaaagacctaatcacactca

SEQ ID NO: 21 - DNA, 32 bp, synthetic construct
  source 1..32: mol_type = other DNA
gctcttttagtctgttacatcagttattgtgt

SEQ ID NO: 22 - DNA, 24 bp, synthetic construct
  source 1..24: mol_type = other DNA
ctatcaacatgcagttcttagata

SEQ ID NO: 23 - DNA, 21 bp, unidentified
  source 1..21: mol_type = genomic DNA; note = Lachnospiraceae bacterium ND2006
taatttctactaagtgtagat

SEQ ID NO: 24 - DNA, 442 bp, Agrobacterium tumefaciens
  source 1..442: mol_type = genomic DNA
ctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggactttt

SEQ ID NO: 25 - DNA, 34 bp, unidentified
  source 1..34: mol_type = genomic DNA; note = Escherichia virus P1
ataacttcgtatagcatacattatacgaagttat

SEQ ID NO: 26 - DNA, 851 bp, Andropogon gerardi
  source 1..851: mol_type = genomic DNA
tccaagttcttgtggcttcttattttgtttaaattgttaaaactgaaaaatgtccctaggcctctaaaaactttgagaaaactcatataaaatatttttggtacatgagaaaccctaaaaaaatatttagaacacatgaaagtatctataaaagcttaaaaagaatttatcataagaaacatggagacagtatccagaaaagttataaaatttccacaaacatttaatgaatatgtaaacatgtttggaaaaactttccacaacaaaaccatgagatgcaataatcgtacacatttttcaagatttttttgaatatttttccatttcctagagctaaatacaacttttggaagcttttacagatacttccatgagatttaaatatttatttccattttcatgtatcacatatctttagattttttttctaggagcttttataagcttagagatattttcactttttacaaccttattatagttttcttattttaactaaaaaataaaatatttttaaaaaacacttctaatctgaacgggcatggaggtattttaaattataactaacgagtagaccactactacaactttttccttttttctatagaaatgaggatagttttttttttaaatggaggaatgaggataggtactgaagcccaagtcgtcgttttcgagattgggctgtatagcctcttaaacaaggaatcggacctcaaaagcccatcacggaactgccctgctgttatcggtgttggagagcgtgtccaaatggttccagaggctgtgtggtggggctggattgcgccacggcctcatgttcgctgcgcgatttctggttgccttgatga

SEQ ID NO: 27 - DNA, 154 bp, Andropogon gerardi
  source 1..154: mol_type = genomic DNA
ggcgcaggtgggcccctccgttgcccaggataaaagtccacttccggcctccggtttccccaatccatcagccgccaccgatcccaatcgtgagttctccgcaccatcggcacgcagacgaaggaagcaaggctctaccgatcgtctcttgaag

SEQ ID NO: 28 - DNA, 1001 bp, Andropogon gerardi
  source 1..1001: mol_type = genomic DNA
gtacactcttcctcgattcgtcaacctcgatttaggaggtacactcttcctcgattcgtcaacctcgatttgcctaagcgtgtatggtcggttctcgaatctcgattccccgcatagcgtgtatggtcgattgaacgtgtatggtcgattttcgaatctcgattttggattctgatgtcttcccttttacttatgatttgatgtatcacatagcttaatagattctgcaccgatctgttacttatgatttgattgattggttgcttgttgcgttccacaaaggacggtgctattttctgttctggaacgagatcatgtagatattatatagttagggttccaattcaaaaggttgattagtgtattataaaaattaaaagggctaaatcgttcctatcatgttgtctcgagcgtaatcctgcaggtatcatagaattatgtttgaattcaatagattgatcggtgtatcatttaaatcaaggctaaatcactacacttctgttgctacagagcacaatcctgtaggtatcttatcttagaattaagttttgataaattaattagtgtattttttagaacaaattgattagtatattattgtcaaattgagaaggttaaatcatgcctaccatgttgtctccgtttgtgatcatattagtcagaaggatatgcataaatcttctgctaaacttatttgttgtttagccctttcagatttttatgttgaatcctttctctatatttgctgcttgtatatgttgaatcgtcatgctaattgcaaatgtaattagcatattggcagttggtatatgcatcttcctaatctggatggatgcagtgttgtagtatgttctaatgcatggttcatcaaattttgtctgtattcactttttgttgcctctgttccatagttgagacttgaatcattattttctgagcactatgtttttctacacctattatggtagcttaccatgtgacttttgcctctgcaggtgcag

SEQ ID NO: 29 - DNA, 204 bp, Arabidopsis thaliana
  source 1..204: mol_type = genomic DNA
atggcgacggctacgacgactgctacggcggcgtttagtggtgtagtcagtgtaggaacggagactcgaaggatttattcgttttctcatcttcaaccttctgcggcttttccggcgaagcctagttccttcaaatctctcaaattaaagcagagcgcgaggctcacacggcggcttgatcatcggccgttcgttgtccgatgt

SEQ ID NO: 30 - DNA, 537 bp, Enterobacter cloacae
  source 1..537: mol_type = genomic DNA
aaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 31 - DNA, 498 bp, Arundo donax
  source 1..498: mol_type = genomic DNA
atgcttcctggtgctccgtcaggcctgtgtgccgctgctatcctatgatctgcccgagtggctcgtatccgttatgtgtctgtttgcttgaactatcagtcgttgtgtgtggtatggtactatggtttacaacacctgatgttgtaagacttgttatgtttaatccccgctttgctactgggttattatgaacatcgttatggttgtaacatttggtgctctttttttttactttctgctgagttaatgttgctgaggacggccttgacgcctgataaattttctgtgccgtctgttatcaccgctttgcccactgttttgtccctcatgcgagggagactattcgtggtattaaccagtgcttttctgtcgatgctaaccagtgcttttctgtcgatgctaatctccagtgacagtagttgtacccattccagttggttgcttccagtctttgccacctcttaatgtgacattgtcaaagcagttccccgtgtttgt

SEQ ID NO: 32 - DNA, 357 bp, Agrobacterium tumefaciens
  source 1..357: mol_type = genomic DNA
aggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatct

SEQ ID NO: 33 - DNA, 3684 bp, synthetic construct
  source 1..3684: mol_type = other DNA
atgtcgaagctcgagaagttcaccaactgctactcgctgagcaagacgctgcggttcaaggcgatccccgtcgggaagacccaggagaacatcgacaacaagcggctcctggtcgaggacgagaagcgcgccgaggactacaagggcgtcaagaagctgctggaccggtactacctctccttcatcaacgacgtcctgcactcgatcaagctcaagaacctgaacaactacatctcgctgttccgcaagaagacacggaccgagaaggagaacaaggagctcgagaacctcgagatcaacctgcgcaaggagatcgcgaaggcgttcaagggcaacgaggggtacaagagcctgttcaagaaagacatcatcgagaccatcctgccggagttcctggacgacaaggacgagatcgcgctggtgaactcgttcaacgggttcaccacggccttcaccgggtttttcgacaaccgggagaacatgttcagcgaggaggccaagtcgaccagcatcgccttccggtgcatcaacgagaacctcacccgctacatcagcaacatggacatcttcgagaaggtggacgccatcttcgacaagcacgaggtccaggagatcaaggaaaagatcctgaactcggactacgacgtggaagacttctttgagggcgagttcttcaacttcgtcctcacccaggagggcatcgacgtctacaacgccatcatcggcggcttcgtgacggagagcggcgagaagatcaagggcctcaacgagtacatcaacctctacaaccagaagactaagcagaagctcccgaagttcaagccgctgtacaagcaagtcctgagcgaccgggagtccctctcgttctacggcgagggctacacgagcgacgaggaggtgctggaggtgttccgcaacacgctgaacaagaacagcgagatcttcagctcgatcaagaaactcgagaagctgttcaagaacttcgacgagtacagcagcgccggcatcttcgtcaagaacgggcccgcgatcagcaccatcagcaaggacatcttcggggagtggaacgtgatccgcgacaagtggaacgccgagtacgacgacatccacctcaagaaaaaggcggtggtcacggagaagtacgaggacgaccgccggaagtccttcaagaaaatcgggagcttcagcctcgagcagctccaggagtacgcggacgccgacctgagcgtggtggagaagctcaaggagatcatcatccagaaggtcgacgagatctacaaggtctacggctcgagcgagaagctgttcgacgcggacttcgtgctggagaagtccctcaagaagaacgacgccgtggtggccatcatgaaggatctgctcgacagcgtgaagtcgttcgagaactacatcaaggcattctttggggagggcaaggagacgaaccgggacgagtccttctacggggacttcgtgctcgcgtacgacatcctcctgaaggtcgaccacatctacgacgcgatccggaactacgtcacgcagaagccctacagcaaggacaagttcaagctctacttccagaacccgcagttcatgggcgggtgggacaaggacaaggagaccgactaccgggccacgatcctgcggtacgggtccaagtactacctcgccatcatggacaagaagtacgccaagtgcctccagaagattgacaaggacgacgtgaacgggaactacgagaagatcaactacaagctcctcccggggcccaacaagatgctgccgaaggtgttcttcagcaagaagtggatggcctactacaacccctcggaggacatccagaagatatacaagaacggcacgttcaaaaagggggacatgttcaacctgaacgactgccacaagctgatcgactttttcaaggacagcatcagccgctacccgaagtggtcgaacgcctacgacttcaacttctcggagacggagaagtacaaggacattgcgggcttctaccgggaggtggaggagcagggctacaaggtctccttcgagagcgcctccaagaaagaggtggacaagctcgtggaggagggcaagctgtacatgttccagatctacaacaaggacttctcggacaagtcgcacggcaccccgaacctccacacgatgtacttcaagctgctgttcgacgagaacaaccacgggcagatccgcctcagcggcggggcggagctgttcatgcgccgcgcgtccctcaagaaggaggagctggtcgtgcaccccgccaactccccgatcgcgaacaagaaccccgacaaccccaagaagacaaccaccctctcgtacgacgtctacaaggacaagcggttctcggaggaccagtacgagctgcacatcccgatcgccatcaacaagtgccccaagaacatcttcaagatcaacaccgaggtgcgggtgctgctcaagcacgacgacaacccctacgtcatcgggatcgaccgcggcgagcggaacctgctctacatcgtggtcgtggacgggaaggggaacatcgtggagcagtacagcctgaacgagatcatcaacaacttcaacggcatccgcatcaagacggactaccacagcctcctggacaagaaggagaaggagcggttcgaggcgcggcagaactggacctccatcgagaacatcaaggagctgaaggccggctacatcagccaggtcgtgcacaagatctgcgagctcgtggagaagtacgacgcggtgatcgcgctggaggacttgaacagcgggttcaagaactcccgggtcaaggtcgagaagcaggtctaccagaagttcgagaagatgctgatcgacaagctcaactacatggtggacaagaagtccaacccctgcgccaccggcggcgccctcaagggctaccagatcaccaacaagttcgagtccttcaagtcgatgtctacgcagaacgggttcattttctacatcccggcgtggctcaccagcaagatcgacccgagcacgggcttcgtcaacctcctgaagaccaagtacaccagcatcgcggacagcaagaagttcatctcctcgttcgaccgcatcatgtacgtccccgaggaagacctgttcgagttcgccctcgactacaagaacttctcccggacggacgccgactacatcaaaaagtggaagctctacagctacggcaaccggatccgcatcttccgcaaccccaagaagaacaatgtgttcgactgggaggaggtgtgcctgacgagcgcctacaaggagctcttcaacaagtacggcatcaactaccagcaaggggacatccgcgcgctgctctgcgagcagtccgacaaggcgttctactcgtcgttcatggccctgatgagcctcatgctccagatgcgcaacagcatcaccggccggacggacgtggacttcctgatcagcccggtcaagaacagcgacggcattttctacgacagccggaactacgaggcccaggagaacgccatcctccccaagaacgccgacgcgaacggcgcctacaacatcgcgcggaaggtgctgtgggccatcggccagtttaaaaaggcggaggacgagaagctggacaaggtcaagatcgccatcagcaacaaggagtggctcgagtacgcgcagacgagcgtgaagcac

SEQ ID NO: 34 - protein, 1228 aa, unidentified
  source 1..1228: note = Lachnospiraceae bacterium ND2006
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 35 - protein, 1228 aa, unidentified
  source 1..1228: note = Lachnospiraceae bacterium ND2006
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDKETDYRATILRYGSKYYLAIMDKKYAKCLQKIDKDDVNGNYEKINYKLLPGPNKMLPRVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 36 - protein, 1228 aa, unidentified
  source 1..1228: note = Lachnospiraceae bacterium ND2006
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDVETDRRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 37 - protein, 1300 aa, Francisella sp.
  source 1..1300: note = Francisella novicida
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 38 - DNA, 21 bp, synthetic construct
  source 1..21: mol_type = other DNA
gaatttctactaagtgtagat

SEQ ID NO: 39 - DNA, 21 bp, synthetic construct
  source 1..21: mol_type = other DNA
gtaatttctactgttgtagat

SEQ ID NO: 40 - DNA, 51 bp, synthetic construct
  source 1..51: mol_type = other DNA
gtaatttctactgttgtagattcgtcaataactaactagtaagtttttttt

SEQ ID NO: 41 - DNA, 51 bp, synthetic construct
  source 1..51: mol_type = other DNA
gtaatttctactgttgtagatagtgggtctatacgtactgccaattttttt

SEQ ID NO: 42 - DNA, 537 bp, synthetic construct; identical to SEQ ID NO: 30
  source 1..537: mol_type = other DNA

SEQ ID NO: 43 - protein, 178 aa, Enterobacter cloacae
KALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQLIMRMTGGETDTSKEVEYTDWEQVKKFAEDFAKLSYKKAL

SEQ ID NO: 44 - DNA, 50 bp, Zea mays
  source 1..50: mol_type = genomic DNA
gttggatcttctacttgcggcttcatcgtttagcaatgctcaactacaat

SEQ ID NO: 45 - DNA, 50 bp, Zea mays
  source 1..50: mol_type = genomic DNA
ttggaactctcccacgtacctggccacacgacaagtcgcgatgctttgag

SEQ ID NO: 46 - DNA, 50 bp, Zea mays
  source 1..50: mol_type = genomic DNA
agcttcttccccctcccctccttaaaccctgacatcgtgctggtgttgcc

SEQ ID NO: 47 - DNA, 50 bp, Zea mays
  source 1..50: mol_type = genomic DNA
tgtcgctcggagcgtcttcctctccccactccactcaaacatcgacgtct

SEQ ID NO: 48 - DNA, 50 bp, Zea mays
  source 1..50: mol_type = genomic DNA
tctgtccgttcgaagcgactccgagtggagcaccaatccctccaaccaga

SEQ ID NO: 49 - DNA, 50 bp, Zea mays
  source 1..50: mol_type = genomic DNA
caccgccggcatgacccacttccaggaactccctactctccctcatcctc

SEQ ID NO: 50 - DNA, 50 bp, Zea mays
  source 1..50: mol_type = genomic DNA
tgaaacgacaatgccgctgtcgtgtcggcgcgggccagagcaccattccc

SEQ ID NO: 51 - DNA, 50 bp, Zea mays
  source 1..50: mol_type = genomic DNA
tcctctgtgtgctgattccaatcggagctccatccctcccctcccccggc

SEQ ID NO: 52 - DNA, 50 bp, Zea mays
  source 1..50: mol_type = genomic DNA
cgccctcaagcgccaacacgttttgtccgtgtggagtggatccaggtgga

SEQ ID NO: 53 - DNA, 50 bp, Zea mays
  source 1..50: mol_type = genomic DNA
gcaccaggtgaaggcccatcaatttcagtggatggacagtggacgttgaa

SEQ ID NO: 54 - DNA, 50 bp, Zea mays
  source 1..50: mol_type = genomic DNA
ggcccagctatcagcaagagaaacagttagcagacctgcacacgagttgt

SEQ ID NO: 55 - DNA, 50 bp, Zea mays
  source 1..50: mol_type = genomic DNA
cgtcaataactaactagtaagttccaagttcactagcacgtgttctataa

SEQ ID NO: 56 - DNA, 50 bp, Zea mays
  source 1..50: mol_type = genomic DNA
ccttattccattgaaattgatttcccttcttttctgtttgtcacgtggtt

SEQ ID NO: 57 - DNA, 50 bp, Zea mays
  source 1..50: mol_type = genomic DNA
aatgtgatgcaagtttataaataatgaagatgctgaagttgagaaagctc

SEQ ID NO: 58 - DNA, 50 bp, Zea mays
  source 1..50: mol_type = genomic DNA
actaatactttatagcattgtctacatttcgatgcttttcttcatatcca

SEQ ID NO: 59 - DNA, 50 bp, Zea mays
  source 1..50: mol_type = genomic DNA
gctgtcttttcaaaccttgaaaaaatactactttggttttgatagactac

SEQ ID NO: 60 - DNA, 50 bp, Zea mays
  source 1..50: mol_type = genomic DNA
ctaaacacaaatcacaaagaaaagatgatgcatgtcataatgatataact

SEQ ID NO: 61 - DNA, 50 bp, Zea mays
  source 1..50: mol_type = genomic DNA
aatctttccaaagtagtgtaccaaattttatttaacccagaaaaatacca

SEQ ID NO: 62 - DNA, 50 bp, Zea mays
  source 1..50: mol_type = genomic DNA
ccccttaattagagaaagaatcctaaactaatgcaaattataattttgat

SEQ ID NO: 63 - DNA, 50 bp, Zea mays
  source 1..50: mol_type = genomic DNA
aaacaaaatagtaaagacctaatcacactcagcataataggttttgccac

SEQ ID NO: 64 - DNA, 50 bp, Zea mays
  source 1..50: mol_type = genomic DNA
aaaagtgatccaaatatgccaaatcctatgtgagatgaatttgtcttcaa

SEQ ID NO: 65 - DNA, 50 bp, Zea mays
  source 1..50: mol_type = genomic DNA
tttgatgtgttggcataaatcaccaaaaagggggagatttaaaaggaaat

SEQ ID NO: 66 - DNA, 50 bp, Zea mays
  source 1..50: mol_type = genomic DNA
attgttttggttgatatgcgctaagtgtcaaagagatgcaaatcaacaat

SEQ ID NO: 67 - DNA, 50 bp, Zea mays
  source 1..50: mol_type = genomic DNA
tctaagtgctaggaaccttgtcgaatcaaatgagattggatcggtttggc

SEQ ID NO: 68 - DNA, 50 bp, Zea mays
  source 1..50: mol_type = genomic DNA
tgcccaagctggcgcacgcgacaaacttgctgctcttgggaaaagttggg

SEQ ID NO: 69 - DNA, 50 bp, Zea mays
  source 1..50: mol_type = genomic DNA
cgctaacggtcggcagcgcaatcagcgggcgacgtatggcccgcgccaac

SEQ ID NO: 70 - DNA, 50 bp, Zea mays
  source 1..50: mol_type = genomic DNA
caatggtcggcttcaccagaaaaagaaggagatcacgcactgttcactgt

SEQ ID NO: 71 - DNA, 50 bp, Zea mays
  source 1..50: mol_type = genomic DNA
tgccttccaaatggagctccaacggctcctagttgccttagggctataaa

SEQ ID NO: 72 - DNA, 50 bp, Zea mays
  source 1..50: mol_type = genomic DNA
taagatgcctagactctgcaagcacgtattcaattcattgtgtttgagat

SEQ ID NO: 73 - DNA, 50 bp, Zea mays
  source 1..50: mol_type = genomic DNA
ttcttgacttgtgtgcgtgtcgttgctgcgattctagctcttgcatgtgt

SEQ ID NO: 74 - DNA, 50 bp, Zea mays
  source 1..50: mol_type = genomic DNA
ggtgagaggctccaacttgtagagattcctcacaaagggaatacttgaga

SEQ ID NO: 75 - DNA, 50 bp, Zea mays
  source 1..50: mol_type = genomic DNA
gggattgagtgcaatccttgaccaaaggaggttgccacaacgtggagtag

SEQ ID NO: 76 - DNA, 50 bp, Zea mays
  source 1..50: mol_type = genomic DNA
tattgtgatttctttcttccttagagttttacttaatcacttgcgctatt

SEQ ID NO: 77 - DNA, 50 bp, Zea mays
  source 1..50: mol_type = genomic DNA
ctctctctctctccattcatagcaacttggttttgatctcactaacattt

SEQ ID NO: 78 - DNA, 50 bp, Zea mays
  source 1..50: mol_type = genomic DNA
tcacccccccccccctctaggtgctcttagtaagcatggtttttcttatc

SEQ ID NO: 79 - DNA, 50 bp, Zea mays
  source 1..50: mol_type = genomic DNA
tgatgttggaggccttttggctctttaagtcattggttcccctttggaaa

SEQ ID NO: 80 - DNA, 50 bp, Zea mays
  source 1..50: mol_type = genomic DNA
agcaaatttaactttatcacattcatctttgcatattttaagttgagcat

SEQ ID NO: 81 - DNA, 50 bp, Zea mays
  source 1..50: mol_type = genomic DNA
ttacaagcatctacataaaaaaaaactttacatctattgcaaatagaaac

SEQ ID NO: 82 - DNA, 50 bp, Zea mays
  source 1..50: mol_type = genomic DNA
aatcaacatttacacttttaaggctagaaatagaataatggcaagaagac

SEQ ID NO: 83 - DNA, 50 bp, Zea mays
  source 1..50: mol_type = genomic DNA
aacattaacaaatttatcatgttcttcataaagaatgtcctcttgttttt

SEQ ID NO: 84 - DNA, 50 bp, Zea mays
  source 1..50: mol_type = genomic DNA
tcaactttggatctatctaagcccttaaagagatcgctataatctagatc

SEQ ID NO: 85 - DNA, 50 bp, Zea mays
  source 1..50: mol_type = genomic DNA
tacataccttcttctccttagccatgaggcatgtatgtcattcgttggag

SEQ ID NO: 86 - DNA, 50 bp, Zea mays
  source 1..50: mol_type = genomic DNA
gtcagatgaagagcagtccgagtcccattccttgccaatatgtgcctcgc

SEQ ID NO: 87 - DNA, 50 bp, Zea mays
  source 1..50: mol_type = genomic DNA
tgttcctggtcattttcattatagagacattgtgtaataaaatgaccagg

SEQ ID NO: 88 - DNA, 50 bp, Zea mays
  source 1..50: mol_type = genomic DNA
ggaagtccttgtgtcccttcaatgcggtcttgaagcgcttaatcacaagc

SEQ ID NO: 89 - DNA, 50 bp, Zea mays
  source 1..50: mol_type = genomic DNA
tggtagcgtctccttgctagtcgttgccttgagtgcaacgagttgcggct

SEQ ID NO: 90 - DNA, 50 bp, Zea mays
  source 1..50: mol_type = genomic DNA
ttcacaattatgcgtctgcttacgaattttcctagaattttctctagcga

SEQ ID NO: 91 - DNA, 50 bp, Zea mays
  source 1..50: mol_type = genomic DNA
taaatgatcttagcatcagtcgcacgacatcatgatctgtccatcttgtt

SEQ ID NO: 92 - DNA, 50 bp, Zea mays
  source 1..50: mol_type = genomic DNA
ttcagttggctccacaagttggagtctctcacaccttacacaaatgatct

SEQ ID NO: 93 - DNA, 50 bp, Zea mays
  source 1..50: mol_type = genomic DNA
tcgcaataacacacgaacacaagtcgagagaagagcataagagacgacgc

SEQ ID NO: 94 - DNA, 50 bp, Zea mays
  source 1..50: mol_type = genomic DNA
atagtatggttgcaatgtctcgatgttgtaggatgttcaaaggatgcttg

SEQ ID NO: 95 - DNA, 50 bp, Zea mays
  source 1..50: mol_type = genomic DNA
atgagccgttggagctccatatggaaggcactggttgccttctgcccgcg

SEQ ID NO: 96 - DNA, 50 bp, Zea mays
  source 1..50: mol_type = genomic DNA
ggtttcctattctgggggcaccgtaccgtccggtgggtggcactggactg

SEQ ID NO: 97 - DNA, 50 bp, Zea mays
  source 1..50: mol_type = genomic DNA
gttggctggctggcacaccggactgtctggcactccgcgcggattgtcca

SEQ ID NO: 98 - DNA, 50 bp, Zea mays
  source 1..50: mol_type = genomic DNA
cagaccgtgcaccggactgtctatcacaccggactgtctggtgggtggca

SEQ ID NO: 99 - DNA, 50 bp, Zea mays
  source 1..50: mol_type = genomic DNA
agaatctttttgcttcttttggcttgacttcataaagtccctagcactta

SEQ ID NO: 100 - DNA, 50 bp, Zea mays
  source 1..50: mol_type = genomic DNA
ctttttcacttggtaccatatagatttgtgttatgccctctttcaagctc

SEQ ID NO: 101 - DNA, 50 bp, Zea mays
  source 1..50: mol_type = genomic DNA
taaacataatgtgttgagcatttaatcaccaaaacaaaatagaaatggcc

SEQ ID NO: 102 - DNA, 50 bp, Zea mays
  source 1..50: mol_type = genomic DNA
cccatcaatttcattggatggacagtggaccgttgaaggaccagctatca

SEQ ID NO: 103 - DNA, 50 bp, Zea mays
  source 1..50: mol_type = genomic DNA
ccagtaagttccaagttcactagcaattctattctatatattaagcgccg

SEQ ID NO: 104 - DNA, 50 bp, Zea mays
  source 1..50: mol_type = genomic DNA
gcctatcaacatgcagttcttagataaaatcctaactgtcagaaattgaa

SEQ ID NO: 105 - DNA, 50 bp, Zea mays
  source 1..50: mol_type = genomic DNA
aacacacaataactgatgtaacagactaaaagagcaagttcatttacccc

SEQ ID NO: 106 - DNA, 50 bp, Zea mays
  source 1..50: mol_type = genomic DNA
attaacaataaataagatttcatggaataggctttatccgtttcgtttta

SEQ ID NO: 107 - DNA, 50 bp, Zea mays
  source 1..50: mol_type = genomic DNA
tatgttcagcaattcatctagcttgcatcttttattatacaacatgtaaa

SEQ ID NO: 108 - DNA, 50 bp, Zea mays
  source 1..50: mol_type = genomic DNA
tggaagagaacaagaatatttcaatagaaatgactgattagcaatcagta

SEQ ID NO: 109 - DNA, 50 bp, Zea mays
  source 1..50: mol_type = genomic DNA
cacttattcacagtgagtacaaacagtggcggccaatagcatttatgatt

SEQ ID NO: 110 - DNA, 50 bp, Zea mays
  source 1..50: mol_type = genomic DNA
aagtgggtctatacgtactgccaaatatacatagatatatacattggaga

SEQ ID NO: 111 - DNA, 50 bp, Zea mays
  source 1..50: mol_type = genomic DNA
ctgaagtctaatatttataggttgttttgtttgaggaataagctagttca

SEQ ID NO: 112 - DNA, 50 bp, Zea mays
  source 1..50: mol_type = genomic DNA
tcatcttctcactcacttttttgtttggtttgtgaaattgaatgagttga

SEQ ID NO: 113 - DNA, 50 bp, Zea mays
  source 1..50: mol_type = genomic DNA
tctatcaccacctcattcttcatagttagttagtattaacatgaagaatt

SEQ ID NO: 114 - DNA, 50 bp, Zea mays
  source 1..50: mol_type = genomic DNA
agaccatcccactaaatttaaggaatgtacttatgatgcaacaccttaca

SEQ ID NO: 115 - DNA, 50 bp, Zea mays
  source 1..50: mol_type = genomic DNA
attacattgacattgtcgtaatagttaccggcactataatttaccacaat

SEQ ID NO: 116 - DNA, 50 bp, Zea mays
  source 1..50: mol_type = genomic DNA
taatcattatattcttaatagttatgcaccattgacatcataaaaaggtt

SEQ ID NO: 117 - DNA, 50 bp, Zea mays
  source 1..50: mol_type = genomic DNA
caataggataataatatgcacatgggcttctgtacaaaaccatgattctt

SEQ ID NO: 118 - DNA, 50 bp, Zea mays
  source 1..50: mol_type = genomic DNA
aaagtaaataacacaatgaacattatctgcaatgttcacatcatattaaa

SEQ ID NO: 119 - DNA, 50 bp, Zea mays
  source 1..50: mol_type = genomic DNA
tataaaaaaataggtaaaacatatgtctattgtgcttgatcgtagacttc

SEQ ID NO: 120 - DNA, 50 bp, Zea mays
  source 1..50: mol_type = genomic DNA
ggcccattgttggacacaacatggcccgccatccatccatgtcttgtctc

SEQ ID NO: 121 - DNA, 50 bp, Zea mays
  source 1..50: mol_type = genomic DNA
ggttcgacaaaatcggctaggcttgtggcaagctcgtgctgagctggccc

SEQ ID NO: 122 - DNA, 50 bp, Zea mays
  source 1..50: mol_type = genomic DNA
gacacgacctattgcctatatatacttctatgtgaacctgtccatatata

SEQ ID NO: 123 - DNA, 50 bp, Zea mays
  source 1..50: mol_type = genomic DNA
ttggtgctccaatatatttacaaaattgatctttgagtcagtggatgcat

SEQ ID NO: 124 - DNA, 50 bp, Zea mays
  source 1..50: mol_type = genomic DNA
aaaggaacaaaaattgtggtactcaagtttgatctttggatcacttgaga

SEQ ID NO: 125 - DNA, 50 bp, Zea mays
  source 1..50: mol_type = genomic DNA
gcattggctgaaccacgggataaaatcaccgtgtctgttgtgcgactttc

SEQ ID NO: 126 - DNA, 50 bp, Zea mays
  source 1..50: mol_type = genomic DNA
gttcctaagttcaatacacaatctaaaggaacaatcaagtgaagagttct

SEQ ID NO: 127 - DNA, 50 bp, Zea mays
  source 1..50: mol_type = genomic DNA
tataaaccaagtttgttttgtttagtcttgatttttataggatcacctat

SEQ ID NO: 128 - DNA, 50 bp, Zea mays
  source 1..50: mol_type = genomic DNA
atgaacagaatgcgagctactagccaagggtgccttccctttctccttaa

SEQ ID NO: 129 - DNA, 50 bp, Zea mays
  source 1..50: mol_type = genomic DNA
ccaagtccatccttaatggagggatgtctaccaatggtaaaagcatccct

SEQ ID NO: 130 - DNA, 50 bp, Zea mays
  source 1..50: mol_type = genomic DNA
gaaggttggcaatatcatcattcaacttagcaattgtagagatatgttta

SEQ ID NO: 131 - DNA, 50 bp, Zea mays
  source 1..50: mol_type = genomic DNA
atgttccaaggatgaactagtatcattcaatttttctaccctaacattta

SEQ ID NO: 132 - DNA, 50 bp, Zea mays
  source 1..50: mol_type = genomic DNA
aactcaaaagcaagcatttcattcttaatttccaaagcaagagatttttc

SEQ ID NO: 133 - DNA, 50 bp, Zea mays
  source 1..50: mol_type = genomic DNA
ctaacaacatatccttatcattaagagcatcaatcaattcattaatttta

SEQ ID NO: 134 - DNA, 50 bp, Zea mays
  source 1..50: mol_type = genomic DNA
atcatcggaatcctcatcgctagaagaagtgtacttgggagtatcttgtg

SEQ ID NO: 135 - DNA, 50 bp, Zea mays
  source 1..50: mol_type = genomic DNA
aagagggaggacctgtcgaaggtggaggcaacaagtccttcgttgtcgga

SEQ ID NO: 136 - DNA, 50 bp, Zea mays
  source 1..50: mol_type = genomic DNA
cctttgccttactgtagttcttcttcttttccttcttacctttcttttct

SEQ ID NO: 137 - DNA, 50 bp, Zea mays
  source 1..50: mol_type = genomic DNA
cttatcacatttgaagcatgaacgctttctccttgatttgttcttgttgg

SEQ ID NO: 138 - DNA, 50 bp, Zea mays
  source 1..50: mol_type = genomic DNA
gccattttttcctaattaagcccggtagcctccacttgtgctaccttgtt

SEQ ID NO: 139 - DNA, 50 bp, Zea mays
  source 1..50: mol_type = genomic DNA
tatagaggggaagaggtatgttggtgatgtcgtcaacgtaccatgcctcc

SEQ ID NO: 140 - DNA, 50 bp, Zea mays
  source 1..50: mol_type = genomic DNA
catcttggggttttcacgaataaggtttacaagatggggatcaattatag

SEQ ID NO: 141 - DNA, 50 bp, Zea mays
  source 1..50: mol_type = genomic DNA
ctttcacaacttctaatcttgtttaccagggtcttgagcctattgtatgt

SEQ ID NO: 142 - DNA, 50 bp, Zea mays
  source 1..50: mol_type = genomic DNA
caatcaaacacatgagtaagggggagaagaaacacacacaagagacaaaa

SEQ ID NO: 143 - DNA, 50 bp, Zea mays
  source 1..50: mol_type = genomic DNA
gggagagttacaactcaaaacacgtgctcaaatctctatctagcatatca

SEQ ID NO: 144 - DNA, 50 bp, Zea mays
  source 1..50: mol_type = genomic DNA
gtatgctgctccatgcgcctaaggggtccttttatagccccaagggacct

SEQ ID NO: 145 - DNA, 50 bp, Zea mays
  source 1..50: mol_type = genomic DNA
ggtgcaccggacactgcacagtgcaatgtttgcaaatcctctgattggct

SEQ ID NO: 146 - DNA, 50 bp, Zea mays
  source 1..50: mol_type = genomic DNA
tccggtgcctccatgtggtcgttggccctaggccgaggtgtccactagcc

SEQ ID NO: 147 - DNA, 50 bp, Zea mays
  source 1..50: mol_type = genomic DNA
gtgatttatagttggccctagctgattttactcgagagcagccccttggt

SEQ ID NO: 148 - DNA, 50 bp, Zea mays
  source 1..50: mol_type = genomic DNA
ccggactgtccggtgctacgtagggcagtctaactttctcttttctattt

SEQ ID NO: 149 - DNA, 50 bp, Zea mays
  source 1..50: mol_type = genomic DNA
gacaaatatgattagtacccaaaataattgactaagtatccggagcttac

SEQ ID NO: 150 - DNA, 50 bp, Zea mays
  source 1..50: mol_type = genomic DNA
aatttgctttatgtacctatgctcatttctcatgtaagataattttagtc

SEQ ID NO: 151 - DNA, 50 bp, Zea mays
  source 1..50: mol_type = genomic DNA
taaggacacatttccctttcagtggatccgggtggagcaccagttgaagg

SEQ ID NO: 152 - DNA, 50 bp, Zea mays
  source 1..50: mol_type = genomic DNA
gcaagagaaacaattagcagacctgcacacgagttgccatcaacaactaa

SEQ ID NO: 153 - DNA, 50 bp, Zea mays
  source 1..50: mol_type = genomic DNA
acacatatccggtgtctccttgagccacgtcacctccaaaaatcttagtc

SEQ ID NO: 154 - DNA, 50 bp, Zea mays; identical to SEQ ID NO: 45
  source 1..50: mol_type = genomic DNA

SEQ ID NO: 155 - DNA, 50 bp, Zea mays; identical to SEQ ID NO: 47
  source 1..50: mol_type = genomic DNA

SEQ ID NO: 156 - DNA, 50 bp, Zea mays; identical to SEQ ID NO: 49
  source 1..50: mol_type = genomic DNA

SEQ ID NO: 157 - DNA, 50 bp, Zea mays; identical to SEQ ID NO: 51
  source 1..50: mol_type = genomic DNA

SEQ ID NO: 158 - DNA, 50 bp, Zea mays; identical to SEQ ID NO: 53
  source 1..50: mol_type = genomic DNA

SEQ ID NO: 159 - DNA, 50 bp, Zea mays; identical to SEQ ID NO: 55
  source 1..50: mol_type = genomic DNA

SEQ ID NO: 160 - DNA, 50 bp, Zea mays; identical to SEQ ID NO: 57
  source 1..50: mol_type = genomic DNA

SEQ ID NO: 161 - DNA, 50 bp, Zea mays; identical to SEQ ID NO: 59
  source 1..50: mol_type = genomic DNA

SEQ ID NO: 162 - DNA, 50 bp, Zea mays; identical to SEQ ID NO: 61
  source 1..50: mol_type = genomic DNA

SEQ ID NO: 163 - DNA, 50 bp, Zea mays; identical to SEQ ID NO: 63
  source 1..50: mol_type = genomic DNA

SEQ ID NO: 164 - DNA, 5000 bp, Zea mays
  source 1..5000: mol_type = genomic DNA
aaaagtgatccaaatatgccaaatcctatgtgagatgaatttgtcttcaatttggactctttagcttatctttgaaaatgtgttgcattttgagttgctttttgatgtgttggcataaatcaccaaaaagggggagatttaaaaggaaatgtgtcattgggccatttctataaatgttctggtgattagatgccaaacacattgttttggttgatatgcgctaagtgtcaaagagatgcaaatcaacaatcaaggtatgactctagccttagtgaattgtttttggtactaacatattcctctaagtgctaggaaccttgtcgaatcaaatgagattggatcggtttggctaagtccagccagacttgcaccagcctgcggtgcaccggacaatgtccgctgcccaagctggcgcacgcgacaaacttgctgctcttgggaaaagttgggggagtcgtgactataattcaccggactgtccggttagccaaccgcgcctgcgctaacggtcggcagcgcaatcagcgggcgacgtatggcccgcgccaacggtcaccaggtcgctccggactgtccggtgtgccaagggggctatggctgcaatggtcggcttcaccagaaaaagaaggagatcacgcactgttcactgtccggtggtgcaccggactgtccggtgcgcccacagatagaaggcaactattgccttccaaatggagctccaacggctcctagttgccttagggctataaaagggacccctaggcgcacggagcagtacaccaagcacctcttgaacatcctaagatgcctagactctgcaagcacgtattcaattcattgtgtttgagatttgagcacttgtttgagctgtgaacttgctgcgctgtgttgtgtgctcatttcttgacttgtgtgcgtgtcgttgctgcgattctagctcttgcatgtgtttctatttcctcccttactcttgtggtttaattgtgatcaatcttgtaagggtgagaggctccaacttgtagagattcctcacaaagggaatacttgagaaaaggaacaaaaattgtggtactcaagtttgatctttggatcacttgagagggattgagtgcaatccttgaccaaaggaggttgccacaacgtggagtaggcattggctgaaccacgggataaaatcaccgtgtctgttgtgcgactttctattgtgatttctttcttccttagagttttacttaatcacttgcgctattgttcctaagttcaatacacaatctaaaggaacaatcaagtgaagagttctctctctctctctccattcatagcaacttggttttgatctcactaacattttataaaccaagtttgttttgtttagtcttgatttttataggatcacctattcacccccccccccctctaggtgctcttagtaagcatggtttttcttatcatgaacagaatgcgagctactagccaagggtgccttccctttctccttaatgatgttggaggccttttggctctttaagtcattggttcccctttggaaaccaagtccatccttaatggagggatgtctaccaatggtaaaagcatccctagcaaatttaactttatcacattcatctttgcatattttaagttgagcatgaaggttggcaatatcatcattcaacttagcaattgtagagatatgtttattacaagcatctacataaaaaaaaactttacatctattgcaaatagaaacatgttccaaggatgaactagtatcattcaatttttctaccctaacatttaaatcaacatttacacttttaaggctagaaatagaataatggcaagaagacaactcaaaagcaagcatttcattcttaatttccaaagcaagagatttttcaacattaacaaatttatcatgttcttcataaagaatgtcctcttgtttttctaacaacatatccttatcattaagagcatcaatcaattcattaattttatcaactttggatctatctaagcccttaaagagatcgctataatctagatcatcatcggaatcctcatcgctagaagaagtgtacttgggagtatcttgtgtacataccttcttctccttagccatgaggcatgtatgtcattcgttggagaagagggaggacctgtcgaaggtggaggcaacaagtccttcgttgtcggagtcagatgaagagcagtccgagtcccattccttgccaatatgtgcctcgccctttgccttactgtagttcttcttcttttccttcttacctttcttttcttgttcctggtcattttcattatagagacattgtgtaataaaatgaccaggcttatcacatttgaagcatgaacgctttctccttgatttgttcttgttggggaagtccttgtgtcccttcaatgcggtcttgaagcgcttaatcacaagcgccattttttcctaattaagcccggtagcctccacttgtgctaccttgtttggtagcgtctccttgctagtcgttgccttgagtgcaacgagttgcggcttatagaggggaagaggtatgttggtgatgtcgtcaacgtaccatgcctccttcacaattatgcgtctgcttacgaattttcctagaattttctctagcgacatcttggggttttcacgaataaggtttacaagatggggatcaattatagtaaatgatcttagcatcagtcgcacgacatcatgatctgtccatcttgttctttcacaacttctaatcttgtttaccagggtcttgagcctattgtatgtttcagttggctccacaagttggagtctctcacaccttacacaaatgatctcaatcaaacacatgagtaagggggagaagaaacacacacaagagacaaaatcgcaataacacacgaacacaagtcgagagaagagcataagagacgacgcgggagagttacaactcaaaacacgtgctcaaatctctatctagcatatcaatagtatggttgcaatgtctcgatgttgtaggatgttcaaaggatgcttggtatgctgctccatgcgcctaaggggtccttttatagccccaagggacctatgagccgttggagctccatatggaaggcactggttgccttctgcccgcgggtgcaccggacactgcacagtgcaatgtttgcaaatcctctgattggctggtttcctattctgggggcaccgtaccgtccggtgggtggcactggactgtccggtgcctccatgtggtcgttggccctaggccgaggtgtccactagccgttggctggctggcacaccggactgtctggcactccgcgcggattgtccagtgatttatagttggccctagctgattttactcgagagcagccccttggtcagaccgtgcaccggactgtctatcacaccggactgtctggtgggtggcaccggactgtccggtgctacgtagggcagtctaactttctcttttctatttagaatctttttgcttcttttggcttgacttcataaagtccctagcacttagacaaatatgattagtacccaaaataattgactaagtatccggagcttacctttttcacttggtaccatatagatttgtgttatgccctctttcaagctcaatttgctttatgtacctatgctcatttctcatgtaagataattttagtctaaacataatgtgttgagcatttaatcaccaaaacaaaatagaaatggcctaaggacacatttccctttcagtggatccgggtggagcaccagttgaaggcccatcaatttcattggatggacagtggaccgttgaaggaccagctatcagcaagagaaacaattagcagacctgcacacgagttgccatcaacaactaaccagtaagttccaagttcactagcaattctattctatatattaagcgccgacacatatccggtgtctccttgagccacgtcacctccaaaaatcttagtcgttggatcttctacttgcggcttcatcgtttagcaatgctcaactacaatttggaactctcccacgtacctggccacacgacaagtcgcgatgctttgagagcttcttccccctcccctccttaaaccctgacatcgtgctggtgttgcctgtcgctcggagcgtcttcctctccccactccactcaaacatcgacgtcttctgtccgttcgaagcgactccgagtggagcaccaatccctccaaccagacaccgccggcatgacccacttccaggaactccctactctccctcatcctctgaaacgacaatgccgctgtcgtgtcggcgcgggccagagcaccattccctcctctgtgtgctgattccaatcggagctccatccctcccctcccccggccgccctcaagcgccaacacgttttgtccgtgtggagtggatccaggtggagcaccaggtgaaggcccatcaatttcagtggatggacagtggacgttgaaggcccagctatcagcaagagaaacagttagcagacctgcacacgagttgtcgtcaataactaactagtaagttccaagttcactagcacgtgttctataaccttattccattgaaattgatttcccttcttttctgtttgtcacgtggttaatgtgatgcaagtttataaataatgaagatgctgaagttgagaaagctcactaatactttatagcattgtctacatttcgatgcttttcttcatatccagctgtcttttcaaaccttgaaaaaatactactttggttttgatagactacctaaacacaaatcacaaagaaaagatgatgcatgtcataatgatataactaatctttccaaagtagtgtaccaaattttatttaacccagaaaaataccaccccttaattagagaaagaatcctaaactaatgcaaattataattttgataaacaaaatagtaaagacctaatcacactcagcataataggttttgccac

SEQ ID NO: 165 - DNA, 5000 bp, Zea mays
  source 1..5000: mol_type = genomic DNA
gcctatcaacatgcagttcttagataaaatcctaactgtcagaaattgaaaacacacaataactgatgtaacagactaaaagagcaagttcatttaccccattaacaataaataagatttcatggaataggctttatccgtttcgttttatatgttcagcaattcatctagcttgcatcttttattatacaacatgtaaatggaagagaacaagaatatttcaatagaaatgactgattagcaatcagtacacttattcacagtgagtacaaacagtggcggccaatagcatttatgattaagtgggtctatacgtactgccaaatatacatagatatatacattggagactgaagtctaatatttataggttgttttgtttgaggaataagctagttcatcatcttctcactcacttttttgtttggtttgtgaaattgaatgagttgatctatcaccacctcattcttcatagttagttagtattaacatgaagaattagaccatcccactaaatttaaggaatgtacttatgatgcaacaccttacaattacattgacattgtcgtaatagttaccggcactataatttaccacaattaatcattatattcttaatagttatgcaccattgacatcataaaaaggttcaataggataataatatgcacatgggcttctgtacaaaaccatgattcttaaagtaaataacacaatgaacattatctgcaatgttcacatcatattaaatataaaaaaataggtaaaacatatgtctattgtgcttgatcgtagacttcggcccattgttggacacaacatggcccgccatccatccatgtcttgtctcggttcgacaaaatcggctaggcttgtggcaagctcgtgctgagctggcccgacacgacctattgcctatatatacttctatgtgaacctgtccatatatattggtgctccaatatatttacaaaattgatctttgagtcagtggatgcattggtcaaagactctgcggagcacgagaaaggtactagttatgtaagacgagtatttaatggcagtaagagaactcgaggtttcagaaccaaggtggacacactgtaacattagattacctttagtttttttatttagtcctcactaacgtagatcagtattcagtgtcggagcacgtatgtttcagaaacaagtagatcaatatataaaagataatctgctcactgacaacgacatcagtatccagcggtttcagaaacaaacagatgtaatttatattaacaaagttgtgcatagccaacaacatcagagtggcgcagtggaagcgtggtgggcccataacccacaggtcccaggatcgaaacctggctctgataattttttttgcttttgcttttttccctgattttttccactttccttttcttgcaataccataaaaatacaggtgccttaaatgctcagaaagagctttttcactttgctattctagttcagttggcttcctgatttcaaccttttattctgaaattttcactttgctgttctagcaataccataaaaatgcagtcgtctggaatgctcaaaaagagttgaattcttcacattgttgtactatttcagcggccttcctgatttatatcttttcgatgcataagtttttacttagctgttctagcaaataccataaaactgcaggtgtcctgaatctcagaaagagtaattaatatttatatttgcgtttcactggcgtagttcgcgtatagttgaattatcagccaataacttaggaggatgacatcacatttaaaatgagtgacgaccgttttgtcgttccaagtaacttttctattggatgtttgggttagaaattaggttctttgaacacacaatcacaagcacaacaataacatgtaagaacacagagatgtagaagggaaggggagcttctgtattcattttgtttcttaaattacataaggtgaaagtgaccccttgttcatactaaaaggggctgaggtgaatacaattaccattgtgtgaactctctaaatttccctctcatgtttcattaatatggccaacatttcaaagtcatgttcttatactccctgtgaggtaatgaaggtttgtcctaagtcaaagttttaaaattttgaccaacaatatctacaaaattaattatttttaaattaaataaataatatattgttctatatgttttaataataaatgtaatagtttcgtttttatttagtcaatgtttatgaatattttattattaatggtcaaagtttcaaaactttgacttagaataaactttcgtgaccttaagatagggaacggagggagtactatttttggacttgatttacaaaactcctcttttgccccatttatgctacgtggatgctttgttaaaaactctcaaaaatagtgaaaatgagaaaaagtgcatcacatgcaactgctgcctcattaaaaaacatgcatgagaaaacggatgtctatatgtatattggaaaacacatataaacagtaaaaaagatgaattatacagtgagttgtccgctgtatgtgcggtttttaattcatttgctagtctttaatacaaatgatgcaatatacaaatgatctttgtaaccaatatgtttcttgctaattgatacacacaaatataatatattactttttttgtaaattagcaggaatacaagacattgtatatcattttaattcaaaaagcaccatatacataatatatattataattggtaaatggcatgataacttcatgtagatttatttgcgatgggaccatatttttgccatatattttgaaccaaatccctcttcagttgagtatgagtcgagcgagcttgaatagcggaattcctttgtagcactccgacgaagcgagggtcgggattgtcattagtttgcccttccagtggtagcacaattgtttcacttgagttttcgttcaagtccataggaactctaacttattctttttcatcttcgacagtcatgttatgaaggataatacaagctattacgatgttgacaacatcccctcgcttccataaacgggcttctatcggtgaacaatatcaaaacgggaccgtaacactccaaacgcgcactcaacattcttcctcgtcttttcttgatgctttacatatactgcccaccctccatcagcaagataccgcccactctccatcagcaagataatatggagtttaaccttttaacatgttaatgttgtaatattagagaaataagtgacatgctttctacatatttaaatagattaaatattctaattccgaataaaacactcatataactcagataaataccatatgaaattatagcagcaatgaacagtagcaattctaaaacatgtaaacatgtaaaatagctacacaggtccatttcaaaattaaacatggcttgcagatgaagaaggcagattaaactaataaacataattgtttatcttaaactattgctctcaaagtagcgggttgctgtaataaacagccctccaacgctaaaaggtgatcagagatcctcgactaacgtgacagtcctatcttaccaaataagggttttagatcagatgtaataagcctaataatcaaatataaatactgatagtaaggagctgtgtattaaataaattaacagaatttaacacaggtaaacagcctcaataaagagagatttaattaggcacaaattatatcaatgatgaaaatgataatacactaaaacccagactgtcacgttgtctcactacaccgctatcatcatcgagcacacaaatcgcccacaacagtgcagtcacacccaagtatttaatactagcagattgcacaaaattaaacagtaaataaaatgaggcaacgaataactcacagcacgtagattgtctacgtgggaagaccagctcagcgaacacaaggttctgtcccagaaaaccaattccgccgcccacgtccgggcctgcacggcgggaggttgacggagatactagagccgctgccggagccgagggagacgtccacggcaccagcccgagaagacgaacaccgcgcagcagggagcccaggcaccagaccacggtggacaataatcagatcagacagagcaccacacaggcgcactgtaaatcaacggagcacaggagaaccttgctgcacaagaacagcaccacctctactagcttccaatcaccaccagcgattaaacacaggaacactagggaattatggagctagaagaatggaacagccagctgcagagaggagtagaacacacttctacacgggatttaccgcccatcctttaaggagacgaggtggggagaggaggccaactgctttcccgtggatctcggacgaacgagccgcagagctcggcctccgttgaagcgccgccgccccagcttttgctgctctgctcgttctaccgatctccaagtcgtggcctcttcatgccctagccaactgccagcccgtgggctgctcggtcagctgggccttgtagcggcccacccggctggccccctcctctctctcttttttttaataaagtgaaaacacaaaaatcggatagcaatccccaccattttcactcttattttctgatcaatgtcattgagctcccgtactgtttatatactacttttcggcagaggtacttgttaggtttgaaccaaagcctatcccagtgtactccaaccctacacgagtaagcggaggactacgccttgatccacaaaccctagtgtgagaatcttcgcacaaaaggcacatagtactaggcagattatctgcttctcttacggggcaaggcgttacggtcatgccatttaatatctattcatgagctcactagagagaaaaccccattctctcctggactgcgaccccatagtcagctcatataggtgaaatcgttaaggaaattctgtaggacaatttccctgc

SEQ ID NO: 166 - DNA, 552 bp, Streptomyces viridochromogenes
  source 1..552: mol_type = genomic DNA
atgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatccacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatctga

SEQ ID NO: 167 - protein, 183 aa, Streptomyces viridochromogenes
MSPERRPVEIRPATAADMAAVCDIVNHYIETSTVNFRTEPQTPQEWIDDLERLQDRYPWLVAEVEGVVAGIAYAGPWKARNAYDWTVESTVYVSHRHQRLGLGSTLYTHLLKSMEAQGFKSVVAVIGLPNDPSVRLHEALGYTARGTLRAAGYKHGGWHDVGFWQRDFELPAPPRPVRPVTQI

SEQ ID NO: 168 - DNA, 1023 bp, synthetic construct
  source 1..1023: mol_type = other DNA
atgctcaccttcgttaggaacgcctggtacgtcgccgctctccctgaggagctgagcgagaagcccttgggtcgcaccatcctagacactccgttagccctttaccgccagcctgacggcgtagtggcggccctgcttgacatctgcccgcataggttcgctccgctcagcgacggcatcctcgtcaacgggcatcttcagtgcccgtaccacgggctggaatttgacggcggtgggcagtgtgtccacaacccgcacggcaacggcgcacggccagcttccctcaacgttaggtcgttccctgttgtcgagcgcgacgcactgatctggatctggcctggcgacccagctctggccgatccaggagccattcccgacttcggttgccgcgtggacccagcctatcggacggtcggcggttacgggcacgtcgattgtaactataagctccttgtggacaaccttatggatttgggccacgctcagtacgtgcaccgggctaacgctcagactgacgcctttgaccgtctcgaaagggaggtcatcgtcggcgacggagagattcaggcgctgatgaagatccctggaggcacgccctctgtgctcatggcgaagtttctcagaggcgcgaacacgcccgtggacgcctggaacgacatccgctggaataaggtctccgcgatgctgaacttcatcgccgttgcgcccgagggcacacccaaagagcagtcaatccacagcagagggacccatattcttacaccggaaaccgaggctagttgccactacttcttcggctcgtcacggaatttcgggatagacgatccggagatggacggtgttcttcgatcttggcaagcgcaagctctcgtcaaggaagataaggtggtcgtggaggctatcgagcgtaggcgcgcctacgttgaggcgaacggtattaggcccgcgatgctgtcctgcgacgaggccgcagttagagtgtcgcgcgagatagaaaagctggagcagctagaggccgcctga

SEQ ID NO: 169 - protein, 340 aa, Pseudomonas maltophilia
MLTFVRNAWYVAALPEELSEKPLGRTILDTPLALYRQPDGVVAALLDICPHRFAPLSDGILVNGHLQCPYHGLEFDGGGQCVHNPHGNGARPASLNVRSFPVVERDALIWIWPGDPALADPGAIPDFGCRVDPAYRTVGGYGHVDCNYKLLVDNLMDLGHAQYVHRANAQTDAFDRLEREVIVGDGEIQALMKIPGGTPSVLMAKFLRGANTPVDAWNDIRWNKVSAMLNFIAVAPEGTPKEQSIHSRGTHILTPETEASCHYFFGSSRNFGIDDPEMDGVLRSWQAQALVKEDKVVVEAIERRRAYVEANGIRPAMLSCDEAAVRVSREIEKLEQLEAA

SEQ ID NO: 170 - DNA, 888 bp, synthetic construct
  source 1..888: mol_type = other DNA
atgcacgcggcgctgactcctctcaccaacaagtatcgctttatcgacgtgcagccgctgacaggcgtcctcggtgcagagattacaggcgtggatctgcgggagcctctcgatgacagcacttggaatgagatcctggacgcctttcacacctaccaagtgatctactttccgggtcaagctatcactaacgagcagcacatcgcgttctcccgccggttcggccctgtggacccggtgccgatcttaaagagtatcgagggctatccagaggtgcagatgatacggcgcgaggcgaacgagagcagccggttcatcggagatgactggcacaccgattccaccttcctggacgctccgcctgccgccgtggtgatgagagctatcgaagtgccggagtatggaggtgacacaggcttcctctccatgtacagtgcctgggagacactctcgcctacgatgcaagctaccatcgaaggcttaaacgtggtccactcggcgacgaaggtcttcgggtcattgtaccaggcgactaattggcgcttctcgaacaccagcgtgaaagtgatggacgtggacgccggagatagagagactgtgcacccactcgtcgtgacgcatcctgttacgggaaggcgcgcactctactgcaaccaggtgtactgccagaagatccagggaatgacggacgcggagtcgaagtccctgttgcaattcctttacgagcacgccaccaagttcgacttcacctgccgggtccggtggaagaaggaccaagtcctggtgtgggacaacctgtgtaccatgcaccgcgccgtcccggactacgctgggaaattcagatacctgacccgcaccaccgtggcgggagacaagccgtcgcgttga

SEQ ID NO: 171 - protein, 295 aa, Sphingobium herbicidovorans
MHAALTPLTNKYRFIDVQPLTGVLGAEITGVDLREPLDDSTWNEILDAFHTYQVIYFPGQAITNEQHIAFSRRFGPVDPVPILKSIEGYPEVQMIRREANESSRFIGDDWHTDSTFLDAPPAAVVMRAIEVPEYGGDTGFLSMYSAWETLSPTMQATIEGLNVVHSATKVFGSLYQATNWRFSNTSVKVMDVDAGDRETVHPLVVTHPVTGRRALYCNQVYCQKIQGMTDAESKSLLQFLYEHATKFDFTCRVRWKKDQVLVWDNLCTMHRAVPDYAGKFRYLTRTTVAGDKPSR

SEQ ID NO: 172 - DNA, 1368 bp, synthetic construct
  source 1..1368: mol_type = other DNA
atgcttcatggagcttcatctaggccagctactgccaggaagtctagcgggctcagtggcaccgtgcgcatccctggcgataaaagtatttcacacaggagcttcatgttcggaggacttgctagtggagagacgagaatcactggtttgcttgagggcgaagatgttatcaacaccggtaaggcgatgcaagcaatgggtgccagaatccgaaaagagggcgatacgtggatcatcgacggtgttggtaacggaggattgctcgctcccgaagcgccacttgactttgggaacgcagctacggggtgccgtcttactatgggactggtaggcgtgtatgactttgactctaccttcatcggtgacgcgagcctcactaagagaccaatgggacgagtgctgaatcccctgagggagatgggtgtccaggtgaaatctgaggatggtgatcgtcttccggttactctgcgaggccccaagacccccacgccaatcacgtacagggttccgatggcgtcagcacaggtcaagtcagcggtactcctggcgggcctcaacacacctggaatcacaaccgtgattgaacccatcatgactagagaccacacggagaagatgttgcagggtttcggcgctaatctaacggtcgaaaccgacgccgacggcgtgaggacaatccgcttggagggcagaggtaaactgactggccaagtcatcgatgtgcctggagatccctcgtccacagcgtttcccctcgtagctgcgttgctcgtccctggatctgatgtgacgatcctgaatgtcctcatgaatccaactagaaccggcctcatcctcacattgcaggagatgggtgctgacatcgaggttatcaatcctaggttggcaggtggagaggatgtggccgatctgcgcgtgcgttctagtacactcaaaggcgtgaccgtccctgaggatcgcgctccatccatgatcgacgagtaccccattctcgccgttgctgctgcgtttgccgagggcgcaactgtaatgaacggccttgaggagttgagggttaaggagagtgacaggctgtccgcggtggcgaatggcctgaagctaaacggcgtggactgcgacgaaggtgaaacgtcccttgtagtccgtggtcgcccagacgggaaggggttggggaatgcttcgggagctgctgtggcgacgcaccttgatcatagaatcgccatgtcatttctggtgatgggacttgtctccgagaatccggtgaccgttgacgatgctaccatgatcgccacctcctttcctgagttcatggacctcatggcaggcttgggggccaagatcgagctgtctgatactaaggccgcttga

SEQ ID NO: 173 - protein, 455 aa, Agrobacterium tumefaciens
MLHGASSRPATARKSSGLSGTVRIPGDKSISHRSFMFGGLASGETRITGLLEGEDVINTGKAMQAMGARIRKEGDTWIIDGVGNGGLLAPEAPLDFGNAATGCRLTMGLVGVYDFDSTFIGDASLTKRPMGRVLNPLREMGVQVKSEDGDRLPVTLRGPKTPTPITYRVPMASAQVKSAVLLAGLNTPGITTVIEPIMTRDHTEKMLQGFGANLTVETDADGVRTIRLEGRGKLTGQVIDVPGDPSSTAFPLVAALLVPGSDVTILNVLMNPTRTGLILTLQEMGADIEVINPRLAGGEDVADLRVRSSTLKGVTVPEDRAPSMIDEYPILAVAAAFAEGATVMNGLEELRVKESDRLSAVANGLKLNGVDCDEGETSLVVRGRPDGKGLGNASGAAVATHLDHRIAMSFLVMGLVSENPVTVDDATMIATSFPEFMDLMAGLGAKIELSDTKAA

SEQ ID NO: 174 - DNA, 27 bp, Zea mays
  source 1..27: mol_type = genomic DNA
tttaaccgacttgggcatatattattt

SEQ ID NO: 175 - DNA, 27 bp, Zea mays
  source 1..27: mol_type = genomic DNA
tttaactctaaatttagttggagacaa

SEQ ID NO: 176 - DNA, 27 bp, Zea mays
  source 1..27: mol_type = genomic DNA
tttacgtttcgacaacagtaatatatc

SEQ ID NO: 177 - DNA, 27 bp, Zea mays
  source 1..27: mol_type = genomic DNA
tttcaacctgctgaagttgataactct

SEQ ID NO: 178 - DNA, 27 bp, Zea mays
  source 1..27: mol_type = genomic DNA
tttggcagtacgtatagacccacttaa

SEQ ID NO: 179 - DNA, 27 bp, Zea mays
  source 1..27: mol_type = genomic DNA
tttcatcgtctagggagcctgacgaaa

SEQ ID NO: 180 - DNA, 27 bp, Zea mays
  source 1..27: mol_type = genomic DNA
tttcctttaggctcccttggaaatgca

SEQ ID NO: 181 - DNA, 27 bp, Zea mays
  source 1..27: mol_type = genomic DNA
tttaagcctatagtcaagaatacaata

SEQ ID NO: 182 - DNA, 27 bp, Zea mays
  source 1..27: mol_type = genomic DNA
tttgagcaaatatcacatcattgggcc

SEQ ID NO: 183 - DNA, 27 bp, Zea mays
  source 1..27: mol_type = genomic DNA
tttgccacaaggctattcactccctat

SEQ ID NO: 184 - DNA, 27 bp, Zea mays
  source 1..27: mol_type = genomic DNA
tttcgttaactattctatcaactagag

SEQ ID NO: 185 - DNA, 27 bp, Zea mays
  source 1..27: mol_type = genomic DNA
tttattgtttgctacagatgacatatc

SEQ ID NO: 186 - DNA, 27 bp, Zea mays
  source 1..27: mol_type = genomic DNA
tttgcctcgaggactacattcatagga

SEQ ID NO: 187 - DNA, 27 bp, Zea mays
  source 1..27: mol_type = genomic DNA
tttgagaggaaccaccatttcatgtga

SEQ ID NO: 188 - DNA, 27 bp, Zea mays
  source 1..27: mol_type = genomic DNA
tttcgacaacagtaatatatcgttcaa

SEQ ID NO: 189 - DNA, 27 bp, Zea mays
  source 1..27: mol_type = genomic DNA
tttggataaccaaaccagggcctatag

SEQ ID NO: 190 - DNA, 27 bp, Zea mays
  source 1..27: mol_type = genomic DNA
tttaaggcggacagagggagactatcc

SEQ ID NO: 191 - DNA, 30 bp, Solanum lycopersicum
  source 1..30: mol_type = genomic DNA
ggatctaagaagagaagaattaaacaagat

SEQ ID NO: 192 - DNA, 877 bp, Zea mays
  source 1..877: mol_type = genomic DNA
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttcccc

SEQ ID NO: 193 - DNA, 298 bp, Oryza sativa
  source 1..298: mol_type = genomic DNA
taatcgatcctccgatcccttaattaccataccattacaccatgcatcaatatccatatatatataaaccctttcgcacgtacttatactatgttttgtcatacatatatatgtgtcgaacgatcgatctatcactgatatgatatgattgatccatcagcctgatctctgtatcttgttatttgtataccgtcaaataaaagtttcttccacttgtgttaataattagctactctcatctcatgaaccctatatataactagtttaatttgctgtcaattgaacatgatgatcgatg

SEQ ID NO: 194 - DNA, 36 bp, unidentified
  source 1..36: mol_type = genomic DNA; note = Lachnospiraceae bacterium ND2006
gtttcaaagattaaataatttctactaagtgtagat

SEQ ID NO: 195 - DNA, 23 bp, Zea mays
  source 1..23: mol_type = genomic DNA
accgacttgggcatatattattt

SEQ ID NO: 196 - DNA, 23 bp, Zea mays
  source 1..23: mol_type = genomic DNA
actctaaatttagttggagacaa

SEQ ID NO: 197 - DNA, 23 bp, Zea mays
  source 1..23: mol_type = genomic DNA
cgtttcgacaacagtaatatatc

SEQ ID NO: 198 - DNA, 23 bp, Zea mays
  source 1..23: mol_type = genomic DNA
aacctgctgaagttgataactct

SEQ ID NO: 199 - DNA, 23 bp, Zea mays
  source 1..23: mol_type = genomic DNA
gcagtacgtatagacccacttaa

SEQ ID NO: 200 - DNA, 23 bp, Zea mays
  source 1..23: mol_type = genomic DNA
atcgtctagggagcctgacgaaa

SEQ ID NO: 201 - DNA, 23 bp, Zea mays
  source 1..23: mol_type = genomic DNA
ctttaggctcccttggaaatgca

SEQ ID NO: 202 - DNA, 23 bp, Zea mays
  source 1..23: mol_type = genomic DNA
agcctatagtcaagaatacaata

SEQ ID NO: 203 - DNA, 23 bp, Zea mays
  source 1..23: mol_type = genomic DNA
agcaaatatcacatcattgggcc

SEQ ID NO: 204 - DNA, 23 bp, Zea mays
  source 1..23: mol_type = genomic DNA
ccacaaggctattcactccctat

SEQ ID NO: 205 - DNA, 23 bp, Zea mays
  source 1..23: mol_type = genomic DNA
gttaactattctatcaactagag

SEQ ID NO: 206 - DNA, 23 bp, Zea mays
  source 1..23: mol_type = genomic DNA
ttgtttgctacagatgacatatc

SEQ ID NO: 207 - DNA, 23 bp, Zea mays
  source 1..23: mol_type = genomic DNA
cctcgaggactacattcatagga

SEQ ID NO: 208 - DNA, 23 bp, Zea mays
  source 1..23: mol_type = genomic DNA
agaggaaccaccatttcatgtga

SEQ ID NO: 209 - DNA, 23 bp, Zea mays
  source 1..23: mol_type = genomic DNA
gacaacagtaatatatcgttcaa

SEQ ID NO: 210 - DNA, 23 bp, Zea mays
  source 1..23: mol_type = genomic DNA
gataaccaaaccagggcctatag

SEQ ID NO: 211 - DNA, 23 bp, Zea mays
  source 1..23: mol_type = genomic DNA
aggcggacagagggagactatcc

SEQ ID NO: 212 - DNA, 16068 bp, synthetic construct
  source 1..16068: mol_type = other DNA
atctacagggcgcgcagcggctgacacatgggacccacgggcaaaggcagcggcagactcgcgcgcgagagcacgcaagcaccgattgacgcgcgtgccccttttgtcggagaacgagggtgcgcgatcgtccgtgcatggttgagacggccaggcggggcccacccattggcgccatgccgcgcgagcagcagctggcccgcacaggtgcaaaggttgagtgggctagaagcgaagacctagtccaagcgacggtttcttccttttttctctttttttctttttcctttatattttcttgttttattttattttcatttttttattttcaaatctaaatttgaactcaagtgtgagattcatactttgaattaaatgctcacattcaaatatcagtaggaatagaatatttttaactatatgtttattttctctattttatacaatcttttccttttttcatttttaaacctcaatttgtaaattatatctaaattccaattttggtcattagtatactcctactaatatcattttattgttatgaaatgcacacacaataaactccaacttgatgaatagatatctatttattctaattaatttatttgtttggtagatgttcaaaatatgaaacacacacatattgttttctttctttttagaaaaatggtatttttattgtgggacaagaactaaaagtcacctcaaaattaaatactcatgtataatttgggatatctgaatttactatttttatttcttcatatatttaatttattattaattttttttcttattgggccttacacttatgagatcatagaggcatgtggatcagtggcatacaagttgaaattactgccaaaaatgtctgtcatacacaatccgtgtgtttggtttgtggaccagcagagcctggctactctaatccgtatgtagagaaccaagctcccacgagacagactcaatgtatccgagatactcttgtttggttacacttgcaaaaatagaccactcttgtttggtttcatgtccgggaaatctacatggatcagcaatgagtatgatggtcaatatggagaaaaagaaagagtaattaccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaacttcacgatttggcgcgactaactaagcactagcgtacgggacccagatatcgaattcaagctgaccgccaccggcaaacaaccacgaatttgtaatggtactaggcaaattctccgtttggcggtgtgtgccggccaattacacgtttttgcggtgtcctccgacaaaatttgccttttaaaaacaattttataagagaagctccggagataaaaggccgtcaatgttacaagagtgaagtcgtctactccctccatcccaaaaaatgtaattctaagtatgagttgtattattatttttggacaaaaggagtataccacaagaatgatatcatcgtcatgcttagatcctttttagtaaagcttgagcttctctaaaagtagagaaattagaaaaaaatcacgtttttgtggtcttgatttctagcctccacaaaatctttggttttacattttttgtttgattttggtttcagaagtccttatttatatgtgctagtttggcagcacttaaaatcgttagagagagcctaaacaaaagccttttcaaaacgaccttgagccagattggttgatggccaaaatttgattgtcaaaacttaggcaagccaagattttagcagctatttggtttggtaccaaaatttgccaatgatctgttcttttgccttttcaaccggtttatcagccgtacttcagcttattctctctcacagaacactattgaatcagccgaaaagccaccgcagaacaggaccagtatctcacaaatggcatgccaaatatactcaccgtcagtgagcccgtttaacggcgtcgacaagtctaacggccaccaaccagcgaaccaccagcgtcaagctagccaagcgaagcagacggccgagacgttgacaccttggcgcgggcatctctctggccccctctcgagagttccgctccacctccactggtggcggtttccaagtccgttccgcctcctgctcctcctcacacggcacgaaaccgtcacggcaccggcagcacgggggattcctttcccaccgctccttccctttcccttcctcgcccgccgttttaaatagccagccccatccccagcttctctccccaacctcagcttctctcgttgttcggagcgcacacacaacccgatccccaatcccctcgtctctcctcgcgagcctcgtcgatccccgcttcaaggtacggcgatcatcctccctttctctaccttctcttctctagactaggtcggcgatccatggttagggcctgctagttctgttcctgtttttccgtggctgcgaggtacaatagatctgatggcgttatgatggttaacttgtcatactcctgcggtgtgcggtctatagtgcttttaggacatcaatttgacctggctcgttcgagatcggcgatccatggttaggaccctaggcggtggagtcgggttagatccgcgctgtttgtgttagtagatggatgcgacctttacttcagacacgttctgattgttaacttgtcagcacctgggagtcctgggatggttctagctggttcgcagatgagatcgatttcatgatctgctgtatcttgtttcgttaggttccttttaatctatccgtggtattatgctaacctatgatatggttcgatcgtgctagctacgtcctgtgtcataatttttagcatgcccttttttgtttggttttgtctgattgggctgtagatcagagtatactgtttcaaactacctactggatatatttattaaatttgaatctgtatgtgtgtcacatatatcttcataattaaaatggatggaaagatatatggataggtacatgtgttgctgtgggttttactggtactttgttagatatacatgcttagatacatgaagcaacatgatgttacagttcaataattcttgtttacctaataaacaaataaggataggtgtatgttgctgtgggttttgctggtactttgttagatatatatgcttagatatatgaagcaacatcctgctacggtttaataattattgtttatatctaatagacaagcctgctttttaattattttgatatacttggatgatggcatacagcagctatgtgtggatttttaaatacccagcatcatgagcatgcatgaccctgccttagtatgctgtttatttgcttgagacttctttttttgttggtactcaccttttgtagtttggtgactcttctgcaggtgcaaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatccacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatctgatacgcgctgctagtagccaagtacctacctcgagggcacactgtaggcagtgtgccatattacaggttcagattggccgggacaaagagtacactgcgattttactatcctcggtatgctggtactacaggttcacattcacatggttactacctggcctgtggtatgctggtactacaggtgttgctctgaatagctcaggcttgacccaagtaagagcagtgcaaattcccaatttccagaaaacaggaggacctccaaaggacttcaatgtaattcaaatttctttcagaggtccttttgcaaggggctggatgtaaagtacttttataataatccaagccattgtgcttctcaaaaaaaggaaaagaaaaaaatttgttgcggcccggccgtgacggccacgagcgaactcctgcaggagcagactcgcattatcgatggagctctaccaaactggccctaggcattaacctaccatggatcacatcgtaaaaaaaaaaccctaccatggatcctatctgttttctttttgccctgaaagagtgaagtcatcatcatatttaccatggcgcgcgtaggagcgcttcgtcgaagacccataggggggcggtactcgcaccgtggttgtttcctgttatgtaatatcggatgggggagcagtcggctaggttggtcccatcggtactggtcgtcccctagtgcgctagatgcgcgatgtttgtcctcaaaaactcttttcttcttaataacaatcatacgcaaattttttgcgtattcgagaaaaaaagaagattctatctgttttttttttgaaatggctccaatttataggaggagcccgtttaacggcgtcgacaaatctaacggacaccaaccagcgaatgagcgaacccaccagcgccaagctagccaagcgaagcagacggccgagacgctgacacccttgccttggcgcggcatctccgtcgctggctcgctggctctggccccttcgcgagagttccggtccacctccacctgtgtcggtttccaactccgttccgccttcgcgtgggacttgttccgttcatccgttggcggcatccggaaattgcgtggcgtagagcacggggccctcctctcacacggcacggaaccgtcacgagctcacggcaccggcagcacggcggggattccttccccaccaccgctccttccctttcccttcctcgcccgccatcataaatagccacccctcccagcttccttcgccacatcctctcatcatcttctctcgtgtagcacgcgcagcccgatccccaatcccctctcctcgcgagcctcgtcgatccctcgcttcaaggtatggctatcgtccttcctctctctctctttaccttatctagatcggcgatccatggttagggcctgctagttctccgttcgtgtttgtcgatggctgtgaggcacaatagatccgtcggcgttatgatggttagcctgtcatgctcttgcgatctgtggttcctttaggaaaggcattaatttaatccctgatggttcgagatcggtgatccatggttagtaccctaagctgtggagtcgggtttagatccgcgctgttcgtaggcgatctgttctgattgttaacttgtcagtacctgcgaatcctcggtggttctagctggttcggagatcagatcgattccattatctgctatacatcttgtttcgttgcctaggctccgtttaatctatccatcgtatgatgttagcctttgatatgattcgatcgtgctagctatgtcctgtggacttaattgtcaggtcctaatttttaggaagactgttccaaaccatctgctggatttattaaatttggatctggatgtgtcacatacaccttcataattaaaatggatggaaatatctcttatcttttagatatggataggcatttatatgatgctgtgagttttactagtactttcttagaatatatgtacttttttagacggaatattgatatgtatacatgtgtagatacatgaagcaacatgctgctgtagtctaataattcctgttcatctaataatcaagtatgtatatgttctgtgtgttttattggtatttgattagatatatacatgcttagatacatacatgaagcagcatgctgctacagtttaatcattattgtttatccaataaacaaacatgctttttaatttatcttgatatgcttggatgacggaatatgcagagattttaagtacccagcatcatgagcatgcatgaccctgcgttagtatgctgtttatttgcttgagactctttcttttgtagatactcaccctgttttctggtgatcctactgcaggtgaccggtcgccatggccaccgccaccactaccgccaccgctgcgttctccggcgtggtgagcgtcggcactgagacgcgcaggatctactccttcagccacctccagccttctgctgcgttccccgctaagccgtcttcgttcaagagcctgaagctgaaacagtccgcacgccttacccggcgcctggaccataggccattcgttgtcaggtgcatgctcaccttcgttaggaacgcctggtacgtcgccgctctccctgaggagctgagcgagaagcccttgggtcgcaccatcctagacactccgttagccctttaccgccagcctgacggcgtagtggcggccctgcttgacatctgcccgcataggttcgctccgctcagcgacggcatcctcgtcaacgggcatcttcagtgcccgtaccacgggctggaatttgacggcggtgggcagtgtgtccacaacccgcacggcaacggcgcacggccagcttccctcaacgttaggtcgttccctgttgtcgagcgcgacgcactgatctggatctggcctggcgacccagctctggccgatccaggagccattcccgacttcggttgccgcgtggacccagcctatcggacggtcggcggttacgggcacgtcgattgtaactataagctccttgtggacaaccttatggatttgggccacgctcagtacgtgcaccgggctaacgctcagactgacgcctttgaccgtctcgaaagggaggtcatcgtcggcgacggagagattcaggcgctgatgaagatccctggaggcacgccctctgtgctcatggcgaagtttctcagaggcgcgaacacgcccgtggacgcctggaacgacatccgctggaataaggtctccgcgatgctgaacttcatcgccgttgcgcccgagggcacacccaaagagcagtcaatccacagcagagggacccatattcttacaccggaaaccgaggctagttgccactacttcttcggctcgtcacggaatttcgggatagacgatccggagatggacggtgttcttcgatcttggcaagcgcaagctctcgtcaaggaagataaggtggtcgtggaggctatcgagcgtaggcgcgcctacgttgaggcgaacggtattaggcccgcgatgctgtcctgcgacgaggccgcagttagagtgtcgcgcgagatagaaaagctggagcagctagaggccgcctgattaattaaggccaaggcgatctatgactgaattgccaatgcaccagcctgtctacatgatgaataaataaagagtccatccagtgtgatggctcatgcctgtgtgagtgtgactgaatccatcagtgtgtgtgtgtgtttgtgtcaaccatgtgtgaatcaggtgtcaaaaatcgtggctggaaatccatgtggtttctagctttatgtaaatgttgtttgtgaaatataaatattgttttgtgtatgtgaattttactctctcatttttctcttgcactcaccattctattatagtaatttttttaagcggccgctgatgtatccgtccacggtggtgtgtccaatcagtgaataatctagttagtgaagccagaagtccatagtgccccttgctctgtcaccatatatccagttcaaccgcaccaatttgccatctcgaactggttcatgttttattcaggttggtaaatgaattttgccaattcaatgtagttagatatttccatgtcattttagtacatttaccaattttttatattctggctagaaaaggagaatggtgacgtctttcggaagatcaagatcaattatcaagtatcagcaacagcacctgaaggttggagtgcattagttgtcattgagaataatgctagctattcattgcactggcattagagacagagagggcgagccagtttgacatggcaaattagcacagtcaaactggatacgtggtgacggagggaggggcactatgaatttttggtgacggagggaggggcactatgaatttttggctttgctgacgggacacgccactatggatgaaattggacaaaatacgaatattcaaggatgaaagtggtcggtttgatagttcagggatgaaatgtgtctttgggcaaactttgaggacgaagttgcctattttgcattaaacgaatatatttatataccccaaaaaaaagaatacacatctccactccgagccggcatgtggggtccccactagtcagccactgtatggcgccgactagctcaacggccacgaaccagccaaccaccagcgcaacctaaacggcgtaaacgttgacggcatctctctctcgccccgtctcgaagcttccgcaccgctcgctggtcgctgcccggcgccgctcgtgctggactctttccgtggcggcttccgcgaaattgcgtggtggagaggagagacggaaccgtcacggcactggattccttccccacccggcttggccggcccctcctcgcctccataaataggcaccccgtcctcgcctcctctccccacctcatctcctcctttcccgtgaaccgtgaacacaacccgacccagatcccctcttgcgagcttcgtcgatccctcctccgcgtcaaggtacggagcttctcctcccccttcttctctagatcggcgtgttatgttgtttccgtggttgcttggttggatgaatcgaatgattcttagggcctaggaggctggttagatctgttgcgttctgtttcgtagatggattttggtgtaagatcaggtcggttccgctgtttaacttgtgatgctagtgtgatttttgggaggatttgagttgttaatctgggagttgttgggaggttctcgtaggcggattgtagatgaagtcgcccgcacgatttgcgtggcttgttgggtagctagggttagatctgctcggatttttcattgttacttattgagagataatgtagctaacctttacttgttcatctatgtatctcgtattcgtattcatctggttcgatggtgctagatagatgcgcctgatttgtccgatcgaattgggtagcatccgcggcttgtttggtagtgttctgattgatttgtcgctctagatctgagtggaataatattacatctcaacatgttactagaaacttggtttatagctccggatttacatgtttattcttatgtaaggttttaaatgaaagatttatgctactgctgctcgttgatcctttagcatccacctgaggaacatgcatgcatctgttacttcttttgatatatgcttagatagttgttagtatatactgctgttgttcgatgatccttcaggatgaacatgcatgatcatgttacttgtttttatatgcttctgctgttcgttgattctttagtactacctacctgatcatcttgcatgtttcctgcttgttagagattaattgattaggcttaccttgttgcctggtgattcttccttgcaggtgggtacccggaccgatggcaacagcaacatcagcttctctgttttcaactgtttcttcatcttactccaaagctagctccataccacattcaagactccaatctgtgaaattcaactcagtccctagcttcaccggtctcaaatcaacctctctcatctccggatctgattcctcttccttagccaagactctacgcggttccgtaacgaaagcacaaacatctgacaagaagccttacggattcaaaatcaacgctatgcacgcggcgctgactcctctcaccaacaagtatcgctttatcgacgtgcagccgctgacaggcgtcctcggtgcagagattacaggcgtggatctgcgggagcctctcgatgacagcacttggaatgagatcctggacgcctttcacacctaccaagtgatctactttccgggtcaagctatcactaacgagcagcacatcgcgttctcccgccggttcggccctgtggacccggtgccgatcttaaagagtatcgagggctatccagaggtgcagatgatacggcgcgaggcgaacgagagcagccggttcatcggagatgactggcacaccgattccaccttcctggacgctccgcctgccgccgtggtgatgagagctatcgaagtgccggagtatggaggtgacacaggcttcctctccatgtacagtgcctgggagacactctcgcctacgatgcaagctaccatcgaaggcttaaacgtggtccactcggcgacgaaggtcttcgggtcattgtaccaggcgactaattggcgcttctcgaacaccagcgtgaaagtgatggacgtggacgccggagatagagagactgtgcacccactcgtcgtgacgcatcctgttacgggaaggcgcgcactctactgcaaccaggtgtactgccagaagatccagggaatgacggacgcggagtcgaagtccctgttgcaattcctttacgagcacgccaccaagttcgacttcacctgccgggtccggtggaagaaggaccaagtcctggtgtgggacaacctgtgtaccatgcaccgcgccgtcccggactacgctgggaaattcagatacctgacccgcaccaccgtggcgggagacaagccgtcgcgttgacggtccgttaattaatcgagtgtgaaggaagtgaactgagtctcaagaataaaaactgcaagtatggcgctccattccatggaagacctgcatgcatcctatatatgtgcttttatgttcaagttggagaactatgtgtgttgtctttaagttggagtactctaagctattcgatctggagattatgctgtttttaataaaagtttgaagtgtatctgtcctgtttaattagtttcaagaaagccaagcgtgtttgggttctaggatgaacaaagtcgtccaagatgcaagaagttctagagaggtagctacatttctggaaattgttgcatcttcctttcttcaatatatatatattgtcctatttcataatgtcattctagccagctatctatattctatatatccatttccgttgccatactttattcactatgttgcatcgagtgagctgcattgctctgtactacatgttacttgatatgtgttgttcataaacacacacattaataatgatcagattgtgaaaaatacgcgtccgatcctacctgtcacttcatcaaaaggacagtagaaaaggaaggtggcacctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgatacttccactgacgtaagggatgacgcacaatcccactatccttcgcaagacccttcctctatataaggaagttcatttcatttggagaggacacgctgaaatcaccagtctctctctacaagatcggggatctctagccctagaaccatcttccacacactcaagccacactattggagaacacacagggacaacacaccataagatccaagggaggcctccgccgccgccggtaaccaccccgcccctctcctctttctttctccgtttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccgtgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaagtgatcaaccatggcgcaagttagcagaatctgcaatggtgtgcagaacccatctcttatctccaatctctcgaaatccagtcaacgcaaatctcccttatcggtttctctgaagacgcagcagcatccacgagcttatccgatttcgtcgtcgtggggattgaagaagagtgggatgacgttaattggctctgagcttcgtcctcttaaggtcatgtcttctgtttccacggcgtgcatgcttcatggagcttcatctaggccagctactgccaggaagtctagcgggctcagtggcaccgtgcgcatccctggcgataaaagtatttcacacaggagcttcatgttcggaggacttgctagtggagagacgagaatcactggtttgcttgagggcgaagatgttatcaacaccggtaaggcgatgcaagcaatgggtgccagaatccgaaaagagggcgatacgtggatcatcgacggtgttggtaacggaggattgctcgctcccgaagcgccacttgactttgggaacgcagctacggggtgccgtcttactatgggactggtaggcgtgtatgactttgactctaccttcatcggtgacgcgagcctcactaagagaccaatgggacgagtgctgaatcccctgagggagatgggtgtccaggtgaaatctgaggatggtgatcgtcttccggttactctgcgaggccccaagacccccacgccaatcacgtacagggttccgatggcgtcagcacaggtcaagtcagcggtactcctggcgggcctcaacacacctggaatcacaaccgtgattgaacccatcatgactagagaccacacggagaagatgttgcagggtttcggcgctaatctaacggtcgaaaccgacgccgacggcgtgaggacaatccgcttggagggcagaggtaaactgactggccaagtcatcgatgtgcctggagatccctcgtccacagcgtttcccctcgtagctgcgttgctcgtccctggatctgatgtgacgatcctgaatgtcctcatgaatccaactagaaccggcctcatcctcacattgcaggagatgggtgctgacatcgaggttatcaatcctaggttggcaggtggagaggatgtggccgatctgcgcgtgcgttctagtacactcaaaggcgtgaccgtccctgaggatcgcgctccatccatgatcgacgagtaccccattctcgccgttgctgctgcgtttgccgagggcgcaactgtaatgaacggccttgaggagttgagggttaaggagagtgacaggctgtccgcggtggcgaatggcctgaagctaaacggcgtggactgcgacgaaggtgaaacgtcccttgtagtccgtggtcgcccagacgggaaggggttggggaatgcttcgggagctgctgtggcgacgcaccttgatcatagaatcgccatgtcatttctggtgatgggacttgtctccgagaatccggtgaccgttgacgatgctaccatgatcgccacctcctttcctgagttcatggacctcatggcaggcttgggggccaagatcgagctgtctgatactaaggccgcttgacacgtgagacgacgaccgtgcatggtgatgaatcacagacgacgaacacgcatgtcgcatcgccgtcctctttgtatcacacagcatcaccttcttcgctctactactcccccgagcaatcaccgaccaataacaccaaccatcaacctcccccgtcgccgccgccttcaccgtcctcccctcacaccatagaactgcaaatgtccgcctgcagggtggggcccatcgtggccagttatccttagctatccgtgtcagaatcatcttatcatcgagtcgagtcgttatcgtgtccagtggctctctcgagtcgagaagccctctatccatccatccagtgttaggtgttcttcgtccgtgatgttaccatgaattgagttcgctttggttatggtgtttgaactgcttgttgctatctatcggaatgaaatgaaatagaagacaaggagaaaaaaaagagttcgaaagttttgttcgcataccatatatttccttccggtgcgcgctgtttattcctcgctcagcagcaagattgtttgatcgatattgcagcaagcaattacacaataaatatattgctacactggtacttcaaactacactggtggtcggtgattttcaatagcatgaaccttaattgaacatctgtgtagcttacatctccttcgaaagctgcaatgcttgagaacttggaaagaaattcttgtgatggcagaagctattcactgtccttcgctgcatttacagtccatacagacacagcatttccattttgcacaagatagagaacaacaatcagccttttaggtcaatcccaagtgtgcatgacgtcccgcgatcgcccgcacaacaaacgcaccggtgcacaaactatagcaagtgtggtctatttttatatagcaagtgaggtcagataaaataataaataaaatcattaaattttatatagcaagtgtggtctatttttgcttgtaaactacagagaatatatatatgttaaatcggtagaacaattggctatttaaactaaaagatttaaaaaaaacaaagataaaaaatgtttttgaactgcgtgtctcgagagcagccagcatgcttccgacctgcatgagcagcgatgctcctggtgaccgcacgaacggaacctggcccgatggatgattttgcatcaacagcgcctgtatgcaacgatcatccaaccaaacatacttcgctttgaggatccacgcctacaacgtccgcgctcgtccatgcaaccaaacacacggaatgtgttccatgtgtcccaattgaaagaagtgtattcggttaacaacggaagtcataaccgagccagatatagagatagaaccagatctatcataccaagaacacccctccaagattctagactgcaaggaaagatccactcgtgcgaagacgaccaagatgtataagatccaatggagcaaccatacggaagaagaggctacgtgggagactgaggattatctatgcaaatactaccccgattgtctacctaaggaagtcagtacgtaaccatgccccagccccctgccctccgattccaaatatagaaaagatactcttaatgaaaactgaattaagaaatgaaattaagcgaagaaggacttccttctgaagttgcaaagaggatggcattcgaagagcagatttttctcgcgaaccttaaaaagctaaccaatactatgcaagcaagctataaccacctcctcactcctgggtgatctcgactcgaatctcggggcgagattcttttaaggggggagagctgtaacaccccaggtgttacttagggtttcccccttagtacctccatttgtgacctattatcacatgtggtttggtttaagaaaaaggcaccaaacttgaggggccaagccc

SEQ ID NO: 213 - DNA, 14008 bp, synthetic construct
  source 1..14008: mol_type = other DNA
ttcatgtccgggaaatctacatggatcagcaatgagtatgatggtcaatatggagaaaaagaaagagtaattaccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaacttcacgatttggcgcgactaactaagcactagcgtacgggacccagatatcgaattcaagctgaccgccaccggcaaacaaccacgaatttgtaatggtactaggcaaattctccgtttggcggtgtgtgccggccaattacacgtttttgcggtgtcctccgacaaaatttgccttttaaaaacaattttataagagaagctccggagataaaaggccgtcaatgttacaagagtgaagtcgtctactccctccatcccaaaaaatgtaattctaagtatgagttgtattattatttttggacaaaaggagtataccacaagaatgatatcatcgtcatgcttagatcctttttagtaaagcttgagcttctctaaaagtagagaaattagaaaaaaatcacgtttttgtggtcttgatttctagcctccacaaaatctttggttttacattttttgtttgattttggtttcagaagtccttatttatatgtgctagtttggcagcacttaaaatcgttagagagagcctaaacaaaagccttttcaaaacgaccttgagccagattggttgatggccaaaatttgattgtcaaaacttaggcaagccaagattttagcagctatttggtttggtaccaaaatttgccaatgatctgttcttttgccttttcaaccggtttatcagccgtacttcagcttattctctctcacagaacactattgaatcagccgaaaagccaccgcagaacaggaccagtatctcacaaatggcatgccaaatatactcaccgtcagtgagcccgtttaacggcgtcgacaagtctaacggccaccaaccagcgaaccaccagcgtcaagctagccaagcgaagcagacggccgagacgttgacaccttggcgcgggcatctctctggccccctctcgagagttccgctccacctccactggtggcggtttccaagtccgttccgcctcctgctcctcctcacacggcacgaaaccgtcacggcaccggcagcacgggggattcctttcccaccgctccttccctttcccttcctcgcccgccgttttaaatagccagccccatccccagcttctctccccaacctcagcttctctcgttgttcggagcgcacacacaacccgatccccaatcccctcgtctctcctcgcgagcctcgtcgatccccgcttcaaggtacggcgatcatcctccctttctctaccttctcttctctagactaggtcggcgatccatggttagggcctgctagttctgttcctgtttttccgtggctgcgaggtacaatagatctgatggcgttatgatggttaacttgtcatactcctgcggtgtgcggtctatagtgcttttaggacatcaatttgacctggctcgttcgagatcggcgatccatggttaggaccctaggcggtggagtcgggttagatccgcgctgtttgtgttagtagatggatgcgacctttacttcagacacgttctgattgttaacttgtcagcacctgggagtcctgggatggttctagctggttcgcagatgagatcgatttcatgatctgctgtatcttgtttcgttaggttccttttaatctatccgtggtattatgctaacctatgatatggttcgatcgtgctagctacgtcctgtgtcataatttttagcatgcccttttttgtttggttttgtctgattgggctgtagatcagagtatactgtttcaaactacctactggatatatttattaaatttgaatctgtatgtgtgtcacatatatcttcataattaaaatggatggaaagatatatggataggtacatgtgttgctgtgggttttactggtactttgttagatatacatgcttagatacatgaagcaacatgatgttacagttcaataattcttgtttacctaataaacaaataaggataggtgtatgttgctgtgggttttgctggtactttgttagatatatatgcttagatatatgaagcaacatcctgctacggtttaataattattgtttatatctaatagacaagcctgctttttaattattttgatatacttggatgatggcatacagcagctatgtgtggatttttaaatacccagcatcatgagcatgcatgaccctgccttagtatgctgtttatttgcttgagacttctttttttgttggtactcaccttttgtagtttggtgactcttctgcaggtgcaaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatccacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatctgatacgcgctgctagtagccaagtacctacctcgagggcacactgtaggcagtgtgccatattacaggttcagattggccgggacaaagagtacactgcgattttactatcctcggtatgctggtactacaggttcacattcacatggttactacctggcctgtggtatgctggtactacaggtgttgctctgaatagctcaggcttgacccaagtaagagcagtgcaaattcccaatttccagaaaacaggaggacctccaaaggacttcaatgtaattcaaatttctttcagaggtccttttgcaaggggctggatgtaaagtacttttataataatccaagccattgtgcttctcaaaaaaaggaaaagaaaaaaatttgttgcggcccggccgtgacggccacgagcgaactcctgcaggagcagactcgcattatcgatggagctctaccaaactggccctaggcattaacctaccatggatcacatcgtaaaaaaaaaaccctaccatggatcctatctgttttctttttgccctgaaagagtgaagtcatcatcatatttaccatggcgcgcgtaggagcgcttcgtcgaagacccataggggggcggtactcgcaccgtggttgtttcctgttatgtaatatcggatgggggagcagtcggctaggttggtcccatcggtactggtcgtcccctagtgcgctagatgcgcgatgtttgtcctcaaaaactcttttcttcttaataacaatcatacgcaaattttttgcgtattcgagaaaaaaagaagattctatctgttttttttttgaaatggctccaatttataggaggagcccgtttaacggcgtcgacaaatctaacggacaccaaccagcgaatgagcgaacccaccagcgccaagctagccaagcgaagcagacggccgagacgctgacacccttgccttggcgcggcatctccgtcgctggctcgctggctctggccccttcgcgagagttccggtccacctccacctgtgtcggtttccaactccgttccgccttcgcgtgggacttgttccgttcatccgttggcggcatccggaaattgcgtggcgtagagcacggggccctcctctcacacggcacggaaccgtcacgagctcacggcaccggcagcacggcggggattccttccccaccaccgctccttccctttcccttcctcgcccgccatcataaatagccacccctcccagcttccttcgccacatcctctcatcatcttctctcgtgtagcacgcgcagcccgatccccaatcccctctcctcgcgagcctcgtcgatccctcgcttcaaggtatggctatcgtccttcctctctctctctttaccttatctagatcggcgatccatggttagggcctgctagttctccgttcgtgtttgtcgatggctgtgaggcacaatagatccgtcggcgttatgatggttagcctgtcatgctcttgcgatctgtggttcctttaggaaaggcattaatttaatccctgatggttcgagatcggtgatccatggttagtaccctaagctgtggagtcgggtttagatccgcgctgttcgtaggcgatctgttctgattgttaacttgtcagtacctgcgaatcctcggtggttctagctggttcggagatcagatcgattccattatctgctatacatcttgtttcgttgcctaggctccgtttaatctatccatcgtatgatgttagcctttgatatgattcgatcgtgctagctatgtcctgtggacttaattgtcaggtcctaatttttaggaagactgttccaaaccatctgctggatttattaaatttggatctggatgtgtcacatacaccttcataattaaaatggatggaaatatctcttatcttttagatatggataggcatttatatgatgctgtgagttttactagtactttcttagaatatatgtacttttttagacggaatattgatatgtatacatgtgtagatacatgaagcaacatgctgctgtagtctaataattcctgttcatctaataatcaagtatgtatatgttctgtgtgttttattggtatttgattagatatatacatgcttagatacatacatgaagcagcatgctgctacagtttaatcattattgtttatccaataaacaaacatgctttttaatttatcttgatatgcttggatgacggaatatgcagagattttaagtacccagcatcatgagcatgcatgaccctgcgttagtatgctgtttatttgcttgagactctttcttttgtagatactcaccctgttttctggtgatcctactgcaggtgaccggtcgccatggccaccgccaccactaccgccaccgctgcgttctccggcgtggtgagcgtcggcactgagacgcgcaggatctactccttcagccacctccagccttctgctgcgttccccgctaagccgtcttcgttcaagagcctgaagctgaaacagtccgcacgccttacccggcgcctggaccataggccattcgttgtcaggtgcatgctcaccttcgttaggaacgcctggtacgtcgccgctctccctgaggagctgagcgagaagcccttgggtcgcaccatcctagacactccgttagccctttaccgccagcctgacggcgtagtggcggccctgcttgacatctgcccgcataggttcgctccgctcagcgacggcatcctcgtcaacgggcatcttcagtgcccgtaccacgggctggaatttgacggcggtgggcagtgtgtccacaacccgcacggcaacggcgcacggccagcttccctcaacgttaggtcgttccctgttgtcgagcgcgacgcactgatctggatctggcctggcgacccagctctggccgatccaggagccattcccgacttcggttgccgcgtggacccagcctatcggacggtcggcggttacgggcacgtcgattgtaactataagctccttgtggacaaccttatggatttgggccacgctcagtacgtgcaccgggctaacgctcagactgacgcctttgaccgtctcgaaagggaggtcatcgtcggcgacggagagattcaggcgctgatgaagatccctggaggcacgccctctgtgctcatggcgaagtttctcagaggcgcgaacacgcccgtggacgcctggaacgacatccgctggaataaggtctccgcgatgctgaacttcatcgccgttgcgcccgagggcacacccaaagagcagtcaatccacagcagagggacccatattcttacaccggaaaccgaggctagttgccactacttcttcggctcgtcacggaatttcgggatagacgatccggagatggacggtgttcttcgatcttggcaagcgcaagctctcgtcaaggaagataaggtggtcgtggaggctatcgagcgtaggcgcgcctacgttgaggcgaacggtattaggcccgcgatgctgtcctgcgacgaggccgcagttagagtgtcgcgcgagatagaaaagctggagcagctagaggccgcctgattaattaaggccaaggcgatctatgactgaattgccaatgcaccagcctgtctacatgatgaataaataaagagtccatccagtgtgatggctcatgcctgtgtgagtgtgactgaatccatcagtgtgtgtgtgtgtttgtgtcaaccatgtgtgaatcaggtgtcaaaaatcgtggctggaaatccatgtggtttctagctttatgtaaatgttgtttgtgaaatataaatattgttttgtgtatgtgaattttactctctcatttttctcttgcactcaccattctattatagtaatttttttaagcggccgctgatgtatccgtccacggtggtgtgtccaatcagtgaataatctagttagtgaagccagaagtccatagtgccccttgctctgtcaccatatatccagttcaaccgcaccaatttgccatctcgaactggttcatgttttattcaggttggtaaatgaattttgccaattcaatgtagttagatatttccatgtcattttagtacatttaccaattttttatattctggctagaaaaggagaatggtgacgtctttcggaagatcaagatcaattatcaagtatcagcaacagcacctgaaggttggagtgcattagttgtcattgagaataatgctagctattcattgcactggcattagagacagagagggcgagccagtttgacatggcaaattagcacagtcaaactggatacgtggtgacggagggaggggcactatgaatttttggtgacggagggaggggcactatgaatttttggctttgctgacgggacacgccactatggatgaaattggacaaaatacgaatattcaaggatgaaagtggtcggtttgatagttcagggatgaaatgtgtctttgggcaaactttgaggacgaagttgcctattttgcattaaacgaatatatttatataccccaaaaaaaagaatacacatctccactccgagccggcatgtggggtccccactagtcagccactgtatggcgccgactagctcaacggccacgaaccagccaaccaccagcgcaacctaaacggcgtaaacgttgacggcatctctctctcgccccgtctcgaagcttccgcaccgctcgctggtcgctgcccggcgccgctcgtgctggactctttccgtggcggcttccgcgaaattgcgtggtggagaggagagacggaaccgtcacggcactggattccttccccacccggcttggccggcccctcctcgcctccataaataggcaccccgtcctcgcctcctctccccacctcatctcctcctttcccgtgaaccgtgaacacaacccgacccagatcccctcttgcgagcttcgtcgatccctcctccgcgtcaaggtacggagcttctcctcccccttcttctctagatcggcgtgttatgttgtttccgtggttgcttggttggatgaatcgaatgattcttagggcctaggaggctggttagatctgttgcgttctgtttcgtagatggattttggtgtaagatcaggtcggttccgctgtttaacttgtgatgctagtgtgatttttgggaggatttgagttgttaatctgggagttgttgggaggttctcgtaggcggattgtagatgaagtcgcccgcacgatttgcgtggcttgttgggtagctagggttagatctgctcggatttttcattgttacttattgagagataatgtagctaacctttacttgttcatctatgtatctcgtattcgtattcatctggttcgatggtgctagatagatgcgcctgatttgtccgatcgaattgggtagcatccgcggcttgtttggtagtgttctgattgatttgtcgctctagatctgagtggaataatattacatctcaacatgttactagaaacttggtttatagctccggatttacatgtttattcttatgtaaggttttaaatgaaagatttatgctactgctgctcgttgatcctttagcatccacctgaggaacatgcatgcatctgttacttcttttgatatatgcttagatagttgttagtatatactgctgttgttcgatgatccttcaggatgaacatgcatgatcatgttacttgtttttatatgcttctgctgttcgttgattctttagtactacctacctgatcatcttgcatgtttcctgcttgttagagattaattgattaggcttaccttgttgcctggtgattcttccttgcaggtgggtacccggaccgatggcaacagcaacatcagcttctctgttttcaactgtttcttcatcttactccaaagctagctccataccacattcaagactccaatctgtgaaattcaactcagtccctagcttcaccggtctcaaatcaacctctctcatctccggatctgattcctcttccttagccaagactctacgcggttccgtaacgaaagcacaaacatctgacaagaagccttacggattcaaaatcaacgctatgcacgcggcgctgactcctctcaccaacaagtatcgctttatcgacgtgcagccgctgacaggcgtcctcggtgcagagattacaggcgtggatctgcgggagcctctcgatgacagcacttggaatgagatcctggacgcctttcacacctaccaagtgatctactttccgggtcaagctatcactaacgagcagcacatcgcgttctcccgccggttcggccctgtggacccggtgccgatcttaaagagtatcgagggctatccagaggtgcagatgatacggcgcgaggcgaacgagagcagccggttcatcggagatgactggcacaccgattccaccttcctggacgctccgcctgccgccgtggtgatgagagctatcgaagtgccggagtatggaggtgacacaggcttcctctccatgtacagtgcctgggagacactctcgcctacgatgcaagctaccatcgaaggcttaaacgtggtccactcggcgacgaaggtcttcgggtcattgtaccaggcgactaattggcgcttctcgaacaccagcgtgaaagtgatggacgtggacgccggagatagagagactgtgcacccactcgtcgtgacgcatcctgttacgggaaggcgcgcactctactgcaaccaggtgtactgccagaagatccagggaatgacggacgcggagtcgaagtccctgttgcaattcctttacgagcacgccaccaagttcgacttcacctgccgggtccggtggaagaaggaccaagtcctggtgtgggacaacctgtgtaccatgcaccgcgccgtcccggactacgctgggaaattcagatacctgacccgcaccaccgtggcgggagacaagccgtcgcgttgacggtccgttaattaatcgagtgtgaaggaagtgaactgagtctcaagaataaaaactgcaagtatggcgctccattccatggaagacctgcatgcatcctatatatgtgcttttatgttcaagttggagaactatgtgtgttgtctttaagttggagtactctaagctattcgatctggagattatgctgtttttaataaaagtttgaagtgtatctgtcctgtttaattagtttcaagaaagccaagcgtgtttgggttctaggatgaacaaagtcgtccaagatgcaagaagttctagagaggtagctacatttctggaaattgttgcatcttcctttcttcaatatatatatattgtcctatttcataatgtcattctagccagctatctatattctatatatccatttccgttgccatactttattcactatgttgcatcgagtgagctgcattgctctgtactacatgttacttgatatgtgttgttcataaacacacacattaataatgatcagattgtgaaaaatacgcgtccgatcctacctgtcacttcatcaaaaggacagtagaaaaggaaggtggcacctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgatacttccactgacgtaagggatgacgcacaatcccactatccttcgcaagacccttcctctatataaggaagttcatttcatttggagaggacacgctgaaatcaccagtctctctctacaagatcggggatctctagccctagaaccatcttccacacactcaagccacactattggagaacacacagggacaacacaccataagatccaagggaggcctccgccgccgccggtaaccaccccgcccctctcctctttctttctccgtttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccgtgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaagtgatcaaccatggcgcaagttagcagaatctgcaatggtgtgcagaacccatctcttatctccaatctctcgaaatccagtcaacgcaaatctcccttatcggtttctctgaagacgcagcagcatccacgagcttatccgatttcgtcgtcgtggggattgaagaagagtgggatgacgttaattggctctgagcttcgtcctcttaaggtcatgtcttctgtttccacggcgtgcatgcttcatggagcttcatctaggccagctactgccaggaagtctagcgggctcagtggcaccgtgcgcatccctggcgataaaagtatttcacacaggagcttcatgttcggaggacttgctagtggagagacgagaatcactggtttgcttgagggcgaagatgttatcaacaccggtaaggcgatgcaagcaatgggtgccagaatccgaaaagagggcgatacgtggatcatcgacggtgttggtaacggaggattgctcgctcccgaagcgccacttgactttgggaacgcagctacggggtgccgtcttactatgggactggtaggcgtgtatgactttgactctaccttcatcggtgacgcgagcctcactaagagaccaatgggacgagtgctgaatcccctgagggagatgggtgtccaggtgaaatctgaggatggtgatcgtcttccggttactctgcgaggccccaagacccccacgccaatcacgtacagggttccgatggcgtcagcacaggtcaagtcagcggtactcctggcgggcctcaacacacctggaatcacaaccgtgattgaacccatcatgactagagaccacacggagaagatgttgcagggtttcggcgctaatctaacggtcgaaaccgacgccgacggcgtgaggacaatccgcttggagggcagaggtaaactgactggccaagtcatcgatgtgcctggagatccctcgtccacagcgtttcccctcgtagctgcgttgctcgtccctggatctgatgtgacgatcctgaatgtcctcatgaatccaactagaaccggcctcatcctcacattgcaggagatgggtgctgacatcgaggttatcaatcctaggttggcaggtggagaggatgtggccgatctgcgcgtgcgttctagtacactcaaaggcgtgaccgtccctgaggatcgcgctccatccatgatcgacgagtaccccattctcgccgttgctgctgcgtttgccgagggcgcaactgtaatgaacggccttgaggagttgagggttaaggagagtgacaggctgtccgcggtggcgaatggcctgaagctaaacggcgtggactgcgacgaaggtgaaacgtcccttgtagtccgtggtcgcccagacgggaaggggttggggaatgcttcgggagctgctgtggcgacgcaccttgatcatagaatcgccatgtcatttctggtgatgggacttgtctccgagaatccggtgaccgttgacgatgctaccatgatcgccacctcctttcctgagttcatggacctcatggcaggcttgggggccaagatcgagctgtctgatactaaggccgcttgacacgtgagacgacgaccgtgcatggtgatgaatcacagacgacgaacacgcatgtcgcatcgccgtcctctttgtatcacacagcatcaccttcttcgctctactactcccccgagcaatcaccgaccaataacaccaaccatcaacctcccccgtcgccgccgccttcaccgtcctcccctcacaccatagaactgcaaatgtccgcctgcagggtggggcccatcgtggccagttatccttagctatccgtgtcagaatcatcttatcatcgagtcgagtcgttatcgtgtccagtggctctctcgagtcgagaagccctctatccatccatccagtgttaggtgttcttcgtccgtgatgttaccatgaattgagttcgctttggttatggtgtttgaactgcttgttgctatctatcggaatgaaatgaaatagaagacaaggagaaaaaaaagagttcgaaagttttgttcgcataccatatatttccttccggtgcgcgctgtttattcctcgctcagcagcaagattgtttgatcgatattgcagcaagcaattacacaataaatatattgctacactggtacttcaaactacactggtggtcggtgattttcaatagcatgaaccttaattgaacatctgtgtagcttacatctccttcgaaagctgcaatgcttgagaacttggaaagaaattcttgtgatggcagaagctattcactgtccttcgctgcatttacagtccatacagacacagcatttccattttgcacaagatagagaacaacaatcagccttttaggtcaatcccaagtgtgcatgacgtcccgcgatcgcccgcacaacaaacgcaccggtgcacaaactat

SEQ ID NO: 214 - DNA, 30 bp, synthetic construct
  source 1..30: mol_type = other DNA
ccactcttgtttggtttcatgtccgggaaa

SEQ ID NO: 215 - DNA, 60 bp, synthetic construct
  source 1..60: mol_type = other DNA
acttgcaaaaatagaccactcttgtttggtttcatgtccgggaaatctacatggatcagc

SEQ ID NO: 216 - DNA, 100 bp, synthetic construct
  source 1..100: mol_type = other DNA
agatactcttgtttggttacacttgcaaaaatagaccactcttgtttggtttcatgtccgggaaatctacatggatcagcaatgagtatgatggtcaata

SEQ ID NO: 217 - DNA, 1350 bp, synthetic construct
  source 1..1350: mol_type = other DNA
atctacagggcgcgcagcggctgacacatgggacccacgggcaaaggcagcggcagactcgcgcgcgagagcacgcaagcaccgattgacgcgcgtgccccttttgtcggagaacgagggtgcgcgatcgtccgtgcatggttgagacggccaggcggggcccacccattggcgccatgccgcgcgagcagcagctggcccgcacaggtgcaaaggttgagtgggctagaagcgaagacctagtccaagcgacggtttcttccttttttctctttttttctttttcctttatattttcttgttttattttattttcatttttttattttcaaatctaaatttgaactcaagtgtgagattcatactttgaattaaatgctcacattcaaatatcagtaggaatagaatatttttaactatatgtttattttctctattttatacaatcttttccttttttcatttttaaacctcaatttgtaaattatatctaaattccaattttggtcattagtatactcctactaatatcattttattgttatgaaatgcacacacaataaactccaacttgatgaatagatatctatttattctaattaatttatttgtttggtagatgttcaaaatatgaaacacacacatattgttttctttctttttagaaaaatggtatttttattgtgggacaagaactaaaagtcacctcaaaattaaatactcatgtataatttgggatatctgaatttactatttttatttcttcatatatttaatttattattaattttttttcttattgggccttacacttatgagatcatagaggcatgtggatcagtggcatacaagttgaaattactgccaaaaatgtctgtcatacacaatccgtgtgtttggtttgtggaccagcagagcctggctactctaatccgtatgtagagaaccaagctcccacgagacagactcaatgtatccgagatactcttgtttggttacacttgcaaaaatagaccactcttgtttggtttcatgtccgggaaatctacatggatcagcaatgagtatgatggtcaatatggagaaaaagaaagagtaattaccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaacttcacgatttggcgcgactaactaagcactagcgtacgggacccagatatcgaattca

SEQ ID NO: 218 - DNA, 30 bp, synthetic construct
  source 1..30: mol_type = other DNA
cggtgcacaaactatagcaagtgtggtcta

SEQ ID NO: 219 - DNA, 60 bp, synthetic construct
  source 1..60: mol_type = other DNA
gcacaacaaacgcaccggtgcacaaactatagcaagtgtggtctatttttatatagcaag

SEQ ID NO: 220 - DNA, 100 bp, synthetic construct
  source 1..100: mol_type = other DNA
atgacgtcccgcgatcgcccgcacaacaaacgcaccggtgcacaaactatagcaagtgtggtctatttttatatagcaagtgaggtcagataaaataata

SEQ ID NO: 221 - DNA, 1069 bp, synthetic construct
  source 1..1069: mol_type = other DNA
gatcgcccgcacaacaaacgcaccggtgcacaaactatagcaagtgtggtctatttttatatagcaagtgaggtcagataaaataataaataaaatcattaaattttatatagcaagtgtggtctatttttgcttgtaaactacagagaatatatatatgttaaatcggtagaacaattggctatttaaactaaaagatttaaaaaaaacaaagataaaaaatgtttttgaactgcgtgtctcgagagcagccagcatgcttccgacctgcatgagcagcgatgctcctggtgaccgcacgaacggaacctggcccgatggatgattttgcatcaacagcgcctgtatgcaacgatcatccaaccaaacatacttcgctttgaggatccacgcctacaacgtccgcgctcgtccatgcaaccaaacacacggaatgtgttccatgtgtcccaattgaaagaagtgtattcggttaacaacggaagtcataaccgagccagatatagagatagaaccagatctatcataccaagaacacccctccaagattctagactgcaaggaaagatccactcgtgcgaagacgaccaagatgtataagatccaatggagcaaccatacggaagaagaggctacgtgggagactgaggattatctatgcaaatactaccccgattgtctacctaaggaagtcagtacgtaaccatgccccagccccctgccctccgattccaaatatagaaaagatactcttaatgaaaactgaattaagaaatgaaattaagcgaagaaggacttccttctgaagttgcaaagaggatggcattcgaagagcagatttttctcgcgaaccttaaaaagctaaccaatactatgcaagcaagctataaccacctcctcactcctgggtgatctcgactcgaatctcggggcgagattcttttaaggggggagagctgtaacaccccaggtgttacttagggtttcccccttagtacctccatttgtgacctattatcacatgtggtttggtttaagaaaaaggcaccaaacttgaggggccaagccc